(19) United States
(12) Patent Application Publication
(10) Pub. No.: US 2025/0230140 A1
(43) Pub. Date: Jul. 17, 2025

(54) NOVEL TRIAZOLE-PYRIDINE SUBSTITUTED PYRROLIDINYL AND TETRAHYDRO-2H-PYRANYL ACETIC ACID COMPOUNDS AS LPA ANTAGONISTS

(71) Applicant: Viva Star Biosciences (Suzhou) Co., Ltd., Suzhou, Jiangsu (CN)

(72) Inventors: Hongjian ZHANG, Plainsboro, NJ (US); Ping CHEN, Shanghai (CN); Zhenwei CAI, Shanghai (CN); Fei JIANG, Shanghai (CN); Peihua SUN, Shanghai (CN)

(73) Assignee: Viva Star Biosciences (Suzhou) Co., Ltd., Suzhou, Jiangsu (CN)

(21) Appl. No.: 18/557,918

(22) PCT Filed: Apr. 28, 2022
(86) PCT No.: PCT/US2022/026832

§ 371 Date: Oct. 27, 2023

(30) Foreign Application Priority Data
Apr. 30, 2021 (WO) PCT/CN2021/091510

(51) Int. Cl. C07D 401/14; C07D 405/14

(52) U.S. Cl. CPC C07D 401/14; C07D 405/14

(57) ABSTRACT

This application relates to novel triazole-pyridine substituted pyrrolidinyl, tetrahydro-2H-pyranyl, cyclohexyl, and piperidinyl acetic acid compounds, their manufacture, pharmaceutical compositions comprising them, and their use as medicaments for treating a disease associated with dysregulation of lysophosphatidic acid receptors (LPA).

This application claims priority to PCT International Application No. PCT/CN2021/091510, filed Apr. 30, 2021, the entire contents of which are incorporated herein by reference.

### FIELD

This application relates to novel triazole-pyridine substituted pyrrolidinyl, tetrahydro-2H-pyranyl, cyclohexyl, and piperidinyl acetic acid compounds and related compounds, their manufacture, pharmaceutical compositions comprising them, and their use as medicaments for treating a disease associated with dysregulation of lysophosphatidic acid receptors (LPA).

### BACKGROUND

Lysophosphatidic acid (LPA) is a small glycerolphospholipid (1- or 2-acyl-sn-glycerol 3phosphate) with a molecular weight of 430-480 Dalton, consisting of a glycerol backbone which is esterified with a phosphate group and a fatty acid with variable chain length and degree of saturation (Yang and Chen, World J Gastroenterol 24:4132-4151, 2018). LPA can be formed from precursor molecules in plasma, serum or tissues (membrane phospholipids) via several pathways: (1) hydrolysis of the choline group off lysophosphatidylcholine by lysophospholipase D (lysoPLD or autotaxin); (2) hydrolysis of a fatty acyl chain from phosphatidic acid to produce 2-acyl or 1acyl LPA by phospholipase A1 or A2; and (3) de novo synthesis from glycerol-3-phosphate by acyltransferases (Kihara et al., Experimental Cell Res 333:171-177, 2015). In tissues or cells, LPA represents a mixture of 1- or 2-acyl-sn-glycerol 3-phosphates.

Lysophosphatidic acid (LPA) acts as a signaling molecule and exerts its effects by binding to G protein-coupled receptors, termed LPA receptors (LPAR). To date, there are six identified LPA receptors (LPAR1-6), which are expressed in various tissues and/or cells. LPA, through binding to its receptors, plays important roles in pathophysiological processes, such as autoimmune diseases, fibrotic diseases, cancer, inflammation, neuropathic pain, etc. (Budd and Qian, Future Med Chem 5:1935-52, 2013; Valdes-Rives and González-Arenas, Mediators Inflamm 2017:9173090, 2017; Lopane et al., Biochim Biophys Acta Rev Cancer 1868:277-282, 2017; Ueda H. Pain 158 Suppl 1:S55-S65, 2017).

Fibrosis is a reparative (or “healing”) process characterized by the excessive accumulation of extracellular matrix (ECM). When tissue injury (caused by infections, autoimmune reactions, mechanical injuries, etc.) is chronic, a sustained production of pro-fibrotic mediators leads to an uncontrolled healing process, and the replacement of injured cells occurs with connective tissues associated with over production of ECM (Weiskirchen et al., Molecular Aspects Med. 65:2-15, 2019). Because it alters organs' architecture and function, fibrosis is tightly associated with and often responsible for morbidity and mortality. It is estimated that 45% of all deaths in the developed world are attributed to some type of chronic fibrosis, such as idiopathic pulmonary fibrosis, systemic sclerosis, liver cirrhosis, chronic cardiovascular diseases, progressive kidney diseases (kidney fibrosis) or diabetes (Wynn T. A. Nat. Rev. Immunol. 4:583-594, 2004).

The profibrotic action of LPA via binding to its receptor LPAR1 has been established in lung, liver and other organs or tissues with two main characteristics: 1) the existence of a positive correlation between the apparition of fibrosis markers and the increased production of LPA associated with an increased expression of the LPAR1; 2) the attenuation of fibrosis in LPAR1−/− mice or by treatment with LPAR antagonists (Rancoule et al., Expert Opin. Investig. Drugs 20:657667, 2011). For example, in the bleomycin model of pulmonary fibrosis, LPA levels increased significantly in the bronchoaveolar lavage fluid following lung injury, and mice lacking the LPAR1 gene (LPAR1−/− mice) were remarkably protected from fibrosis and mortality (Tager et al., Nat. Med. 14:45-54, 2008). Treatment with small molecule LPAR1 antagonists could reduce the lung fibrosis in the bleomycin mouse model (Swanet et al., Br. J. Pharmacol. 160:1699-1713, 2010). More recently, in a chemical-induced cirrhosis and HCC rat model, the expression of Lapr1 was markedly increased in hepatic stellate cells, while lysoPLD (autotaxin) was higher in hepatocytes (Nakagawa et al., Cancer Cell. 30:879-890, 2016). Transcriptome analysis of human and rat liver tissues indicated that the LPA pathway via the activation of LPAR1 was a functional driver of cirrhosis and HCC. Consequently, inhibition of LPAR1 and lysoPLD by chemical inhibitors attenuated fibrosis progression and reduced HCC nodules in the cirrhosis-driven HCC rat model. (Id.).

### SUMMARY

In one aspect, the present invention relates to compounds of formula (I),

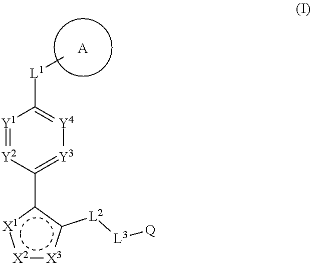

or a pharmaceutically acceptable salt, tautomer and/or stereoisomer thereof,
wherein:
A ring is a 5-membered heterocyclyl or 6-membered cyclohexyl or heterocyclyl selected from:

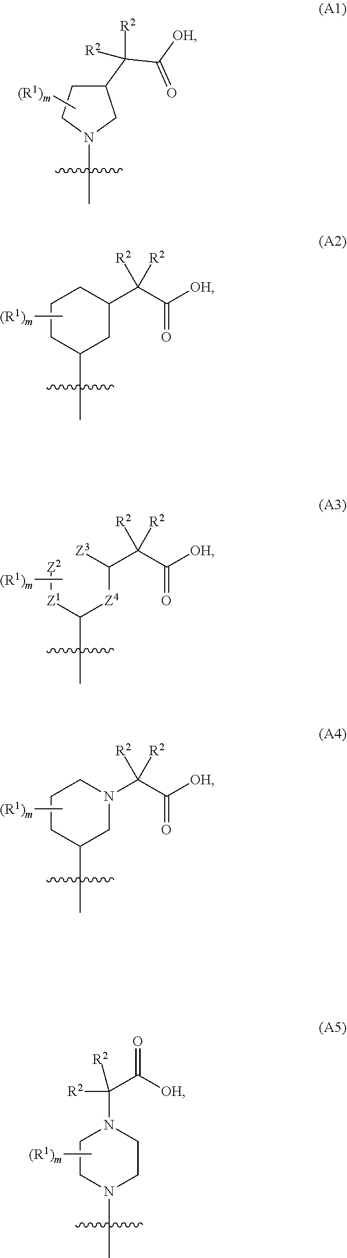

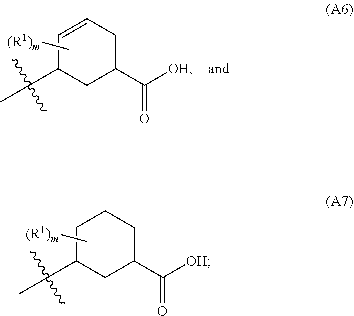

L¹ is a covalent bond, NH, O or S; provided that when L¹ is a covalent bond, A ring is selected from Formulae (A1), (A2), (A3), (A4) or (A5); further provided that when L¹ is NH or S, A ring is selected from Formulae (A6) or (A7); and further provided that when L¹ is O, A ring is selected from Formula (A6);
L² is a covalent bond or (CR⁷R⁷)ₚ;
L³ is a covalent bond, O or NR⁷, provided that at least one of L² and L³ is not a covalent bond;
Q is C(═O)NR⁹R¹⁰, C(═O)OR¹⁰, or a ring selected from a 5- or 6-membered heteroaryl group or a 5- or 6-membered heterocyclyl group, wherein the ring comprises at least one carbon atom, at least one nitrogen atom, and optionally 1-4 additional heteroatoms selected from nitrogen, oxygen and sulfur wherein oxygen may be a ring member and/or an oxo group (═O) attached to a ring member, and wherein the ring is substituted with (R³)ₙ and one R⁴;
X¹ is N, O or CR⁶a;
X² is N or NR⁶;
X³ is N, NR⁶ or CR⁶, and the dashed circle denotes bonds forming a five-membered aromatic ring;
Y¹, Y², Y³ and Y⁴ are each independently N or CR⁵, provided that at least one but no more than two of Y¹, Y², Y³ and Y⁴ are N;
Z¹, Z², Z³ and Z⁴ are each independently CH₂ or O, provided that only one of Z¹, Z², Z³ and Z⁴ is O;
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, (CH₂)_{q}-5-6-membered heteroaryl ring substituted with 1-4 R¹¹, or (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁵ at each occurrence is hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
each of R^{6a} and R⁶ is independently hydrogen, halogen, CN, C₁₋₄ alkyl, or cyclopropyl;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
each occurrence of R⁹ and R¹⁰ is independently hydrogen, C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, or O(CH₂)ₚ—C₃₋₇cycloalkyl, wherein each phenyl is independently optionally substituted with 1-3 halogen, C₁₋₆alkyl, or C₁₋₆alkoxy;
R¹² at each occurrence is independently hydrogen, C₁₋₄alkyl, or C₃₋₇cycloalkyl; or R¹² and R¹², together with the carbon atom to which they are attached, form a 3-6-membered cycloalkyl ring;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups, which may be the same or different, selected from the group consisting of C₁₋₄alkyl, phenyl and benzyl;
m is 0, 1, 2 or 3;
n is 0, 1, or 2;
p at each occurrence is independently 1, 2, 3 or 4;
q at each occurrence is independently 0, 1, 2, 3 or 4.

In another aspect, the present invention relates to compounds of Formula (IV),

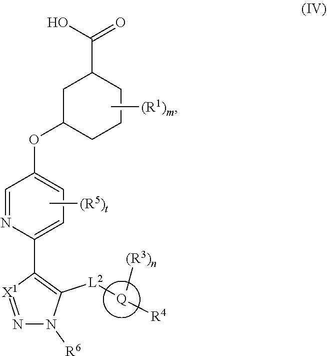

or a pharmaceutically acceptable salt, tautomer and/or stereoisomer thereof,
wherein:
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR⁶a;
R^{6a} is hydrogen, or methyl;
R⁶ is hydrogen, halogen, CN, methyl, ethyl, propyl, or cyclopropyl;
L² is (CR⁷R⁷)ₚ;
Q ring is

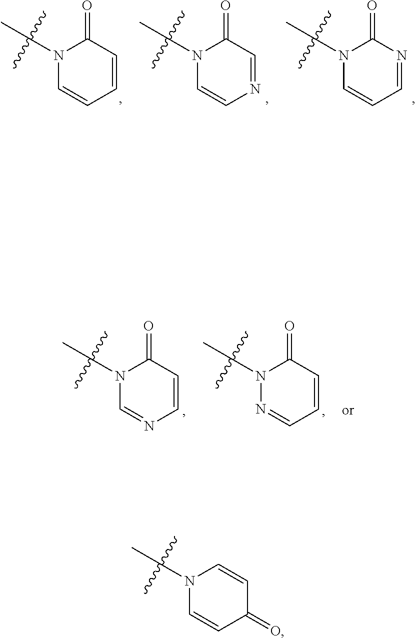 [SMILES: *n1ccccc1=O ; *n1ccncc1=O ; *n1cccnc1=O ; *n1cnccc1=O ; *n1ncccc1=O ; *n1ccc(=O)cc1]

each of which is substituted with (R³)ₙ and one R⁴ at any available carbon position;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, (CH₂)_{q}-5-6-membered heteroaryl ring substituted with 1-4 R¹¹, (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring; R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, wherein each phenyl is independently optionally substituted with 1-3 halogen, C₁₋₆alkyl, or C₁₋₆alkoxy;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
n is 0, 1 or 2;
t is 0, 1, 2 or 3;
p at each occurrence is independently 1, 2, 3 or 4;
and q at each occurrence is independently 0, 1, 2, 3 or 4.

The invention also relates to a pharmaceutical composition comprising a compound of formulae (I) and (IV), its manufacture and use as medicaments for treating a disease associated with dysregulation of lysophosphatidic acid receptor 1 (LPAi). Accordingly, the compounds of formulae (I) and (IV) are useful for treatment of pathological fibrosis (e.g., pulmonary, liver, renal, cardiac, dernal, ocular, or pancreatic fibrosis), idiopathic pulmonary fibrosis (IPF), non-alcoholic steatohepatitis (NASH), non-alcoholic fatty liver disease (NAFLD), chronic kidney disease, diabetic kidney disease, or systemic sclerosis.

### DETAILED DESCRIPTION

In one aspect, the present invention provides compounds, and their pharmaceutically acceptable forms, including, but not limited to, salts, hydrates, solvates, isomers, sterioisomers, enantiomers, prodrugs, and isotopically labeled derivatives thereof.

In another aspect, the present invention provides methods of treating and/or managing various diseases and disorders, which comprises administering to a patient a therapeutically effective amount of a compound provided herein, or a pharmaceutically acceptable form (e.g., salts, hydrates, solvates, isomers, sterioisomers, enantiomers, prodrugs, and isotopically labeled derivatives) thereof. Non-limiting examples of diseases and disorders are described herein.

In another aspect, the present invention provides methods of preventing various diseases and disorders, which comprises administering to a patient in need of such prevention a prophylactically effective amount of a compound provided herein, or a pharmaceutically acceptable form (e.g., salts, hydrates, solvates, isomers, sterioisomers, prodrugs, and isotopically labeled derivatives) thereof. Non-limiting examples of diseases and disorders are described herein.

In another aspect, a compound provided herein, or a pharmaceutically acceptable form (e.g., salts, hydrates, solvates, isomers, sterioisomers, prodrugs, and isotopically labeled derivatives) thereof, can be administered in combination with another drug (“second active agent”) or treatment. Second active agents include small molecules and large molecules (e.g., proteins and antibodies).

Also provided herein are pharmaceutical compositions (e.g., single unit dosage forms) that can be used in the methods provided herein. In one embodiment, pharmaceutical compositions comprise a compound provided herein, or a pharmaceutically acceptable form (e.g., salts, hydrates, solvates, isomers, sterioisomers, prodrugs, and isotopically labeled derivatives) thereof, and optionally one or more second active agents.

While specific embodiments have been discussed, the specification is illustrative only and not restrictive. Many variations of this disclosure will become apparent to those skilled in the art upon review of this specification.

Unless defined otherwise, all technical and scientific terms used herein have the same meaning as is commonly understood by one of ordinary skill in the art to which this specification pertains.

### Definitions

As used in the specification and claims, the singular form “a”, “an” and “the” includes plural references unless the context clearly dictates otherwise.

As used herein, “agent” or “biologically active agent” or “second active agent” refers to a biological, pharmaceutical, or chemical compound or another moiety. Non-limiting examples include simple or complex organic or inorganic molecules, a peptide, a protein, an oligonucleotide, an antibody, an antibody derivative, an antibody fragment, a vitamin, a vitamin derivative, a carbohydrate, a toxin, or a chemotherapeutic compound, and metabolites thereof. Various compounds can be synthesized, for example, small molecules and oligomers (e.g., oligopeptides and oligonucleotides), and synthetic organic compounds based on various core structures. In addition, various natural sources can provide active compounds, such as plant or animal extracts, and the like. A skilled artisan can readily recognize that there is no limit as to the structural nature of the agents of this disclosure.

“Administration” of a disclosed compound encompasses the delivery to a subject of a compound as described herein, or a prodrug or other pharmaceutically acceptable derivative thereof, using any suitable formulation or route of administration, as discussed herein.

The term “co-administration,” “administered in combination with,” and their grammatical equivalents, as used herein, encompasses administration of two or more agents to the subject so that both agents and/or their metabolites are present in the subject at the same time. Co-administration includes simultaneous administration in separate compositions, administration at separate times in separate compositions, or administration in a composition in which both agents are present.

The term “effective amount” or “therapeutically effective amount” refers to that amount of a compound or pharmaceutical composition described herein that is sufficient to affect the intended application including, but not limited to, disease treatment, as illustrated below. In some embodiments, the amount is that effective for detectable inhibition of LPA1, which, for example, can be determined in an LPA1 functional antagonist assay. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a response in target cells, e.g., reduction of cell migration. The specific dose will vary depending on, for example, the compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

All methods described herein can be performed in any suitable order unless otherwise indicated herein or otherwise clearly contradicted by context.

As used herein, the terms “treatment”, “treating”, “palliating” “managing” and “ameliorating” are used interchangeably herein. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a disease, or to a patient reporting one or more of the physiological symptoms of a disease, even though a diagnosis of this disease may not have been made.

The terms “preventing” and “prophylaxis” as used herein refer to administering a pharmaceutical compound or medicament or a composition including the pharmaceutical compound or medicament to a subject before a disease, disorder, or condition fully manifests itself, to forestall the appearance and/or reduce the severity of one or more symptoms of the disease, disorder or condition. The person of ordinary skill in the art recognizes that the term “prevent” is not an absolute term. In the medical art it is understood to refer to the prophylactic administration of a drug to substantially diminish the likelihood or seriousness of a disease, disorder or condition, or a symptom thereof, and this is the sense that such terms are used in this disclosure.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The “Subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

The term “in vivo” refers to an event that takes place in a subject's body. In vivo also includes events occurring in rodents, such as rats, mice, guinea pigs, and the like.

The term “in vitro” refers to an event that takes places outside of a subject's body. For example, an in vitro assay encompasses any assay conducted outside of a subject. In vitro assays encompass cell-based assays in which cells, alive or dead, are employed. In vitro assays also encompass a cell-free assay in which no intact cells are employed.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphor sulfonate, citrate, cyclopentane propionate, digluconate, dodecyl sulfate, ethane sulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In some embodiments, the pharmaceutically acceptable form is a prodrug. As used herein, the term “prodrug” refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

The term “prodrug” is also meant to include any covalently bonded carriers, which release the active compound in vivo when such prodrug is administered to a subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. Other examples of prodrugs include compounds that comprise —NO, —NO₂, —ONO, or —ONO₂ moieties. Prodrugs can typically be prepared using well known methods, such as those described in Burger's Medicinal Chemistry and Drug Discovery, 172-178, 949-982 (Manfred E. Wolff ed., 5th ed., 1995), and Design of Prodrugs (H. Bundgaard ed., Elselvier, New York, 1985).

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₁₋₈)alkyl, (C₁₋₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 10 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as [3-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a disclosed compound contains an alcohol functional group, a prodrug can be formed by the replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, α-amino(C₁₋₄)alkanoyl, arylacyl, and α-aminoacyl, or α-aminoacyl-α-aminoacyl, where each α-aminoacyl group is independently selected from the naturally occurring L-amino acids, —P(O)(OH)₂, —P(O)(O(C₁₋₆)alkyl)₂ or glycosyl (the radical resulting from the removal of a hydroxyl group of the hemiacetal form of a carbohydrate).

If a disclosed compound incorporates an amine functional group, a prodrug can be formed by the replacement of a hydrogen atom in the amine group with a group such as R-carbonyl, RO-carbonyl, NRR′-carbonyl where R and R′ are each independently selected from (C₁₋₁₀)alkyl, (C₃₋₇)cycloalkyl, benzyl, a natural α-aminoacyl or natural α-aminoacyl-natural-α-aminoacyl, —C(OH)C(O)OY¹ wherein Y¹ is H, (C₁₋₆)alkyl or benzyl; —C(OY²)Y³ wherein Y² is (C₁₋₄)alkyl and Y³ is (C₁₋₆)alkyl, carboxy(C₁₋₆)alkyl, amino(C₁₋₄)alkyl or mono-N- or di-N,N—(C₁₋₆)alkylaminoalkyl; and —C(Y⁴)Y⁵ wherein Y⁴ is H or methyl andY⁵ is mono-N- or di-N—(C₁₋₆)alkylamino, morpholino, piperidin-1-yl or pyrrolidin-1-yl.

In some embodiments, the disclosed compounds may encompass an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

Geometric isomers can be represented by the symbol [symbol] which denotes a bond that can be a single, double or triple bond as described herein. Provided herein are various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or“trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

“Enantiomers” are a pair of stereoisomers that are non-superimposable mirror images of each other. A mixture of a pair of enantiomers in any proportion can be known as a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

In some embodiments, an enantiomer is provided partly or substantially free of the corresponding enantiomer, and may be referred to as “optically enriched,” “enantiomerically enriched,” “enantiomerically pure,” and “non-racemic,” as used interchangeably herein. The “enantiomeric excess” or “% enantiomeric excess” of a composition can be calculated using the equation shown below. In the example shown below, a composition contains 90% of one enantiomer, e.g., the S enantiomer, and 10% of the other enantiomer, e.g., the R enantiomer.

$$ee = ( 90 - 10 ) / 100 = 80 \% .$$

Thus, a composition containing 90% of one enantiomer and 10% of the other enantiomer is said to have an enantiomeric excess of 80%. In some embodiments, compositions described herein contain an enantiomeric excess of at least about 50%, at least about 60%, at least about 70%, at least about 80%, at least about 90%, at least about 95%, at least about 98%, at least about 99%, or at least about 99.5% of the S enantiomer, or a range between and including any two of the foregoing values (e.g., 50-99.5% ee). In other words, the compositions contain an enantiomeric excess of the S enantiomer over the R enantiomer. In other embodiments, some compositions described herein contain an enantiomeric excess of at least about 50%, at least about 60%, at least about 70%, at least about 80%, at least about 90%, at least about 95%, at least about 98%, at least about 99%, or at least about 99.5% of the R enantiomer or a range between any two of the foregoing values (e.g., 50-99.5% ee). In other words, the compositions contain an enantiomeric excess of the R enantiomer over the S enantiomer. Where the enrichment of one enantiomer is much greater than about 80% by weight, the compositions are referred to as “substantially enantiomerically enriched,” “substantially enantiomerically pure” or a “substantially non-racemic” preparation.

Thus, a composition containing 90% of one enantiomer and 10% of the other enantiomer is said to have an enantiomeric excess of 80%. In some embodiments, compositions described herein contain an enantiomeric excess of at least about 50%, at least about 60%, at least about 70%, at least about 80%, at least about 90%, at least about 95%, at least about 98%, at least about 99%, or at least about 99.5% of the S enantiomer, or a range between and including any two of the foregoing values (e.g., 50-99.5% ee). In other words, the compositions contain an enantiomeric excess of the S enantiomer over the R enantiomer. In other embodiments, some compositions described herein contain an enantiomeric excess of at least about 50%, at least about 60%, at least about 70%, at least about 80%, at least about 90%, at least about 95%, at least about 98%, at least about 99%, or at least about 99.5% of the R enantiomer or a range between any two of the foregoing values (e.g., 50-99.5% ee). In other words, the compositions contain an enantiomeric excess of the R enantiomer over the S enantiomer. Where the enrichment of one enantiomer is much greater than about 80% by weight, the compositions are referred to as “substantially enantiomerically enriched,” “substantially enantiomerically pure” or a “substantially non-racemic” preparation.

Optical isomers can be obtained by resolution of the racemic mixtures according to conventional processes, e.g., by formation of diastereoisomeric salts, by treatment with an optically active acid or base. Examples of appropriate acids include, but are not limited to, tartaric, diacetyltartaric, dibenzoyltartaric, ditoluoyltartaric, and camphorsulfonic acid. The separation of the mixture of diastereoisomers by crystallization followed by liberation of the optically active bases from these salts affords separation of the isomers. Another method involves synthesis of covalent diastereoisomeric molecules by reacting disclosed compounds with an optically pure acid in an activated form or an optically pure isocyanate. The synthesized diastereoisomers can be separated by conventional means such as chromatography, distillation, crystallization or sublimation, and then hydrolyzed to deliver the enantiomerically enriched compound. Optically active compounds can also be obtained by using active starting materials. In some embodiments, these isomers can be in the form of a free acid, a free base, an ester or a salt.

In any embodiments, the pharmaceutically acceptable form is a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The disclosure also embraces pharmaceutically acceptable forms that are “isotopically labeled derivatives” which are compounds that are identical to those recited herein, except that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorus, fluorine and chlorine, such as ²H, ³H, ¹³C ¹⁴C, ¹⁵N, ¹⁸O, ¹⁷O, ³¹P, ³²P, ³⁵S, ¹⁸F, and ³⁶Cl, respectively. Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure. In some embodiments, radiolabeled compounds are useful for studying metabolism and/or tissue distribution of the compounds or to alter the rate or path of metabolism or other aspects of biological functioning.

“Pharmaceutically acceptable carrier” or “pharmaceutically acceptable excipient” includes any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. The use of such media and agents for pharmaceutically active substances is well known in the art. Except insofar as any conventional media or agent is incompatible with the active ingredient, its use in the therapeutic compositions as disclosed herein is contemplated. Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of compounds described herein.

Definitions of specific functional groups and chemical terms are described in more detail below. The chemical elements are identified in accordance with the Periodic Table of the Elements, CAS version, Handbook of Chemistry and Physics, 75th ed., inside cover, and specific functional groups are generally defined as described therein. Additionally, general principles of organic chemistry, as well as specific functional moieties and reactivity, are described in Organic Chemistry, Thomas Sorrell, University Science Books, Sansalito, 1999; Smith and March March's Advanced Organic Chemistry, 5th ed., John Wiley & Sons, Inc., NewYork, 2001; Larock, Comprehensive Organic Transformations, VCH Publishers, Inc., NewYork, 1989; and Carruthers, Some Modern Methods of Organic Synthesis, 3rd ed., Cambridge University Press, Cambridge, 1987.

Recitation of ranges of values herein are merely intended to serve as a shorthand method of referring individually to each separate value and sub-range falling within the range, unless otherwise indicated herein, and each separate value and sub-range is incorporated into the specification as if it were individually recited herein. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C_{3- 4}, C₄₋₆, C₄₋₅, and C₅₋₆ alkyl.

“Alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1, 2, 3, 4, 5, 6, 7, 8, 9, or 10 carbon atoms, although the present definition also covers the occurrence of the term “alkyl” where no numerical range is designated. In some embodiments, alkyl groups have 1 to 10, 1 to 8, 1 to 6, or 1 to 3 carbon atoms. Representative saturated straight chain alkyls include, but are not limited to, methyl, ethyl, n-propyl, n-butyl, n-pentyl, and n-hexyl groups; while saturated branched alkyls include, but are not limited to, isopropyl, sec-butyl, isobutyl, tert-butyl, isopentyl, 2-methylbutyl, 3-methylbutyl, 2-methylpentyl, 3-methylpentyl, 4-methylpentyl, 2methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group may be optionally substituted by one or more of substituents disclosed herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3hydroxypropyl, benzyl, and phenethyl.

“Alkenyl” refers to a straight or branched hydrocarbon chain radical group consisting solely of carbon and hydrogen atoms, containing at least one double bond, and having from two to ten carbon atoms (i.e., C₂₋₁₀ alkenyl). Whenever it appears herein, a numerical range such as “2 to 10” refers to each integer in the given range; e.g., “2 to 10 carbon atoms” means that the alkenyl group can consist of 2 carbon atoms, 3 carbon atoms, etc., up to and including 10 carbon atoms. In any embodiments, an alkenyl comprises two to eight carbon atoms. In other embodiments, an alkenyl comprises two to six carbon atoms (e.g., C₂₋₆ alkenyl). The alkenyl is attached to the parent molecular structure by a single bond, for example, ethenyl (i.e., vinyl), prop-1-enyl (i.e., allyl), but-1-enyl, pent-1-enyl, penta-1,4-dienyl, and the like. The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), 2-methylprop-2-enyl (C₄), butadienyl (C₄) and the like. Examples of C₂₋₆ alkenyl groups include the aforementioned C₂₋₄ alkenyl groups as well as pentenyl (C₅), pentadienyl (C₅), hexenyl (C₆), 2,3-dimethyl-2-butenyl (C₆) and the like. Additional examples of alkenyl include heptenyl (C₇), octenyl (C₈), octatrienyl (C₈) and the like. Unless stated otherwise in the specification, an alkenyl group may be optionally substituted by one or more of substituents disclosed herein.

“Alkynyl” refers to a straight or branched hydrocarbon chain radical group consisting solely of carbon and hydrogen atoms, containing at least one triple bond, having from two to ten carbon atoms (i.e., C₂₋₁₀ alkynyl). Whenever it appears herein, a numerical range such as “2 to 10” refers to each integer in the given range; e.g., “2 to 10 carbon atoms” means that the alkynyl group can consist of 2 carbon atoms, 3 carbon atoms, etc., up to and including 10 carbon atoms. In any embodiments, an alkynyl comprises two to eight carbon atoms. In other embodiments, an alkynyl has two to six carbon atoms (e.g., C₂₋₆ alkynyl). The alkynyl is attached to the parent molecular structure by a single bond, for example, ethynyl, propynyl, butynyl, pentynyl, 3-methyl-4-pentenyl, hexynyl, and the like. Unless stated otherwise in the specification, an alkynyl group may be optionally substituted by one or more of substituents disclosed herein.

“Alkoxy” refers to the group —O-alkyl, including from 1 to 10 carbon atoms of a straight, branched, saturated cyclic configuration and combinations thereof, attached to the parent molecular structure through an oxygen. Examples include methoxy, ethoxy, propoxy, isopropoxy, butoxy, tbutoxy, pentoxy, cyclopropyloxy, cyclohexyloxy and the like. “Lower alkoxy” refers to alkoxy groups containing one to six carbons. In some embodiments, C₁₋₄alkoxy is an alkoxy group which encompasses both straight and branched chain alkyls of from 1 to 4 carbon atoms. Unless stated otherwise in the specification, an alkoxy group may be optionally substituted by one or more of substituents disclosed herein. The terms “alkenoxy” and “alkynoxy” mirror the above description of “alkoxy” wherein the prefix “alk” is replaced with “alken” or “alkyn” respectively, and the parent “alkenyl” or “alkynyl” terms are as described herein.

“Aromatic” or “aryl” refers to a radical with 6 to 14 ring atoms (e.g., C₆₋₁₄ aromatic or C₆₋₁₄ aryl) which has at least one ring having a conjugated pi electron system which is carbocyclic (e.g., phenyl, fluorenyl, and naphthyl). In some embodiments, the aryl is a C₆₋₁₀ aryl group. For example, bivalent radicals formed from substituted benzene derivatives and having the free valences at ring atoms are named as substituted phenylene radicals. In other embodiments, bivalent radicals derived from univalent polycyclic hydrocarbon radicals whose names end in “-yl” by removal of one hydrogen atom from the carbon atom with the free valence are named by adding “idene” to the name of the corresponding univalent radical, e.g., a naphthyl group with two points of attachment is termed naphthylidene. Whenever it appears herein, a numerical range such as “6 to 14 aryl” refers to each integer in the given range; e.g., “6 to 14 ring atoms” means that the aryl group can consist of 6 ring atoms, 7 ring atoms, etc., up to and including 14 ring atoms. The term includes monocyclic or fused-ring polycyclic (i.e., rings which share adjacent pairs of ring atoms) groups. Polycyclic aryl groups include bicycles, tricycles, tetracycles, and the like. In a multi-ring group, only one ring is required to be aromatic, so groups such as indanyl are encompassed by the aryl definition. Non-limiting examples of aryl groups include phenyl, phenalenyl, naphthalenyl, tetrahydronaphthyl, phenanthrenyl, anthracenyl, fluorenyl, indolyl, indanyl, and the like. Unless stated otherwise in the specification, an aryl group may be optionally substituted by one or more of substituents disclosed herein.

“Cycloalkyl” and “carbocyclyl” each refer to a monocyclic or polycyclic radical that contains only carbon and hydrogen, and can be saturated or partially unsaturated. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₋₁₃ cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl (C₇), cycloheptadienyl (C₇), cycloheptatrienyl (C₇), cyclooctyl (C₈), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group may be optionally substituted by one or more of substituents disclosed herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof, preferably substituted with one, two, or three halo groups. For example, the terms “fluoroalkyl” and “fluoroalkoxy” include haloalkyl and haloalkoxy groups, respectively, in which the halo is fluorine, such as, but not limited to, trifluoromethyl, difluoromethyl, 2,2,2trifluoroethyl, 1-fluoromethyl-2-fluoroethyl, —O—CHF₂, and the like. Each of the alkyl, alkenyl, alkynyl and alkoxy groups are as defined herein and can be optionally further substituted as defined herein.

“Heteroaryl” or, alternatively, “heteroaromatic” refers to a refers to a radical of a 5-18 membered monocyclic or polycyclic (e.g., bicyclic, tricyclic, tetracyclic and the like) aromatic ring system (e.g., having 6, 10 or 14 π electrons shared in a cyclic array) having ring carbon atoms and 1-6 ring heteroatoms provided in the aromatic ring system, wherein each heteroatom is independently selected from nitrogen, oxygen, phosphorous and sulfur (“5-18 membered heteroaryl”). Heteroaryl polycyclic ring systems can include one or more heteroatoms in one or both rings. Whenever it appears herein, a numerical range such as “5 to 18” refers to each integer in the given range; e.g., “5 to 18 ring atoms” means that the heteroaryl group can consist of 5 ring atoms, 6 ring atoms, etc., up to and including 18 ring atoms. In some instances, a heteroaryl can have 5 to 14 ring atoms. In some embodiments, the heteroaryl has, for example, bivalent radicals derived from univalent heteroaryl radicals whose names end in “-yl” by removal of one hydrogen atom from the atom with the free valence are named by adding “-ene” to the name of the corresponding univalent radical, e.g., a pyridyl group with two points of attachment is a pyridylene.

For example, an N-containing “heteroaromatic” or “heteroaryl” moiety refers to an aromatic group in which at least one of the skeletal atoms of the ring is a nitrogen atom. One or more heteroatom(s) in the heteroaryl radical can be optionally oxidized. One or more nitrogen atoms, if present, can also be optionally quaternized. Heteroaryl also includes ring systems substituted with one or more nitrogen oxide (—O—) substituents, such as pyridinyl N-oxides. The heteroaryl is attached to the parent molecular structure through any atom of the ring(s).

“Heteroaryl” also includes ring systems wherein the heteroaryl ring, as defined above, is fused with one or more aryl groups wherein the point of attachment to the parent molecular structure is either on the aryl or on the heteroaryl ring, or wherein the heteroaryl ring, as defined above, is fused with one or more cycloalkyl or heterocyclyl groups wherein the point of attachment to the parent molecular structure is on the heteroaryl ring. For polycyclic heteroaryl groups wherein one ring does not contain a heteroatom (e.g., indolyl, quinolinyl, carbazolyl and the like), the point of attachment to the parent molecular structure can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, phosphorous, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, phosphorous, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, phosphorous, and sulfur (“5-6 membered heteroaryl”). In some embodiments, the 5-6 membered heteroaryl has 1-3 ring heteroatoms selected from nitrogen, oxygen, phosphorous, and sulfur. In some embodiments, the 5-6 membered heteroaryl has 1-2 ring heteroatoms selected from nitrogen, oxygen, phosphorous, and sulfur. In some embodiments, the 5-6 membered heteroaryl has 1 ring heteroatom selected from nitrogen, oxygen, phosphorous, and sulfur.

Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzopyranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno [2,3-d]pyrimidinyl, 5,6-dihydrobenzo[h]quinazolinyl, 5,6-dihydrobenzo[h]cinnolinyl, 6,7-dihydro-5H benzo[6,7]cyclohepta[1,2-c]pyridazinyl, dibenzofuranyl, dibenzothiophenyl, furanyl, furazanyl, furanonyl, furo [3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo [4,5]thieno [2,3-d]pyrimdinyl, 6,7,8,9-tetrahydro-5H-cyclohepta[4,5]thieno [2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno [2,3-c]pridinyl, and thiophenyl (i.e., thienyl). Unless stated otherwise in the specification, a heteroaryl group may be optionally substituted by one or more of substituents disclosed herein.

“Heterocyclyl”, “heterocycloalkyl” or “heterocarbocyclyl” each refer to any 3 to 18-membered non-aromatic radical monocyclic or polycyclic moiety comprising at least one carbon atom and at least one heteroatom selected from nitrogen, oxygen, phosphorous and sulfur. A heterocyclyl group can be a monocyclic, bicyclic, tricyclic or tetracyclic ring system, wherein the polycyclic ring systems can be a fused, bridged or spiro ring system. Heterocyclyl polycyclic ring systems can include one or more heteroatoms in one or both rings. A heterocyclyl group can be saturated or partially unsaturated. Partially unsaturated heterocycloalkyl groups can be termed “heterocycloalkenyl” if the heterocyclyl contains at least one double bond, or “heterocycloalkynyl” if the heterocyclyl contains at least one triple bond. Whenever it appears herein, a numerical range such as “5 to 18” refers to each integer in the given range; e.g., “5 to 18 ring atoms” means that the heterocyclyl group can consist of 5 ring atoms, 6 ring atoms, etc., up to and including 18 ring atoms. For example, bivalent radicals derived from univalent heterocyclyl radicals whose names end in “-yl” by removal of one hydrogen atom from the atom with the free valence are named by adding “-ene” to the name of the corresponding univalent radical, e.g., a piperidine group with two points of attachment is a piperidylene.

An N-containing heterocyclyl moiety refers to a non-aromatic group in which at least one of the ring atoms is a nitrogen atom. The heteroatom(s) in the heterocyclyl radical can be optionally oxidized. One or more nitrogen atoms, if present, can be optionally quaternized. Heterocyclyl also includes ring systems substituted with one or more nitrogen oxide (—O—) substituents, such as piperidinyl N-oxides. The heterocyclyl is attached to the parent molecular structure through any atom of any of the ring(s).

“Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment to the parent molecular structure is on the heterocyclyl ring. In some embodiments, a heterocyclyl group is a 5-14 membered non-aromatic ring system having ring carbon atoms and 1-4 ring heteroatoms, wherein each heteroatom is independently selected from nitrogen, oxygen, phosphorous and sulfur (“5-14 membered heterocyclyl”). In some embodiments, a heterocyclyl group is a 3-10 membered non-aromatic ring system having ring carbon atoms and 1-4 ring heteroatoms, wherein each heteroatom is independently selected from nitrogen, oxygen, phosphorous and sulfur (“3-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, phosphorous and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, phosphorous and sulfur (“5-6 membered heterocyclyl”). In some embodiments, the 5-6 membered heterocyclyl has 1-3 ring heteroatoms selected from nitrogen, oxygen phosphorous and sulfur. In some embodiments, the 5-6 membered heterocyclyl has 1-2 ring heteroatoms selected from nitrogen, oxygen, phosphorous and sulfur. In some embodiments, the 5-6 membered heterocyclyl has 1 ring heteroatom selected from nitrogen, oxygen, phosphorous and sulfur.

“Heterocyclyl” may include one or more ketone group (—C(═O)—) as part of the ring. Examples of a ketone-containing heterocycle include, without limitation, pyridin-2(1H)-one, pyrazin-2(1H)-one, pyrimidin-2(1H)-one, pyrimidin-4(3H)-one, pyridazin-3(2H)-one, pyridin-4(1H)-one, imidazolidin-2-one, 1,3-dihydro-2H-imidazol-2-one, 2,4-dihydro-3H-1,2,4-triazol-3-one, oxazol-2(3H)-one, and oxazolidin-2-one. A ketone-containing heterocyclyl is obtainable by removing a hydrogen atom from its corresponding ketone-containing heterocycle at any available N—H or C—H position.

Exemplary 3-membered heterocyclyls containing 1 heteroatom include, without limitation, azirdinyl, oxiranyl, and thiorenyl. Exemplary 4-membered heterocyclyls containing 1 heteroatom include, without limitation, azetidinyl, oxetanyl and thietanyl. Exemplary 5-membered heterocyclyls containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyls containing 2 heteroatoms include, without limitation, dioxolanyl, oxathiolanyl, thiazolidinyl, and dithiolanyl. Exemplary 5-membered heterocyclyls containing 3 heteroatoms include, without limitation, triazolinyl, diazolonyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6 membered heterocyclyl groups containing 2 heteroatoms include, without limitation, piperazinyl, morpholinyl, thiomorpholinyl, dithianyl, dioxanyl, and triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, benzoxanyl, benzopyrrolidinyl, benzopiperidinyl, benzoxolanyl, benzothiolanyl, benzothianyl, tetrahydroindolyl, tetrahydroquinolinyl, tetrahydroisoquinolinyl, decahydroquinolinyl, decahydroisoquinolinyl, 3-1H-benzimidazol-2-one, (1-substituted)-2-oxo-benzimidazol-3-yl, octahydrochromenyl, octahydroisochromenyl, decahydronaphthyridinyl, decahydro-1,8-naphthyridinyl, octahydropyrrolo[3,2-b]pyrrole, phenanthridinyl, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo [3,2-b]pyranyl, 5,7-dihydro-4H-thieno [2,3-c]pyranyl, 2,3-dihydro-1H-pyrrolo[2,3-b]pyridinyl, hydrofuro[2,3-b]pyridinyl, 4,5,6,7 tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

Unless stated otherwise in the specification, a heterocyclyl group may be optionally substituted by one or more of substituents disclosed herein.

Where substituent groups are specified by their conventional chemical Formulae, written from left to right, they equally encompass the chemically identical substituents that would result from writing the structure from right to left, e.g., —CH₂O— is equivalent to —OCH₂—.

A “leaving group or atom” is any group or atom that will, under the reaction conditions, cleave from the starting material, thus promoting reaction at a specified site. Suitable non-limiting examples of such groups unless otherwise specified include halogen atoms, mesyloxy, p-nitrobenzensulphonyloxy, trifluoromethyloxy, and tosyloxy groups.

“Protecting group” has the meaning conventionally associated with it in organic synthesis, i.e., a group that selectively blocks one or more reactive sites in a multifunctional compound such that a chemical reaction can be carried out selectively on another unprotected reactive site and such that the group can readily be removed after the selective reaction is complete. Non-limiting embodiments of functional groups that can be masked with a protecting group include an amine, hydroxy, thiol, carboxylic acid, and aldehyde. For example, a hydroxy protected form is where at least one of the hydroxy groups present in a compound is protected with a hydroxy protecting group. A variety of protecting groups are disclosed, for example, Greene's Protective Groups in Organic Synthesis, Fifth Edition, Wiley (2014), incorporated herein by reference in its entirety. For additional background information on protecting group methodologies (materials, methods and strategies for protection and deprotection) and other synthetic chemistry transformations useful in producing the compounds described herein, see in R. Larock, Comprehensive organic Transformations, VCH Publishers (1989); Greene's Protective Groups in Organic Synthesis, Fifth Edition, Wiley (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995). These references are incorporated herein by reference in their entirety.

The terms “substituted” or “substitution” mean that at least one hydrogen present on a group atom (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. Unless otherwise indicated, a “substituted” group can have a substituent at one or more substitutable positions of the group, and when more than one position in any given structure is substituted, the substituent is either the same or different at each position. Substituents include one or more group(s) individually and independently selected from acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties (other than hydrogen) can be optionally substituted with one or more substituents (up to six, valence permitting) selected from OH, NH₂, oxo, halo, nitro, COOH, C(O)NH₂ or cyano. For example, a cycloalkyl substituent can have a halide substituted at one or more ring carbons, and the like. The protecting groups that can form the protective derivatives of the above substituents are known to those of skill in the art and can be found in references such as Greene and Wuts, above.

Suitable substituents include, but are not limited to, haloalkyl and trihaloalkyl, alkoxyalkyl, halophenyl, -M-heteroaryl, -M-heterocycle, -M-aryl, -M-OR^{a}, -M-SR^{a}, -M-N(R^{a})₂, -M-OC(O)N(R^{a})₂, -M-C(═NR^{a})N(R^{a})₂, -M-C(═NR^{a})OR^{a}, -M-P(O)(R^{a})₂, Si(R^{a})₃, -M-NR^{a}C(O)R^{a}, -M-NR^{a}C(O)OR^{a}, -M-C(O)R^{a}, -M-C(═S)R^{a}, -M-C(═S)NR^{a}R^{a}, -M-C(O)N(R^{a})₂, -M-C(O)NR^{a}-M-N(R^{a})₂, -M-NR^{a}C(NR^{a})N(R^{a})₂, -M-NR^{a}C(S)N(R^{a})₂, -M-S(O)₂R^{a}, -M C(O)R^{a}, -M-OC(O)R^{a}, -MC(O)SR^{a}, -M-S(O)₂N(R^{a})₂, —C(O)-M-C(O)R^{a}, -MCO₂R^{a}, -MC(═O)N(R^{a})₂, -M-C(═NH)N(R^{a})₂, and -M-OC(═NH)N(R^{a})₂ (wherein M is a C₁₋₆ alkyl group).

When a ring system (e.g., cycloalkyl, heterocyclyl, aryl, or heteroaryl) is substituted with several substituents varying within an expressly defined range, it is understood that the total number of substituents does not exceed the normal available valencies under the existing conditions. Thus, for example, a phenyl ring substituted with “p” substituents (where “p” ranges from 0 to 5) can have 0 to 5 substituents, whereas it is understood that a pyridinyl ring substituted with “p” substituents has several substituents ranging from 0 to 4. The maximum number of substituents that a group in the disclosed compounds can have can be easily determined. The substituted group encompasses only those combinations of substituents and variables that result in a stable or chemically feasible compound. A stable compound or chemically feasible compound is one that, among other factors, has stability sufficient to permit its preparation and detection. In some embodiments, disclosed compounds are sufficiently stable that they are not substantially altered when kept at a temperature of 40° C. or less, in the absence of moisture (e.g., less than about 10%, less than about 5%, less than about 2%, less than about 1%, or less than about 0.5%) or other chemically reactive conditions, for e.g., at least about 3 days, at least about a week, at least about 2 weeks, at least about 4 weeks, or at least about 6 weeks.

The terms “combine, combining, to combine, combination” refer to the action of adding at least one chemical substance to another chemical substance(s) either sequentially or simultaneously. In some embodiments, bringing these chemical substances together can result in transformation of the initial chemical substances into one or more different chemical substances. This transformation can occur through one or more chemical reactions, e.g., where covalent bonds are formed, broken, rearranged and the like. A non-limiting example can include hydrolysis of an ester into an alcohol and carboxylic acid which can result from the combination of the ester with a suitable base. In another non-limiting example, an aryl fluoride can be combined with an amine to provide an aryl amine through a substitution process. These terms also include changes in association of charged chemical substances and creation of charged chemical substances, such as, but not limited to, N-oxide formation, acid addition salt formation, basic addition salt formation, and the like. These terms include the creation and/or transformation of radical chemical substances and isotopically labeled chemical substances.

The terms “convert, converting, to convert, conversion” refer to a subset of “combination” and its grammatical equivalents, where the action of one or more reagents transforms one or more functional groups on a chemical substance to another functional group(s). For example, a conversion includes, but is not limited to, transforming a nitro functional group on a chemical substance to an amine with a reducing agent. Conversions also include changes in charged chemical substances, radical chemical substances and isotopically labeled chemical substances. However, the term “convert” does not include alteration of conserved bonds in disclosed genuses and compounds.

### Compounds

In one aspect, the present invention relates to a compound of formula (I),

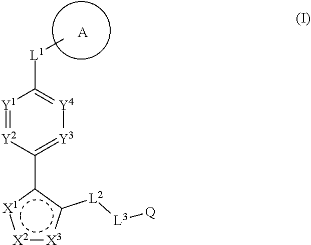

or a pharmaceutically acceptable salt, tautomer and/or stereoisomer thereof,
wherein:
A ring is a 5-membered heterocyclyl or 6-membered cyclohexyl or heterocyclyl selected from:

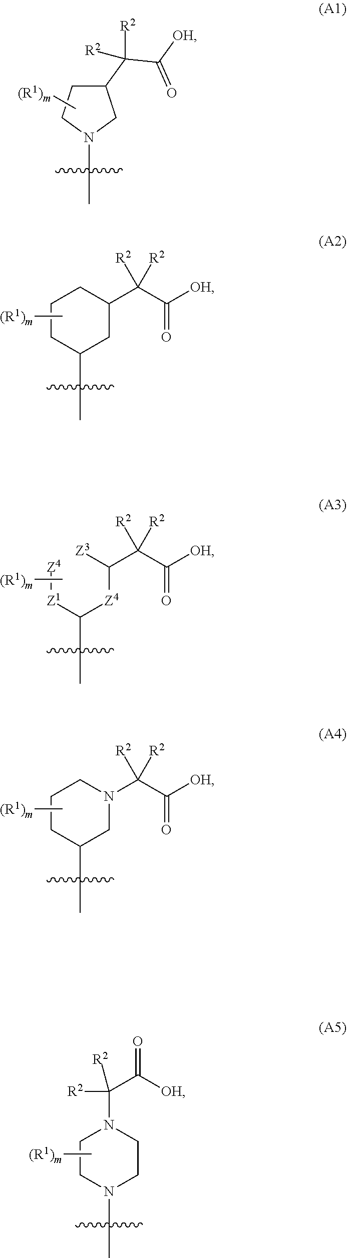

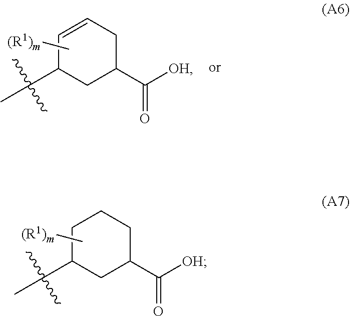

L¹ is a covalent bond, NH, O or S; provided that when L¹ is a covalent bond, A ring is selected from Formulae (A1), (A2), (A3), (A4) or (A5); further provided that when L¹ is NH or S, A ring is selected from Formulae (A6) or (A7); and further provided that when L¹ is O, A ring is selected from Formula (A6);
L² is a covalent bond or (CR⁷R⁷)ₚ;
L³ is a covalent bond, O or NR⁷, provided that at least one of L² and L³ is not a covalent bond;
Q is C(═O)NR⁹R¹⁰, C(═O)OR¹⁰, or a ring selected from a 5- or 6-membered heteroaryl group or a 5- or 6-membered heterocyclyl group, wherein the ring comprises at least one carbon atom, at least one nitrogen atom, and optionally 1-4 additional heteroatoms selected from nitrogen, oxygen and sulfur wherein oxygen may be a ring member and/or an oxo (═O) group attached to a ring member, and wherein the ring is substituted with (R³)ₙ and one R⁴;
X¹ is N, O or CR^{6a};
X² is N or NR⁶;
X³ is N, NR⁶ or CR⁶, and the dashed circle denotes bonds forming a five-membered aromatic ring;
Y¹, Y², Y³ and Y⁴ are each independently N or CR⁵, provided that at least one but no more than two of Y¹, Y², Y³ and Y⁴ are N;
Z¹, Z², Z³ and Z⁴ are each independently CH₂ or O, provided that only one of Z¹, Z², Z³ and Z⁴ is O;
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, (CH₂)_{q}-5-6-membered heteroaryl ring substituted with 1-4 R¹¹, or (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
each of R^{6a} and R⁶ is independently hydrogen, halogen, CN, C₁₋₄ alkyl, or cyclopropyl;
R⁷ at each occurrence is independently hydrogen, C₁₋₄alkyl, or C₃₋₅cycloalkyl, or R⁷ and R⁷, together with the carbon atom to which they are attached, form a 3-5-membered cycloalkyl ring;
each occurrence of R⁹ and R¹⁰ is independently hydrogen, C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, or O(CH₂)ₚ—C₃₋₇cycloalkyl, wherein each phenyl is independently optionally substituted with 1-3 halogen, C₁₋₆alkyl, or C₁₋₆alkoxy;
R¹² at each occurrence is independently hydrogen, C₁₋₄alkyl, C₃₋₇cycloalkyl, or R¹² and R¹², together with the carbon atom to which they are attached, form a 3-6-membered cycloalkyl ring;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
n is 0, 1, or 2;
p at each occurrence is independently 1, 2, 3 or 4;
q at each occurrence is independently 0, 1, 2, 3 or 4.

In some embodiments, A ring is selected from Formula (A1). In certain other embodiments, A ring is selected from Formula (A2). In yet other embodiments, A ring is selected from Formula (A3). In yet other embodiments, A ring is selected from Formula (A4). In yet other embodiments, A ring is selected from Formula (A5). In yet other embodiments, A ring is selected from Formula (A6). In yet other embodiments, A ring is selected from Formula (A7).

In some embodiments, Formula (A1) is Formula (Ala):

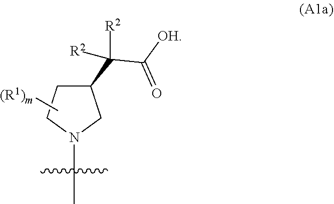

In some other embodiments, Formula (A1) is Formula (Alb):

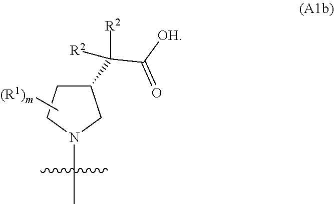

In some embodiments, Formula (A3) is Formula (A3a):

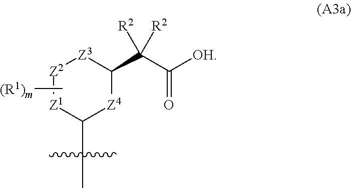

In some other embodiments, Formula (A3) is Formula (A3b):

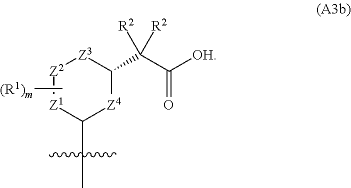

In any embodiments, L¹ is a covalent bond. In certain other embodiments, L¹ is NH. In yet other embodiments, L¹ is O. In yet other embodiments, L¹ is S.

In any embodiments, R¹ at each occurrence is independently hydrogen. In certain other embodiments, R¹ at each occurrence is independently halogen. In yet other embodiments, m is 2, one R¹ is hydrogen, and the other R¹ is halogen. In yet other embodiments, m is 2, one R¹ is hydrogen, and the other R¹ is F. In yet other embodiments, m is 2, R¹ at each occurrence is independently F.

In any embodiments, R² at each occurrence is independently hydrogen or deuterium. In certain other embodiments, R² at each occurrence is independently C₁₋₄alkyl. In yet other embodiments, R² at each occurrence is independently C₃₋₅cycloalkyl. In yet other embodiments, R² and R², together with the carbon atom to which they are attached, form a 3-5-membered cycloalkyl ring. In yet other embodiments, one R² is hydrogen, and the other R² is methyl. In yet other embodiments, one R² is hydrogen, and the other R² is ethyl or propyl. In yet other embodiments, one R² is hydrogen, and the other R² is cyclopropyl. In yet other embodiments, R² and R², together with the carbon atom to which they are attached, form a cyclopropyl ring.

In any embodiments, Y² is N, and each of Y¹, Y³ and Y⁴ is independently CR⁵. In certain other embodiments, Y¹ is CR⁵, Y² is N, and each of Y³ and Y⁴ is independently CH. In yet other embodiments, Y¹ is CR⁵, Y² is N, Y³ is N, and Y⁴ CH.

In any embodiments, R⁵ at each occurrence is independently hydrogen. In certain other embodiments, R⁵ at each occurrence is independently methyl. In yet other embodiments, R⁵ at each occurrence is independently ethyl. In yet other embodiments, R⁵ at each occurrence is independently halogen. In yet other embodiments, R⁵ at each occurrence is independently CHF₂ or CF₃. In yet other embodiments, R⁵ at each occurrence is independently CN. In any embodiments, R⁵ at each occurrence is independently hydrogen or C₁₋₆alkyl.

In any embodiments, X¹ is N, X² is N, and X³ is NR⁶. In certain other embodiments, X¹ is CH, X² is N, and X³ is NR⁶. In yet other embodiments, X¹ is O, X² is N, and X³ is CR⁶.

In any embodiments, R⁶ is methyl, ethyl, or propyl. In any embodiments, R⁶ is methyl. In certain other embodiments, R⁶ is hydrogen. In yet other embodiments, R⁶ is ethyl.

In any embodiments, R^{6a} is methyl, ethyl, or propyl. In any embodiments, R^{6a} is methyl. In certain other embodiments, R^{6a} is hydrogen. In yet other embodiments, R^{6a} is ethyl.

In any embodiments, L² is a covalent bond. In certain other embodiments, L² is (CR⁷R⁷)ₚ. In yet other embodiments, L² is CH₂.

In any embodiments, L³ is a covalent bond. In certain other embodiments, L³ is O. In yet other embodiments, L³ is NR⁷.

In any embodiments, R⁹ is C₁₋₄alkyl.

In any embodiments, R¹⁰ is C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹. In certain other embodiments, R¹⁰ is C₁₋₆alkyl. In yet other embodiments, R¹⁰ is (CH₂)ₚ—C₃₋₇cycloalkyl. In yet other embodiments, R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur.

In any embodiments, Q is C(═O)NR⁹R¹⁰. In certain other embodiments, Q is C(═O)OR¹⁰. In yet other embodiments, Q is a ring selected from a 5- or 6-membered heteroaryl group or a 5- or 6-membered heterocyclyl group, wherein the ring members comprises at least one carbon atom, at least one nitrogen atom, and optionally 1-4 additional heteroatoms selected from nitrogen, oxygen and sulfur, wherein oxygen may be a ring member and/or an oxo group (═O) attached to a ring member, and wherein the ring is substituted with (R³)ₙ and one R⁴. In yet other embodiments, the Q ring is

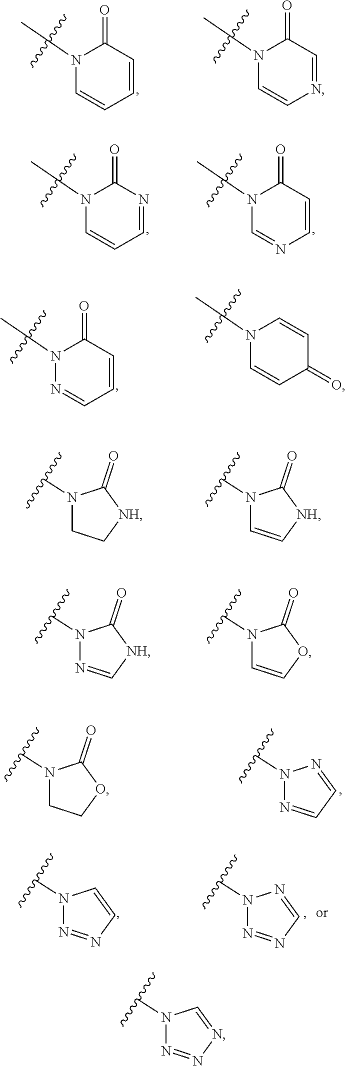

each of which is substituted with (R³)ₙ and one R⁴ at any available carbon or nitrogen position. In yet other embodiments, the Q ring is

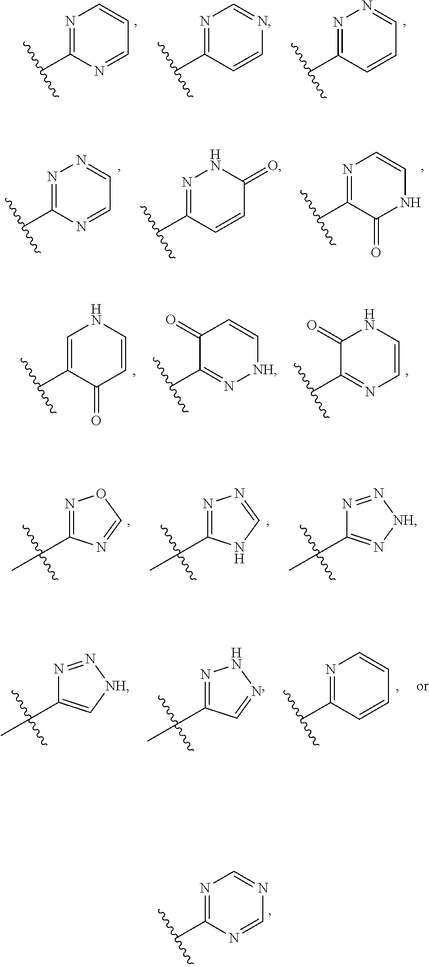

each of which is substituted with (R³)ₙ and one R⁴ at any available carbon or nitrogen position.

In any embodiments, R³ at each occurrence is independently hydrogen, halogen, or C₁₋₄alkyl. In certain other embodiments, R³ at each occurrence is independently C₁₋₄alkyl. In yet other embodiments, R³ at each occurrence is methyl.

In any embodiments, R⁴ is hydrogen, C₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, in which each phenyl is independently optionally substituted with 1-3 halogen, C₁₋₆alkyl, or C₁₋₆alkoxy. In certain other embodiments, R⁴ is (CH₂)_{q}-5-6-membered heteroaryl ring substituted with 1-4 R¹¹, or (CH₂)_{q}-5-7-membered heterocyclyl ring substituted with 1-4 R¹¹. In yet other embodiments, R⁴ is C₁₋₆alkyl, C₂₋₆alkynyl, C₁₋₆alkoxy, (CH₂)ₚ—C₁₋₆alkoxy, C₃₋₇cycloalkyl, (CH₂)ₚ—C₃₋₇cycloalkyl, or C₂₋₆alkenyl-C₃₋₇cycloalkyl.

In any embodiments, m is 0. In certain other embodiments, m is 1. In yet other embodiments, m is 2. In yet other embodiments, m is 3.

In any embodiments, n is 0. In certain other embodiments, n is 1. In yet other embodiments, n is 2.

In any embodiments, p is 1. In certain other embodiments, p is 2. In yet other embodiments, p is 3. In yet other embodiments, p is 4.

In any embodiments, q is 0. In certain other embodiments, q is 1. In yet other embodiments, q is 2. In yet other embodiments, q is 3. In yet other embodiments, q is 4.

In some embodiments, the compound of Formula (I) includes a compound of Formula (II),

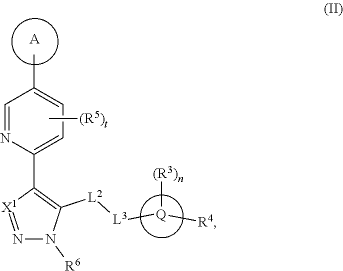

or a pharmaceutically acceptable salt, tautomer and/or stereoisomer thereof,
wherein:
A ring is a 5-membered heterocyclyl or 6-membered cyclohexyl or heterocyclyl selected from:

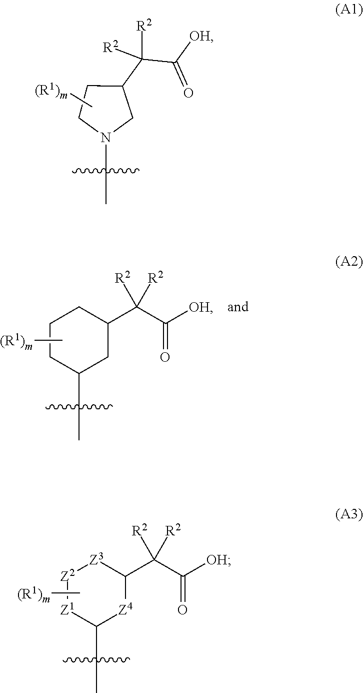

R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
each one of Z¹, Z², Z³ and Z⁴ is independently CH₂ or O, provided that only one of Z¹, Z², Z³ and Z⁴ is O;
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR⁶a;
R^{6a} is hydrogen, or methyl;
R⁶ is hydrogen, halogen, CN, methyl, ethyl, propyl, or cyclopropyl;
L² is a covalent bond or (CR⁷R⁷)ₚ;
L³ is a covalent bond, O or NR⁷, provided that at least one of L² and L³ is not a covalent bond;
the Q ring is selected from 5-membered heteroaryl or heterocyclyl and 6-membered heteroaryl or heterocyclyl, wherein the Q ring contains one nitrogen atom and optionally contains 1-4 additional heteroatoms selected from nitrogen, oxygen and sulfur, and wherein the Q ring is substituted with (R³)ₙ and one R⁴;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, (CH₂)_{q}-5-6-membered heteroaryl ring substituted with 1-4 R¹¹, (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
n is 0, 1 or 2;
t is 0, 1, 2 or 3;
p at each occurrence is independently 1, 2, 3 or 4; and
q at each occurrence is independently 0, 1, 2, 3 or 4.

In some embodiments, the compound of Formula (II) includes a compound of Formula (IIa),

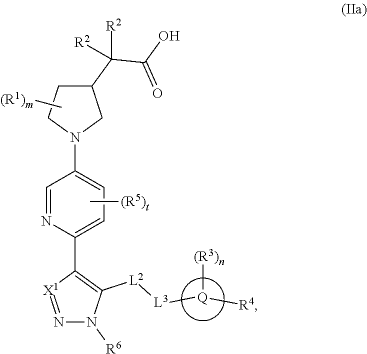

or a pharmaceutically acceptable salt, tautomer and/or stereoisomer thereof,
wherein each of the variables (Q, X¹, L², L³, R¹, R², R³, R⁴, R⁵, R⁶, m, n, t) may have any of the values disclosed herein.

In some embodiments, the compound of Formula (II) includes a compound of Formula (IIb),

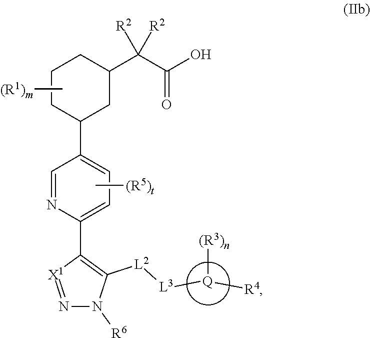

or a pharmaceutically acceptable salt, tautomer and/or stereoisomer thereof,
wherein each of the variables (Q, X¹, L², L³, R¹, R², R³, R⁴, R⁵, R⁶, m, n, t) may have any of the values disclosed herein.

In some embodiments, the compound of Formula (II) includes a compound of Formula (IIc),

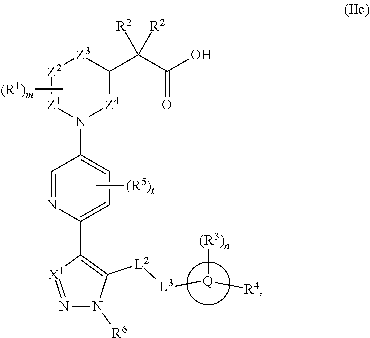

or a pharmaceutically acceptable salt, tautomer and/or stereoisomer thereof,
wherein each of the variables (Q, X¹, Z¹, Z², Z³, Z⁴, L², L³, R¹, R², R³, R⁴, R⁵, R⁶, m, n, t) may have any of the values disclosed herein.

In any embodiments (including but not limited to compounds of Formulae I, II, II(a), II(b), and II(c)), R¹ at each occurrence is independently hydrogen. In certain other embodiments, R¹ at each occurrence is independently halogen. In yet other embodiments, m is 2, one R¹ is hydrogen, and the other R¹ is halogen. In yet other embodiments, m is 2, one R¹ is hydrogen, and the other R¹ is F. In yet other embodiments, m is 2, and R¹ at each occurrence is independently F.

In any embodiments, R² at each occurrence is independently hydrogen or deuterium. In certain other embodiments, R² at each occurrence is independently C₁₋₄alkyl. In yet other embodiments, R² at each occurrence is independently C₃₋₅cycloalkyl. In yet other embodiments, R² and R², together with the carbon atom to which they are attached, form a 3-5-membered cycloalkyl ring. In yet other embodiments, one R² is hydrogen, and the other R² is methyl. In yet other embodiments, one R² is hydrogen, and the other R² is ethyl or propyl. In yet other embodiments, one R² is hydrogen, and the other R² is cyclopropyl. In yet other embodiments, R² and R², together with the carbon atom to which they are attached, form a cyclopropyl ring.

In any embodiments, the moiety

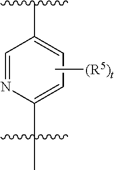

is

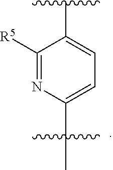

In any embodiments, R⁵ at each occurrence is independently hydrogen, halogen, C₁₋₆alkyl or haloC₁₋₆alkyl. In certain other embodiments, R⁵ at each occurrence is independently C₁₋₆alkyl. In certain other embodiments, R⁵ at each occurrence is independently methyl. In yet other embodiments, R⁵ at each occurrence is independently ethyl. In yet other embodiments, R⁵ at each occurrence is independently CHF₂ or CF₃. In yet other embodiments, R⁵ at each occurrence is independently hydrogen. In yet other embodiments, R⁵ at each occurrence is independently halogen. In yet other embodiments, R⁵ at each occurrence is independently CN.

In any embodiments, X¹ is N. In certain other embodiments, X¹ is CH.

In any embodiments, R⁶ is methyl. In certain other embodiments, R⁶ is ethyl.

In any embodiments, R^{6a} is methyl. In certain other embodiments, R^{6a} is ethyl.

In any embodiments, L² is L² is (CR⁷R⁷)ₚ. In certain other embodiments, L² is CH₂.

In any embodiments, L³ is a covalent bond. In certain other embodiments, L³ is O. In yet other embodiments, L³ is NR⁷.

In any embodiments, the Q ring is

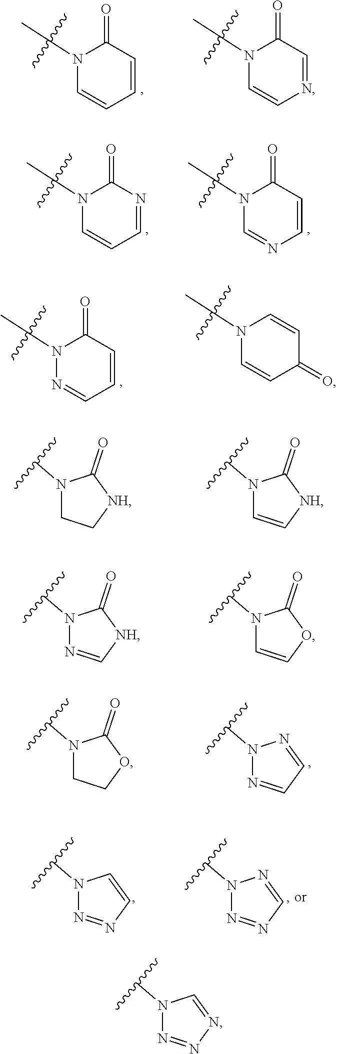

each of which is substituted with (R³)ₙ and one R⁴ at any available carbon or nitrogen position, and n is 0, 1 or 2. In certain other embodiments, the Q ring is

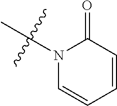 [SMILES: *n1ccccc1=O]

substituted with (R³)ₙ and one R⁴ at any available carbon position, and n is 0, 1 or 2. In yet other embodiments, the Q ring is

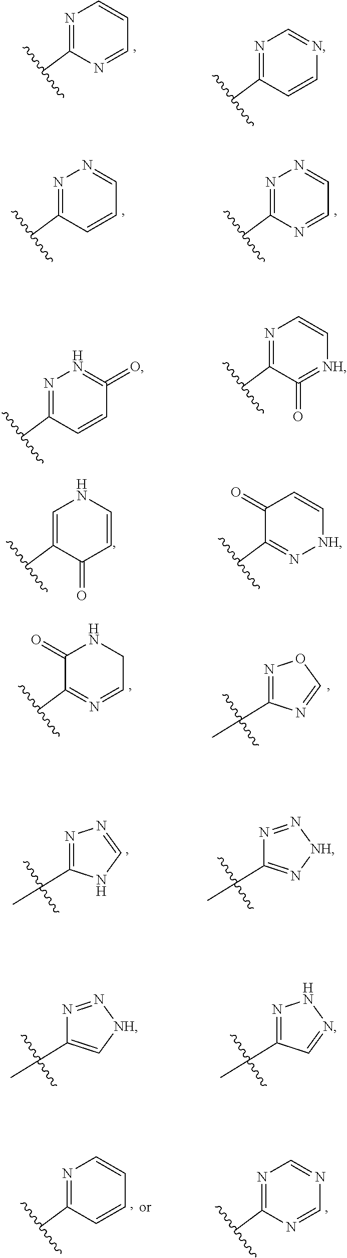

each of which is substituted with (R³)ₙ and one R⁴ at any available carbon or nitrogen position, and n is 0, 1 or 2.

In any embodiments, R³ at each occurrence is independently hydrogen, halogen, or C₁₋₄alkyl. In certain other embodiments, R³ at each occurrence is independently C₁₋₄alkyl. In yet other embodiments, R³ at each occurrence is independently methyl.

In any embodiments, R⁴ is hydrogen, C₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy. In certain other embodiments, R⁴ is (CH₂)_{q}-5-6-membered heteroaryl ring substituted with 1-4 R¹¹, or (CH₂)_{q}-5-7-membered heterocyclyl ring substituted with 1-4 R¹¹. In yet other embodiments, R⁴ is C₁₋₆alkyl, C₂₋₆alkynyl, C₁₋₆alkoxy, (CH₂)ₚ—C₁₋₆alkoxy, C₃₋₇cycloalkyl, (CH₂)ₚ—C₃₋₇cycloalkyl, or C₂₋₆ alkynyl-C₃₋₇cycloalkyl.

In some embodiments, the compound of Formula (I) includes a compound of Formula (III),

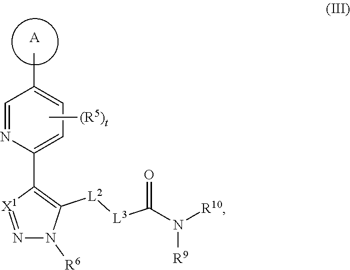

or a pharmaceutically acceptable salt, tautomer and/or stereoisomer thereof,
wherein:
A ring is a 5-membered heterocyclyl or 6-membered cyclohexyl or heterocyclyl selected from:

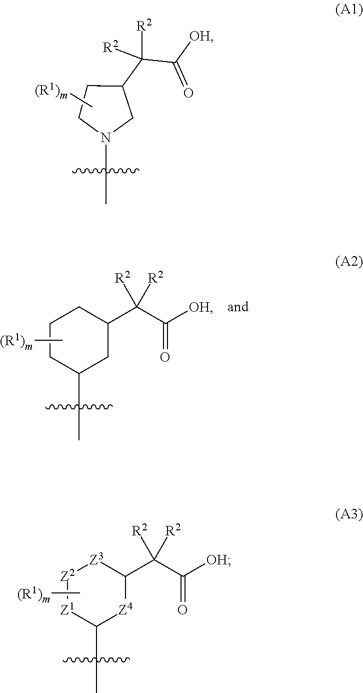

R¹ at each occurrence is independently hydrogen, halogen, keto (═O), C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl;
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
each one of Z¹, Z², Z³ and Z⁴ is independently CH₂ or O, provided that only one of Z¹, Z², Z³ and Z⁴ is O;
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR⁶a;
R^{6a} is hydrogen, or methyl;
R⁶ is hydrogen, halogen, CN, methyl, ethyl, propyl, or cyclopropyl;
L² is a covalent bond or (CR⁷R⁷)ₚ;
L³ is a covalent bond, O or NR⁷, provided that at least one of L² and L³ is not a covalent bond;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
each occurrence of R⁹ and R¹⁰ is independently hydrogen, C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy;
R¹² at each occurrence is independently hydrogen, C₁₋₄alkyl, C₃₋₇cycloalkyl, or R¹² and R¹², together with the carbon atom to which they are attached, form a 3-6-membered cycloalkyl ring;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
t is 0, 1, 2 or 3;
p at each occurrence is independently 1, 2, 3 or 4;
and q at each occurrence is independently 0, 1, 2, 3 or 4.

In some embodiments, the compound of Formula (III) includes a compound of Formula (IIIa),

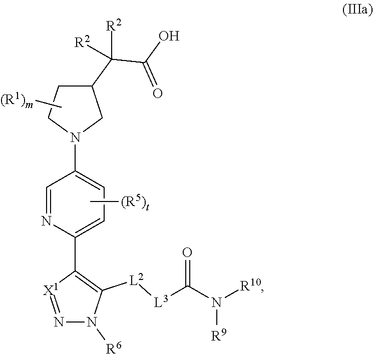

or a pharmaceutically acceptable salt, tautomer and/or stereoisomer thereof,
wherein each of the variables (X¹, L², L³, R¹, R², R⁵, R⁶, R⁹, R¹⁰, m, t) may have any of the values disclosed herein.

In some embodiments, the compound of Formula (III) includes a compound of Formula (IIIb),

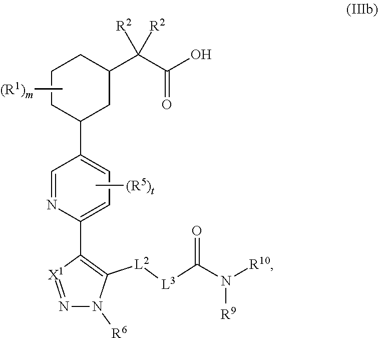

or a pharmaceutically acceptable salt, tautomer and/or stereoisomer thereof,
wherein each of the variables (X¹, L², L³, R¹, R², R⁵, R⁶, R⁹, R¹⁰, m, t) may have any of the values disclosed herein.

In some embodiments, the compound of Formula (III) includes a compound of Formula (IIIc),

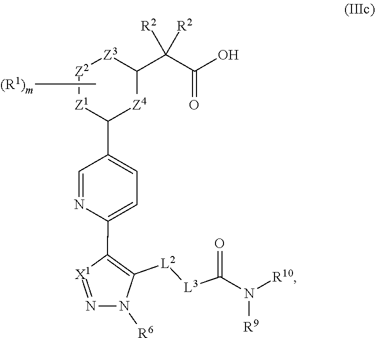

or a pharmaceutically acceptable salt, tautomer and/or stereoisomer thereof,
wherein each of the variables (X¹, Z¹, Z², Z³, Z⁴, L², L³, R¹, R², R⁵, R⁶, R⁹, R¹⁰, m, t) may have any of the values disclosed herein.

In any embodiments of any compounds disclosed herein, R¹ at each occurrence is independently hydrogen. In certain other embodiments, R¹ at each occurrence is independently halogen. In yet embodiments, one R¹ is hydrogen, and the other R¹ is halogen. In yet embodiments, one R¹ is hydrogen, and the other R¹ is F. In yet embodiments, R¹ at each occurrence is independently F.

In any embodiments, the moiety

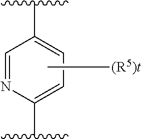

is

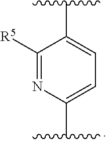

In any embodiments, R⁵ at each occurrence is independently hydrogen, halogen, or C₁₋₆alkyl. In certain other embodiments, R⁵ at each occurrence is independently C₁₋₆alkyl. In yet other embodiments, R⁵ at each occurrence is independently methyl. In yet other embodiments, R⁵ at each occurrence is independently ethyl. In yet other embodiments, R⁵ at each occurrence is independently hydrogen. In yet other embodiments, R⁵ at each occurrence is independently halogen. In yet other embodiments, R⁵ at each occurrence is independently CN.

In any embodiments, X¹ is N. In certain other embodiments, X¹ is CH.

In any embodiments, R⁶ is methyl.

In any embodiments, L² is a (CR⁷R⁷)ₚ. In certain other embodiments, L² is a CH₂.

In any embodiments, L³ is a covalent bond. In certain other embodiments, L³ is O. In yet other embodiments, L³ is NR⁷.

In any embodiments, R⁹ is C₁₋₄alkyl.

In any embodiments, R¹⁰ is C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹. In certain other embodiments, R¹⁰ is C₁₋₆alkyl. In yet other embodiments, R¹⁰ is (CH₂)_{q}—C₃₋₇cycloalkyl. In yet other embodiments, R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur.

In some embodiments, the compound of Formula (I) is selected from:

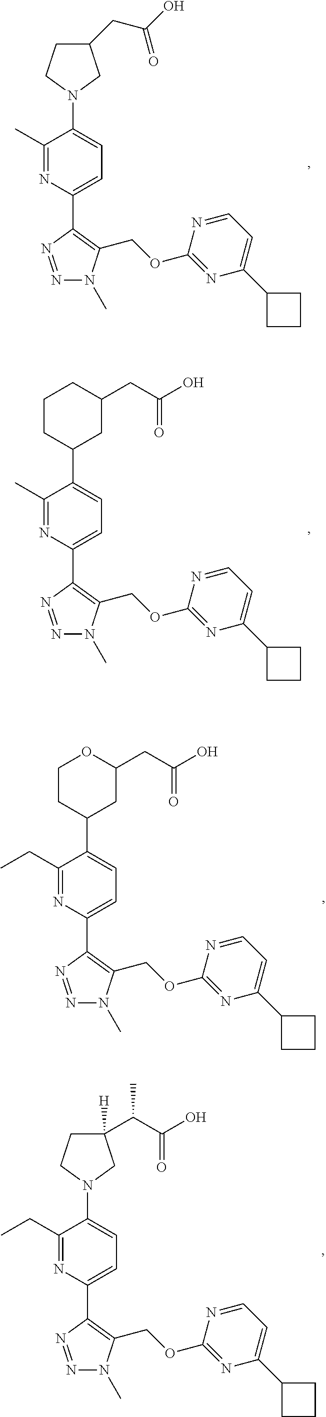 [SMILES: Cc1nc(-c2nnn(C)c2COc2nccc(C3CCC3)n2)ccc1N1CCC(CC(=O)O)C1 ; Cc1nc(-c2nnn(C)c2COc2nccc(C3CCC3)n2)ccc1C1CCCC(CC(=O)O)C1 ; CCc1nc(-c2nnn(C)c2COc2nccc(C3CCC3)n2)ccc1C1CCOC(CC(=O)O)C1 ; CCc1nc(-c2nnn(C)c2COc2nccc(C3CCC3)n2)ccc1N1CC[C@@H]([C@H](C)C(=O)O)C1]

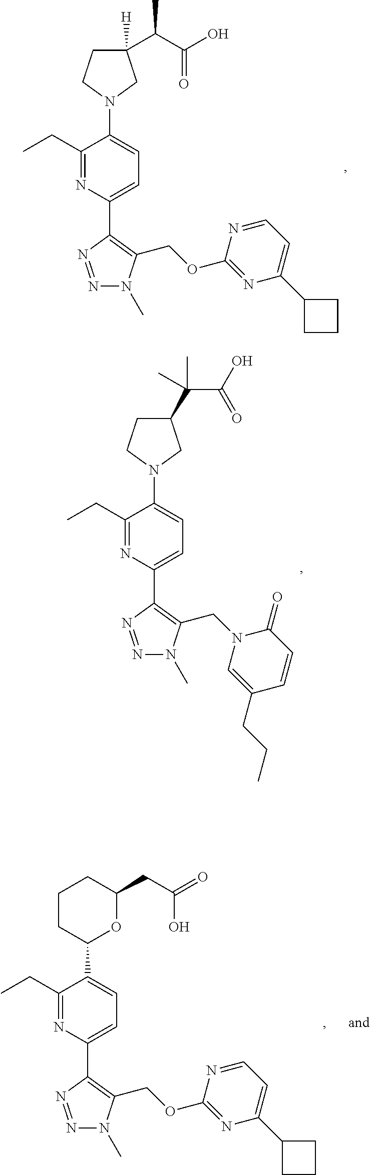 [SMILES: CCc1nc(-c2nnn(C)c2COc2nccc(C3CCC3)n2)ccc1N1CC[C@@H]([C@@H](C)C(=O)O)C1 ; CCCc1ccc(=O)n(Cc2c(-c3ccc(N4CC[C@@H](C(C)(C)C(=O)O)C4)c(CC)n3)nnn2C)c1 ; CCc1nc(-c2nnn(C)c2COc2nccc(C3CCC3)n2)ccc1[C@@H]1CCC[C@@H](CC(=O)O)O1]

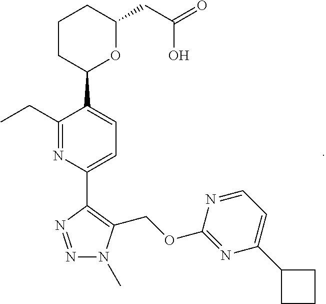 [SMILES: CCc1nc(-c2nnn(C)c2COc2nccc(C3CCC3)n2)ccc1[C@H]1CCC[C@H](CC(=O)O)O1]

In another aspect, the present invention relates to a compound of Formula (IV),

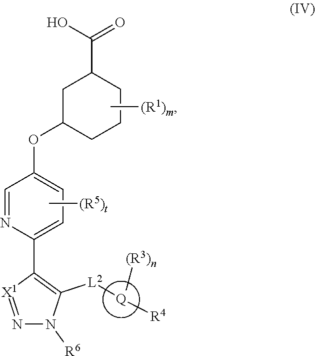

or a pharmaceutically acceptable salt, tautomer and/or stereoisomer thereof,
wherein:
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR⁶a;
R^{6a} is hydrogen, or methyl;
R⁶ is hydrogen, halogen, CN, methyl, ethyl, propyl, or cyclopropyl;
L² is (CR⁷R⁷)ₚ;
Q ring is

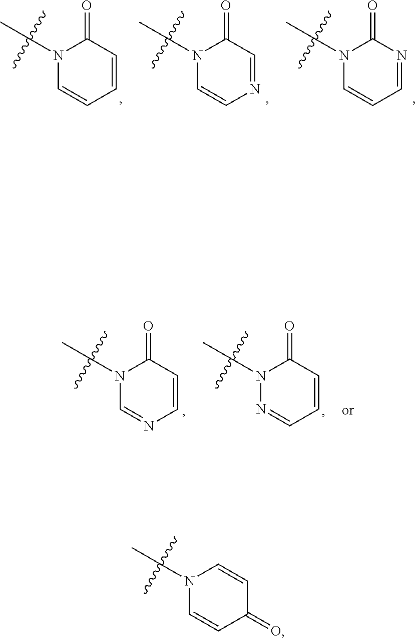 [SMILES: *n1ccccc1=O ; *n1ccncc1=O ; *n1cccnc1=O ; *n1cnccc1=O ; *n1ncccc1=O ; *n1ccc(=O)cc1]

each of which is substituted with (R³)ₙ and one R⁴ at any available carbon position;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, (CH₂)_{q}-5-6-membered heteroaryl ring substituted with 1-4 R¹¹, (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
n is 0, 1 or 2;
t is 0, 1, 2 or 3;
p at each occurrence is independently 1, 2, 3 or 4;
and q at each occurrence is independently 0, 1, 2, 3 or 4.

In some embodiments, the compound of Formula (IV) includes a compound of Formula (IVa),

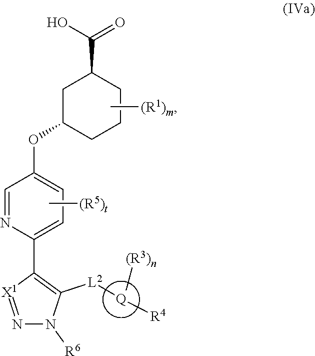

or a pharmaceutically acceptable salt, tautomer and/or stereoisomer thereof,
wherein each of the variables (X¹, L², Q, R¹, R², R³, R⁴, R⁶, R⁹, R¹⁰, n, m, t) may have any of the values disclosed herein.

In some embodiments, m is 0. In certain other embodiments, m is 1.

In some embodiments, the moiety

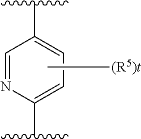

is

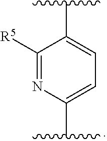

In any embodiments, R⁵ at each occurrence is independently hydrogen, halogen, or C₁₋₆alkyl. In certain other embodiments, R⁵ at each occurrence is independently C₁₋₆alkyl. In yet other embodiments, R⁵ at each occurrence is independently methyl or ethyl. In yet other embodiments, R⁵ at each occurrence is independently CHF₂ or CF₃. In yet other embodiments, R⁵ at each occurrence is independently hydrogen. In yet other embodiments, R⁵ at each occurrence is independently halogen. In yet other embodiments, R⁵ at each occurrence is independently CN.

In any embodiments, X¹ is N. In certain other embodiments, X¹ is CH.

In any embodiments, R⁶ is methyl.

In any embodiments, L² is a CH₂.

In any embodiments, Q ring is

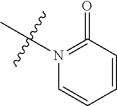

substituted with (R³)ₙ and one R⁴ at any available carbon position.

In any embodiments, R³ at each occurrence is independently hydrogen, halogen, or C₁₋₄alkyl. In certain other embodiments, R³ at each occurrence is independently C₁₋₄alkyl. In yet other embodiments, R³ at each occurrence is independently methyl.

In some embodiments, n is 0. In certain other embodiments, n is 1.

In any embodiments, R⁴ is hydrogen, C₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy. In certain other embodiments, R⁴ is (CH₂)_{q}-5-6-membered heteroaryl ring substituted with 1-4 R¹¹, or (CH₂)_{q}-5-7-membered heterocyclyl ring substituted with 1-4 R¹¹. In yet other embodiments, R⁴ is C₁₋₆alkyl, C₂₋₆alkynyl, C₁₋₆alkoxy, (CH₂)ₚ—C₁₋₆alkoxy, C₃₋₇cycloalkyl, (CH₂)ₚ—C₃₋₇cycloalkyl, or C₂₋₆ alkynyl-C₃₋₇cycloalkyl.

In some embodiments, the compound of Formula (I) is selected from:

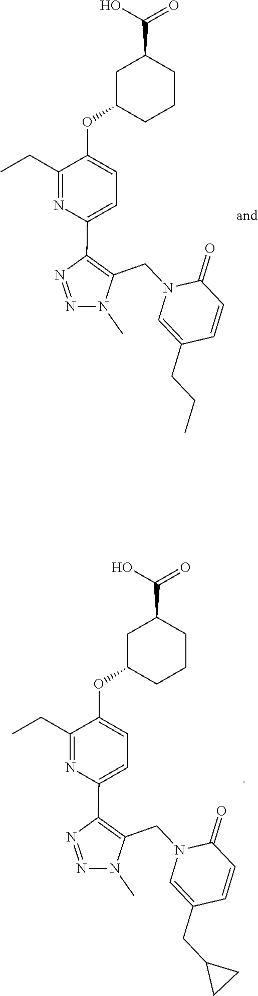 [SMILES: CCCc1ccc(=O)n(Cc2c(-c3ccc(O[C@H]4CCC[C@H](C(=O)O)C4)c(CC)n3)nnn2C)c1 ; CCc1nc(-c2nnn(C)c2Cn2cc(CC3CC3)ccc2=O)ccc1O[C@H]1CCC[C@H](C(=O)O)C1]

In some embodiments, the present invention relates to a compound of Formulae (I), (II), (IIa), (IIb), (IIc), (III), (IIIa), (IIIb), (IIIc), (IV), and (IVa), including each exemplified compound, wherein at least one hydrogen (H) is replaced with deuterium (D). Enriching for deuterium may afford certain therapeutic advantages, such as increasing in vivo half-life or reducing dosage requirements, or may provide a compound useful as a standard for characterization of biological samples. In some other embodiments, a compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In another aspect, the present invention relates to a pharmaceutical composition comprising a compound disclosed herein, and a pharmaceutically acceptable carrier.

In yet another aspect, the present invention relates to a method for treating a disease associated with dysregulation of lysophosphatidic acid receptor 1 (LPAi) in a subject in need thereof, comprising administering an effective amount of a compound disclosed herein to the subject. In some embodiments, the disease is pathological fibrosis (e.g., pulmonary, liver, renal, cardiac, dernal, ocular, or pancreatic fibrosis), idiopathic pulmonary fibrosis (IPF), non-alcoholic steatohepatitis (NASH), non-alcoholic fatty liver disease (NAFLD), chronic kidney disease, diabetic kidney disease, or systemic sclerosis.

In yet another aspect, the present invention relates to a process of making a compound of Formulae (I), (II), (IIa), (IIb), (IIc), (III), (IIIa), (IIIb), (IIIc), (IV), and (IVa), including each exemplified compound and intermediates described herein.

### General Synthetic Methods

The compounds of the present invention can be synthesized using the methods described herein, together with synthetic methods known in the art of synthetic organic chemistry, or by variations thereon as appreciated by those skilled in the art. Preferred methods include, but are not limited to, those exemplary schemes and working examples described below. All substituents are as defined hereinabove unless otherwise indicated. The reactions are performed in a solvent or solvent mixture appropriate to the reagents and materials employed and suitable for the transformations proposed. This will sometimes require a judgment to modify the order of synthetic steps or to select on particular process scheme over another in order to obtain a desired compound of the invention.

It will be recognized that another major consideration in the planning of any synthetic route in this field is the judicious choice of the protecting group used for protection of the reactive functional groups present in the compounds described in this invention. An authoritative account describing the many alternatives to the trained practitioner is by Greene et al., Greene's Protective Groups in Organic Synthesis, Fifth Edition, Wiley (2014). It will also be recognized that the compound names referred to in the descriptions of Schemes 1-3 are for convenience only, and do not necessarily reflect the actual chemical names of those compounds.

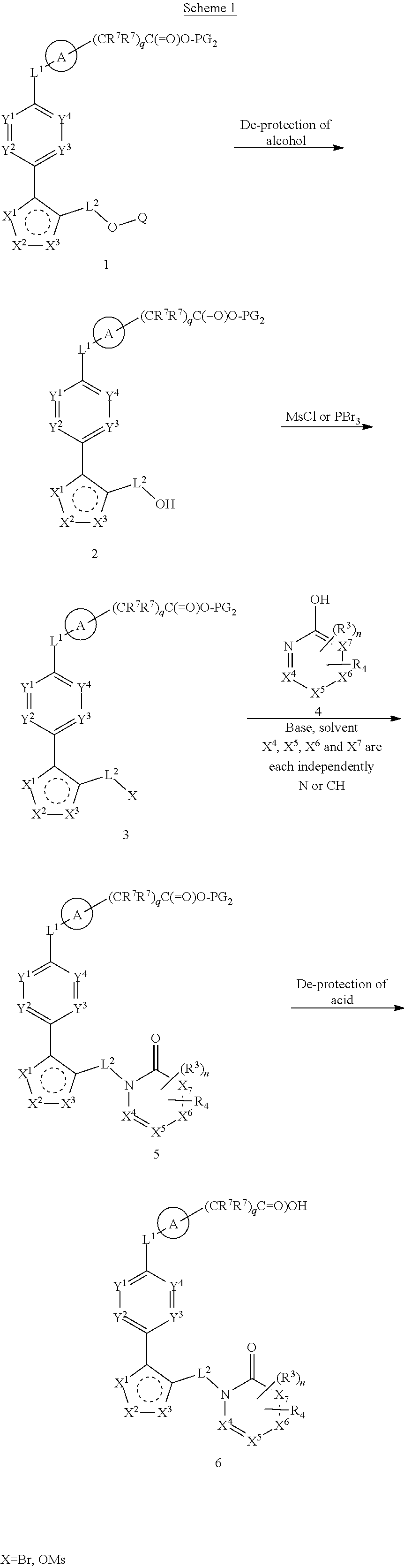

Scheme 1 describes the synthesis of carbonyl azine L²-azole cyclo-acids 6. PG₁ and PG₂ each represents a protecting group, and L² is (CR⁷R⁷)ₚ (e.g., CH₂). Deprotection of the protected hydroxyl-azole 1 provides the hydroxyl-azole 2, which is then reacted with MsCl (or PBr₃) to give the mesylate (or Br) 3. Treatment of the mesylate (or Br) 3 with 2-hydroxy azine 4 in the presence of an appropriate base (e.g. K₂CO₃, nucleophilic substitution reactions) gives the corresponding carbonyl azine azole cyclo-carboxylate 5, which then undergoes ester deprotection to give the desired carbonyl azine L²-azole cyclo-acids 6.

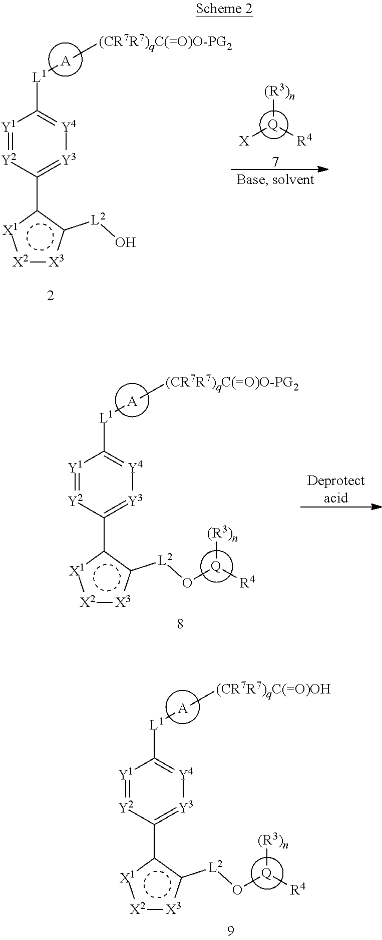

Scheme 2 describes the synthesis of oxy-Q ring N-heteroaryl cyclo-acids 9, wherein Q ring represents a 5-membered heteroaryl or heterocyclyl or 6-membered heteroaryl or heterocyclyl. Base-mediated SNAr reaction of hydroxyl azole 2 with an appropriate halo- or methylsulfonyl-substituted Q ring 7 (X is halo or methylsulfonyl) provides the oxy-Q ring N-heteroaryl cyclo-carboxylate 8, which then undergoes ester deprotection to give the desired oxy-Q ring N-heteroaryl cyclo-acids 9.

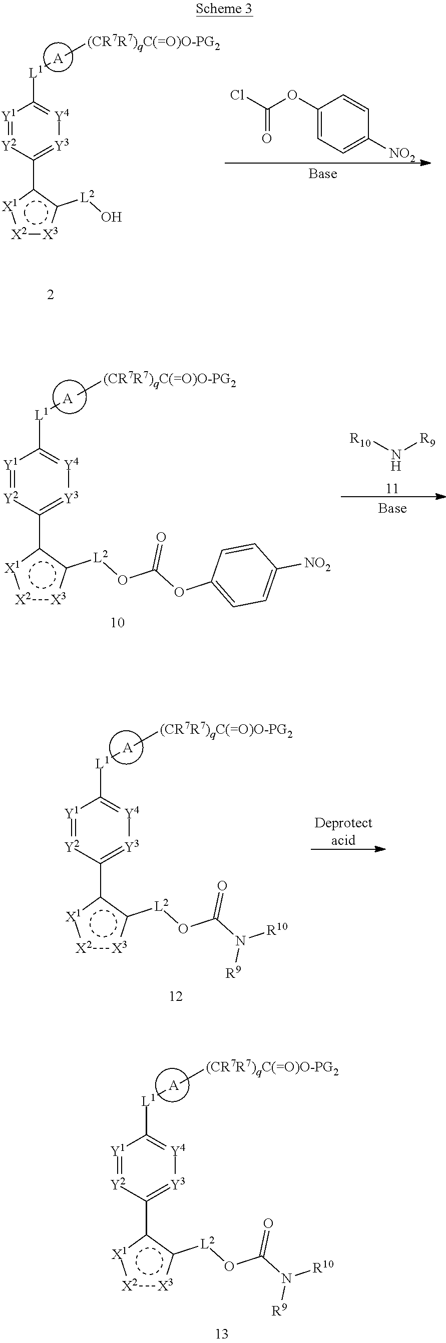

Scheme 3 describes the synthesis of carbamoyloxymethyl azole N-heteroaryl cyclo-acids 13. The hydroxylmethyl azole 4 is reacted with 4-nitrophenyl chloroformate in the presence of an appropriate base to give the corresponding 4-nitrophenyl carbonate 10, which is then reacted with an amine 11 in the presence of an appropriate base to give the carbamate 12. Subsequent deprotection of ester 12 provides the carbamoyloxymethyl azole N-heteroaryl cyclo-acids 13.

### Pharmaceutical Compositions and Methods

The compounds utilized in the methods described herein may be formulated together with a pharmaceutically acceptable carrier or adjuvant into pharmaceutically acceptable compositions prior to be administered to a subject. In another embodiment, such pharmaceutically acceptable compositions further comprise additional therapeutic agents in amounts effective for achieving a modulation of disease or disease symptoms, including those described herein.

The term “pharmaceutically acceptable carrier or adjuvant” refers to a carrier or adjuvant that may be administered to a subject, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

The pharmaceutical compositions described above comprising a compound of formulae (I)—(IV) may further comprise another therapeutic agent useful for treating a disease associated with dysregulation of lysophosphatidic acid receptor 1 (LPAi). In particular, such combination may be useful for treating pathological fibrosis (e.g., pulmonary, liver, renal, cardiac, dernal, ocular, or pancreatic fibrosis), idiopathic pulmonary fibrosis (IPF), non-alcoholic steatohepatitis (NASH), non-alcoholic fatty liver disease (NAFLD), chronic kidney disease, diabetic kidney disease, or systemic sclerosis.

All publications, patents, and patent applications mentioned in this specification are herein incorporated by reference to the same extent as if each individual publication, patent, or patent application was specifically and individually indicated to be incorporated by reference. In case of conflict, the present application, including any definitions herein, will control.

The examples herein are provided to illustrate advantages of the present invention and to further assist a person of ordinary skill in the art with preparing or using the compounds of the present invention or salts, pharmaceutical compositions, derivatives, solvates, metabolites, prodrugs, racemic mixtures or tautomeric forms thereof. The examples herein are also presented in order to more fully illustrate the preferred aspects of the present invention. The examples should in no way be construed as limiting the scope of the present invention, as defined by the appended claims. The examples can include or incorporate any of the variations, aspects or aspects of the present invention described above. The variations, aspects or aspects described above may also further each include or incorporate the variations of any or all other variations, aspects or aspects of the present invention.

### Examples

Abbreviations used herein are as follows:

| Abbrv. | Full Name | Abbrv. | Full Name |
|---|---|---|---|
| anhy. | anhydrous | aq. | aqueous |
| min | minute(s) | satd. | saturated |
| mL | milliliter | hrs | hours |
| mmol | millimole(s) | mol | mole(s) |
| MS | mass spectrometry | NMR | nuclear magnetic resonance |
| TLC | thin layer chromatography | HPLC | high-performance liquid |
| | | | chromatography |
| LCMS | Liquid chromatography-mass | PPTS | Pyridinium p-Toluenesulfonate |
| | spectrometry | | |
| DCE | 1,2-dichloroethane | CHCl₃ | chloroform |
| DCM | dichloromethane | DMF | dimethylformamide |
| Et₂O | diethyl ether | EtOH | ethyl alcohol |
| EtOAc | ethyl acetate | MeOH | methyl alcohol |
| MeCN | acetonitrile | PE | petroleum ether |
| THF | tetrahydrofuran | DMSO | dimethyl sulfoxide |
| AcOH | acetic acid | HCl | hydrochloric acid |
| H₂SO₄ | sulfuric acid | NH₄Cl | ammonium chloride |
| KOH | potassium hydroxide | NaOH | sodium hydroxide |
| K₂CO₃ | potassium carbonate | Na₂CO₃ | sodium carbonate |
| TFA | trifluoroacetic acid | Na₂SO₄ | sodium sulfate |
| NaBH₄ | sodium borohydride | NaHCO₃ | sodium bicarbonate |
| LiHMDS | lithium | NaBH₄ | sodium borohydride |
| | hexamethyldisilylamide | | |
| Et₃N or | Triethylamine | Py or Pyr | pyridine |
| TEA | | | |
| TBAF | Tetrabutylammonium fluoride | MsCl | Methanesulfonyl chloride |
| BnBr | Benzyl bromide | DHP | 3,4-Dihydro-2H-pyran |
| Jones | Chromium trioxide solution in | Cp*RuCl(PPh₃)₂ | Pentamethylcyclopentadienylbis |
| Reagent | sulfuric acid | | (triphenylphosphine)ruthenium(II) |
| | | | chloride |
| Pd(PPh₃)₄ | Tetrakis(triphenylphosphine) | Pd₂(dba)₃ | Tris(dibenzylideneacetone)dipalladium |
| | palladium | | |
| Cbz | carbobenzyloxy | m-CPBA | 3-Chloroperoxybenzoic acid |
| Dess- | 1,1,1-Triacetoxy-1,1-Dihydro- | DIAD | Diisopropyl azodicarboxylate |
| Martin | 1,2-Benziodoxol-3(1H)-On | | |
| DMAP | 4-(dimethylamino)pyridine | DIPEA | N,N-diisopropylethylamine |
| TMSCHN2 | (Trimethylsilyl)diazomethane | TMSCH2N3 | Trimethylsilylmethyl azide |

| | |
|---|---|
| Ruphos | 2-Dicyclohexylphosphino-2′,6′-diisopropoxybiphenyl |
| RuPhosPd | Methanesulfonato(2-dicyclohexylphosphino-2′,6′-di-i-propoxy-1,1′- |
| G3 | biphenyl)(2′-amino-1,1′-biphenyl-2-yl)palladium(II) |

### General Conditions and Procedures

In the following examples, the chemical reagents were purchased from commercial sources (such as Alfa, Acros, Sigma Aldrich, TCI and Shanghai Chemical Reagent Company), and used without further purification. THE was continuously refluxed and freshly distilled from sodium and benzophenone under nitrogen, dichloromethane was continuously refluxed and freshly distilled from CaH₂ under nitrogen.

Flash chromatography was performed on an Ez Purifier III via column with silica gel particles of 200-300 mesh. Analytical and preparative thin layer chromatography plates (TLC) were HSGF 254 (0.15-0.2 mm thickness, Shanghai Anbang Company, China). Nuclear magnetic resonance (NMR) spectra were recorded using Brucker AMX-300 or AMX-400 NMR (Brucker, Switzerland) at around 20-30° C. unless otherwise specified. The following abbreviations are used: s, singlet; d, doublet; t, triplet; q, quartet; m, multiplet; dd, doublet of doublets; ddd, doublet of doublet of doublet; dt, doublet of triplets; bs, broad signal. Chemical shifts were reported in parts per million (ppm, 6) downfield from tetramethylsilane. Mass spectra were run with electrospray ionization (ESI) from a Waters LCT TOF Mass Spectrometer (Waters, USA). Compound purification was carried out as needed using a variety of traditional methods including, but not limited to, preparative chromatography under acidic, neutral, or basic conditions using either normal phase or reverse phase HPLC or flash columns or Prep-TLC plates.

Preparative HPLC: unless otherwise described, the compounds were purified using a WATERS Fractionlynx system equipped with a YMC Pack Pro ds Column (5 μm, 120A, 50×20 mm) and the following solvent system: H₂O, AcCN, and 2% TFA in H₂O. Specific elution gradients were based on the retention times obtained with an analytical LC/MS, however, in general all elution gradients of H₂O and MeCN were run over a 7 minute run time with a flow rate of 35 mL/min. An autoblend method was used to ensure a concentration of 0.1% TFA throughout each run. Specific elution gradients were based on the retention times obtained with an analytical LC/MS, however, in general, all elution gradients of H₂O and MeCN were run over at 8 minute run time with a flow rate of 50 mL/min.

Analytical LC/MS: analytical LC/MS was performed on a WATERS Acquity UPLC/MS instrument equipped with a ACQUITY UPLC BEH Cis Column (2.1×50 mm, 1.7 μm), a column temperature of 45° C. and using the following solvent system: Solvent A: 0.1% HCOOH in H₂O; and Solvent B: 0.1% HCOOH in AcCN. All compounds were run using the same elution gradient, i.e., 5% to 95% Solvent B over a 1.5 min run time with a flow rate of 0.6 mL/min.

Preparative Chiral SFC Separation: stereoisomer mixtures were separated using a Berger Minigram SFC instrument on one of the following columns: ChiralPak AS-H (10×250 mm), ChiralPak IA (10×250 mm), ChiralPak AD-H (21×250 mm), Phenomenex Lux-2 (21.2×250 mm), or ChiralPak IC (10×250 mm); eluting with either 0.1% diethylamine in MeOH/CO₂, or 0.1% diethylamine in EtOH/CO₂ or 0.1% diethylamine in isopropanol/CO₂ with a flow rate of 2.5 mL/min and a column temperature of 35° C.

Analytical Chiral SFC Separation: stereoisomer mixtures or single enantiomers were analyzed using a JASCO analytical SFC instrument on one of the following columns: ChiralPak AS-H (4.6×250 mm), ChiralPak IA (4.6×250 mm), ChiralPak AD-H (4.6×250 mm), Phenomenex Lux-2 (4.6×250 mm), or ChiralPak IC (4.6×250 mm); eluting with either 0.1% diethylamine in MeOH/CO₂, or 0.1% diethylamine in EtOH/CO₂ or 0.1% diethylamine in isopropanol/CO₂, with a flow rate of 6.0 imL/min and a column temperature of 35° C.

### Intermediate 1: 3-bromo-2-ethyl-6-(1-methyl-5-(((tetrahydro-2H-pyran-2-yl)oxy)methyl)-1H-1,2,3-triazol-4-yl)pyridine

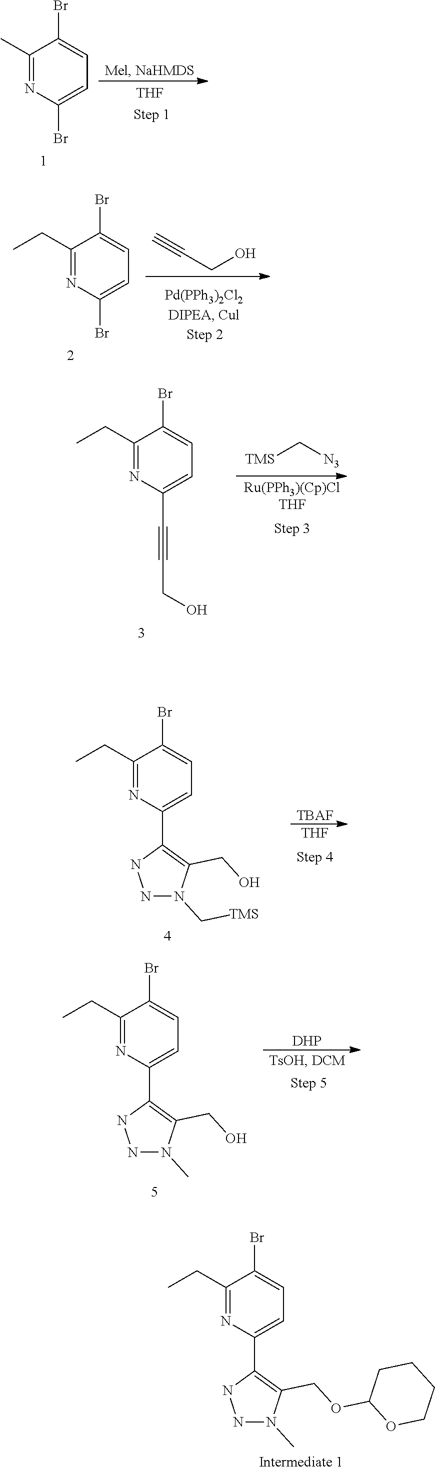

### Step 1: 3,6-dibromo-2-ethylpyridine

To a solution of 3,6-dibromo-2-methylpyridine (75 g, 0.299 mol) in THE (1 L) was added NaHMDS (180 mL, 0.36 mol, 2M in THF) drop-wisely at −50° C. and the mixture was stirred at this temperature for 30 mins. Mel (46.5 mL, 0.75 mol) was added to the above mixture and the resulting mixture was stirred at −50° C. to room temperature for 16 hrs. The reaction mixture was quenched with saturated aq. NH₄Cl solution (500 mL) at 0° C. and extracted with EtOAc (2×500 mL). The combined organic layers were washed with brine, dried over Na₂SO₄, filtered and concentrated under reduced pressure to dryness. The residue was purified by silica column chromatography (PE:DCM=300:1 to 200:1) to give the title compound (47 g, 59.1% yield) as yellow oil. ¹H NMR (400 MHz, CD₃OD) δ 7.61-7.59 (d, J=8 Hz, 1H), 7.61-7.59 (d, J=8.4 Hz, 1H), 2.94-2.88 (q, 2H), 1.38-1.27 (t, 3H).

### Step 2: 3-(5-bromo-6-ethylpyridin-2-yl)prop-2-yn-1-ol

To a solution of 3,6-dibromo-2-ethylpyridine (47 g, 0.177 mol) in THE (0.65 L) was added prop-2-yn-1-ol (11.9 g, 0.212 mol), CuI (3.4 g, 17.7 mmol), DIPEA (35.1 mL, 0.212 mol) and Pd(PPh₃)₂Cl₂ (12.4 g, 17.7 mmol), the mixture was degassed under N₂ atmosphere for three times and stirred under N₂ atmosphere at r.t. for 16 hrs. The mixture was diluted with EtOAc (500 mL) and filtered. The filtrate was washed with water and brine, dried over Na₂SO₄, filtered and concentrated to dryness. The residue was purified by silica column chromatography (DCM:EtOAc=10:1 to 4:1) to give the title compound (40 g, 94.1% yield) as brown solid. LC/MS (ESI) m/z: 240/242 (M+H)⁺.

### Step 3: (4-(5-bromo-6-ethylpyridin-2-yl)-1-((trimethylsilyl)methyl)-1H-1,2,3-triazol-5-yl)methanol

To a solution of intermediate 3-(5-bromo-6-ethylpyridin-2-yl)prop-2-yn-1-ol (20 g, 0.083 mol) in THF (400 mL) was added Chlorocyclopentadienylbis(triphenylphosphine)ruthenium(II) (3.5 g, 4.16 mmol) and TMS-methyl azide (14 g, 0.108 mol) at 0° C. under N₂ atmosphere. The mixture was degassed under N₂ atmosphere for three times and stirred at room temperature for 16 hrs. The mixture was diluted with EtOAc (500 mL) and filtered. The filtrate was concentrated to dryness to give crude product, which was triturated with PE/EtOAc (1000 mL, 10/1 v/v) to give the title compound (28 g, 91.3% yield) as white solid. ¹H NMR (400 MHz, CDCl₃) δ 8.03-8.01 (d, J=8.4 Hz, 1H), 7.95-7.93 (d, J=8.4 Hz, 1H), 6.77-6.74 (t, J=6.8 Hz, 1H), 4.79-4.78 (d, J=6.8 Hz, 1H), 3.80 (s, 3H), 3.03-3.01 (q, 1H), 1.34-1.30 (t, J=7.6 Hz, 3H), 0.20 (s, 9H).

### Step 4: (4-(5-bromo-6-ethylpyridin-2-yl)-1-methyl-1H-1,2,3-triazol-5-yl)methanol

To a solution of (4-(5-bromo-6-ethylpyridin-2-yl)-1-((trimethylsilyl)methyl)-1H-1,2,3-triazol-5-yl)methanol (25 g, 67.7 mmol) in THE (300 mL) was added TBAF·3H₂O (25.6 g, 81.2 mmol) and the mixture was stirred at room temperature overnight. The mixture was diluted with EtOAc (500 mL), washed with saturated aq. NH₄Cl solution (3×50 mL) and brine, dried over Na₂SO₄, filtered and concentrated to dryness. The residue was purified by silica column chromatography (DCM:EtOAc=8:1 to 4:1) to give the title compound (19 g, 94.4% yield) as gray solid. LC/MS (ESI) m/z: 297/299 (M+H)⁺.

### Step 5: 3-bromo-2-ethyl-6-(1-methyl-5-(((tetrahydro-2H-pyran-2-yl)oxy)methyl)-1H-1,2,3-triazol-4-yl)pyridine

To the mixture of (4-(5-bromo-6-ethylpyridin-2-yl)-1-methyl-1H-1,2,3-triazol-5-yl) methanol (25 g, 0.084 mol) and TosOH (2.2 g, 12.6 mmol) in DCM (300 mL) was added DHP (10.6 g, 0.126 mol) at 0° C. and the mixture was stirred at room temperature for 16 hrs. The mixture was washed with water and brine, dried over Na₂SO₄, filtered and concentrated to dryness. The residue was purified by silica column chromatography (DCM:EtOAc=8:1) to give the title compound (30 g, 93.6% yield) as yellow solid. LC/MS (ESI) m/z: 381/383 (M+H)⁺. ¹H NMR (400 MHz, CDCl₃) δ 7.91 (d, J=8.3 Hz, 1H), 7.86 (d, J=8.3 Hz, 1H), 5.36 (dd, J=36.5, 12.8 Hz, 2H), 4.75-4.67 (m, 1H), 4.16 (s, 3H), 3.88-3.80 (m, 1H), 3.55-3.46 (m, 1H), 3.00 (q, J=7.5 Hz, 2H), 1.84-1.67 (m, 2H), 1.65-1.58 (m, 2H), 1.54-1.45 (m, 2H), 1.34 (t, J=7.5 Hz, 3H).

### Intermediate 2: 3-bromo-2-methyl-6-(1-methyl-5-(((tetrahydro-2H-pyran-2-yl)oxy)methyl)-1H-1,2,3-triazol-4-yl)pyridine

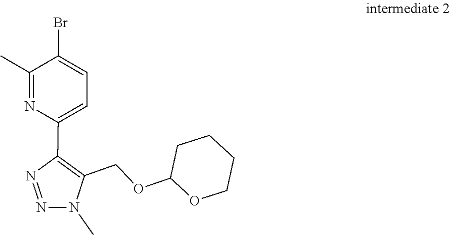 [SMILES: Cc1nc(-c2nnn(C)c2COC2CCCCO2)ccc1Br]

Intermediate 2 was synthesized according the same sequence as was used for the synthesis of intermediate 1. LC/MS (ESI) m/z: 367/369 (M+H)⁺. ¹H NMR (400 MHz, CDCl₃) δ 7.73 (d, J=8.3 Hz, 1H), 7.69 (d, J=8.3 Hz, 1H), 5.15 (q, J=12.7 Hz, 2H), 4.61-4.54 (m, 1H), 3.99 (s, 3H), 3.74-3.64 (m, 1H), 3.38-3.30 (m, 1H), 2.51 (s, 3H), 1.66-1.50 (m, 2H), 1.46-1.40 (m, 2H), 1.39-1.32 (m, 2H).

### Intermediate 3: 2-methyl-6-(1-methyl-5-(((tetrahydro-2H-pyran-2-yl)oxy)methyl)-1H-1,2,3-triazol-4-yl)-3-(4,4,5,5-tetramethyl-1,3,2-dioxaborolan-2-yl)pyridine

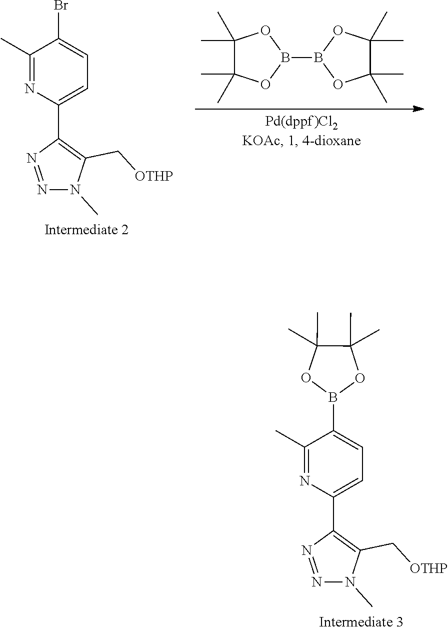

To a solution of 3-bromo-2-methyl-6-(1-methyl-5-(((tetrahydro-2H-pyran-2-yl)oxy)methyl)-1H-1,2,3-triazol-4-yl)pyridine (1 g, 2.72 mmol, intermediate 2) and 4,4,4′,4′,5,5,5′,5′-octamethyl-2,2′-bi(1,3,2-dioxaborolane) (840 mg, 3.3 mmol) in 1,4-dioxane (20 mL) was added Pd(dppf)Cl₂ (99 mg, 0.136 mmol) and KOAc (400 mg, 4.08 mmol), the mixture was degassed under N₂ atmosphere for three times and stirred at 100° C. for 16 hrs. The mixture was diluted with EtOAc (50 mL), washed with water and brine, dried over Na₂SO₄, filtered and concentrated to dryness. The residue was purified by flash chromatography (silica gel, 0-20% EtOAc in PE) to give the title compound (890 mg, 78.9% yield) as light yellow solid. LC/MS (ESI) m/z: 415 (M+H)⁺.

### Intermediate 4: 2-methyl-6-(1-methyl-5-(((tetrahydro-2H-pyran-2-yl)oxy)methyl)-1H-1,2,3-triazol-4-yl)-3-(4,4,5,5-tetramethyl-1,3,2-dioxaborolan-2-yl)pyridine

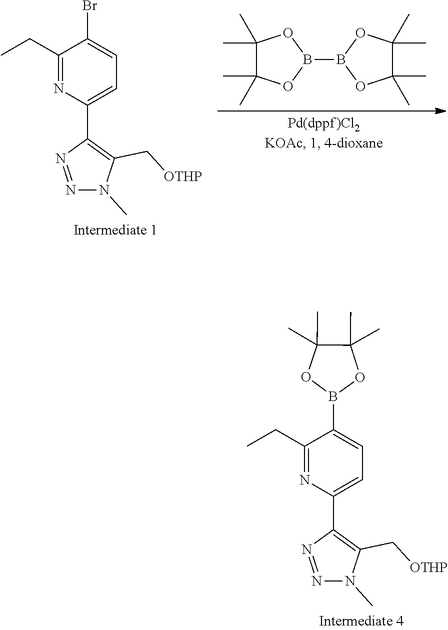

To a solution of 3-bromo-2-methyl-6-(1-methyl-5-(((tetrahydro-2H-pyran-2-yl)oxy)methyl)-1H-1,2,3-triazol-4-yl)pyridine (2 g, 5.25 mmol) and 4,4,4′,4′,5,5,5′,5′-octamethyl-2,2′-bi(1,3,2-dioxaborolane) (1.73 g, 6.82 mmol) in 1,4-dioxane (30 mL) was added Pd(dppf)Cl₂ (200 mg, 0.27 mmol) and KOAc (800 mg, 8.2 mmol), the mixture was degassed under N₂ atmosphere for three times and stirred at 100° C. for 16 hrs. The mixture was diluted with EtOAc (50 mL), washed with water and brine, dried over Na₂SO₄, filtered and concentrated to dryness. The residue was purified by flash chromatography (silica gel, 0-20% EtOAc in PE) to give the title compound (1.91 g, 84.9% yield) as light yellow solid. LC/MS (ESI) m/z: 429 (M+H)⁺.

### Intermediate 5: 4-(cyclopropylmethyl)-3-methylpyridin-2(1H)-one

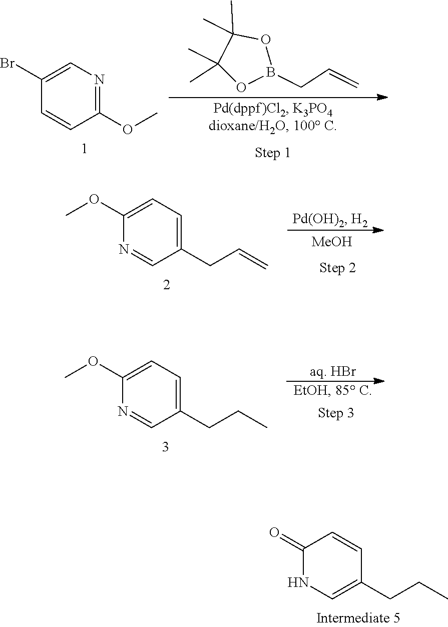

### Step 1: 5-allyl-2-methoxypyridine

To a solution of 5-bromo-2-methoxypyridine (4.2 g, 22.3 mmol) and 2-allyl-4,4,5,5-tetramethyl-1,3,2-dioxaborolane (5.63 g, 33.5 mmol) in 1,4-dioxane (42 mL) and water (8.4 mL) were added K₃PO₄ (14.23 g, 67.0 mmol), Pd(dppf)Cl₂ (1.63 g, 2.23 mmol) under N₂ atmosphere and the mixture was degassed under N₂ atmosphere for three times and stirred under N₂ atmosphere at 100° C. for 2 hrs. The mixture was diluted EtOAc (50 mL), washed with water and brine, dried over anhydrous Na₂SO₄, filtered and concentrated to dryness. The residue was purified by flash chromatography (eluted with PE:EtOAc=50:1 to 10:1) to give the title compound (3 g, 90% yield) as light yellow oil. LC/MS (ESI) m/z: 150 (M+H)⁺.

### Step 2: 2-methoxy-5-propylpyridine

To a solution of 5-allyl-2-methoxypyridine (3 g, 20.1 mmol) in MeOH (30 mL) was added Pd(OH)₂/C (0.28 g, 10% wt) under N₂ atmosphere. After addition, the mixture was degassed under N₂ atmosphere for three times and stirred under a H₂ balloon at 25° C. for 16 hrs. The mixture was filtered and the filtrate was concentrated to dryness to give the title compound (2.74 g, 90.1% yield) as colorless oil, which was used in next step directly. LC/MS (ESI) m/z: 152 (M+H)⁺.

### Step 3: 5-propylpyridin-2(1H)-one (4)

To a solution of 2-methoxy-5-propylpyridine (2.74 g, 18.12 mmol) in EtOH (27 mL) was added aq. HBr (27 mL, 40% wt) and the mixture was stirred at 85° C. for 16 hrs. The mixture was quenched with saturated aq. NaHCO₃ solution and extracted with DCM (2×20 mL). The combined organic layers were washed with brine, dried over anhydrous Na₂SO₄, filtered and concentrated to dryness. The residue was purified by flash chromatography (eluted with PE:EtOAc=10:1 to 1:1) to give the title compound (723 mg, 29.1% yield) as white solid. ¹H NMR (400 MHz, CDCl₃) δ 13.21 (s, 1H), 7.35 (dd, J=9.2, 2.5 Hz, 1H), 7.14 (d, J=2.2 Hz, 1H), 6.55 (d, J=9.2 Hz, 1H), 2.35 (t, J=7.6 Hz, 1H), 1.60-1.48 (m, 1H), 0.92 (t, J=7.2 Hz, 2H). LC/MS (ESI) m/z: 138 (M+H)⁺.

### Intermediate 6: 2-chloro-4-cyclobutylpyrimidine

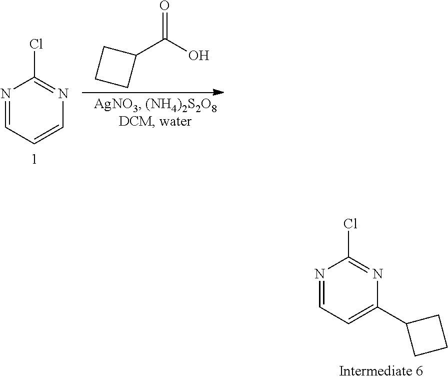

To a mixture of 2-chloropyrimidine (3 g, 26.2 mmol) and cyclobutanecarboxylic acid (2.3 mL, 23.6 mmol) in DCM (15 mL) and water (15 mL) was added AgNO₃ (890 mg, 5.2 mmol), followed by in portions addition of (NH₄)₂S₂O₈ (6.0 g, 26.2 mmol) at room temperature and the mixture was stirred at 25° C. for 16 hrs. The mixture was diluted with DCM (50 mL), washed with water and brine, dried over anhydrous Na₂SO₄, filtered and concentrated to dryness. The residue was purified by flash chromatography (PE:EtOAc=5:1 to 1:1) to give the title compound (2.2 g, 49.8% yield) as colorless oil. LC/MS (ESI) m/z: 169 (M+H)⁺. ¹H NMR (400 MHz, CDCl₃) δ 8.49 (d, J=5.2 Hz, 1H), 7.11 (d, J=5.2 Hz, 1H), 3.70-3.53 (m, 1H), 2.41-2.30 (m, 4H), 2.15-2.03 (m, 1H), 1.99-1.92 (m, 1H).

### Example 1: 2-[(3S)-1-[6-(5-{[(4-cyclobutylpyrimidin-2-yl)oxy]methyl}-1-methyl-1H-1,2,3-triazol-4-yl)-2-ethylpyridin-3-yl]pyrrolidin-3-yl]acetic acid

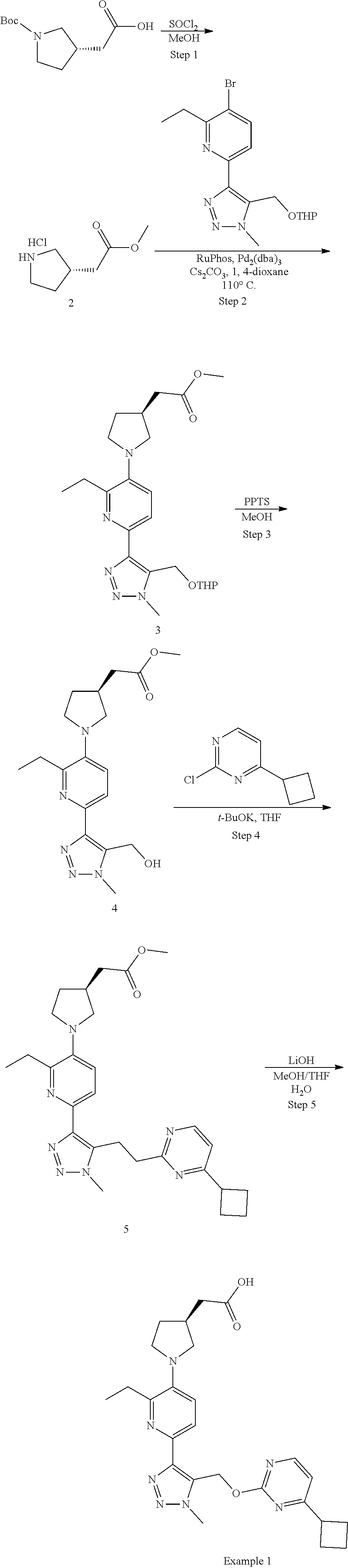

### Step 1: methyl 2-[(3S)-pyrrolidin-3-yl]acetate hydrochloride

To a solution of 2-[(3S)-1-[(tert-butoxy)carbonyl]pyrrolidin-3-yl]acetic acid (1 g, 4.36 mmol) in MeOH (10 mL) was added SOCl₂ (2 mL, 27.6 mmol) drop-wisely at 0° C. under N₂ atmosphere and the reaction was stirred at r.t. for 16 hrs. The reaction mixture was concentrated under reduced pressure to dryness to give title compound (720 mg, 92% yield) as light yellow solid. LC/MS (ESI) m/z: 144 (M+H)⁺.

### Step 2: methyl 2-[(3S)-1-(2-ethyl-6-{1-methyl-5-[(oxan-2-yloxy)methyl]-1H-1,2,3-triazol-4-yl}pyridin-3-yl)pyrrolidin-3-yl]acetate

To a solution of 3-bromo-2-ethyl-6-{1-methyl-5-[(oxan-2-yloxy)methyl]-1H-1,2,3-triazol-4-yl}pyridine (300 mg, 0.79 mmol) in 1,4-dioxane (20 mL) was added methyl 2-[(3S)-pyrrolidin-3-yl]acetate (169 mg, 1.18 mmol) followed by addition of Cs₂CO₃ (769 mg, 2.36 mmol), Pd₂(dba)₃ (64 mg, 0.08 mmol) and Ru-phos (37 mg, 0.08 mmol) under N₂ atmosphere. The mixture was degassed under N₂ atmosphere for three times and stirred at 120° C. for 16 hrs. The reaction mixture was filtered through a Celite pad and the filtrate was concentrated to dryness. The residue was purified by flash chromatography (silica gel, 0-50% of EtOAc in PE) to give the title compound (260 mg, 74.5% yield) as yellow solid. LC/MS (ESI) m/z: 444 (M+H)⁺.

### Step 3: methyl 2-[(3S)-1-{2-ethyl-6-[5-(hydroxymethyl)-1-methyl-1H-1,2,3-triazol-4-yl]pyridin-3-yl}pyrrolidin-3-yl]acetate

To a solution of methyl 2-[(3S)-1-(2-ethyl-6-{1-methyl-5-[(oxan-2-yloxy)methyl]-1H-1,2,3-triazol-4-yl}pyridin-3-yl)pyrrolidin-3-yl]acetate (260 mg, 0.58 mmol) in MeOH (15 mL) was added PPTS (295 mg, 1.17 mmol). The reaction mixture was stirred at 50° C. for 16 hrs. The mixture was diluted with EtOAc, washed with water and brine, dried over Na₂SO₄, filtered and concentrated to dryness. The residue was purified by silica gel column chromatography (0˜80% of EtOAc in PE) to give the title compound (130 mg, 61.7% yield) as yellow solid. LC/MS (ESI) m/z: 360 (M+H)⁺.

### Step 4: methyl 2-[(3S)-1-[6-(5-{[(4-cyclobutylpyrimidin-2-yl)oxy]methyl}-1-methyl-1H-1,2,3-triazol-4-yl)-2-ethylpyridin-3-yl]pyrrolidin-3-yl]acetate

To a solution of methyl 2-[(3S)-1-{2-ethyl-6-[5-(hydroxymethyl)-1-methyl-1H-1,2,3-triazol-4-yl]pyridin-3-yl}pyrrolidin-3-yl]acetate (60 mg, 0.17 mmol) in THE (5 mL) was added t-BuOK (0.37 mL, 0.33 mmol, 1.0 M in THF) drop-wisely at 0° C. The reaction mixture was stirred at 0° C. for 30 min until the disappearance of the starting material was confirmed by TLC analysis. The reaction was quenched with saturated aq. NH₄Cl solution at 0° C. and the mixture was extracted with EtOAc (2×10 mL). The combined organic layers were washed with brine, dried over anhydrous Na₂SO₄, filtered and concentrated to dryness. The residue was purified by flash chromatography (silica gel, 0˜70% of EtOAc in PE) to give the title compound (60 mg, 73.1% yield) as white solid. LC/MS (ESI) m/z: 492 (M+H)⁺.

### Step 5: 2-[(3S)-1-[6-(5-{[(4-cyclobutylpyrimidin-2-yl)oxy]methyl}-1-methyl-1H-1,2,3-triazol-4-yl)-2-ethylpyridin-3-yl]pyrrolidin-3-yl]acetic acid

To a solution of methyl 2-[(3S)-1-[6-(5-{[(4-cyclobutylpyrimidin-2-yl)oxy]methyl}-1-methyl-1H-1,2,3-triazol-4-yl)-2-ethylpyridin-3-yl]pyrrolidin-3-yl]acetate (70 mg, 0.14 mmol) in THE (4 mL)/H₂O (1 mL)/MeOH (1 mL) was added LiOH·H₂O (60 mg, 1.42 mmol) and the reaction was stirred at r.t. for 2 hrs. Volatiles were removed under vacuum and the residue was diluted with H₂O (5 mL). The mixture was adjusted with 1N aq. HCl to pH˜4 and extracted with EtOAc (3×10 mL). The combined organic layers were washed with brine, dried over anhydrous Na₂SO₄, filtered and concentrated to dryness. The residue was purified by prep-HPLC (C18, 5-95%, MeCN in H₂O with 0.1% HCOOH) to give the title compound (25 mg, 36.8% yield) as white solid. LC/MS (ESI) m/z: 478 (M+H)⁺. ¹H NMR (400 MHz, CD₃OD) δ 8.41 (d, J=5.1 Hz, 1H), 7.76 (d, J=8.4 Hz, 1H), 7.32 (d, J=8.5 Hz, 1H), 6.98 (d, J=5.1 Hz, 1H), 6.23-6.14 (m, 2H), 4.22 (s, 3H), 3.57-3.47 (m, 1H), 3.40-3.33 (m, 2H), 3.28-3.21 (m, 1H), 3.09-3.02 (m, 1H), 2.88-2.78 (m, 2H), 2.72-2.60 (m, 1H), 2.49 (d, J=7.3 Hz, 2H), 2.28-2.18 (m, 5H), 2.08-1.96 (m, 1H), 1.88-1.76 (m, 1H), 1.74-1.64 (m, 1H), 1.22 (t, J=7.4 Hz, 3H).

### Example 2: 2-(1-(2-methyl-6-(1-methyl-5-((2-oxo-5-propylpyridin-1(2H)-yl)methyl)-1H-1,2,3-triazol-4-yl)pyridin-3-yl)pyrrolidin-3-yl)acetic acid

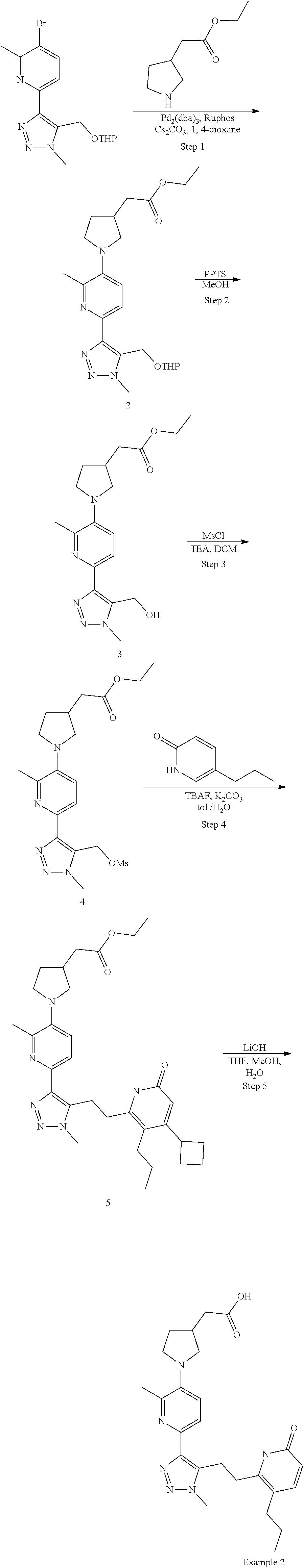

### Step 1: ethyl 2-(1-(2-methyl-6-(1-methyl-5-(((tetrahydro-2H-pyran-2-yl)oxy)methyl)-1H-1,2,3-triazol-4-yl)pyridin-3-yl)pyrrolidin-3-yl)acetate

To a solution of 3-bromo-2-methyl-6-{1-methyl-5-[(oxan-2-yloxy)methyl]-1H-1,2,3-triazol-4-yl}pyridine (0.3 g, 0.82 mmol) in 1,4-dioxane (30 mL) was added ethyl 2-(pyrrolidin-3-yl)acetate (0.13 g, 0.82 mmol) followed by Cs₂CO₃ (0.80 g, 2.45 mmol), Pd₂(dba)₃ (0.07 g, 0.08 mmol) and Ru-phos (0.04 g, 0.08 mmol) under N₂ atmosphere with stirring and the mixture was stirred at 120° C. for 16 hrs. The reaction mixture was filtered through a Celite pad and the filtrate was concentrated to dryness to give a residue, which was purified by flash chromatography (silica gel, 0-50% of EtOAc in PE) to give the title compound (280 mg, 77.3% yield) as yellow solid.

### Step 2: ethyl 2-(1-(6-(5-(hydroxymethyl)-1-methyl-1H-1,2,3-triazol-4-yl)-2-methylpyridin-3-yl)pyrrolidin-3-yl)acetate

To a solution of ethyl 2-(1-(2-methyl-6-(1-methyl-5-(((tetrahydro-2H-pyran-2-yl)oxy)methyl)-1H-1,2,3-triazol-4-yl)pyridin-3-yl)pyrrolidin-3-yl)acetate (260 mg, 0.59 mmol) in MeOH (10 mL) was added PPTS (147 mg, 0.59 mmol) and the reaction mixture was stirred at 60° C. for 2 hrs. The mixture was concentrated to dryness and the residue was diluted with EtOAc. The organic layers were washed with brine, dried over Na₂SO₄, filtered and concentrated to dryness. The residue was purified with silica gel column chromatography (0˜50% of EtOAc in PE) to give the title compound (170 mg, 80.7% yield) as yellow solid. LC/MS (ESI) m/z: 360 (M+H)⁺.

### Step 3: ethyl 2-(1-(2-methyl-6-(1-methyl-5-(((methylsulfonyl)oxy)methyl)-1H-1,2,3-triazol-4-yl)pyridin-3-yl)pyrrolidin-3-yl)acetate

To a stirred solution of ethyl 2-(1-(6-(5-(hydroxymethyl)-1-methyl-1H-1,2,3-triazol-4-yl)-2-methylpyridin-3-yl)pyrrolidin-3-yl)acetate (50 mg, 0.14 mmol) in DCM (5 mL) was added MsCl (38 mg, 0.28 mmol) followed by TEA (42 mg, 0.42 mmol) at 0° C. under N₂ atmosphere. The reaction mixture was stirred at r.t. for 30 min until the disappearance of the starting material was confirmed by TLC analysis. The reaction was quenched with saturated aq. NaHCO₃ solution at 0° C. and the mixture was extracted with DCM (2×20 mL). The combined organic layers were washed with brine, dried over anhydrous Na₂SO₄, filtered and concentrated to dryness to give the title compound (60 mg, 98.6% yield) as yellow oil, which was used directly in the next step. LC/MS (ESI) m/z: 438 (M+H)⁺.

### Step 4: ethyl 2-(1-(2-methyl-6-(1-methyl-5-((2-oxo-5-propylpyridin-1(2H)-yl)methyl)-1H-1,2,3-triazol-4-yl)pyridin-3-yl)pyrrolidin-3-yl)acetate

To a mixture of ethyl 2-(1-(2-methyl-6-(1-methyl-5-(((methylsulfonyl)oxy)methyl)-1H-1,2,3-triazol-4-yl)pyridin-3-yl)pyrrolidin-3-yl)acetate (50 mg, 0.114 mmol) and 5-propyl-1,2-dihydropyridin-2-one (19 mg, 0.14 mmol) in toluene (5 mL) and H₂O (1 mL) were added K₂CO₃ (47 mg, 0.34 mmol) and TBAF (3 mg, 0.011 mmol). The reaction was stirred at 100° C. for 16 hrs. The reaction mixture was concentrated under reduced pressure to dryness. The residue was diluted with EtOAc (10 mL), washed with water and brine, dried over anhydrous Na₂SO₄, filtered and concentrated dryness to give the title compound (50 mg, ⁹1.4% yield) as yellow solid, which was used directly in the next step. LC/MS (ESI) m/z: 479 (M+H)⁺.

### Step 5: 2-(1-(2-methyl-6-(1-methyl-5-((2-oxo-5-propylpyridin-1(2H)-yl)methyl)-1H-1,2,3-triazol-4-yl)pyridin-3-yl)pyrrolidin-3-yl)acetic acid

To a solution of ethyl 2-(1-(2-methyl-6-(1-methyl-5-((2-oxo-5-propylpyridin-1(2H)-yl)methyl)-1H-1,2,3-triazol-4-yl)pyridin-3-yl)pyrrolidin-3-yl)acetate (50 mg, 0.10 mmol) in THE (4 mL)/H₂O (1 mL)/MeOH (1 mL) was added LiOH·H₂O (44 mg, 1.0 mmol) and the reaction was stirred at r.t. for 2 hrs. Volatiles were removed under vacuum and the residue was diluted with H₂O (5 mL). The mixture was adjusted with 1N aq. HCl to pH˜4 and extracted with EtOAc (3×10 mL). The combined organic layers were washed with brine, dried over anhydrous Na₂SO₄, filtered and concentrated to dryness. The residue was purified by prep-HPLC (C18, 5-95%, MeCN in H₂O with 0.1% HCOOH) to give the title compound (10 mg, 21.2% yield) as white solid. LC/MS (ESI) m/z: 451 (M+H)⁺. ¹H NMR (400 MHz, CD₃OD) δ 7.83 (d, J=2.0 Hz, 1H), 7.70 (d, J=8.4 Hz, 1H), 7.36 (dd, J=9.2, 2.5 Hz, 1H), 7.27 (d, J=8.5 Hz, 1H), 6.47 (d, J=9.2 Hz, 1H), 5.75 (s, 2H), 4.22 (s, 3H), 3.49-3.36 (m, 2H), 3.17-3.08 (m, 1H), 2.77-2.65 (s, 1H), 2.63 (s, 3H), 2.51 (d, J=7.4 Hz, 2H), 2.24 (t, J=7.4 Hz, 3H), 1.77-1.65 (m, 1H), 1.44-1.30 (m, 2H), 0.78 (t, J=7.3 Hz, 3H). LC/MS (ESI) m/z: 451 (M+H)⁺.

The following examples in Table 1 were prepared from appropriate starting materials by using a method analogous to that used to prepare the examples as described herein.

TABLE 1

| Example | | | |
|---|---|---|---|
| No. | Structure & name | Analytical data | Method |
| 3 | 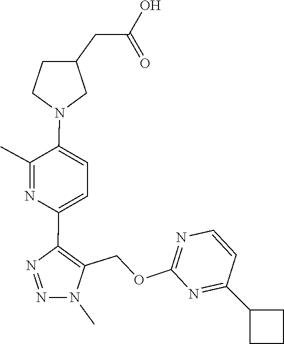 [SMILES: Cc1nc(-c2nnn(C)c2COc2nccc(C3CCC3)n2)ccc1N1CCC(CC(=O)O)C1] | LC/MS (ESI) m/z: 464 (M + H)⁺. ¹H NMR (400 MHz, CD₃OD) δ 8.40 (d, J = 5.1 Hz, 1H), 7.72 (d, J = 8.5 Hz, 1H), 7.28 (d, J = 8.5 Hz, 1H), 6.98 (d, J = 5.1 Hz, 1H), 6.13 (s, 2H), 4.22 (s, 3H), 3.55 − 3.47 (m, 1H), 3.45 − 3.37 (m, 2H), 3.26 (s, 3H), 3.12 − 3.05 (m, 1H), 2.72 − 2.63 (m, 1H), 2.50 (d, J = 5.6 Hz, 5H), 2.26 − 2.17 (m, 5H), 2.07 − 1.96 (m, 1H), 1.86 − 1.75 (m, 1H), 1.74 − 1.61 (m, 1H). | Example 1 |
| | 2-(1-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | methylpyridin-3- | | |
| | yl)pyrrolidin-3-yl)acetic | | |
| | acid | | |
| 4 | 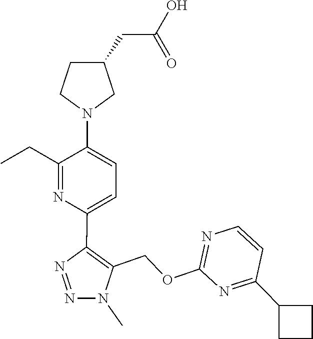 [SMILES: CCc1nc(-c2nnn(C)c2COc2nccc(C3CCC3)n2)ccc1N1CC[C@H](CC(=O)O)C1] | LC/MS (ESI) m/z: 478 (M + H)⁺. ¹H NMR (400 MHz, CD₃OD) δ 8.41 (d, J = 5.1 Hz, 1H), 7.76 (s, 1H), 7.32 (d, J = 8.6 Hz, 1H), 6.98 (d, J = 5.1 Hz, 1H), 6.19 (s, 2H), 4.22 (s, 3H), 3.73 (d, J = 5.8 Hz, 1H), 3.55 − 3.47 (m, 1H), 3.40 − 3.34 (m, 2H), 3.28 − 3.22 (m, 1H), 3.05 (dd, J = 9.0, 7.1 Hz, 1H), 2.87 − 2.78 (m, 2H), 2.73 − 2.62 (m, 1H), 2.49 (d, J = 7.3 Hz, 2H), 2.25 − 2.21 (m, 4H), 2.08 − 1.98 (m, 1H), 1.85 − 1.76 (m, 1H), 1.74 − 1.64 (m, 1H), 1.22 (t, J = 6.9 Hz, 3H). | Example 1 |
| | (R)-2-(1-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)pyrrolidin-3-yl)acetic | | |
| | acid | | |
| 5 | 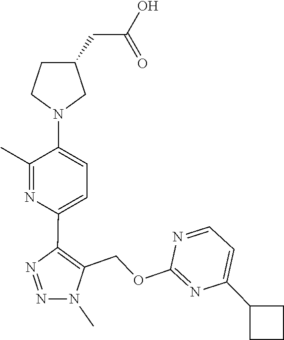 [SMILES: Cc1nc(-c2nnn(C)c2COc2nccc(C3CCC3)n2)ccc1N1CC[C@H](CC(=O)O)C1] | LC/MS (ESI) m/z: 464 (M + H)⁺. ¹H NMR (400 MHz, CD₃OD) δ 8.40 (d, J = 5.1 Hz, 1H), 7.71 (s, 1H), 7.27 (d, J = 8.4 Hz, 1H), 6.97 (d, J = 5.1 Hz, 1H), 6.13 (s, 2H), 4.22 (s, 3H), 3.53 − 3.47 (m, 1H), 3.43 (d, J = 3.0 Hz, 2H), 3.28 − 3.21 (m, 1H), 3.12 − 3.06 (m, 1H), 2.66 (dd, J = 14.3, 7.1 Hz, 1H), 2.50 (d, J = 6.4 Hz, 5H), 2.25 − 2.19 (m, 5H), 2.06 − 1.98 (m, 1H), 1.83 − 1.76 (m, 1H), 1.73 − 1.64 (m, 1H). | Example 1 |
| | (R)-2-(1-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | methylpyridin-3- | | |
| | yl)pyrrolidin-3-yl)acetic | | |
| | acid | | |
| 6 | 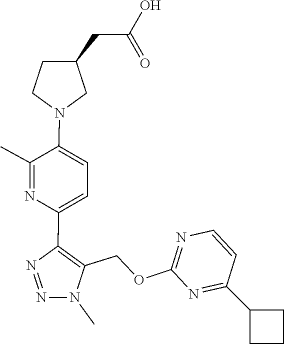 [SMILES: Cc1nc(-c2nnn(C)c2COc2nccc(C3CCC3)n2)ccc1N1CC[C@@H](CC(=O)O)C1] | LC/MS (ESI) m/z: 464 (M + H)⁺. ¹H NMR (400 MHz, CD₃OD) δ 8.40 (d, J = 5.1 Hz, 1H), 7.72 (d, J = 8.5 Hz, 1H), 7.27 (d, J = 8.5 Hz, 1H), 6.97 (d, J = 5.1 Hz, 1H), 6.13 (s, 2H), 4.22 (s, 3H), 3.55 − 3.46 (m, 1H), 3.43 − 3.36 (m, 2H), 3.28 − 3.26 (m, 1H), 3.11 − 3.07 (m, 1H), 2.71 − 2.64 (m, 1H), 2.49 (s, 3H), 2.52 − 2.46 (m, 2H), 2.25 − 2.19 (m, 5H), 2.07 − 1.95 (m, 1H), 1.84 − 1.77 (m, 1H), 1.74 − 1.65 (m, 1H). | Example 1 |
| | (S)-2-(1-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | methylpyridin-3- | | |
| | yl)pyrrolidin-3-yl)acetic | | |
| | acid | | |
| 7 | 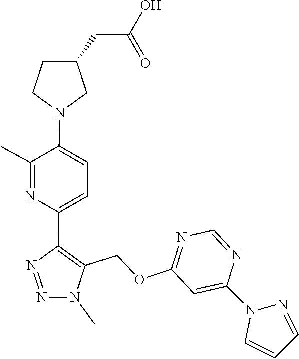 [SMILES: Cc1nc(-c2nnn(C)c2COc2cc(-n3cccn3)ncn2)ccc1N1CC[C@H](CC(=O)O)C1] | LC/MS (ESI) m/z: 476 (M + H)⁺. ¹H NMR (400 MHz, CD₃OD) δ 8.64 (d, J = 0.8 Hz, 1H), 8.60 (d, J = 2.7 Hz, 1H), 7.77 (d, J = 1.1 Hz, 1H), 7.71 (d, J = 8.5 Hz, 1H), 7.30 (t, J = 4.0 Hz, 1H), 7.25 (d, J = 8.5 Hz, 1H), 6.55 (dd, J = 2.7, 1.7 Hz, 1H), 6.15 (d, J = 14.1 Hz, 2H), 4.22 (d, J = 7.1 Hz, 3H), 3.49 − 3.35 (m, 2H), 3.29 − 3.24 (m, 2H), 3.08 (dd, J = 9.2, 7.2 Hz, 1H), 2.66 (dd, J = 14.7, 7.2 Hz, 1H), 2.50 (s, 1H), 2.48 (s, 3H), 2.21 (dt, J = 11.4, 5.7 Hz, 1H), 1.79 − 1.59 (m, 1H). | Example 1 |
| | (R)-2-(1-(6-(5-(((6-(1H- | | |
| | pyrazol-1-yl)pyrimidin-4- | | |
| | yl)oxy)methyl)-1-methyl- | | |
| | 1H-1,2,3-triazol-4-yl)-2- | | |
| | methylpyridin-3- | | |
| | yl)pyrrolidin-3-yl)acetic | | |
| | acid | | |

### Example 8: (R)-2-((S)-1-(6-(5-(((4-cyclobutylpyrimidin-2-yl)oxy)methyl)-1-methyl-1H-1,2,3-triazol-4-yl)-2-ethylpyridin-3-yl)pyrrolidin-3-yl)propanoic acid

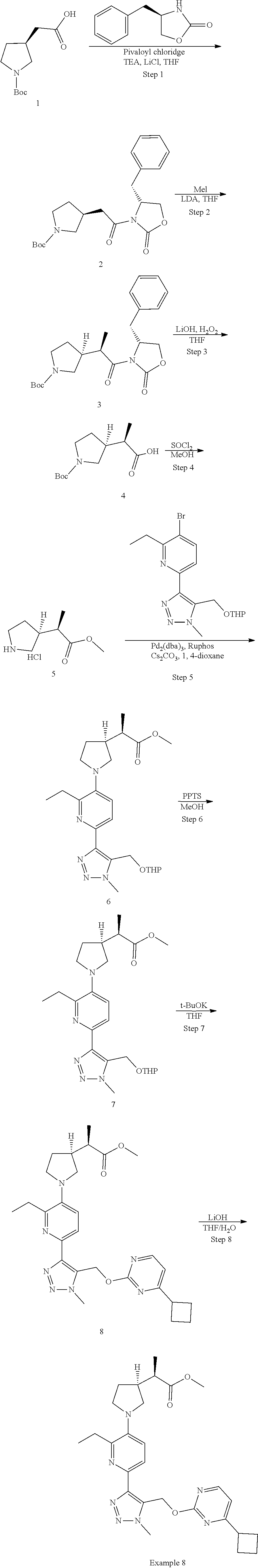

### Step 1: tert-butyl (3S)-3-{2-[(4R)-4-benzyl-2-oxo-1,3-oxazolidin-3-yl]-2-oxoethyl}pyrrolidine-1-carboxylate

To a solution of 2-[(3S)-1-[(tert-butoxy)carbonyl]pyrrolidin-3-yl]acetic acid (3.7 g, 16.1 mmol) in THF (80 mL) was added TEA (2.71 g, 26.8 mmol) followed by drop-wise addition of pivaloyl chloride (3.23 g, 26.8 mmol) at 0° C. The mixture was stirred at 0° C. for 1 hr and (4R)-4-benzyl-1,3-oxazolidin-2-one (1.9 g, 10.7 mmol) and LiCl (40 mg, 0.872 mmol) were added to the mixture. The resulting mixture was stirred at 70° C. for 16 hrs. The mixture was poured into saturated aq. NH₄Cl solution and extracted with EtOAc (3×30 mL). The combined organic layers were washed with brine, dried over anhydrous Na₂SO₄, filtered and concentrated to dryness. The residue was purified by flash chromatography (silica gel, 0˜35% of EtOAc in PE) to give the title compound (2.9 g, 69.6% yield) as colorless oil. LC/MS (ESI) m/z: 389 (M+H)⁺.

### Step 2: tert-butyl (3R)-3-{1-[(4R)-4-benzyl-2-oxo-1,3-oxazolidin-3-yl]-1-oxopropan-2-yl}pyrrolidine-1-carboxylate

To a solution of tert-butyl (3R)-3-{2-[(4R)-4-benzyl-2-oxo-1,3-oxazolidin-3-yl]-2-oxoethyl}pyrrolidine-1-carboxylate (1.5 g, 3.9 mmol) in THE (30 mL) was added LDA (2.3 mL, 5.8 mmol) drop-wisely at −78° C. under N₂ atmosphere over 30 mins and the mixture was stirred at −78° C. for 1 hr. Mel (28.3 g, 199.3 mmol) was added drop-wisely to the mixture over a period of 30 mins while maintaining the temperature below −70° C. After stirring at −70° C. for 1 hr, the reaction mixture was further stirred at r.t. for 2 hrs until the disappearance of the starting material was confirmed by TLC analysis. The reaction was quenched with saturated aq. NH₄Cl solution at 0° C. and extracted with EtOAc (2×50 mL). The combined organic layers were washed with water and brine, dried over anhydrous Na₂SO₄, filtered and concentrated to dryness. The residue was purified by flash chromatography (silica gel, 0˜50% of EtOAc in PE) to give the title compound (850 mg, 54.7% yield) as white solid. LC/MS (ESI) m/z: 403 (M+H)⁺.

### Step 3: 2-[(3R)-1-[(tert-butoxy)carbonyl]pyrrolidin-3-yl]propanoic acid

To a solution of tert-butyl (3R)-3-{1-[(4R)-4-benzyl-2-oxo-1,3-oxazolidin-3-yl]-1-oxopropan-2-yl}pyrrolidine-1-carboxylate (400 mg, 0.99 mmol) in THE (10 mL) was added LiOH (125 mg, 2.98 mmol) followed by H₂O₂ (2 mL, 65.3 mmol, 30% wt) and the mixture was stirred at 0° C. for 2 hrs. The mixture was concentrated to 1/5 volume and diluted with water (10 mL). The mixture were washed with EtOAc (2×10 mL) and the aqueous layer was acidified with 1N aq. HCl to pH-4 and extracted with DCM (3×20 mL). The combined organic layers were washed with brine, dried over anhydrous Na₂SO₄, filtered and concentrated to dryness to give the title compound (200 mg, 82.7% yield) as white solid. LC/MS (ESI) m/z: 244 (M+H)⁺.

### Step 4: methyl (2R)-2-[(3S)-pyrrolidin-3-yl]propanoate hydrochloride

To a solution of (2R)-2-[(3S)-1-[(tert-butoxy)carbonyl]pyrrolidin-3-yl]propanoic acid (200 mg, 0.82 mmol) in MeOH (10 mL) was added SOCl₂ (1 mL, 12.35 mmol) at 0° C. under N₂ atmosphere. After addition, the resulting solution was stirred at r.t. for another 16 hrs. The reaction was concentrated under reduced pressure to dryness to give the title compound (160 mg, 100% yield) as colorless solid, which was directly used in the next reaction without purification. LC/MS (ESI): m/z: 158 (M+H)⁺.

### Step 5: methyl (2R)-2-[(3S)-1-(2-ethyl-6-{1-methyl-5-[(oxan-2-yloxy)methyl]-1H-1,2,3-triazol-4-yl}pyridin-3-yl)pyrrolidin-3-yl]propanoate

To a mixture of 3-bromo-2-ethyl-6-{1-methyl-5-[(oxan-2-yloxy)methyl]-1H-1,2,3-triazol-4-yl}pyridine (300 mg, 0.79 mmol) and (2R)-2-[(3S)-pyrrolidin-3-yl]propanoate hydrochloride (153 mg, 0.79 mmol) in 1,4-dioxane (10 mL) was added Cs₂CO₃ (769 mg, 2.36 mmol), Pd₂(dba)₃ (64 mg, 0.08 mmol) and Ru-Phos (37 mg, 0.08 mmol) under N₂ atmosphere. The mixture was degassed under N₂ atmosphere for three times and stirred at 120° C. for 16 hrs. The reaction mixture was filtered through a Celite pad and the filtrate was concentrated to dryness. The residue was purified by flash chromatography (silica gel, 0-50% of EtOAc in PE) to give the title compound (270 mg, 75% yield) as yellow solid. LC/MS (ESI) m/z: 458 (M+H)⁺.

### Step 6: methyl (2R)-2-[(3S)-1-{2-ethyl-6-[5-(hydroxymethyl)-1-methyl-11H-1,2,3-triazol-4-yl]pyridin-3-yl}pyrrolidin-3-yl]propanoate

To a solution of methyl (2R)-2-[(3S)-1-(2-ethyl-6-{1-methyl-5-[(oxan-2-yloxy)methyl]-1H-1,2,3-triazol-4-yl}pyridin-3-yl)pyrrolidin-3-yl]propanoate (270 mg, 0.590 mmol) in MeOH (5 mL) was added PPTS (297 mg, 1.18 mmol) and the reaction mixture was stirred at 60° C. for 2 hrs. The reaction mixture was concentrated to dryness and the residue was dissolved in EtOAc (20 mL). The mixture was washed with brine, dried over Na₂SO₄, filtered and concentrated to dryness. The residue was purified with silica gel column chromatography (0˜80% of EtOAc in PE) to give the title compound (170 mg, 80.7% yield) as yellow solid. LC/MS (ESI) m/z: 374 (M+H)⁺.

### Step 7: (2S)—N-(6-bromo-3-methylpyridin-2-yl)-2-(methylamino)hex-5-enamide

To a solution of methyl (2R)-2-[(3S)-1-{2-ethyl-6-[5-(hydroxymethyl)-1-methyl-1H-1,2,3-triazol-4-yl]pyridin-3-yl}pyrrolidin-3-yl]propanoate (80 mg, 0.21 mmol) in THE (5 mL) was added t-BuOK (0.43 mL, 0.43 mmol, 1.0 M in THF) drop-wisely at 0° C. The reaction mixture was stirred at 0° C. for 30 min until the disappearance of the starting material was confirmed by TLC analysis. The reaction was quenched with saturated aq. NH₄Cl solution at 0° C. and the mixture was extracted with EtOAc (2×20 mL). The combined organic layers were washed with brine, dried over anhydrous Na₂SO₄, filtered and concentrated to dryness. The residue was purified by flash chromatography (silica gel, 0˜60% of EtOAc in PE) to give the title compound (80 mg, 73.9% yield) as yellow solid. LC/MS (ESI) m/z: 506 (M+H)⁺.

### Step 8: (2R)-2-[(3S)-1-[6-(5-{[(4-cyclobutylpyrimidin-2-yl)oxy]methyl}-1-methyl-1H-1,2,3-triazol-4-yl)-2-ethylpyridin-3-yl]pyrrolidin-3-yl]propanoic acid

To a solution of methyl (2R)-2-[(3S)-1-[6-(5-{[(4-cyclobutylpyrimidin-2-yl)oxy]methyl}-1-methyl-1H-1,2,3-triazol-4-yl)-2-ethylpyridin-3-yl]pyrrolidin-3-yl]propanoate (70 mg, 0.14 mmol) in THF (4 mL)/H₂O (1 mL)/MeOH (1 mL) was added LiOH·H₂O (58 mg, 1.38 mmol) and the mixture was stirred at r.t. for 2 hrs. Volatiles were removed under vacuum and the residue was diluted with H₂O (5 mL). The mixture was adjusted with 1N aq. HCl to pH-4 and extracted with EtOAc (3×5 mL). The combined organic layers were washed with brine, dried over anhydrous Na₂SO₄, filtered and concentrated to dryness. The residue was purified by prep-HPLC (C18, 5-95%, MeCN in H₂O with 0.1% HCOOH) to give the title compound (25 mg, 36.7% yield) as white solid. LC/MS (ESI) m/z: 492 (M+H)⁺. ¹H NMR (400 MHz, CD₃OD) δ 8.41 (d, J=5.1 Hz, 1H), 7.76 (d, J=8.4 Hz, 1H), 7.30 (d, J=8.5 Hz, 1H), 6.98 (d, J=5.1 Hz, 1H), 6.18 (d, J=4.5 Hz, 2H), 4.22 (s, 3H), 3.57-3.45 (m, 1H), 3.42-3.33 (m, 1H), 3.28-3.23 (m, 1H), 3.21-3.10 (m, 2H), 2.81 (q, J=7.4 Hz, 2H), 2.48-2.37 (m, 2H), 2.27-2.14 (m, 5H), 2.08-1.95 (m, 1H), 1.85-1.76 (m, 1H), 1.75-1.64 (m, 1H), 1.26-1.19 (m, 6H).

The following examples in Table 2 were prepared from appropriate starting materials using a method analogous to that used to prepare the examples as describer herein.

TABLE 2

| Example | | | |
|---|---|---|---|
| No. | Structure & name | Analytical data | Method |
| 9 | 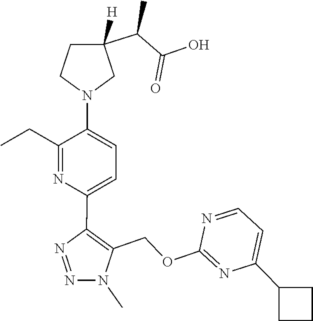 [SMILES: CCc1nc(-c2nnn(C)c2COc2nccc(C3CCC3)n2)ccc1N1CC[C@H]([C@@H](C)C(=O)O)C1] | LC/MS (ESI) m/z: 492 (M + H)⁺. ¹H NMR (400 MHz, CD₃OD) δ 8.38 (d, J = 5.1 Hz, 1H), 7.96 (d, J = 8.9 Hz, 1H), 7.75 (d, J = 8.9 Hz, 1H), 7.02 (d, J = 5.1 Hz, 1H), 5.91 − 5.83 (m, 2H), 4.30 (s, 3H), 3.65 − 3.48 (m, 4H), 3.40 − 3.36 (m, 1H), 3.17 (q, J = 7.6 Hz, 2H), 2.58 − 2.45 (m, 2H), 2.31 − 2.18 (m, 5H), 2.12 − 2.00 (m, 1H), 1.90 − 1.76 (m, 2H), 1.34 (t, J = 7.5 Hz, 3H), 1.30 (d, J = 6.6 Hz, 3H). | Example 8 |
| | (R)-2-((R)-1-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)pyrrolidin-3- | | |
| | yl)propanoic acid | | |
| 10 | 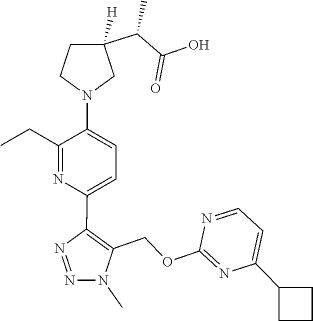 [SMILES: CCc1nc(-c2nnn(C)c2COc2nccc(C3CCC3)n2)ccc1N1CC[C@@H]([C@H](C)C(=O)O)C1] | LC/MS (ESI) m/z: 492 (M + H)⁺. ¹H NMR (400 MHz, CD₃OD) δ 8.39 (d, J = 5.1 Hz, 1H), 7.93 (d, J = 8.8 Hz, 1H), 7.69 (d, J = 8.9 Hz, 1H), 7.01 (d, J = 5.1 Hz, 1H), 5.95 − 5.87 (m, 2H), 4.29 (s, 3H), 3.62 − 3.51 (m, 3H), 3.49 − 3.44 (m, 1H), 3.36 (d, J = 8.4 Hz, 1H), 3.12 (q, J = 7.5 Hz, 2H), 2.56 − 2.44 (m, 2H), 2.31 − 2.18 (m, 5H), 2.12 − 2.00 (m, 1H), 1.89 − 1.74 (m, 2H), 1.32 (t, J = 7.5 Hz, 3H), 1.29 (d, J = 6.5 Hz, 3H). | Example 8 |
| | (R)-2-(1-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)pyrrolidin-3-yl)acetic | | |
| | acid | | |
| 11 | 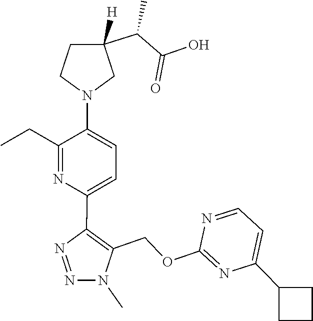 [SMILES: CCc1nc(-c2nnn(C)c2COc2nccc(C3CCC3)n2)ccc1N1CC[C@H]([C@H](C)C(=O)O)C1] | LC/MS (ESI) m/z: 492 (M + H)⁺. ¹H NMR (400 MHz, CD₃OD) δ 8.39 (d, J = 5.1 Hz, 1H), 7.97 (d, J = 8.9 Hz, 1H), 7.79 (d, J = 9.0 Hz, 1H), 7.02 (d, J = 5.1 Hz, 1H), 5.93 − 5.77 (m, 2H), 4.31 (s, 3H), 3.67 − 3.49 (m, 4H), 3.41 − 3.34 (m, 1H), 3.23 − 3.13 (m, 2H), 2.59 − 2.47 (m, 2H), 2.34 − 2.20 (m, 5H), 2.13 − 1.99 (m, 1H), 1.92 − 1.80 (m, 2H), 1.35 (t, J= 7.5 Hz, 3H), 1.27 (d, J = 6.5 Hz, 3H). | Example 8 |
| | (R)-2-(1-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | methylpyridin-3- | | |
| | yl)pyrrolidin-3-yl)acetic | | |
| | acid | | |
| 12 | 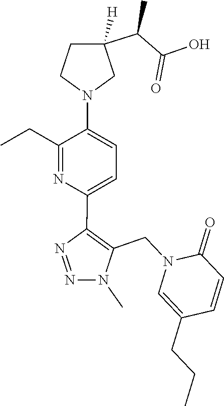 [SMILES: CCCc1ccc(=O)n(Cc2c(-c3ccc(N4CC[C@@H]([C@@H](C)C(=O)O)C4)c(CC)n3)nnn2C)c1] | LC/MS (ESI) m/z: 479 (M + H)⁺. ¹H NMR (400 MHz, CD₃OD) δ 7.92 (d, J = 8.8 Hz, 1H), 7.82 (s, 1H), 7.71 (d, J = 8.9 Hz, 1H), 7.55 (dd, J = 9.2, 2.4 Hz, 1H), 6.60 (d, J = 9.2 Hz, 1H), 5.66 − 5.51 (m, 2H), 4.15 (s, 3H), 3.75 − 3.38 (m, 4H), 3.34 (d, J = 8.7 Hz, 1H), 3.19 (q, J = 7.5 Hz, 2H), 2.57 − 2.48 (m, 2H), 2.45 − 2.35 (m, 2H), 2.30 − 2.19 (m, 1H), 1.90 − 1.75 (m, 1H), 1.62 − 1.50 (m, 2H), 1.36 (t, J = 7.5 Hz, 3H), 1.27 (d, J = 6.5 Hz, 3H), 0.91 (t, J = 7.3 Hz, 3H). | Example 8 & Example 2 |
| | (R)-2-((S)-1-(2-ethyl-6-(1- | | |
| | methyl-5-((2-oxo-5- | | |
| | propylpyridin-1(2H)- | | |
| | yl)methyl)-1H-1,2,3- | | |
| | triazol-4-yl)pyridin-3- | | |
| | yl)pyrrolidin-3- | | |
| | yl)propanoic acid | | |
| 13 | 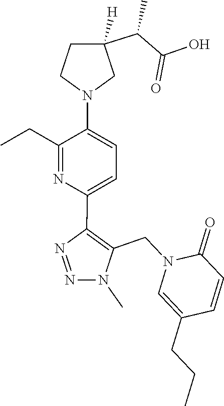 [SMILES: CCCc1ccc(=O)n(Cc2c(-c3ccc(N4CC[C@@H]([C@H](C)C(=O)O)C4)c(CC)n3)nnn2C)c1] | LC/MS (ESI) m/z: 479 (M + H)⁺. ¹H NMR (400 MHz, CD₃OD) δ 7.76 (d, J = 8.5 Hz, 1H), 7.67 (d, J = 2.2 Hz, 1H), 7.39 − 7.32 (m, 2H), 6.52 (d, J = 9.2 Hz, 1H), 5.90 − 5.82 (m, 2H), 4.16 (s, 3H), 3.48 − 3.39 (m, 1H), 3.27 − 3.18 (m, 3H), 2.94 (q, J = 7.4 Hz, 2H), 2.50 − 2.43 (m, 2H), 2.20 (t, J = 7.5 Hz, 3H), 1.78 − 1.69 (m, 1H), 1.40 − 1.33 (m, 2H), 1.31 − 1.26 (m, 6H), 0.76 (t, J = 7.3 Hz, 3H). | Example 8 & Example 2 |
| | (S)-2-((S)-1-(2-ethyl-6-(1- | | |
| | methyl-5-((2-oxo-5- | | |
| | propylpyridin-1(2H)- | | |
| | yl)methyl)-1H-1,2,3- | | |
| | triazol-4-yl)pyridin-3- | | |
| | yl)pyrrolidin-3- | | |
| | yl)propanoic acid | | |

### Example 14: 2-[(3S)-1-(2-ethyl-6-{1-methyl-5-[(2-oxo-5-propyl-1,2-dihydropyridin-1-yl)methyl]-4,5-dihydro-1H-1,2,3-triazol-4-yl}pyridin-3-yl)pyrrolidin-3-yl]-2-methylpropanoic acid

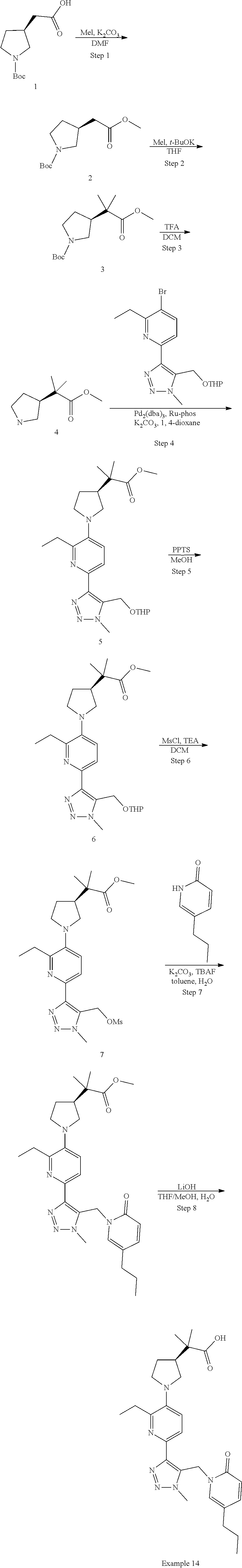

### Step 1: tert-butyl (3S)-3-(2-methoxy-2-oxoethyl)pyrrolidine-1-carboxylate

To a solution of 2-[(3S)-1-[(tert-butoxy)carbonyl]pyrrolidin-3-yl]acetic acid (1.5 g, 6.5 mmol) in DMF (20 mL) was added K₂CO₃ (2.71 g, 19.6 mmol) followed by Mel (0.81 mL, 13.1 mmol) at 0° C. under N₂ atmosphere. The reaction was stirred at r.t. for 16 hrs. The mixture was quenched with saturated aq. NH₄Cl solution (10 mL) and extracted with EtOAc (3×10 mL). The combined organic layers were washed with brine, dried over anhydrous Na₂SO₄, filtered and concentrated to dryness. The residue was purified by flash chromatography (silica gel, 0˜50% of EtOAc in PE) to give the title compound (1.5 g, 94.2% yield) as yellow oil. LC/MS (ESI) m/z: 244 (M+H)⁺.

### Step 2: tert-butyl (3S)-3-(1-methoxy-2-methyl-1-oxopropan-2-yl)pyrrolidine-1-carboxylate

To a solution of tert-butyl (3S)-3-(2-methoxy-2-oxoethyl)pyrrolidine-1-carboxylate (200 mg, 0.82 mmol) in THE (10 mL) was added t-BuOK (2.5 mL, 2.5 mmol, 1 M in THF) drop-wisely at −70° C. under N₂ atmosphere over 20 mins and the mixture was stirred at −70° C. for 1 hr. Mel (583 mg, 4.11 mmol) was added drop-wisely to the mixture over a period of 10 mins while maintaining the temperature below −70° C. The reaction mixture was further stirred at r.t. for 16 hrs until the disappearance of the starting material was confirmed by TLC analysis. The reaction was quenched with saturated aq. NH₄Cl solution at 0° C. and extracted with EtOAc (2×10 mL). The combined organic layers were washed with water and brine, dried over anhydrous Na₂SO₄, filtered and concentrated to dryness. The residue was purified by flash chromatography (silica gel, 0˜20% of EtOAc in PE) to give the title compound (120 mg, 53.8% yield) as yellow oil. LC/MS (ESI) m/z: 272 (M+H)⁺.

### Step 3: methyl 2-methyl-2-[(3S)-pyrrolidin-3-yl]propanoate TFA salt

To a solution of tert-butyl (3S)-3-(1-methoxy-2-methyl-1-oxopropan-2-yl)pyrrolidine-1-carboxylate (120 mg, 0.44 mmol) in DCM (3 mL) was added TFA (1 mL) and the mixture was stirred at r.t. for 2 hrs. The mixture was concentrated to dryness to give the title compound (110 mg, 100% yield) as colorless oil, which was used directly in the next step. LC/MS (ESI) m/z: 172 (M+H)⁺.

### Step 4: methyl 2-[(3S)-1-(2-ethyl-6-{1-methyl-5-[(oxan-2-yloxy)methyl]-1H-1,2,3-triazol-4-yl}pyridin-3-yl)pyrrolidin-3-yl]-2-methylpropanoate

To a solution of 3-bromo-2-ethyl-6-{1-methyl-5-[(oxan-2-yloxy)methyl]-1H-1,2,3-triazol-4-yl}pyridine (167 mg, 0.44 mmol) in 1,4-dioxane (5 mL) was added methyl 2-methyl-2-[(3S)-pyrrolidin-3-yl]propanoate TFA salt (110 mg, 0.44 mmol) followed by the addition of Cs₂CO₃ (428 mg, 1.31 mmol), Pd₂(dba)₃ (40 mg, 0.044 mmol) and RuPhos (20 mg, 0.044 mmol) under N₂ atmosphere. The reaction mixture was degassed under N₂ atmosphere for three times and stirred at 120° C. for 16 hrs. The mixture was filtered and the filtrate was concentrated under reduced pressure to dryness. The residue was purified by flash chromatography (eluted with PE:EtOAc=10:1 to 1:2) to give the title compound (80 mg, 38.7% yield) as yellow solid. LC/MS (ESI) m/z: 472 (M+H)⁺.

### Step 5: methyl 2-[(3S)-1-{2-ethyl-6-[5-(hydroxymethyl)-1-methyl-4,5-dihydro-1H-1,2,3-triazol-4-yl]pyridin-3-yl}pyrrolidin-3-yl]-2-methylpropanoate

To a solution of methyl 2-[(3S)-1-(2-ethyl-6-{1-methyl-5-[(oxan-2-yloxy)methyl]-4,5-dihydro-1H-1,2,3-triazol-4-yl}pyridin-3-yl)pyrrolidin-3-yl]-2-methylpropanoate (80 mg, 0.17 mmol) in MeOH (3 mL) was added PPTS (128 mg, 0.51 mmol). The reaction was stirred at 60° C. for 16 hrs. The mixture was concentrated to dryness and the residue was diluted with EtOAc (10 mL). The organic layer was washed with brine, dried over Na₂SO₄, filtered and concentrated to dryness. The residue was purified with silica gel column chromatography (0˜70% of EtOAc in PE) to give the title compound (50 mg, 76.0% yield) as yellow solid. LC/MS (ESI) m/z: 388 (M+H)⁺.

### Step 6: methyl 2-[(3S)-1-(2-ethyl-6-{5-[(methanesulfonyloxy)methyl]-1-methyl-1H-1,2,3-triazol-4-yl}pyridin-3-yl)pyrrolidin-3-yl]-2-methylpropanoate

To a stirred solution of methyl 2-[(3S)-1-{2-ethyl-6-[5-(hydroxymethyl)-1-methyl-1H-1,2,3-triazol-4-yl]pyridin-3-yl}pyrrolidin-3-yl]-2-methylpropanoate (50 mg, 0.13 mmol) in DCM (10 mL) was added MsCl (30 mg, 0.26 mmol) followed by TEA (39 mg, 0.39 mmol) at 0° C. under N₂ atmosphere. The reaction mixture was stirred at r.t. for 30 min until the disappearance of the starting material was confirmed by TLC analysis. The reaction was quenched with saturated aq. NaHCO₃ solution at 0° C. and extracted with DCM (2×10 mL). The combined organic layers were washed with brine, dried over anhydrous Na₂SO₄, filtered and concentrated to dryness to give the title compound (50 mg, 83.2% yield) as yellow solid, which was used directly in the next step. LC/MS (ESI) m/z: 466 (M+H)⁺.

### Step 7: methyl 2-[(3S)-1-(2-ethyl-6-{1-methyl-5-[(2-oxo-5-propyl-1,2-dihydropyridin-1-yl)methyl]-1H-1,2,3-triazol-4-yl}pyridin-3-yl)pyrrolidin-3-yl]-2-methylpropanoate

To mixture of methyl 2-[(3S)-1-(2-ethyl-6-{5-[(methanesulfonyloxy)methyl]-1-methyl-4,5-dihydro-1H-1,2,3-triazol-4-yl}pyridin-3-yl)pyrrolidin-3-yl]-2-methylpropanoate (50 mg, 0.11 mmol) and 5-propyl-1,2-dihydropyridin-2-one (29 mg, 0.22 mmol) in Toluene (5 mL) and H₂O (1 mL) were added K₂CO₃ (44.5 mg, 0.32 mmol) followed by TBAF (3 mg, 0.01 mmol). The reaction mixture was stirred at 100° C. for 16 hrs. The reaction mixture was concentrated under reduced pressure to dryness. The residue was diluted with EtOAc (10 mL), washed with water and brine, dried over anhydrous Na₂SO₄, filtered and concentrated to dryness to give the title compound (50 mg, 91.9% yield) as yellow sloid, which was used directly in the next step. LC/MS (ESI) m/z: 507 (M+H)⁺.

### Step 8: 2-[(3S)-1-(2-ethyl-6-{1-methyl-5-[(2-oxo-5-propyl-1,2-dihydropyridin-1-yl)methyl]-4,5-dihydro-1H-1,2,3-triazol-4-yl}pyridin-3-yl)pyrrolidin-3-yl]-2-methylpropanoic acid

To a solution of methyl 2-[(3S)-1-(2-ethyl-6-{1-methyl-5-[(2-oxo-5-propyl-1,2-dihydropyridin-1-yl)methyl]-4,5-dihydro-1H-1,2,3-triazol-4-yl}pyridin-3-yl)pyrrolidin-3-yl]-2-methylpropanoate (50 mg, 0.098 mmol) in THE (4 mL)/H₂O (1 mL)/MeOH (1 mL) was added LiOH·H₂O (41 mg, 0.98 mmol), and the reaction was stirred at r.t. for 2 hrs. Volatiles were removed under vacuum and the residue was diluted with H₂O (5 mL). The mixture was adjusted with 1N aq. HCl to pH-4 and extracted with EtOAc (3×5 mL). The combined organic layers were washed with brine, dried over anhydrous Na₂SO₄, filtered and concentrated to dryness. The residue was purified by prep-HPLC (C18, 5-95%, MeCN in H₂O with 0.1% HCOOH) to give the title compound (13 mg, 26.7% yield) as white solid. LC/MS (ESI) m/z: 493 (M+H)⁺. ¹H NMR (400 MHz, CD₃OD) δ 7.77 (d, J=8.5 Hz, 1H), 7.66 (d, J=2.0 Hz, 1H), 7.40-7.33 (m, 2H), 6.52 (d, J=9.2 Hz, 1H), 5.92-5.79 (m, 2H), 4.16 (s, 3H), 3.40-3.32 (m, 1H), 3.23-3.10 (m, 2H), 2.94 (q, J=7.5 Hz, 2H), 2.70-2.57 (m, 1H), 2.20 (t, J=7.5 Hz, 2H), 2.12-2.02 (m, 1H), 1.94-1.81 (m, 1H), 1.36 (dd, J=14.9, 7.4 Hz, 2H), 1.29 (t, J=7.5 Hz, 3H), 1.25 (s, 6H), 0.76 (t, J=7.3 Hz, 3H).

### Example 15: (S)-2-methyl-2-(1-(2-methyl-6-(1-methyl-5-((2-oxo-5-propylpyridin-1(2H)-yl)methyl)-1H-1,2,3-triazol-4-yl)pyridin-3-yl)pyrrolidin-3-yl)propanoic acid

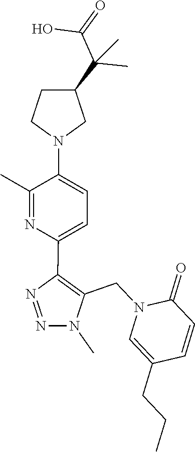 [SMILES: CCCc1ccc(=O)n(Cc2c(-c3ccc(N4CC[C@@H](C(C)(C)C(=O)O)C4)c(C)n3)nnn2C)c1]

The title compound was synthesized using the same synthetic sequence that was used to synthesize Example 14 from appropriate starting materials. LC/MS (ESI) (m/z): 479 (M+H)⁺. ¹H NMR (400 MHz, CD₃OD) δ 7.82 (d, J=2.0 Hz, 1H), 7.71 (d, J=8.4 Hz, 1H), 7.36 (dd, J=9.2, 2.5 Hz, 1H), 7.29 (d, J=8.5 Hz, 1H), 6.47 (d, J=9.2 Hz, 1H), 5.79-5.70 (m, 2H), 4.22 (s, 3H), 3.42-3.34 (m, 2H), 3.25-3.14 (m, 3H), 2.68-2.59 (m, 4H), 2.24 (t, J=7.5 Hz, 2H), 2.10-2.02 (m, 1H), 1.93-1.83 (m, 1H), 1.42-1.33 (m, 2H), 1.25 (s, 6H), 0.78 (t, J=7.3 Hz, 3H).

### Example 16: (R)-2-(1-(2-ethyl-6-(1-methyl-5-((2-oxo-5-propylpyridin-1(2H)-yl)methyl)-1H-1,2,3-triazol-4-yl)pyridin-3-yl)pyrrolidin-3-yl)-2-methylpropanoic acid

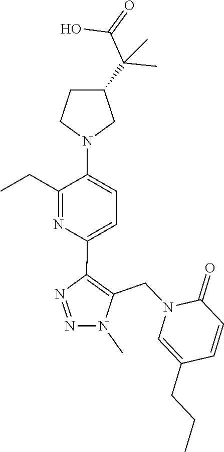 [SMILES: CCCc1ccc(=O)n(Cc2c(-c3ccc(N4CC[C@H](C(C)(C)C(=O)O)C4)c(CC)n3)nnn2C)c1]

The title compound was synthesized using the same synthetic sequence that was used to synthesize Example 14 from appropriate starting materials. LC/MS (ESI) (m/z): 493 (M+H)⁺. ¹H NMR (400 MHz, CD₃OD) δ 7.79 (d, J=8.5 Hz, 1H), 7.68 (d, J=2.1 Hz, 1H), 7.43-7.32 (m, 2H), 6.53 (d, J=9.2 Hz, 1H), 5.88 (d, J=3.0 Hz, 2H), 4.17 (s, 3H), 3.37 (dd, J=8.8, 6.7 Hz, 1H), 3.31-3.30 (m, 1H), 3.21 (td, J=8.4, 3.0 Hz, 1H), 3.16 (t, J=8.7 Hz, 1H), 2.96 (q, J=7.5 Hz, 2H), 2.72-2.61 (m, 1H), 2.22 (t, J=7.5 Hz, 2H), 2.12-2.04 (m, 1H), 1.94-1.85 (m, 1H), 1.38 (dd, J=14.9, 7.4 Hz, 2H), 1.31 (t, J=7.5 Hz, 3H), 1.27 (d, J=0.5 Hz, 6H), 0.78 (t, J=7.3 Hz, 3H).

### Example 17& Example 18: (S) or (R)-2-((S)-1-(2-ethyl-6-(1-methyl-5-((2-oxo-5-propylpyridin-1(2H)-yl)methyl)-1H-1,2,3-triazol-4-yl)pyridin-3-yl)pyrrolidin-3-yl)butanoic acid and (R) or (S)-2-((S)-1-(2-ethyl-6-(1-methyl-5-((2-oxo-5-propylpyridin-1(2H)-yl)methyl)-1H-1,2,3-triazol-4-yl)pyridin-3-yl)pyrrolidin-3-yl)butanoic acid

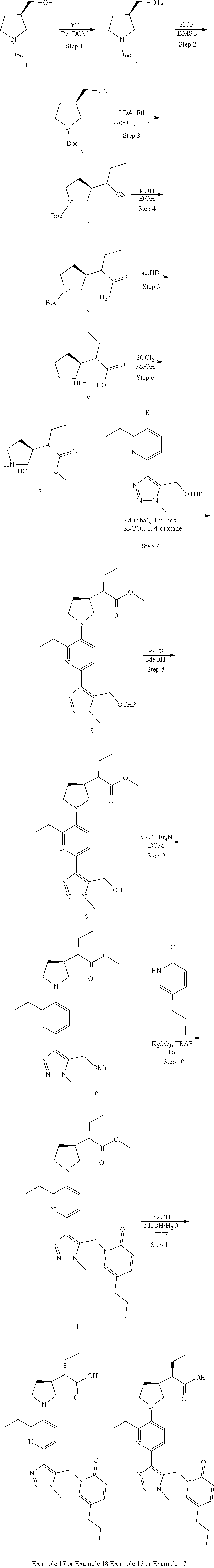

### Step 1: tert-butyl (R)-3-((tosyloxy)methyl)pyrrolidine-1-carboxylate

To a solution of tert-butyl (R)-3-(hydroxymethyl)pyrrolidine-1-carboxylate (2.3 g, 11.4 mmol) in DCM (30 mL) were added pyridine (2.8 mL, 34.3 mmol) and TosCl (3.3 g, 17.2 mmol) at 0° C. and the mixture was stirred at r.t. for 16 hrs. The mixture was quenched with water (20 mL) and extracted with DCM (3×20 mL). The combined organic layers were washed with brine, dried over anhydrous Na₂SO₄, filtered and concentrated to dryness. The residue was purified by flash chromatography (silica gel, 0˜20% of EtOAc in PE) to give the title compound (3.7 g, 91.1% yield) as white solid. LC/MS (ESI) m/z: 300 (M+H-56)⁺.

### Step 2: tert-butyl (R)-3-(cyanomethyl)pyrrolidine-1-carboxylate

To a solution of tert-butyl (R)-3-((tosyloxy)methyl)pyrrolidine-1-carboxylate (3.7 g, 10.4 mmol) in DMSO (20 mL) was added KCN (1.36 g, 20.8 mmol) and the reaction mixture was stirred at 100° C. for 16 hrs. The mixture was diluted with EtOAc (50 mL), washed with water and brine, dried over anhydrous Na₂SO₄, filtered and concentrated to dryness. The residue was purified by flash chromatography (silica gel, 0˜25% of EtOAc in PE) to give the title compound (1.9 g, 87% yield) as white solid. LC/MS (ESI) m/z: 155 (M+H-56)⁺.

### Step 3: tert-butyl (3S)-3-(1-cyanopropyl)pyrrolidine-1-carboxylate

To a solution of tert-butyl (R)-3-(cyanomethyl)pyrrolidine-1-carboxylate (1 g, 4.76 mmol) in THE (15 mL) was added LDA (7.1 mL, 7.1 mmol, 1 M in THF) drop-wisely at −70° C. Then a solution of EtI (965 mg, 6.2 mmol) in THE (3 mL) was added drop-wisely and the resulting mixture was stirred at −70° C. to r.t. for 16 hrs. The mixture was poured into saturated aq. NH₄Cl solution and extracted with EtOAc (3×40 mL). The combined organic layers were washed with brine, dried over anhydrous Na₂SO₄, filtered and concentrated to dryness. The residue was purified by flash chromatography (silica gel, 0-10% of EtOAc in PE) to give the title compound (400 mg, 35.3% yield) as yellow oil. LC/MS (ESI) m/z: 183 (M+H-56)⁺.

### Step 4: tert-butyl (3S)-3-(1-amino-1-oxobutan-2-yl)pyrrolidine-1-carboxylate

To a solution of tert-butyl (3S)-3-(1-cyanopropyl)pyrrolidine-1-carboxylate (400 mg, 1.68 mmol) in EtOH (5 mL) and H₂O (3 mL) was added KOH (282 mg, 5.04 mmol) and the mixture was stirred at 120° C. for 16 hrs. The mixture was diluted with water and extracted with EtOAc (3×30 mL). The combined organic layers were washed with brine, dried over anhydrous Na₂SO₄, filtered and concentrated to give the title compound (220 mg, 51.2% yield) as yellow oil. LC/MS (ESI) m/z: 201 (M+H-56)⁺.

### Step 5: 2-((S)-pyrrolidin-3-yl)butanoic acid hydrobromide

A solution of tert-butyl (S)-3-((S)-1-amino-1-oxobutan-2-yl)pyrrolidine-1-carboxylate (220 mg, 0.86 mmol) in aq. HBr (4 mL, 40% wt) was stirred at 120° C. for 16 hrs. The mixture was concentrated to dryness to give the title compound (150 mg, ⁸1.5% yield) as yellow solid. LC/MS (ESI) m/z: 158 (M+H)⁺.

### Step 6: methyl 2-((S)-pyrrolidin-3-yl)butanoate hydrochloride

To a solution of 2-((S)-pyrrolidin-3-yl)butanoic acid hydrobromide (100 mg, 0.64 mmol) in MeOH (3 mL) was added SOCl₂ (0.14 mL, 1.91 mmol) at 0° C. The mixture reaction was stirred at r.t. for 2 hrs. The mixture was concentrated to dryness to give the title compound (120 mg, 92% yield) as yellow solid. LC/MS (ESI) m/z: 172 (M+H)⁺.

### Step 7: methyl 2-((3S)-1-(2-ethyl-6-(1-methyl-5-(((tetrahydro-2H-pyran-2-yl)oxy)methyl)-1H-1,2,3-triazol-4-yl)pyridin-3-yl)pyrrolidin-3-yl)butanoate

To a mixture of 3-bromo-2-ethyl-6-(1-methyl-5-(((tetrahydro-2H-pyran-2-yl)oxy)methyl)-1H-1,2,3-triazol-4-yl)pyridine (180 mg, 0.47 mmol) and methyl 2-((S)-pyrrolidin-3-yl)butanoate hydrochloride (116 mg, 0.56 mmol) in 1,4-dioxane (8 mL) was added Cs₂CO₃ (308 mg, 0.94 mmol) followed by Ru-Phos (44 mg, 0.1 mmol), Pd₂(dba)₃ (43 mg, 0.1 mmol) under N₂ atmosphere. Then the mixture was degassed under N₂ atmosphere for three times and stirred at 120° C. for 16 hrs. The mixture was filtered and the filtrate was concentrated under reduced pressure to dryness. The residue was purified by flash chromatography (silica gel, 0˜50% of EtOAc in PE) to give the title compound (150 mg, 67.4% yield) as yellow oil. LC/MS (ESI) m/z: 472 (M+H)⁺.

### Step 8: methyl 2-((S)-1-(2-ethyl-6-(5-(hydroxymethyl)-1-methyl-1H-1,2,3-triazol-4-yl)pyridin-3-yl)pyrrolidin-3-yl)butanoate

To a solution of methyl 2-((3S)-1-(2-ethyl-6-(1-methyl-5-(((tetrahydro-2H-pyran-2-yl)oxy)methyl)-1H-1,2,3-triazol-4-yl)pyridin-3-yl)pyrrolidin-3-yl)butanoate (150 mg, 0.32 mmol) in MeOH (6 mL) was added PPTS (240 mg, 0.95 mmol) and the mixture was stirred at 60° C. for 3 hrs. The mixture was diluted with DCM (10 mL), washed with water and brine, dried with anhydrous Na₂SO₄, filtered and concentrated to dryness. The residue was purified by flash chromatography (silica gel, 0˜60% of EtOAc in PE) to give the title compound (100 mg, 81.4% yield) as yellow oil. LC/MS (ESI) m/z: 388 (M+H)⁺.

### Step 9: methyl 2-((S)-1-(2-ethyl-6-(1-methyl-5-(((methylsulfonyl)oxy)methyl)-1H-1,2,3-triazol-4-yl)pyridin-3-yl)pyrrolidin-3-yl)butanoate

To a solution of methyl 2-((S)-1-(2-ethyl-6-(5-(hydroxymethyl)-1-methyl-1H-1,2,3-triazol-4-yl)pyridin-3-yl)pyrrolidin-3-yl)butanoate (50 mg, 0.13 mmol) in DCM (3 mL) was added TEA (39.2 mg, 0.39 mmol) followed by MsCl (22 mg, 0.19 mmol) at 0° C. and the mixture was stirred at r.t. for 1 hr. The mixture was quenched with saturated aq. NH₄Cl solution (10 mL) and extracted with DCM (3×10 mL). The combined organic layers were washed with brine, dried over anhydrous Na₂SO₄, filtered and concentrated to dryness to give the title compound (55 mg, 92% yield) as yellow oil. LC/MS (ESI) m/z: 466 (M+H)⁺.

### Step 10: methyl 2-((S)-1-(2-ethyl-6-(1-methyl-5-((2-oxo-5-propylpyridin-1(2H)-yl)methyl)-1H-1,2,3-triazol-4-yl)pyridin-3-yl)pyrrolidin-3-yl)butanoate

To a mixture of methyl 2-((S)-1-(2-ethyl-6-(1-methyl-5-(((methylsulfonyl)oxy)methyl)-1H-1,2,3-triazol-4-yl)pyridin-3-yl)pyrrolidin-3-yl)butanoate (55 mg, 0.12 mmol) and 5-propylpyridin-2(1H)-one (24 mg, 0.18 mmol) in toluene (3 mL) and H₂O (0.3 mL) were added K₂CO₃ (49 mg, 0.35 mmol) and TBAF (3 mg, 0.012 mmol) under N₂ atmosphere. The reaction mixture was stirred at 100° C. for 16 hrs. The mixture was diluted with water and extracted with EtOAc (3×5 mL). The combined organic layers were washed with brine, dried over anhydrous Na₂SO₄, filtered and concentrated to dryness. The residue was purified by flash chromatography (silica gel, 0˜10% of EtOAc in PE) to give the title compound (45 mg, 73.8% yield) as white solid. LC/MS (ESI) m/z: 507 (M+H)⁺.

### Step 11: (S) or (R)-2-((S)-1-(2-ethyl-6-(1-methyl-5-((2-oxo-5-propylpyridin-1(2H)-yl)methyl)-1H-1,2,3-triazol-4-yl)pyridin-3-yl)pyrrolidin-3-yl)butanoic acid and (R) or (S)-2-((S)-1-(2-ethyl-6-(1-methyl-5-((2-oxo-5-propylpyridin-1(2H)-yl)methyl)-1H-1,2,3-triazol-4-yl)pyridin-3-yl)pyrrolidin-3-yl)butanoic acid

To a solution of methyl 2-((S)-1-(2-ethyl-6-(1-methyl-5-((2-oxo-5-propylpyridin-1(2H)-yl)methyl)-1H-1,2,3-triazol-4-yl)pyridin-3-yl)pyrrolidin-3-yl)butanoate (45 mg, 0.09 mmol) in MeOH (2 mL), THE (2 mL) and H₂O (1 mL) was added NaOH (36 mg, 0.9 mmol) and the mixture was stirred at room temperature for 16 hrs. The mixture was concentrated to dryness and the residue was dissolved in water (5 mL). The mixture was washed with EtOAc (2×5 mL), acidified with 1N aq. HCl to pH-3 and extracted with DCM (3×5 mL). The combined organic layers were washed with brine, dried over anhydrous Na₂SO₄, filtered and concentrated under reduced pressure to dryness. The residue was purified by prep. HPLC (C18, 45˜95% acetonitrile in H₂O with 0.1% trifluoroacetic acid) to give Example 17 (11 mg, 25% yield) and Example 18 (10 mg, 22.4% yield) as white solid. Example 17: LC/MS (ESI) m/z: 492 (M+H)⁺. ¹H NMR (400 MHz, CD₃OD) δ 7.94 (d, J=8.9 Hz, 1H), 7.85 (s, 1H), 7.74 (d, J=9.0 Hz, 1H), 7.57 (dd, J=9.2, 2.3 Hz, 1H), 6.61 (d, J=9.2 Hz, 1H), 5.56-5.47 (m, 2H), 4.15 (s, 3H), 3.66-3.60 (m, 1H), 3.56-3.49 (m, 2H), 3.42-3.35 (m, 1H), 3.26-3.19 (m, 2H), 2.62-2.52 (m, 1H), 2.47-2.43 (m, 2H), 2.39-2.27 (m, 2H), 1.85-1.66 (m, 3H), 1.62-1.56 (m, 2H), 1.37 (t, J=7.5 Hz, 3H), 0.99 (t, J=7.4 Hz, 3H), 0.94 (t, J=7.3 Hz, 3H). Example 18: LC/MS (ESI) m/z: 492 (M+H)⁺. ¹H NMR (400 MHz, CD₃OD) δ 7.92 (d, J=8.8 Hz, 1H), 7.80 (s, 1H), 7.68 (d, J=8.9 Hz, 1H), 7.56-7.50 (m, 1H), 6.59 (d, J=9.2 Hz, 1H), 5.60 (s, 2H), 4.15 (s, 3H), 3.63-3.50 (m, 2H), 3.47-3.41 (m, 1H), 3.29-3.26 (m, 1H), 3.15 (dt, J=5.0, 4.4 Hz, 2H), 2.58-2.50 (m, 1H), 2.43-2.32 (m, 3H), 2.25-2.14 (m, 1H), 1.88-1.79 (m, 1H), 1.73-1.64 (m, 2H), 1.59-1.49 (m, 2H), 1.35 (t, J=7.5 Hz, 3H), 0.98 (t, J=7.4 Hz, 3H), 0.90 (t, J=7.3 Hz, 3H).

### Example 19 & Example 20: (S) or (R)-2-((S)-1-(2-ethyl-6-(1-methyl-5-(((methyl(propyl)carbamoyl)oxy)methyl)-1H-1,2,3-triazol-4-yl)pyridin-3-yl)pyrrolidin-3-yl)butanoic acid and (R) or (S)-2-((S)-1-(2-ethyl-6-(1-methyl-5-(((methyl(propyl)carbamoyl)oxy)methyl)-1H-1,2,3-triazol-4-yl)pyridin-3-yl)pyrrolidin-3-yl)butanoic acid

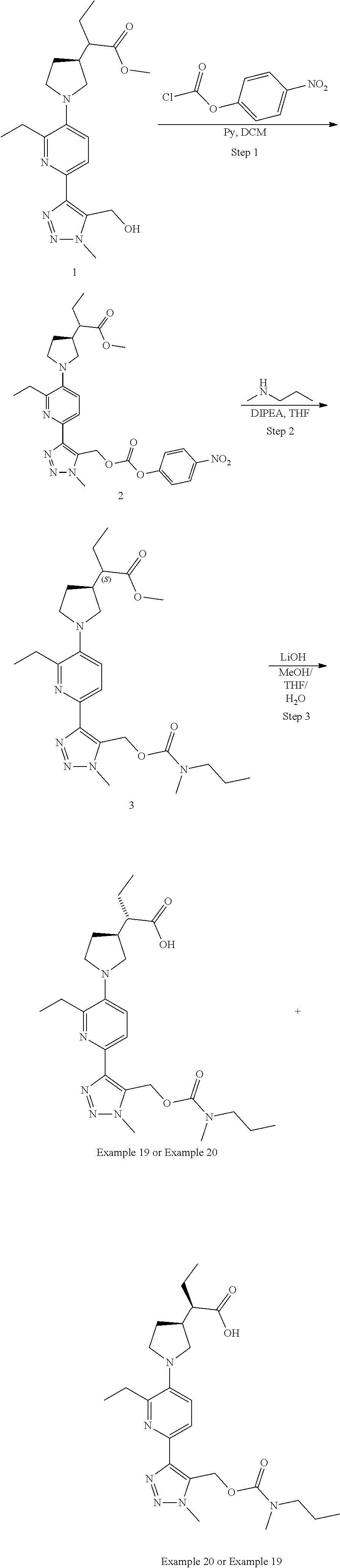

### Step 1: methyl 2-((S)-1-(2-ethyl-6-(1-methyl-5-((((4-nitrophenoxy)carbonyl)oxy)methyl)-1H-1,2,3-triazol-4-yl)pyridin-3-yl)pyrrolidin-3-yl)butanoate

To a solution of methyl 2-((S)-1-(2-ethyl-6-(5-(hydroxymethyl)-1-methyl-1H-1,2,3-triazol-4-yl)pyridin-3-yl)pyrrolidin-3-yl)butanoate (50 mg, 0.13 mmol) in DCM (3 mL) was added pyridine (50 mg, 0.65 mmol) and 4-nitrophenyl chloroformate (78 mg, 0.39 mmol) and the mixture was stirred at r.t. for 2 hrs. The mixture reaction was concentrated to dryness to give the title compound (80 mg, 100% yield) as yellow solid, which was directly used in the next reaction without purification. LC/MS (ESI) m/z: 553 (M+H)⁺.

### Step 2: methyl 2-((S)-1-(2-ethyl-6-(1-methyl-5-(((methyl(propyl)carbamoyl)oxy)methyl)-1H-1,2,3-triazol-4-yl)pyridin-3-yl)pyrrolidin-3-yl)butanoate

To a solution of methyl 2-((S)-1-(2-ethyl-6-(1-methyl-5-((((4-nitrophenoxy) carbonyl)oxy)methyl)-1H-1,2,3-triazol-4-yl)pyridin-3-yl)pyrrolidin-3-yl)butanoate (80 mg, 0.13 mmol) in THF (3 mL) was added DIPEA (65 mg, 0.5 mmol) and N-methylpropan-1-amine (22 mg, 0.3 mmol) and the mixture was stirred at r.t. for 1 hr. The mixture was quenched with saturated aq. NH₄Cl solution and extracted with EtOAc (3×10 mL). The combined organic layers were washed with brine, dried over anhydrous Na₂SO₄, filtered and concentrated to dryness. The residue was purified by flash chromatography (silica gel, 0˜50% of EtOAc in PE) to give the compound (35 mg, 55.7% yield) as yellow solid. LC/MS (ESI) m/z: 487 (M+H)⁺.

### Step 3: (S) or (R)-2-((S)-1-(2-ethyl-6-(1-methyl-5-(((methyl(propyl)carbamoyl)oxy)methyl)-1H-1,2,3-triazol-4-yl)pyridin-3-yl)pyrrolidin-3-yl)butanoic acid and (R) or (S)-2-((S)-1-(2-ethyl-6-(1-methyl-5-(((methyl(propyl)carbamoyl)oxy)methyl)-1H-1,2,3-triazol-4-yl)pyridin-3-yl)pyrrolidin-3-yl)butanoic acid

To a solution of methyl 2-((S)-1-(2-ethyl-6-(1-methyl-5-(((methyl(propyl)carbamoyl)oxy)methyl)-1H-1,2,3-triazol-4-yl)pyridin-3-yl)pyrrolidin-3-yl)butanoate (35 mg, 0.07 mmol) in MeOH (2 mL), THE (2 mL) and H₂O (1 mL) was added NaOH (36 mg, 0.9 mmol) and the mixture was stirred at room temperature for 16 hrs. The mixture was concentrated to dryness and the residue was dissolved in water (5 mL). The mixture was washed with EtOAc (2×5 mL), acidified with 1N aq. HCl to pH-3 and extracted with DCM (3×5 mL). The combined organic layers were washed with brine, dried over anhydrous Na₂SO₄, filtered and concentrated under reduced pressure to dryness. The residue was purified by prep. HPLC (C18, 45˜95% acetonitrile in H₂O with 0.1% TFA) to give compound example 19 (6 mg, 18% yield) and example 20 (1.5 mg, 4.5% yield) as white solid.

Example 19: LC/MS (ESI) m/z: 473 (M+H)⁺. ¹H NMR (400 MHz, CD₃OD) δ 8.00 (dd, J=8.9, 2.9 Hz, 1H), 7.81 (dd, J=8.9, 3.3 Hz, 1H), 5.50 (s, 2H), 4.24 (d, J=2.1 Hz, 3H), 3.68-3.60 (m, 2H), 3.55-3.51 (m, 1H), 3.39-3.34 (m, 1H), 3.27-3.22 (m, 4H), 2.93 (d, J=19.8 Hz, 3H), 2.60-2.50 (m, 1H), 2.38-2.32 (m, 1H), 2.24-2.18 (m, 1H), 1.90-1.80 (m, 1H), 1.73-1.66 (m, 2H), 1.60-1.52 (m, 2H), 1.39 (t, J=7.5 Hz, 3H), 0.99 (t, J=7.4 Hz, 3H), 0.92-0.82 (m, 3H).

Example 20: LC/MS (ESI) m/z: 473 (M+H)⁺. ¹H NMR (400 MHz, CD₃OD) δ 7.72 (d, J=8.5 Hz, 1H), 7.28 (d, J=8.5 Hz, 1H), 5.75 (s, 2H), 4.17 (s, 3H), 3.18-3.08 (m, 3H), 2.89-2.80 (m, 5H), 2.51-2.44 (m, 1H), 2.33-2.13 (m, H), 1.75-1.65 (m, 3H), 1.57-1.50 (m, 1H), 1.38-1.27 (m, 6H), 0.98 (t, J=7.1 Hz, 3H), 0.89-0.68 (m, 3H).

### Example 21: 2-(rel-(2S,6R)-6-(6-(5-(((4-cyclobutylpyrimidin-2-yl)oxy)methyl)-1-methyl-1H-1,2,3-triazol-4-yl)-2-ethylpyridin-3-yl)tetrahydro-2H-pyran-2-yl)acetic acid

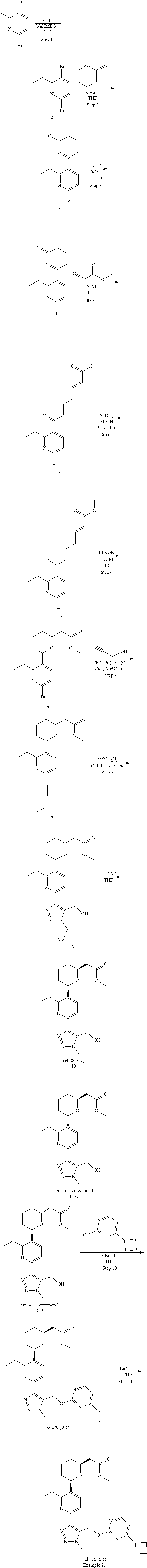

### Step 1: 3,6-dibromo-2-ethylpyridine

To a solution of 3,6-dibromo-2-methylpyridine (20 g, 79.7 mmol) in THE (250 mL) was added NaHMDS (95.6 mol, 47.8 mL, 2 M in THF) drop-wisely at −50° C. under N₂ atmosphere over 30 mins and the mixture was stirred at −50° C. for 1 hr. Mel (28.3 g, 199.3 mmol) was added drop-wisely to the mixture over a period of 30 mins while maintaining the temperature below −40° C. The reaction mixture was further stirred at −50° C. for 1 hr until the disappearance of the starting material was confirmed by TLC analysis. The reaction was quenched with saturated aq. NH₄Cl solution at 0° C. The mixture was extracted with EtOAc (2×200 mL) and the combined organic layers were washed with water and brine, dried over anhydrous Na₂SO₄, filtered and concentrated to dryness. The residue was purified by flash chromatography (silica gel, 0˜2% of EtOAc in PE) to give the title compound (14 g, 66.3% yield) as yellow oil. LC/MS (ESI) m/z: 266 (M+H)⁺. ¹H NMR (400 MHz, CDCl₃) δ 7.62 (d, J=8.3 Hz, 1H), 7.18 (d, J=8.3 Hz, 1H), 2.94 (q, J=7.5 Hz, 2H), 1.28 (t, J=7.5 Hz, 3H).

### Step 2: 1-(6-bromo-2-ethylpyridin-3-yl)-5-hydroxypentan-1-one

To a solution of 3,6-dibromo-2-ethylpyridine (7 g, 26.4 mmol) in THE (100 mL) was added n-BuLi (10.6 mL, 26.4 mmol, 2.5 M in hexanes) drop-wisely at −78° C. under N₂ atmosphere over 30 mins and the mixture was stirred at −78° C. for 1 hr. Tetrahydro-2H-pyran-2-one (5.3 g, 52.8 mmol) in THE (20 mL) added drop-wisely to the mixture over a period of 30 mins while maintaining the temperature below −60° C. The reaction mixture was further stirred at −78° C. for 1 hr until the disappearance of the starting material was confirmed by TLC analysis. The reaction was quenched with saturated aq. NH₄Cl solution at 0° C. The reaction mixture was extracted with EtOAc (2×50 mL) and the combined organic layers were washed with water and brine, dried over anhydrous Na₂SO₄, filtered and concentrated to dryness. The residue was purified by flash chromatography (silica gel, 0˜50% of EtOAc in PE) to give the title compound (3.6 g, 47.6% yield) as yellow solid. LC/MS (ESI) m/z: 287 (M+H)⁺.

### Step 3: 5-(6-bromo-2-ethylpyridin-3-yl)-5-oxopentanal

To a solution of 1-(6-bromo-2-ethylpyridin-3-yl)-5-hydroxypentan-1-one (3.6 g, 12.6 mmol) in DCM (50 mL) was added Dess-Martin periodinane (8.00 g, 18.9 mmol) at 0° C. and the mixture was stirred at r.t. for 2 hrs. The reaction mixture was washed with saturated aq. NaHCO₃ solution and brine, dried over Na₂SO₄, filtered and concentrated to dryness. The residue was purified by flash chromatography (silica gel, 0˜25% of EtOAc in PE) to give the title compound (1.7 g, 47.7% yield) as yellow oil. LC/MS (ESI) m/z: 286 (M+H)⁺. ¹H NMR (400 MHz, CDCl₃) δ 9.81 (t, J=1.2 Hz, 1H), 7.71 (d, J=8.2 Hz, 1H), 7.40 (d, J=8.2 Hz, 1H), 2.96 (q, J=7.5 Hz, 3H), 2.92 (t, J=7.1 Hz, 2H), 2.60 (td, J=6.9, 1.1 Hz, 2H), 2.05 (dd, J=13.9, 7.0 Hz, 2H), 1.28 (t, J=7.5 Hz, 3H).

### Step 4: methyl (E)-7-(6-bromo-2-ethylpyridin-3-yl)-7-oxohept-2-enoate

To a solution of 5-(6-bromo-2-ethylpyridin-3-yl)-5-oxopentanal (1.7 g, 6.0 mmol) in DCM (20 mL) was added methyl 2-(triphenyl-15-phosphanylidene)acetate (2.4 g, 7.2 mmol) and the mixture was stirred at r.t. for 2 hrs. The reaction mixture was concentrated to dryness. The residue was purified by flash chromatography (silica gel, 0˜35% of EtOAc in PE) to give the title compound (1.8 g, 88.4% yield) as yellow oil. LC/MS (ESI) m/z: 341 (M+H)⁺.

### Step 5: methyl (E)-7-(6-bromo-2-ethylpyridin-3-yl)-7-hydroxyhept-2-enoate

To a solution of methyl (E)-7-(6-bromo-2-ethylpyridin-3-yl)-7-oxohept-2-enoate (1.8 g, 5.3 mmol) in MeOH (20 mL) was added NaBH₄ (200 mg, 5.3 mol) in portions at 0° C. and the mixture was stirred at r.t. for 30 min. The reaction was quenched with saturated aq. NH₄Cl solution at 0° C. and extracted with DCM (2×30 mL). The combined organic layers were washed with brine, dried over anhydrous Na₂SO₄, filtered and concentrated to dryness. The residue was purified by flash chromatography (silica gel, 0˜50% of EtOAc in PE) to give the title compound (1.5 g, 82.8% yield) as white solid. LC/MS (ESI) m/z: 343 (M+H)⁺.

### Step 6: methyl 2-(6-(6-bromo-2-ethylpyridin-3-yl)tetrahydro-2H-pyran-2-yl)acetate

To a solution of methyl (E)-7-(6-bromo-2-ethylpyridin-3-yl)-7-hydroxyhept-2-enoate (1.5 g, 4.4 mmol) in THE (20 mL) was added t-BuOK (4.4 mL, 4.4 mmol, 1 M in THF) drop-wisely at 0° C. under N₂ atmosphere over 10 mins and the mixture was stirred at 0° C. for 30 min until the disappearance of the starting material was confirmed by TLC analysis. The reaction was quenched with saturated aq. NH₄Cl solution at 0° C. The mixture was extracted with EtOAc (2×20 mL) and the combined organic layers were washed with water and brine, dried over anhydrous Na₂SO₄, filtered and concentrated to dryness. The residue was purified by flash chromatography (silica gel, 0˜30% of EtOAc in PE) to give the title compound (1.3 g, 86.7% yield) as yellow solid. LC/MS (ESI) m/z: 343 (M+H)⁺.

### Step 7: methyl 2-(6-(2-ethyl-6-(3-hydroxyprop-1-yn-1-yl)pyridin-3-yl)tetrahydro-2H-pyran-2-yl)acetate

To a solution of methyl 2-(6-(6-bromo-2-ethylpyridin-3-yl)tetrahydro-2H-pyran-2-yl)acetate (1.3 g, 3.8 mmol) in MeCN (30 mL) was added triethylamine (1.6 mL, 11.4 mmol) followed by Pd(PPh₃)₂Cl₂ (590 mg, 0.76 mmol) and prop-2-yn-1-ol (0.45 mL, 7.6 mmol) at 0° C. and the mixture was stirred at room temperature under N₂ atmosphere for 16 hrs. The reaction mixture was filtered and concentrated to dryness. The residue was purified by flash chromatography (silica gel, 0˜65% of EtOAc in PE) to give the title compound (1.1 g, 91.2% yield) as yellow oil. LC/MS (ESI) m/z: 318 (M+H)⁺.

### Step 8: methyl 2-(6-(2-ethyl-6-(5-(hydroxymethyl)-1-((trimethylsilyl)methyl)-1H-1,2,3-triazol-4-yl)pyridin-3-yl)tetrahydro-2H-pyran-2-yl)acetate

To a solution of methyl 2-(6-(2-ethyl-6-(3-hydroxyprop-1-yn-1-yl)pyridin-3-yl)tetrahydro-2H-pyran-2-yl)acetate (1.1 g, 3.5 mmol) in 1,4-dioxane (20 mL) was added TMSCH₂N₃ (1.1 g, 8.7 mmol) followed by CuI (70 mg, 0.35 mmol) and pentamethylcyclopentadienylbis(triphenylphosphine)ruthenium(II) chloride (280 mg, 0.35 mmol) at 0° C. and the mixture was stirred at 60° C. under N₂ atmosphere for 16 hrs. The reaction mixture was filtered and the filtrate was concentrated to dryness. The residue was purified by flash chromatography (silica gel, 0˜40% of EtOAc in PE) to give the title compound (750 mg, 48.4% yield) as yellow oil. LC/MS (ESI) m/z: 447 (M+H)⁺.

### Step 9: methyl 2-(rel-(2S,6R)-6-(2-ethyl-6-(5-(hydroxymethyl)-1-methyl-1H-1,2,3-triazol-4-yl)pyridin-3-yl)tetrahydro-2H-pyran-2-yl)acetate (10) & methyl 2-((2S,6S) or (2R,6R)-6-(2-ethyl-6-(5-(hydroxymethyl)-1-methyl-1H-1,2,3-triazol-4-yl)pyridin-3-yl)tetrahydro-2H-pyran-2-yl)acetate (10-1) & methyl 2-((2R,6R) or (2S,6S)-6-(2-ethyl-6-(5-(hydroxymethyl)-1-methyl-1H-1,2,3-triazol-4-yl)pyridin-3-yl)tetrahydro-2H-pyran-2-yl)acetate

To a solution of methyl 2-(6-(2-ethyl-6-(5-(hydroxymethyl)-1-((trimethylsilyl)methyl)-1H-1,2,3-triazol-4-yl)pyridin-3-yl)tetrahydro-2H-pyran-2-yl)acetate (750 mg, 1.7 mmol) in THE (10 mL) was added TBAF (1.7 mL, 1.7 mmol, 1 M in THF) drop-wisely at 0° C. The reaction mixture was stirred at 0° C. for 1 hr. The reaction mixture was diluted with EtOAc (20 mL), washed with water and brine, dried over anhydrous Na₂SO₄, filtered and concentrated under reduced pressure to dryness. The residue was purified by flash chromatography (silica gel, 0˜70% of EtOAc in PE) to give the compound 10 (50 mg, 8.3% yield) and a mixture of compound 10-1 and compound 10-2 (520 mg, 82.7% yield) as white solid. The mixture was separated by chiral SFC to give compound 10-1 (200 mg, 31.8% yield) (Peak 1, retention time: 2.909 min) and compound 10-2 (210 mg, 33.4% yield) (Peak 2, retention time: 3.566 min) as white solids. SFC condition: Column: ChiralPak AD, 250×21.2 mm I.D., 5 μm; Mobile phase: A for CO2 and B for Methanol (0.1% NH₄OH); Gradient: B 30%; Flow rate: 50 mL/min; Column temperature: 35° C.

### Step 10: methyl 2-(rel-(2S,6R)-6-(6-(5-(((4-cyclobutylpyrimidin-2-yl)oxy)methyl)-1-methyl-1H-1,2,3-triazol-4-yl)-2-ethylpyridin-3-yl)tetrahydro-2H-pyran-2-yl)acetate

To a mixture of methyl 2-(rel-(2S,6R)-6-(2-ethyl-6-(5-(hydroxymethyl)-1-methyl-1H-1,2,3-triazol-4-yl)pyridin-3-yl)tetrahydro-2H-pyran-2-yl)acetate (50 mg, 0.13 mmol) and 2-chloro-4-cyclobutylpyrimidine (34 mg, 0.2 mmol) in THE (5 mL) was added t-BuOK (0.25 mL, 0.25 mmol, 1.0 M in THF) at 0° C. The reaction mixture was stirred at 0° C. for 30 min until the disappearance of the starting material was confirmed by TLC analysis. The reaction was quenched with saturated aq. NH₄Cl solution at 0° C. and the mixture was extracted with EtOAc (2×20 mL). The combined organic layers were washed with brine, dried over anhydrous Na₂SO₄, filtered and concentrated to dryness. The residue was purified by flash chromatography (silica gel, 0˜60% of EtOAc in PE) to give the title compound (35 mg, 51.7% yield) as yellow solid. LC/MS (ESI) m/z: 507 (M+H)⁺.

### Step 11: 2-(rel-(2S,6R)-6-(6-(5-(((4-cyclobutylpyrimidin-2-yl)oxy)methyl)-1-methyl-1H-1,2,3-triazol-4-yl)-2-ethylpyridin-3-yl)tetrahydro-2H-pyran-2-yl)acetic acid

To a mixture of methyl 2-(rel-(2S,6R)-6-(6-(5-(((4-cyclobutylpyrimidin-2-yl)oxy)methyl)-1-methyl-1H-1,2,3-triazol-4-yl)-2-ethylpyridin-3-yl)tetrahydro-2H-pyran-2-yl)acetate (35 mg, 0.07 mmol) in THE (4 mL)/H₂O (1 mL)/MeOH (1 mL) was added LiOH·H₂O (29 mg, 0.69 mmol), and the reaction was stirred at r.t. for 2 hrs. Volatiles were removed under vacuum and the residue was diluted with H₂O (5 mL). The mixture was adjusted with 1N aq. HCl to pH˜4 and extracted with EtOAc (3×10 mL). The combined organic layers were washed with brine, dried over anhydrous Na₂SO₄, filtered and concentrated to dryness. The residue was purified by prep-HPLC (C18, 5-95%, MeCN in H₂O with 0.1% HCOOH) to give the title compound (12 mg, 35.3% yield) as white solid. LC/MS (ESI) m/z: 493 (M+H)⁺. ¹H NMR (400 MHz, DMSO-d₆) δ 8.49 (d, J=5.0 Hz, 1H), 7.94 (d, J=8.1 Hz, 1H), 7.81 (d, J=8.1 Hz, 1H), 7.04 (d, J=5.0 Hz, 1H), 6.09 (dd, J=27.1, 12.6 Hz, 2H), 4.58 (d, J=10.9 Hz, 1H), 4.16 (s, 3H), 3.98-3.88 (m, 1H), 3.53 (dd, J=17.0, 8.5 Hz, 1H), 2.82-2.66 (m, 2H), 2.45-2.33 (m, 2H), 2.23-2.15 (m, 4H), 2.02-1.93 (m, 1H), 1.92-1.86 (m, 1H), 1.80-1.65 (m, 4H), 1.50-1.40 (m, 1H), 1.35-1.22 (m, 1H), 1.08 (t, J=7.4 Hz, 3H).

### Example 22: 2-((2S,6S) or (2R,6R)-6-(6-(5-(((4-cyclobutylpyrimidin-2-yl)oxy)methyl)-1-methyl-1H-1,2,3-triazol-4-yl)-2-ethylpyridin-3-yl)tetrahydro-2H-pyran-2-yl)acetic acid

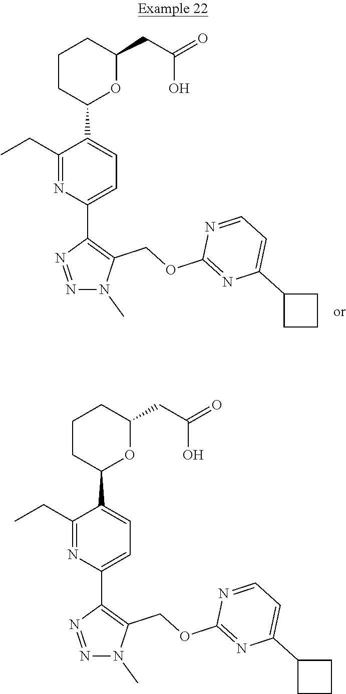 [SMILES: CCc1nc(-c2nnn(C)c2COc2nccc(C3CCC3)n2)ccc1[C@@H]1CCC[C@@H](CC(=O)O)O1 ; CCc1nc(-c2nnn(C)c2COc2nccc(C3CCC3)n2)ccc1[C@H]1CCC[C@H](CC(=O)O)O1]

The title compound was synthesized from compound 10-1 of Example 21 using the same synthetic sequence that was used to synthesize Example 21. LC/MS (ESI) m/z: 493 (M+H)⁺. ¹H NMR (400 MHz, CD₃OD) δ 8.40 (d, J=5.1 Hz, 1H), 7.87 (d, J=8.4 Hz, 2H), 6.98 (d, J=5.1 Hz, 1H), 6.27-6.16 (m, 2H), 4.69-4.63 (m, 1H), 4.23 (s, 3H), 4.07-3.98 (m, 1H), 3.56-3.45 (m, 1H), 2.95-2.73 (m, 2H), 2.58-2.48 (m, 2H), 2.26-2.15 (m, 4H), 2.08-1.97 (m, 2H), 1.87-1.73 (m, 4H), 1.62-1.52 (m, 1H), 1.45-1.35 (m, 1H), 1.22 (t, J=7.5 Hz, 3H).

### Example 23: 2-((2R,6R) or (2S,6S)-6-(6-(5-(((4-cyclobutylpyrimidin-2-yl)oxy)methyl)-1-methyl-1H-1,2,3-triazol-4-yl)-2-ethylpyridin-3-yl)tetrahydro-2H-pyran-2-yl)acetic acid

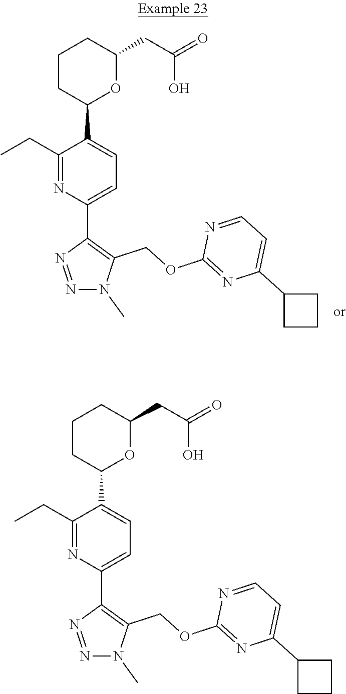 [SMILES: CCc1nc(-c2nnn(C)c2COc2nccc(C3CCC3)n2)ccc1[C@H]1CCC[C@H](CC(=O)O)O1 ; CCc1nc(-c2nnn(C)c2COc2nccc(C3CCC3)n2)ccc1[C@@H]1CCC[C@@H](CC(=O)O)O1]

The title compound was synthesized from compound 10-2 of Example 21 using the same synthetic sequence that was used to synthesize Example 21. LC/MS (ESI) m/z: 493 (M+H)⁺. ¹H NMR (400 MHz, CD₃OD) δ 8.40 (d, J=5.1 Hz, 1H), 7.99-7.82 (m, 2H), 6.98 (d, J=5.1 Hz, 1H), 6.27-6.12 (m, 2H), 4.72-4.62 (m, 1H), 4.23 (s, 3H), 4.09-3.96 (m, 1H), 3.59-3.44 (m, 1H), 2.95-2.73 (m, 2H), 2.57-2.46 (m, 2H), 2.26-2.15 (m, 4H), 2.09-1.95 (m, 2H), 1.86-1.72 (m, 4H), 1.65-1.52 (m, 1H), 1.47-1.34 (m, 1H), 1.22 (t, J=7.5 Hz, 3H).

### Example 24: 2-((2S,6S) or (2R,6R)-6-(2-ethyl-6-(1-methyl-5-((2-oxo-5-propylpyridin-1(2H)-yl)methyl)-1H-1,2,3-triazol-4-yl)pyridin-3-yl)tetrahydro-2H-pyran-2-yl)acetic acid

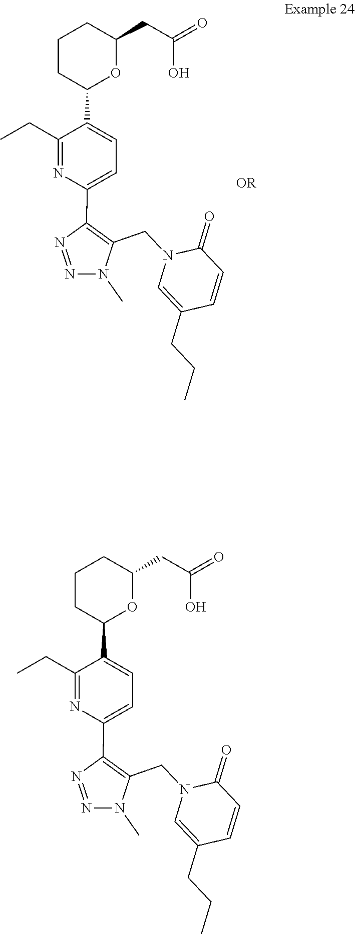 [SMILES: CCCc1ccc(=O)n(Cc2c(-c3ccc([C@@H]4CCC[C@@H](CC(=O)O)O4)c(CC)n3)nnn2C)c1 ; CCCc1ccc(=O)n(Cc2c(-c3ccc([C@H]4CCC[C@H](CC(=O)O)O4)c(CC)n3)nnn2C)c1]

The title compound was synthesized from compound 10-1 of Example 21 using the same synthetic sequence that was used to synthesize Example 14. LC/MS (ESI) m/z: 480 (M+H)⁺. ¹H NMR (400 MHz, CD₃OD) δ 7.91 (d, J=8.2 Hz, 1H), 7.89 (d, J=8.2 Hz, 1H), 7.66 (d, J=2.2 Hz, 1H), 7.38 (dd, J=9.2, 2.5 Hz, 1H), 6.52 (d, J=9.3 Hz, 1H), 5.88 (s, 2H), 4.72-4.66 (m, 1H), 4.16 (s, 3H), 4.09-4.01 (m, 1H), 3.00-2.87 (m, 2H), 2.52 (d, J=6.5 Hz, 2H), 2.21 (t, J=7.5 Hz, 2H), 2.04-1.98 (m, 1H), 1.88-1.75 (m, 3H), 1.64-1.53 (m, 1H), 1.41 (dd, J=12.0, 4.6 Hz, 1H), 1.38-1.32 (m, 2H), 1.30 (t, J=7.5 Hz, 3H), 0.75 (t, J=7.3 Hz, 3H).

### Example 25: 2-((2S,6S) or (2R,6R)-6-(2-ethyl-6-(1-methyl-5-(((methyl(propyl)carbamoyl)oxy)methyl)-1H-1,2,3-triazol-4-yl)pyridin-3-yl)tetrahydro-2H-pyran-2-yl)acetic acid

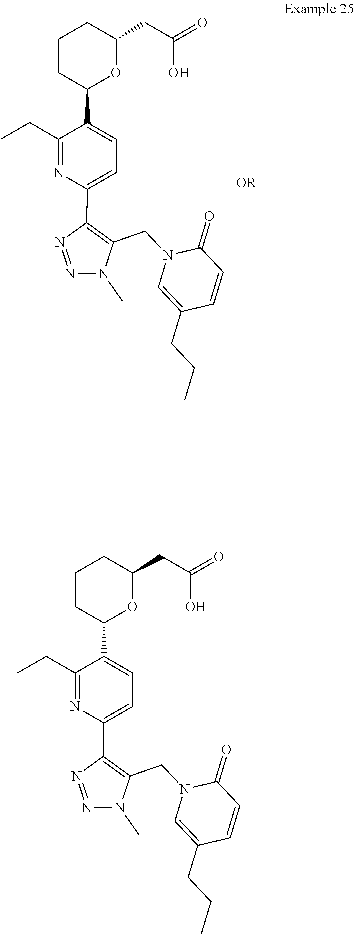 [SMILES: CCCc1ccc(=O)n(Cc2c(-c3ccc([C@H]4CCC[C@H](CC(=O)O)O4)c(CC)n3)nnn2C)c1 ; CCCc1ccc(=O)n(Cc2c(-c3ccc([C@@H]4CCC[C@@H](CC(=O)O)O4)c(CC)n3)nnn2C)c1]

The title compound was synthesized from compound 10-2 of Example 21 using the same synthetic sequence that was used to synthesize Example 14. LC/MS (ESI) m/z: 460 (M+H)⁺. ¹H NMR (400 MHz, CD₃OD) δ 7.85 (s, 2H), 5.76 (s, 2H), 4.69-4.63 (m, 1H), 4.18 (s, 3H), 4.07-3.99 (m, 1H), 3.21 (t, J=7.0 Hz, 1H), 3.09 (t, J=7.0 Hz, 1H), 2.93 (dd, J=14.9, 7.5 Hz, 1H), 2.87 (d, J=7.6 Hz, 1H), 2.85-2.79 (m, 3H), 2.51 (d, J=6.5 Hz, 2H), 2.03-1.96 (m, 1H), 1.87-1.75 (m, 3H), 1.55 (dt, J=12.3, 9.4 Hz, 2H), 1.41 (dd, J=11.6, 7.7 Hz, 1H), 1.36 (d, J=9.6 Hz, 2H), 1.32 (d, J=7.3 Hz, 2H), 0.77 (m, 3H).

### Example 26: 2-(2-(6-(5-(((4-cyclobutylpyrimidin-2-yl)oxy)methyl)-1-methyl-11H-1,2,3-triazol-4-yl)-2-methylpyridin-3-yl)tetrahydro-2H-pyran-4-yl)acetic acid

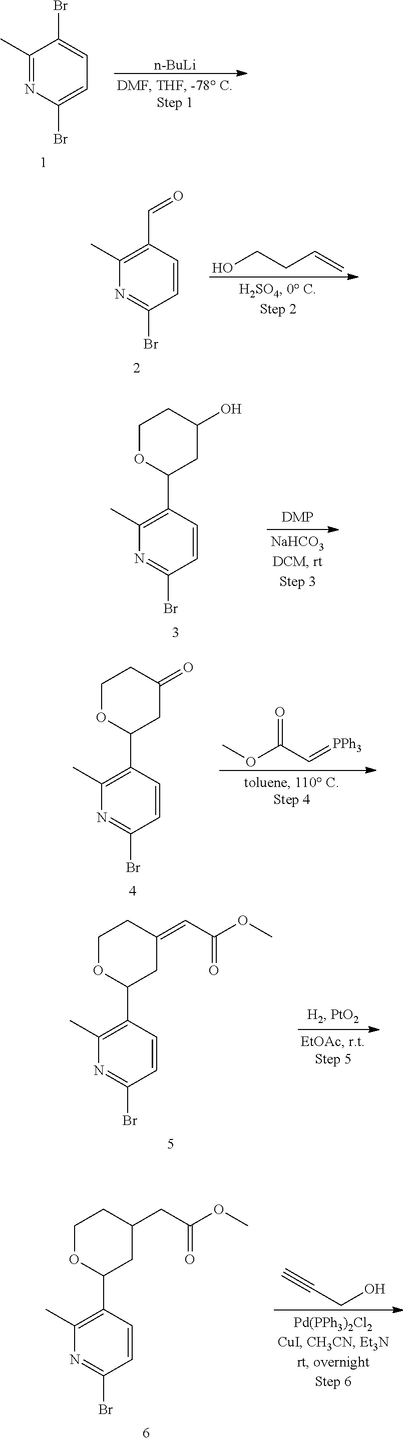

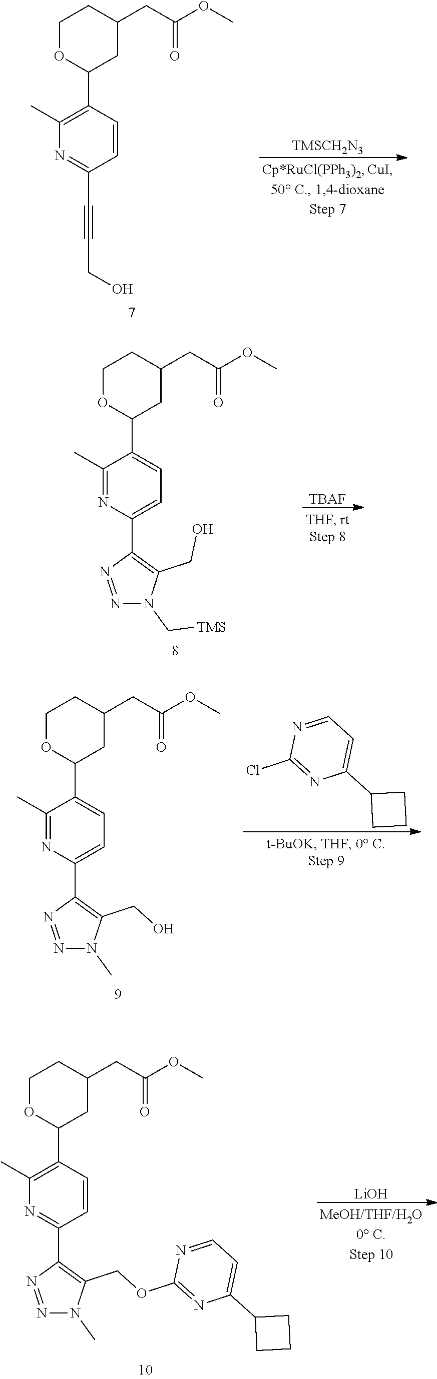

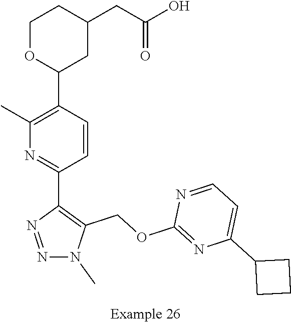 [SMILES: Cc1nc(-c2nnn(C)c2COc2nccc(C3CCC3)n2)ccc1C1CC(CC(=O)O)CCO1]

### Step 1: 6-bromo-2-methylnicotinaldehyde

To a solution of 3,6-dibromo-2-methylpyridine (10 g, 39.9 mmol) in THF (110 mL) was added n-BuLi (20.7 mL, 51.8 mmol, 2.5 M in hexane) at −78° C. under N₂ atmosphere. The mixture reaction was stirred at −78° C. for 20 mins. Then DMF (6.17 mL, 79.7 mmol) was added drop-wisely and the mixture was stirred at −78° C. for 30 mins. The mixture was poured into saturated aq. NH₄Cl solution (100 mL) and extracted with EtOAc (3×100 mL). The combined organic layers were washed with brine, dried over anhydrous Na₂SO₄, filtered and concentrated to dryness. The residue was purified by flash chromatography (silica gel, 0-10% of EtOAc in PE) to give the title compound (4 g, 50.2% yield) as yellow solid. LC/MS (ESI) m/z: 201 (M+H)⁺.

### Step 2: 2-(6-bromo-2-methylpyridin-3-yl)tetrahydro-2H-pyran-4-ol

To a mixture of 6-bromo-2-methylnicotinaldehyde (4 g, 20 mmol) and but-3-en-1-ol (3.48 mL, 40 mmol) was added sulfuric acid (14.25 g, 80 mmol) at 0° C. and the mixture was stirred at 30 mins. The reaction mixture was adjusted with saturated aq. NaOH solution to pH-9 and extracted with EtOAc (3×100 mL). The combined organic layers were washed with brine, dried with anhydrous Na₂SO₄, filtered and concentrated to dryness. The residue was purified by flash chromatography (silica gel, 0-40% EtOAc in PE) to give the title compound (870 mg, 16.1% yield) as yellow oil. LC/MS (ESI) m/z: 273 (M+H)⁺.

### Step 3: 2-(6-bromo-2-methylpyridin-3-yl)tetrahydro-4H-pyran-4-one

To a solution of 2-(6-bromo-2-methylpyridin-3-yl)tetrahydro-2H-pyran-4-ol (870 mg, 3.20 mmol) in DCM (10 mL) were added Dess-Martin Periodinate (1.86 g, 9.59 mol) and NaHCO₃ (1.07 g, 12.8 mmol) at room temperature. The reaction was stirred at r.t. for 16 hrs. The mixture was filtered through Celite pad and the filtrate was diluted with saturated aq. NaHCO₃ solution and extracted with EtOAc (3×50 mL). The combined organic layers were washed with brine, dried with anhydrous Na₂SO₄, filtered and concentrated to dryness. The residue was purified by flash chromatography (silica gel, 0˜10% EtOAc in PE) to give the title compound (660 mg, 76.4% yield) as yellow solid. LC/MS (ESI) m/z: 271 (M+H)⁺.

### Step 4: methyl (Z)-2-(2-(6-bromo-2-methylpyridin-3-yl)tetrahydro-4H-pyran-4-ylidene)acetate

To a solution of 2-(6-bromo-2-methylpyridin-3-yl)tetrahydro-4H-pyran-4-one (660 mg, 2.4 mmol) in toluene (8 mL) was added methyl 2-(triphenyl-15-phosphanylidene)acetate (1.6 g, 4.9 mmol) and the mixture was stirred at 100° C. for 3 hrs. The solution was concentrated under reduced pressure to dryness. The residue was purified by flash chromatography (silica gel, 0˜40% EtOAc in PE) to give the title compound (750 mg, 94.6% yield) as yellow solid. LC/MS (ESI) m/z: 326 (M+H)⁺.

### Step 5: methyl 2-(2-(6-bromo-2-methylpyridin-3-yl)tetrahydro-2H-pyran-4-yl)acetate

To a solution of methyl (Z)-2-(2-(6-bromo-2-methylpyridin-3-yl)tetrahydro-4H-pyran-4-ylidene)acetate (750 mg, 2.3 mmol) in EtOAc (10 mL) was added PtO₂ (70 mg, 0.03 mmol) at r.t. The mixture was degassed under N₂ atmosphere for three times and stirred under a H₂ balloon at r.t. for 2 hrs. The reaction mixture was filtered through a pad of Celite and the filtrate was concentrated to dryness to give the title compound (680 mg, 90% yield) as yellow solid, which was used in the next step without further purification. LC/MS (ESI) m/z: 328 (M+H)⁺.

### Step 6: methyl 2-(2-(6-(3-hydroxyprop-1-yn-1-yl)-2-methylpyridin-3-yl)tetrahydro-2H-pyran-4-yl)acetate

To a solution of methyl 2-(2-(6-bromo-2-methylpyridin-3-yl)tetrahydro-2H-pyran-4-yl)acetate (680 mg, 2.1 mmol) in acetonitrile (10 mL) were added bis(triphenylphosphine) palladium(II) chloride (292 mg, 0.42 mmol), CuI (79.2 mg, 0.42 mmol), TEA (0.87 mL, 6.24 mmol) and prop-2-yn-1-ol (0.24 mL, 4.16 mmol) under N₂ atmosphere. The reaction was degassed under N₂ atmosphere for three times and stirred at room temperature for 5 hrs. The mixture was poured into saturated aq. NH₄Cl solution and extracted with EtOAc (3×30 mL). The combined organic layers were washed with brine, dried over anhydrous Na₂SO₄, filtered and concentrated to dryness. The residue was purified by flash chromatography (silica gel, 0˜60% EtOAc in PE) to give the title compound (540 mg, 85.7% yield) as yellow oil. LC/MS (ESI) m/z: 304 (M+H)⁺.

### Step 7: methyl 2-(2-(6-(5-(hydroxymethyl)-1-((trimethylsilyl)methyl)-1H-1,2,3-triazol-4-yl)-2-methylpyridin-3-yl)tetrahydro-2H-pyran-4-yl)acetate

To a solution of methyl 2-(2-(6-(3-hydroxyprop-1-yn-1-yl)-2-methylpyridin-3-yl)tetrahydro-2H-pyran-4-yl)acetate (540 mg, 1.78 mmol) in 1,4-dioxane (8 mL) were added Cp*RuCl(PPh₃)₂ (141.7 mg, 0.18 mmol), cuprous iodide (34 mg, 0.18 mmol) and trimethylsilylmethyl azide (0.56 mL, 4.1 mmol) at r.t. under N₂ atmosphere. The reaction mixture was degassed under N₂ atmosphere for three times and stirred at 50° C. for 2 hrs. The solution was concentrated to dryness and the residue was purified by flash chromatography (silica gel, 0˜30% EtOAc in PE) to give the title compound (320 mg, 41.6% yield) as yellow oil. LC/MS (ESI) m/z: 433 (M+H)⁺.

### Step 8: methyl 2-(2-(6-(5-(hydroxymethyl)-1-methyl-1H-1,2,3-triazol-4-yl)-2-methylpyridin-3-yl)tetrahydro-2H-pyran-4-yl)acetate

To a solution of methyl 2-(2-(6-(5-(hydroxymethyl)-1-((trimethylsilyl)methyl)-1H-1,2,3-triazol-4-yl)-2-methylpyridin-3-yl)tetrahydro-2H-pyran-4-yl)acetate (320 mg, 0.74 mmol) in THE (15 mL) was added TBAF (194 mg, 0.74 mmol) and the mixture reaction was stirred at r.t. for 1 hr. The mixture was poured into saturated aq. NH₄Cl solution and extracted with EtOAc (3×30 mL). The combined organic layers were washed with brine, dried over anhydrous Na₂SO₄, filtered and concentrated under reduced pressure to dryness. The residue was purified by flash chromatography (silica gel, 0˜70% EtOAc in PE) to give the title compound (230 mg, 86.1% yield) as yellow oil. LC/MS (ESI) m/z: 361 (M+H)⁺.

### Step 9: methyl 2-(2-(6-(5-(((4-cyclobutylpyrimidin-2-yl)oxy)methyl)-1-methyl-1H-1,2,3-triazol-4-yl)-2-methylpyridin-3-yl)tetrahydro-2H-pyran-4-yl)acetate

To a mixture of methyl 2-(2-(6-(5-(hydroxymethyl)-1-methyl-1H-1,2,3-triazol-4-yl)-2-methylpyridin-3-yl)tetrahydro-2H-pyran-4-yl)acetate (65 mg, 0.18 mmol) and 2-chloro-4-cyclobutylpyrimidine (45.5 mg, 0.27 mmol) in THE (8 mL) was added t-BuOK (0.36 mL, 0.36 mmol, 1.0 M in THF) drop-wisely at 0° C. and the reaction was stirred at 0° C. for 1 hr. The mixture was quenched with saturated aq. NH₄Cl solution and extracted with EtOAc (3×10 mL). The combined organic layers were washed with brine, dried over anhydrous Na₂SO₄, filtered and concentrated to dryness. The residue was purified by flash chromatography (silica gel, 0˜50% EtOAc in PE) to give the title compound (80 mg, 89.9% yield) as yellow oil. LC/MS (ESI) m/z: 493 (M+H)⁺.

### Step 10: 2-(2-(6-(5-(((4-cyclobutylpyrimidin-2-yl)oxy)methyl)-1-methyl-1H-1,2,3-triazol-4-yl)-2-methylpyridin-3-yl)tetrahydro-2H-pyran-4-yl)acetic acid

To a solution of methyl 2-(2-(6-(5-(((4-cyclobutylpyrimidin-2-yl)oxy)methyl)-1-methyl-1H-1,2,3-triazol-4-yl)-2-methylpyridin-3-yl)tetrahydro-2H-pyran-4-yl)acetate (80 mg, 0.16 mmol) in MeOH (2 mL), THE (2 mL) and H₂O (1 mL) was added LiOH (68 mg, 1.6 mol) and the mixture was stirred at room temperature for 2 hrs. The mixture was concentrated to dryness and the residue was dissolved in water (8 mL). The mixture was washed with EtOAc (2×5 mL), acidified with 1N aq. HCl to pH-3 and extracted with DCM (3×5 mL). The combined organic layers were washed with brine, dried over anhydrous Na₂SO₄, filtered and concentrated under reduced pressure to dryness. The residue was purified by prep-HPLC (C18, 30-95%, MeCN in H₂O with 0.1% HCOOH) to give the title compound (16 mg, 21% yield) as white solid. LC/MS (ESI) m/z: 479 (M+H)⁺. ¹H NMR (400 MHz, CD₃OD) δ 8.40 (d, J=5.1 Hz, 1H), 7.91-7.84 (m, 2H), 6.98 (d, J=5.1 Hz, 1H), 6.23-6.12 (m, 2H), 4.83-4.81 (m, 0.5H), 4.59 (dd, J=11.1, 1.9 Hz, 0.5H), 4.24 (s, 3H), 4.14 (dd, J=11.5, 3.4 Hz, 0.5H), 3.90-3.83 (m, 1H), 3.73-3.66 (m, 0.5H), 3.54-3.44 (m, 1H), 2.77-2.61 (m, 1H), 2.56-2.52 (m, 0.5H), 2.50-2.48 (m, 3H), 2.32-2.26 (m, 1H), 2.24-2.18 (m, 4.5H), 2.07-1.95 (m, 2H), 1.83-1.74 (m, 2.5H), 1.56-1.52 (m, 0.5H), 1.48-1.37 (m, 0.5H), 1.31-1.22 (m, 0.5H).

### Example 27: 2-[2-(2-methyl-6-{1-methyl-5-[(2-oxo-5-propyl-1,2-dihydropyridin-1-yl)methyl]-1H-1,2,3-triazol-4-yl}pyridin-3-yl)oxan-4-yl]acetic acid

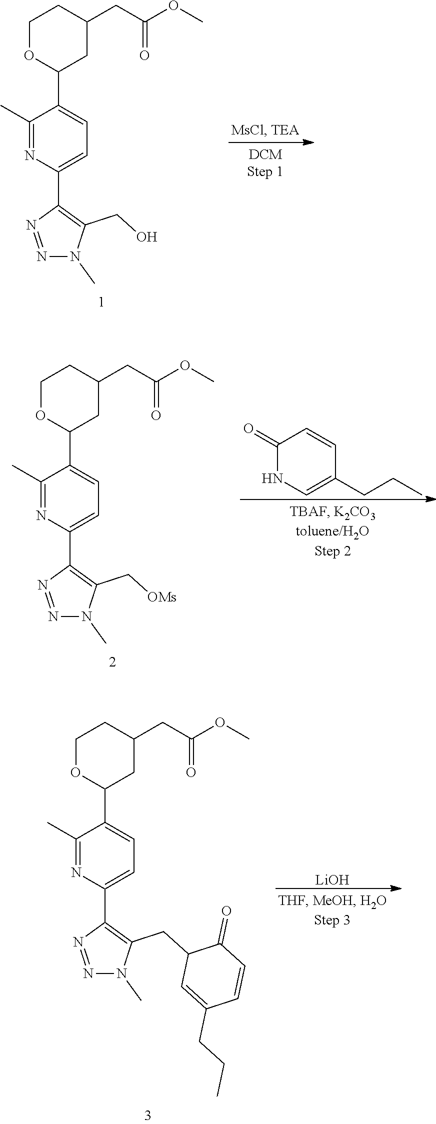

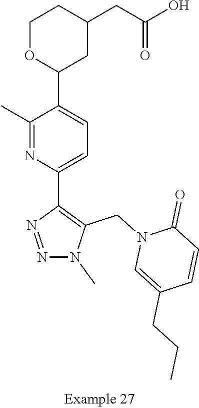 [SMILES: CCCc1ccc(=O)n(Cc2c(-c3ccc(C4CC(CC(=O)O)CCO4)c(C)n3)nnn2C)c1]

### Step 1: ethyl 2-[(4S)-2-(6-{5-[(methanesulfonyloxy)methyl]-1-methyl-1H-1,2,3-triazol-4-yl}-2-methylpyridin-3-yl)oxan-4-yl]acetate

To a stirred solution of ethyl 2-(2-{6-[5-(hydroxymethyl)-1-methyl-1H-1,2,3-triazol-4-yl]-2-methylpyridin-3-yl}oxan-4-yl)acetate (60 mg, 0.16 mmol) in DCM (5 mL) was added MsCl (37 mg, 0.32 mmol) followed by TEA (49 mg, 0.48 mmol) at 0° C. under N₂ atmosphere. The reaction mixture was stirred at r.t. for 30 min until the disappearance of the starting material was confirmed by TLC analysis. The reaction was quenched with saturated aq. NaHCO₃ solution at 0° C. and the mixture was extracted with DCM (2×20 mL). The combined organic layers were washed with brine, dried over anhydrous Na₂SO₄, filtered and concentrated to dryness to give the title compound (65 mg, 89.6% yield) as yellow oil, which was used directly in the next step. LC/MS (ESI) m/z: 453 (M+H)⁺.

### Step 2: ethyl 2-[(4S)-2-(2-methyl-6-{1-methyl-5-[(2-oxo-5-propyl-1,2-dihydropyridin-1-yl)methyl]-1H-1,2,3-triazol-4-yl}pyridin-3-yl)oxan-4-yl]acetate

To mixture of ethyl 2-[(4S)-2-(6-{5-[(methanesulfonyloxy)methyl]-1-methyl-1H-1,2,3-triazol-4-yl}-2-methylpyridin-3-yl)oxan-4-yl]acetate (50 mg, 0.11 mmol) and 5-propyl-1,2-dihydropyridin-2-one (18 mg, 0.13 mmol) in toluene (5 mL) and H₂O (1 mL) were added K₂CO₃ (46 mg, 0.33 mmol) followed by TBAF (3 mg, 0.01 mmol) and the reaction was stirred at 100° C. for 16 hrs. The reaction mixture was concentrated under reduced pressure to dryness. The residue was diluted with EtOAc (10 mL), washed with water and brine, dried over anhydrous Na₂SO₄, filtered and concentrated to dryness to give the title compound (50 mg, 91.7% yield) as yellow solid, which was used directly in the next step. LC/MS (ESI) m/z: 494 (M+H)⁺.

### Step 3: 2-[2-(2-methyl-6-{1-methyl-5-[(2-oxo-5-propyl-1,2-dihydropyridin-1-yl)methyl]-1H-1,2,3-triazol-4-yl}pyridin-3-yl)oxan-4-yl]acetic acid

To a solution of ethyl 2-[2-(2-methyl-6-{1-methyl-5-[(2-oxo-5-propyl-1,2-dihydropyridin-1-yl)methyl]-1H-1,2,3-triazol-4-yl}pyridin-3-yl)oxan-4-yl]acetate (50 mg, 0.10 mmol) in THE (4 mL)/H₂O (1 mL)/MeOH (1 mL) was added LiOH·H₂O (43 mg, 1.01 mmol) and the reaction was stirred at r.t. for 2 hrs. Volatiles were removed under vacuum and the residue was diluted with H₂O (5 mL). The mixture was adjusted with 1N aq. HCl to pH˜4 and extracted with EtOAc (3×5 mL). The combined organic layers were washed with brine, dried over anhydrous Na₂SO₄, filtered and concentrated to dryness. The residue was purified by prep-HPLC (C18, 5-95%, MeCN in H₂O with 0.1% HCOOH) to give the title compound (10 mg, 21.2% yield) as white solid. LC/MS (ESI) m/z: 466 (M+H)⁺. ¹H NMR (400 MHz, CD₃OD) δ 7.92-7.81 (m, 3H), 7.36 (dd, J=9.2, 2.4 Hz, 1H), 6.47 (d, J=9.3 Hz, 1H), 5.77 (s, 2H), 4.66-4.56 (m, 0.5H), 4.23 (d, J=0.5 Hz, 3H), 4.17-4.09 (m, 0.5H), 3.90 (dd, J=7.9, 3.0 Hz, 1H), 2.80-2.65 (m, 1H), 2.63 (d, J=6.4 Hz, 3H), 2.61-2.47 (m, 1H), 2.33-2.18 (m, 4H), 2.09-1.95 (m, 1H), 1.86-1.80 (m, 1H), 1.78 (d, J=14.2 Hz, 0.5H), 1.55 (d, J=14.9 Hz, 0.5H), 1.42-1.34 (m, 2H), 1.34-1.16 (m, 1H), 0.78 (t, J=7.3 Hz, 3H).

### Example 28: 2-{5-[6-(5-{[(4-cyclobutylpyrimidin-2-yl)oxy]methyl}-1-methyl-1H-1,2,3-triazol-4-yl)-2-methylpyridin-3-yl]oxan-3-yl}acetic acid

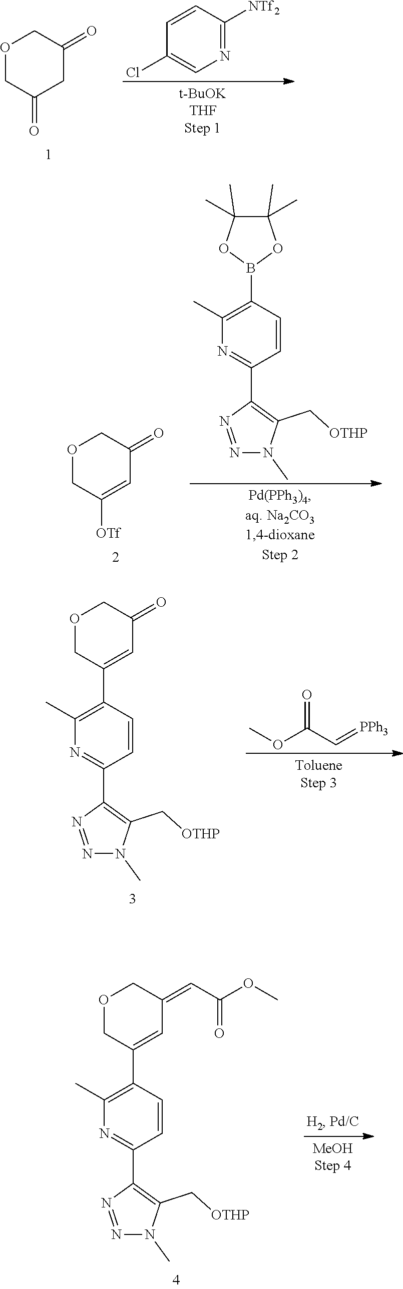

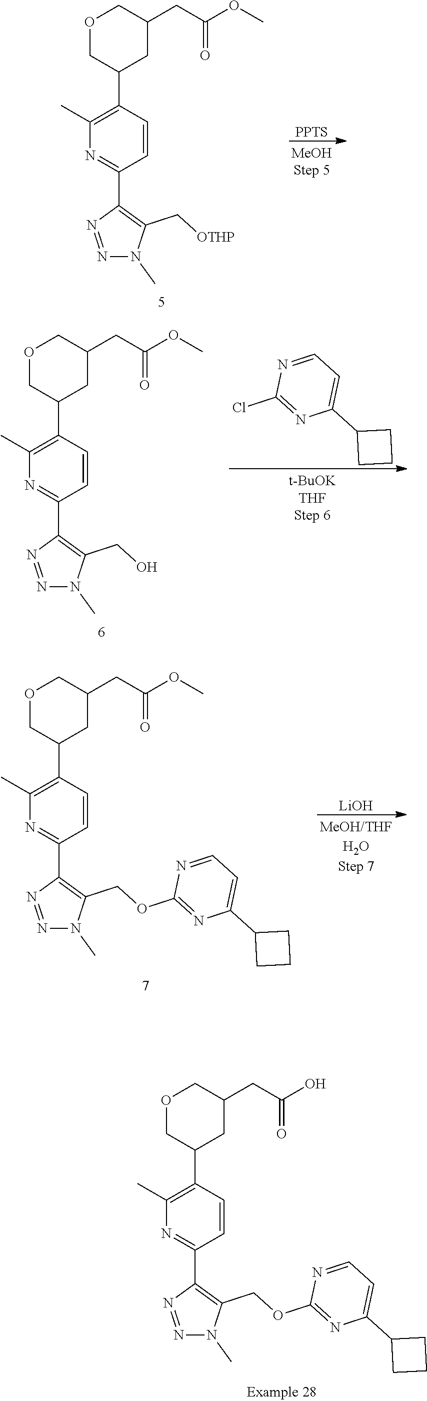

### Step 1: 5-oxo-5,6-dihydro-2H-pyran-3-yl trifluoromethanesulfonate

To a solution of 2H-pyran-3,5(4H,6H)-dione (2.3 g, 20 mmol) in THE (40 mL) was added t-BuOK (22 mL, 1M in THF) drop-wisely at 0° C. After the addition, the mixture was stirred at 0° C. for another 0.5 hr. N-(5-chloropyridin-2-yl)-1,1,1-trifluoro-Ntrifluoromethanesulfonyl methanesulfonamide (8.8 g, 22 mmol) in THF (20 mL) was added drop-wisely to the above mixture at 0° C. and the resulting mixture was stirred at r.t. for 2 hrs. The mixture was diluted with EtOAc (10 mL), washed with water and brine, dried over anhydrous Na₂SO₄, filtered and concentrated to dryness. The residue was purified by flash chromatography (silica gel, 0˜50% of EtOAc in PE) to give the title compound (1.2 g, 25% yield) as colorless oil. LC/MS (ESI) m/z: 247 (M+1)⁺. ¹H NMR (400 MHz, CDCl₃) δ 6.24 (t, J=1.4 Hz, 1H), 4.47 (s, 2H), 4.17 (s, 2H).

### Step 2: 5-(2-methyl-6-(1-methyl-5-(((tetrahydro-2H-pyran-2-yl)oxy)methyl)-1H-1,2,3-triazol-4-yl)pyridin-3-yl)-2H-pyran-3(6H)-one

To a mixture of 5-oxo-5,6-dihydro-2H-pyran-3-yl trifluoromethanesulfonate (1.2 g, 5 mmol), 2-methyl-6-{1-methyl-5-[(oxan-2-yloxy)methyl]-1H-1,2,3-triazol-4-yl}-3-(tetramethyl-1,3,2-dioxaborolan-2-yl)pyridine (1.3 g, 3.2 mmol) and Na₂CO₃ (860 mg, 8.1 mmol) in 1,4-dioxane (20 mL) and H₂O (4 mL) was added Pd(PPh₃)₄ (155 mg, 0.13 mmol) under N₂ atmosphere. After the addition, the mixture was degassed under N₂ atmosphere for three times and stirred at 60° C. for another 3 hrs. The mixture was diluted with EtOAc (30 mL), washed with water and brine, dried over anhydrous Na₂SO₄, filtered and concentrated to dryness. The residue was purified by flash chromatography (silica gel, 0-50% of EtOAc in PE) to give the title compound (920 mg, 78.6% yield) as light yellow solid. LC/MS (ESI) m/z: 385 (M+1)⁺. ¹H NMR (400 MHz, CDCl₃) δ 8.09 (d, J=8.1 Hz, 1H), 7.65 (d, J=1.4 Hz, 1H), 6.22 (t, J=1.6 Hz, 1H), 5.42-5.27 (m, 2H), 4.83-4.71 (m, 1H), 4.56 (d, J=0.9 Hz, 2H), 4.27 (s, 2H), 4.17 (s, 3H), 3.88 (ddd, J=11.3, 8.0, 3.0 Hz, 1H), 3.62-3.48 (m, 1H), 2.60 (d, J=4.8 Hz, 3H), 1.71 (dt, J=5.6, 4.5 Hz, 2H), 1.63-1.49 (m, 4H).

### Step 3: methyl (E)-2-(5-(2-methyl-6-(1-methyl-5-(((tetrahydro-2H-pyran-2-yl)oxy)methyl)-1H-1,2,3-triazol-4-yl)pyridin-3-yl)-2H-pyran-3(6H)-ylidene)acetate

A mixture of 5-(2-methyl-6-(1-methyl-5-(((tetrahydro-2H-pyran-2-yl)oxy)methyl)-1H-1,2,3-triazol-4-yl)pyridin-3-yl)-2H-pyran-3(6H)-one (385 mg, 1.0 mmol) and Methyl (triphenylphosphoranylidene)acetate (500 mg, 1.5 mmol) in toluene (8 mL) was stirred at 110° C. for 16 hrs. The mixture was concentrated under reduced pressure to dryness and the residue was purified by flash chromatography (silica gel, 0˜20% of EtOAc in PE) to give the title compound (310 mg, 70.4% yield) as white solid. LC/MS (ESI) m/z: 441 (M+1)⁺.

### Step 4: methyl 2-(5-(2-methyl-6-(1-methyl-5-(((tetrahydro-2H-pyran-2-yl)oxy)methyl)-1H-1,2,3-triazol-4-yl)pyridin-3-yl)tetrahydro-2H-pyran-3-yl)acetate

To a solution of methyl (E)-2-(5-(2-methyl-6-(1-methyl-5-(((tetrahydro-2H-pyran-2-yl)oxy)methyl)-1H-1,2,3-triazol-4-yl)pyridin-3-yl)-2H-pyran-3(6H)-ylidene)acetate (310 mg, 0.7 mmol) in MeOH (10 mL) was added Pd/C (15 mg, 10% wt) and the mixture was degassed under N₂ atmosphere for three times and stirred under a H₂ balloon at r.t. for 16 hrs. The mixture was filtered and the filtrate was concentrated to dryness. The residue was purified by flash chromatography (silica gel, 0˜20% of EtOAc in PE) to give the title compound (230 mg, 73.5% yield) as white solid. LC/MS (ESI) m/z: 445 (M+H)⁺.

### Step 5: methyl 2-(5-(6-(5-(hydroxymethyl)-1-methyl-1H-1,2,3-triazol-4-yl)-2-methylpyridin-3-yl)tetrahydro-2H-pyran-3-yl)acetate

To a solution of methyl 2-(5-(2-methyl-6-(1-methyl-5-(((tetrahydro-2H-pyran-2-yl)oxy)methyl)-1H-1,2,3-triazol-4-yl)pyridin-3-yl)tetrahydro-2H-pyran-3-yl)acetate (230 mg, 0.5 mmol) in MeOH (10 mL) was added PPTS (262 mg, 1.0 mmol) and the mixture was stirred at 60° C. for 16 hrs. The mixture was diluted with DCM (10 mL), washed with saturated aq. NaHCO₃ solution and brine, dried over anhydrous Na₂SO₄, filtered and concentrated to dryness. The residue was purified by flash chromatography (silica gel, 0˜40% of EtOAc in PE) to give the title compound (75 mg, 40.2% yield) as colorless oil. LC/MS (ESI) m/z: 361 (M+H)⁺.

### Step 6: methyl 2-(5-(6-(5-(((4-cyclobutylpyrimidin-2-yl)oxy)methyl)-1-methyl-1H-1,2,3-triazol-4-yl)-2-methylpyridin-3-yl)tetrahydro-2H-pyran-3-yl)acetate

To a mixture of methyl 2-(5-(6-(5-(hydroxymethyl)-1-methyl-1H-1,2,3-triazol-4-yl)-2-methylpyridin-3-yl)tetrahydro-2H-pyran-3-yl)acetate (20 mg, 0.05 mmol) and 2-chloro-4-cyclobutylpyrimidine (18 mg, 0.1 mmol) in THE (4 mL) was added t-BuOK (0.1 mL, 1M in THF) at 0° C. After addition, the mixture was stirred at r.t. for 16 hrs. The reaction was quenched with ice-cooled saturated aq. NH₄Cl solution and extracted with EtOAc (2×5 mL). The combined organic layers were washed with brine, dried over anhydrous Na₂SO₄, filtered and concentrated to dryness to give the title compound (15 mg, 55.4% yield) as white solid. LC/MS (ESI) m/z: 493 (M+H)⁺.

### Step 7: 2-(5-(6-(5-(((4-cyclobutylpyrimidin-2-yl)oxy)methyl)-1-methyl-1H-1,2,3-triazol-4-yl)-2-methylpyridin-3-yl)tetrahydro-2H-pyran-3-yl)acetic acid

To a solution of methyl 2-(5-(6-(5-(((4-cyclobutylpyrimidin-2-yl)oxy)methyl)-1-methyl-1H-1,2,3-triazol-4-yl)-2-methylpyridin-3-yl)tetrahydro-2H-pyran-3-yl)acetate (15 mg, 0.03 mmol) in THE (2 mL), MeOH (1 mL) and H₂O (1 mL) was added LiOH (10 mg, 0.2 mmol) and the mixture was stirred at r.t. for 3 hrs. The mixture was concentrated to dryness and the residue was dissolved in water (5 mL). The mixture was washed with EtOAc (2×3 mL), acidified with 1N aq. HCl to pH˜4 and extracted with DCM (2×5 mL). The combined organic layers were washed with brine, dried over Na₂SO₄, filtered and concentrated to dryness. The residue was purified by preparative HPLC (C18, 0˜90% acetonitrile in H₂O with 0.1% HCOOH) to give the title compound (4 mg, 27.5% yield) as white solid. LC/MS (ESI) m/z: 479 (M+H)⁺. ¹H NMR (400 MHz, CD₃OD) δ 8.40 (dd, J=5.1, 1.0 Hz, 1H), 7.81-7.84 (m, 2H), 6.97 (d, J=5.1 Hz, 1H), 6.22-6.12 (m, 2H), 4.24 (s, 3H), 4.08-3.78 (m, 2H), 3.71-3.63 (m, 1H), 3.55-3.45 (m, 1H), 3.30-3.11 (m, 3H), 2.60-2.48 (m, 4H), 2.24-2.17 (m, 5H), 2.08-1.96 (m, 2H), 1.95-1.61 (m, 2H).

### Example 29: 2-[(5R)-5-(2-ethyl-6-{1-methyl-5-[(2-oxo-5-propyl-1,2-dihydropyridin-1-yl)methyl]-1H-1,2,3-triazol-4-yl}pyridin-3-yl)oxan-3-yl]acetic acid

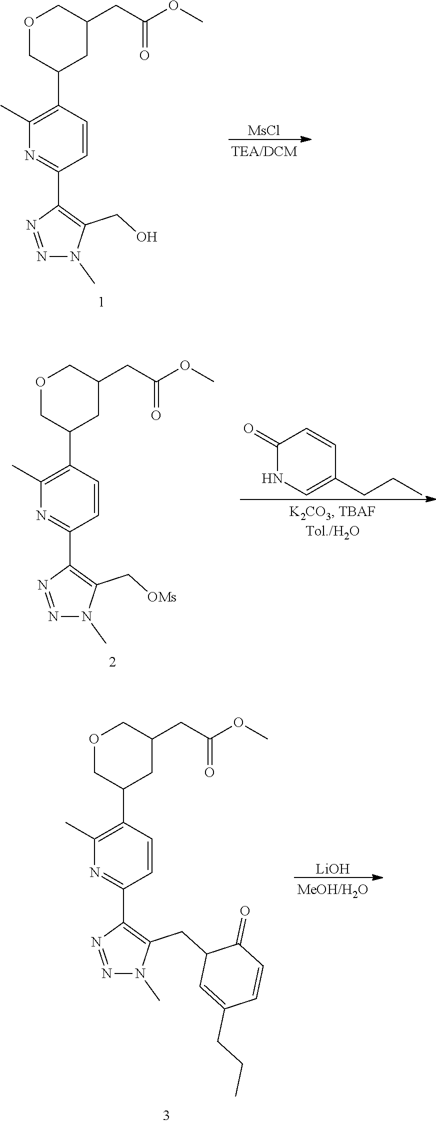

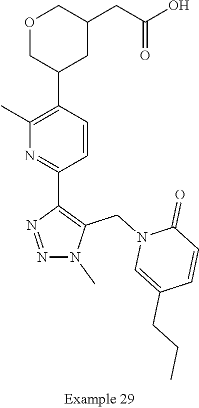 [SMILES: CCCc1ccc(=O)n(Cc2c(-c3ccc(C4COCC(CC(=O)O)C4)c(C)n3)nnn2C)c1]

### Step 1: methyl 2-[5-(6-{5-[(methanesulfonyloxy)methyl]-1-methyl-1H-1,2,3-triazol-4-yl}-2-methylpyridin-3-yl)oxan-3-yl]acetate

To a solution of methyl 2-(5-(6-(5-(hydroxymethyl)-1-methyl-1H-1,2,3-triazol-4-yl)-2-methylpyridin-3-yl)tetrahydro-2H-pyran-3-yl)acetate (30 mg, 0.08 mmol) in DCM (4 mL) was added TEA (25 mg, 0.25 mmol) followed by MsCl (20 mg, 0.18 mmol) at 0° C. After the addition, the mixture was stirred at r.t. for 2 hrs. The mixture was diluted with DCM (10 mL), washed with water and brine, dried over anhydrous Na₂SO₄, filtered and concentrated to dryness to give the title compound (32 mg, 87.7% yield) as colorless oil, which was directly used in the next reaction without purification. LC/MS (ESI) m/z: 439 (M+H)⁺.

### Step 2: methyl 2-(5-(2-methyl-6-(1-methyl-5-((2-oxo-5-propylpyridin-1(2H)-yl)methyl)-1H-1,2,3-triazol-4-yl)pyridin-3-yl)tetrahydro-2H-pyran-3-yl)acetate

To a mixture of methyl 2-[5-(6-{5-[(methanesulfonyloxy)methyl]-1-methyl-1H-1,2,3-triazol-4-yl}-2-methylpyridin-3-yl)oxan-3-yl]acetate (32 mg, 0.07 mmol) and 5-propylpyridin-2(1H)-one (10 mg, 0.07 mmol) in toluene (4 mL) and H₂O (1 mL) was added TBAF (5 mg, 0.019 mmol) and K₂CO₃ (25 mg, 0.18 mmol) and the resulting mixture was stirred at 110° C. for 16 hrs. After cooling to r.t., the mixture was diluted with EtOAc (10 mL), washed with water and brine, dried over anhydrous Na₂SO₄, filtered and concentrated to dryness to give the title compound (30 mg, 83.3% yield) as yellow solid. LC/MS (ESI) m/z: 480 (M+H)⁺.

### Step 3: 2-(5-(2-methyl-6-(1-methyl-5-((2-oxo-5-propylpyridin-1(2H)-yl)methyl)-1H-1,2,3-triazol-4-yl)pyridin-3-yl)tetrahydro-2H-pyran-3-yl)acetic acid

To a solution of methyl 2-(5-(2-methyl-6-(1-methyl-5-((2-oxo-5-propylpyridin-1(2H)-yl)methyl)-1H-1,2,3-triazol-4-yl)pyridin-3-yl)tetrahydro-2H-pyran-3-yl)acetate (30 mg, 0.061 mmol) in THE (2 mL), MeOH (1 mL) and H₂O (1 mL) was added LiOH (10 mg, 0.2 mmol) and the mixture was stirred at r.t. for 3 hrs. The mixture was concentrated to dryness and the residue was dissolved in water (5 mL). The mixture was washed with EtOAc (2×3 mL), acidified with 1N aq. HCl to pH˜4 and extracted with DCM (2×5 mL). The combined organic layers were washed with brine, dried over Na₂SO₄, filtered and concentrated to dryness. The residue was purified by preparative HPLC (C18, 0˜90% acetonitrile in H₂O with 0.1% TFA) to give the title compound (9.5 mg, 32.6% yield) as white solid. LC/MS (ESI) m/z: 466 (M+H)⁺. ¹H NMR (400 MHz, CD₃OD) δ 7.96-7.70 (m, 3H), 7.36 (dd, J=9.3, 2.3 Hz, 1H), 6.47 (d, J=9.3 Hz, 1H), 5.77 (d, J=1.4 Hz, 2H), 4.23 (d, J=1.4 Hz, 3H), 4.07-3.81 (m, 2H), 3.71-3.64 (m, 1H), 3.49-3.33 (m, 1H), 3.23-3.14 (m, 1H), 2.68 (d, J=10.3 Hz, 3H), 2.57-2.45 (m, 1H), 2.30-2.19 (m, 4H), 2.11-1.97 (m, 1H), 1.93-1.53 (m, 1H), 1.44-1.34 (m, 2H), 0.78 (td, J=7.3, 1.6 Hz, 3H).

### Example 30: 2-(4-{2-ethyl-6-[1-methyl-5-({[methyl(propyl)carbamoyl]oxy}methyl)-1H-1,2,3-triazol-4-yl]pyridin-3-yl}oxan-2-yl)acetic acid

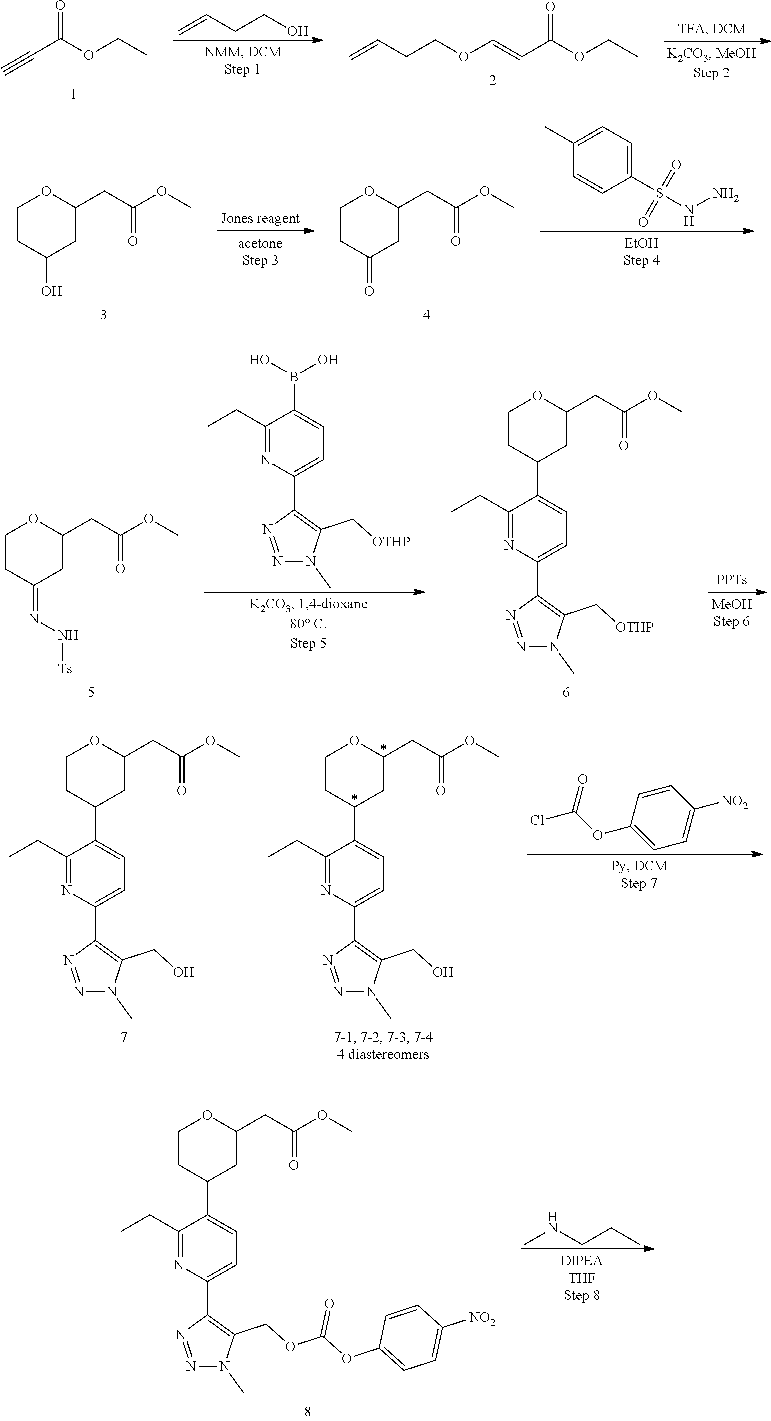

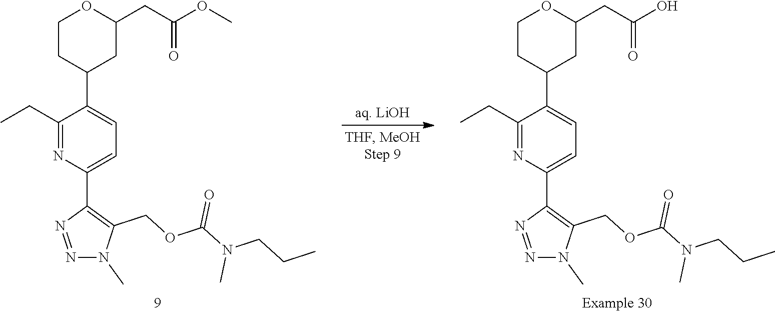 [SMILES: CCCN(C)C(=O)OCc1c(-c2ccc(C3CCOC(CC(=O)OC)C3)c(CC)n2)nnn1C ; CCCN(C)C(=O)OCc1c(-c2ccc(C3CCOC(CC(=O)O)C3)c(CC)n2)nnn1C]

### Step 1: ethyl (E)-3-(but-3-en-1-yloxy)acrylate

To a mixture of ethyl prop-2-ynoate (10 mL, 0.1 mol) and 4-Methylmorpholine (11 mL, 0.1 mol) in DCM (150 mL) was added but-3-en-1-ol (8.7 mL, 0.1 mol) drop-wisely at 0° C. over 15 mins and the mixture was stirred at r.t. for 3 hrs. The mixture was washed with water and brine, dried over anhydrous Na₂SO₄, filtered and concentrated to dryness. The residue was purified by flash chromatography (silica gel, 0˜30% of EtOAc in PE) to give the title compound (12.3 g, 72.3% yield) as colorless oil. LC/MS (ESI) m/z: 171 (M+1)⁺. ¹H NMR (400 MHz, CDCl₃) δ 7.60 (d, J=12.6 Hz, 1H), 5.80 (ddt, J=17.0, 10.2, 6.7 Hz, 1H), 5.20 (d, J=12.6 Hz, 1H), 5.17-5.08 (m, 2H), 4.16 (q, J=7.1 Hz, 2H), 3.89 (t, J=6.6 Hz, 2H), 2.47 (q, J=6.7 Hz, 2H), 1.27 (t, J=7.1 Hz, 3H).

### Step 2: methyl 2-(4-hydroxytetrahydro-2H-pyran-2-yl)acetate

To a solution of ethyl (E)-3-(but-3-en-1-yloxy)acrylate (5.1 g, 30 mmol) in dry DCM (100 mL) was added TFA (12 mL) at 0° C. drop-wisely over 30 mins and the mixture was stirred at 0° C. for 2 hrs. After completion, the reaction mixture was concentrated to dryness and diluted with EtOAc (100 mL). The mixture was washed with ice-cold 1N aq. NaHCO₃ solution and brine, dried over anhydrous Na₂SO₄, filtered and concentrated to dryness. The residue was dissolved in MeOH (80 mL) and K₂CO₃ (13 g, 92 mmol) was added. The resulting mixture was stirred at 25° C. for 30 mins. The pH value of the mixture was adjusted to 7 with 1N aq. AcOH solution and the mixture was extracted DCM (3×30 mL). The combined organic layers were washed with brine, dried over anhydrous Na₂SO₄, filtered and concentrated under vacuum to dryness. The residue was purified by flash chromatography (silica gel, 0˜20% of EtOAc in PE) to give the title compound (2.3 g, 40% yield) as colorless oil.

### Step 3: methyl 2-(4-oxotetrahydro-2H-pyran-2-yl)acetate

To a mixture of ethyl 2-(4-hydroxyoxan-2-yl)acetate (1.5 g, 8.0 mmol) in acetone (20 mL) was added newly prepared Jones reagent (10 mL) and the mixture was stirred at r.t. for 5 hrs. The reaction mixture was diluted with EtOAc (30 mL), washed with saturated aq. NaHSO₃ solution and brine, dried over anhydrous Na₂SO₄, filtered and concentrated to dryness. The residue was purified by flash chromatography (silica gel, 0˜30% of EtOAc in PE) to give the title compound (1.1 g, 74.1% yield) as colorless oil. ¹H NMR (400 MHz, CDCl₃) δ 4.35-4.24 (m, 1H), 4.12-4.01 (m, 1H), 3.72 (s, 3H), 3.72-3.65 (m, 1H), 2.69 (dd, J=15.5, 7.8 Hz, 1H), 2.65-2.56 (m, 1H), 2.56-2.45 (m, 2H), 2.43-2.31 (m, 2H).

### Step 4: methyl (Z)-2-(4-(2-tosylhydrazono)tetrahydro-2H-pyran-2-yl)acetate

A mixture of ethyl 2-(4-oxooxan-2-yl)acetate (900 mg, 5.0 mmol) and 4-methylbenzenesulfonohydrazide (970 mg, 5.0 mmol) in EtOH (20 mL) was stirred at r.t. for 3 hrs. The mixture was diluted with EtOAc (30 mL), washed with water and brine, dried over anhydrous Na₂SO₄, filtered and concentrated to dryness. The residue was purified by flash chromatography (silica gel, 0˜30% of EtOAc in PE) to give the title compound (1.5 g, 78.7% yield) as light yellow solid. LC/MS (ESI) m/z: 341 (M+1)⁺.

### Step 5: methyl 2-[4-(2-ethyl-6-{1-methyl-5-[(oxan-2-yloxy)methyl]-1H-1,2,3-triazol-4-yl}pyridin-3-yl)oxan-2-yl]acetate

To a mixture of 3-bromo-2-ethyl-6-{1-methyl-5-[(oxan-2-yloxy)methyl]-1H-1,2,3-triazol-4-yl}pyridine (760 mg, 2.0 mmol), ethyl 2-[(3R)-piperidin-3-yl]acetate (420 mg, 2.0 mmol) in 1,4-dioxane (20 mL) was added K₂CO₃ (830 mg, 6.0 mmol) and the mixture was stirred at 80° C. overnight. The reaction mixture was diluted with EtOAc (20 mL), washed with water and brine, dried over anhydrous Na₂SO₄, filtered and concentrated to dryness. The residue was purified by flash chromatography (silica gel, 0˜50% of EtOAc in PE) to give the title compound (170 mg, 30.6% yield) as yellow solid. LC/MS (ESI) m/z: 459 (M+1)⁺. ¹H NMR (400 MHz, CDCl₃) δ 8.00 (d, J=8.1 Hz, 1H), 7.55 (s, 1H), 5.46 (dd, J=12.7, 1.9 Hz, 1H), 5.33 (dd, J=12.7, 1.7 Hz, 1H), 4.79-4.69 (m, 1H), 4.16 (s, 3H), 4.15-4.10 (m, 1H), 4.00-3.91 (m, 1H), 3.86 (dd, J=13.9, 5.8 Hz, 1H), 3.71 (s, 3H), 3.65 (td, J=11.7, 2.5 Hz, 1H), 3.50 (dd, J=11.2, 4.2 Hz, 1H), 3.08 (ddd, J=12.0, 8.2, 3.8 Hz, 1H), 2.90 (q, J=7.5 Hz, 2H), 2.63 (dd, J=15.3, 7.8 Hz, 1H), 2.47 (dd, J=15.3, 5.1 Hz, 1H), 1.87-1.74 (m, 3H), 1.70 (td, J=9.5, 4.4 Hz, 3H), 1.59 (dd, J=8.5, 4.1 Hz, 3H), 1.35 (t, J=7.5 Hz, 3H).

### Step 6: ethyl 2-(4-(2-ethyl-6-(5-(hydroxymethyl)-1-methyl-1H-1,2,3-triazol-4-yl)pyridin-3-yl)morpholin-2-yl)acetate

To a mixture of methyl 2-[4-(2-ethyl-6-{1-methyl-5-[(oxan-2-yloxy)methyl]-1H-1,2,3-triazol-4-yl}pyridin-3-yl)oxan-2-yl]acetate (340 mg, 0.8 mmol) in MeOH (10 mL) was added PPTS (600 mg, 2.4 mmol) and the mixture was stirred at 60° C. for 3 hrs. The reaction mixture was diluted with DCM (20 mL), washed with saturated aq. NaHCO₃ solution and brine, dried over anhydrous Na₂SO₄, filtered and concentrated to dryness. The residue was purified by flash chromatography (silica gel: 0˜40% of EtOAc in PE) to give the title compound (210 mg, 75.6% yield) as white solid. LC/MS (ESI) m/z: 375 (M+1])⁺. The title compound was further purified by chiral SFC to give the four diastereomers as follows: compound 7-1 (Peak 1, retention time: 2.510 min), compound 7-2 (Peak 2, retention time: 2.798 min), compound 7-3 (Peak 3, retention time: 3.647 min) and compound 7-4 (Peak 4, retention time: 4.346 min). SFC condition: Column: ChiralCel OJ, 250×21.2 mm I.D., 5 μm; Mobile phase: A for CO₂ and B for MeOH+0.1% NH₃H₂O; Flow rate: 50 mL/min; Column temperature: 35° C.

### Step 7: methyl 2-(4-{2-ethyl-6-[1-methyl-5-({[(4-nitrophenoxy)carbonyl]oxy}methyl)-1H-1,2,3-triazol-4-yl]pyridin-3-yl}oxan-2-yl)acetate

To a mixture of ethyl 2-(4-(2-ethyl-6-(5-(hydroxymethyl)-1-methyl-1H-1,2,3-triazol-4-yl)pyridin-3-yl)morpholin-2-yl)acetate (40 mg, 0.1 mmol) and pyridine (50 mg, 0.6 mmol) in DCM (5 mL) was added a solution of 4-nitrophenyl chloroformate (73 mg, 0.4 mmol) in DCM (1 mL) drop-wisely at 0° C. and the mixture was stirred at r.t. for 2 hrs. The reaction mixture was diluted with EtOAc (10 mL), washed with water and brine, dried over anhydrous Na₂SO₄, filtered and concentrated to dryness to give the title compound (80 mg, 100% yield) as yellow solid, which was directly used in the next reaction without purification. LC/MS (ESI) m/z: 540 (M+1)⁺.

### Step 8: methyl 2-(4-{2-ethyl-6-[1-methyl-5-({[methyl(propyl)carbamoyl]oxy}methyl)-1H-1,2,3-triazol-4-yl]pyridin-3-yl}oxan-2-yl)acetate

To a mixture of methyl 2-(4-{2-ethyl-6-[1-methyl-5-({[(4-nitrophenoxy)carbonyl]oxy}methyl)-1H-1,2,3-triazol-4-yl]pyridin-3-yl}oxan-2-yl)acetate (80 mg, 0.1 mmol) and DIPEA (66 mg, 0.5 mmol) in THF (4 mL) was added methyl(propyl)amine (22 mg, 0.3 mmol) at 0° C. and the mixture was stirred at r.t. for 2 hrs. The reaction mixture was diluted with EtOAc (10 mL), washed with 1N aq. NaOH solution and brine, dried over anhydrous Na₂SO₄, filtered and concentrated to dryness to give the title compound (32 mg, 66.3% yield) as white solid. LC/MS (ESI) m/z: 474 (M+1)⁺.

### Step 9: 2-(4-(2-ethyl-6-(1-methyl-5-(((methyl(propyl)carbamoyl)oxy)methyl)-1H-1,2,3-triazol-4-yl)pyridin-3-yl)tetrahydro-2H-pyran-2-yl)acetic acid

To a solution of methyl 2-(4-{2-ethyl-6-[1-methyl-5-({[methyl(propyl)carbamoyl]oxy}methyl)-1H-1,2,3-triazol-4-yl]pyridin-3-yl}oxan-2-yl)acetate (32 mg, 0.7 mmol) in MeOH (1 mL), water (1 mL) and THE (4 mL) was added LiOH (21 mg, 0.5 mmol) and the mixture was stirred at 25° C. for 1 hr. The mixture was concentrated to dryness and the residue was dissolved in water (10 mL). The mixture was washed with EtOAc, acidified with 1 N aq. HCl to pH-3 and extracted with DCM (3×10 mL). The combined organic layers were washed with brine, dried over Na₂SO₄, filtered and concentrated to dryness. The residue was purified by preparative HPLC (C18, 0˜90% acetonitrile in H₂O with 0.1% TFA) to give the title compound (18.1 mg, 53.3% yield) as white solid. LC/MS (ESI) m/z: 460 (M+1)⁺. ¹H NMR (400 MHz, CD₃OD) δ 7.85 (d, J=7.9 Hz, 1H), 7.75 (dd, J=20.9, 8.2 Hz, 1H), 5.77 (d, J=4.7 Hz, 2H), 4.19 (s, 3H), 4.15-4.00 (m, 1H), 3.99-3.88 (m, 1H), 3.84-3.63 (m, 1H), 3.29-3.05 (m, 4H), 2.96 (dd, J=13.4, 6.0 Hz, 2H), 2.83 (d, J=17.6 Hz, 3H), 2.52-2.48 (m, 1H), 1.88-1.67 (m, 3H), 1.59-1.39 (m, 2H), 1.39-1.29 (m, 4H), 0.77 (dt, J=79.5, 7.5 Hz, 3H).

The following examples in Table 3 were prepared from appropriate starting materials by using a method analogous to that used to prepare the examples as described herein.

TABLE 3

| EX# | Structure & name | Analytical data | Method |
|---|---|---|---|
| 31 | 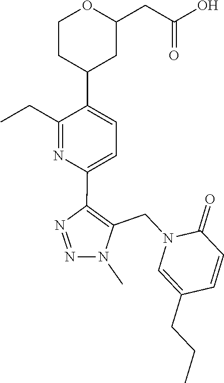 [SMILES: CCCc1ccc(=O)n(Cc2c(-c3ccc(C4CCOC(CC(=O)O)C4)c(CC)n3)nnn2C)c1] | LC/MS (ESI) m/z: 480 (M + H)⁺. ¹H NMR (400 MHz, CD₃OD) δ 7.89 (d, J = 8.2 Hz, 1H), 7.78 (d, J = 8.2 Hz, 1H), 7.67 (s, 1H), 7.38 (dd, J = 9.3, 2.5 Hz, 1H), 6.52 (d, J = 9.2 Hz, 1H), 5.88 (s, 2H), 4.17 (s, 3H), 4.10 (d, J = 11.1 Hz, 1H), 3.93 (m, 1H), 3.73 − 3.63 (m, 1H), 3.21 − 3.11 (m, 1H), 2.99 (t, J = 7.6 Hz, 2H), 2.58 − 2.41 (m, 2H), 2.21 (t, J = 7.5 Hz, 2H), 1.90 − 1.82 (m, 1H), 1.82 − 1.66 (m, 2H), 1.60 − 1.47 (m, 1H), 1.39 − 1.33 (m, 2H), 1.30 (d, J = 7.5 Hz, 3H), 0.75 (t, J = 7.3 Hz, 3H). | Example 2 & Example 30 |
| | 2-(4-(2-ethyl-6-(1-methyl- | | |
| | 5-((2-oxo-5-propylpyridin- | | |
| | 1(2H)-yl)methyl)-1H- | | |
| | 1,2,3-triazol-4-yl)pyridin- | | |
| | 3-yl)tetrahydro-2H-pyran- | | |
| | 2-yl)acetic acid | | |
| 32 | 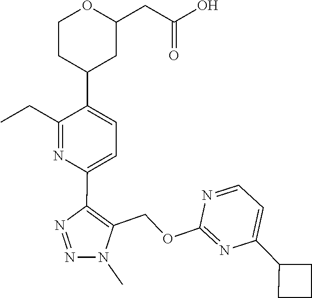 [SMILES: CCc1nc(-c2nnn(C)c2COc2nccc(C3CCC3)n2)ccc1C1CCOC(CC(=O)O)C1] | LC/MS (ESI) m/z: 493 (M + H)⁺. ¹H NMR (400 MHz, CD₃OD) δ 8.41 (d, J = 5.1 Hz, 1H), 7.88 (d, J = 8.1 Hz, 1H), 7.74 (d, J = 8.2 Hz, 1H), 6.99 (d, J = 5.1 Hz, 1H), 6.21 (s, 2H), 4.23 (s, 3H), 4.11 − 4.06 (m, 1H), 3.97 − 3.89 (m, 1H), 3.70 − 3.62 (m, 1H), 3.53 − 3.48 (m, 1H), 3.19 − 3.12 (m, 1H), 2.92 − 2.85 (m, 2H), 2.55 − 2.44 (m, 2H), 2.25 − 2.18 (m, 4H), 2.06 − 1.97 (m, 1H), 1.87 − 1.73 (m, 3H), 1.73 − 1.66 (m, 1H), 1.57 − 1.47 (m, 1H), 1.23 (t, J = 7.5 Hz, 3H). | Example 1 & Example 30 |
| | 2-(4-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)tetrahydro-2H-pyran-2- | | |
| | yl)acetic acid | | |
| 33 | 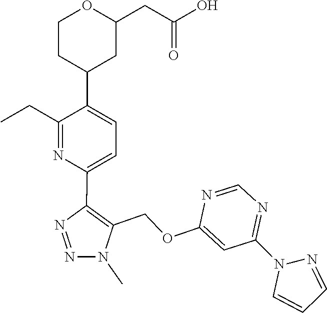 [SMILES: CCc1nc(-c2nnn(C)c2COc2cc(-n3cccn3)ncn2)ccc1C1CCOC(CC(=O)O)C1] | LC/MS (ESI) m/z: 505 (M + H)⁺. ¹H NMR (400 MHz, CD₃OD) δ 8.64 − 8.60 (m, 2H), 7.88 (d, J = 8.2 Hz, 1H), 7.78 − 7.72 (m, 2H), 7.28 (s, 1H), 6.55 − 6.54 (m, 1H), 6.21 (s, 2H), 4.24 (s, 3H), 4.07 (dd, J = 11.5, 2.8 Hz, 1H), 3.95 − 3.88 (m, 1H), 3.68 − 3.62 (m, 1H), 3.18 − 3.12 (m, 1H), 2.86 (q, J = 7.5 Hz, 2H), 2.54 − 2.44 (m, 2H), 1.84 − 1.66 (m, 3H), 1.56 − 1.45 (m, 1H), 1.22 (t, J = 7.5 Hz, 3H). | Example 1 & Example 30 |
| | 2-(4-(6-(5-(((6-(1H- | | |
| | pyrazol-1-yl)pyrimidin-4- | | |
| | yl)oxy)methyl)-1-methyl- | | |
| | 1H-1,2,3-triazol-4-yl)-2- | | |
| | ethylpyridin-3- | | |
| | yl)tetrahydro-2H-pyran-2- | | |
| | yl)acetic acid | | |
| 34 | 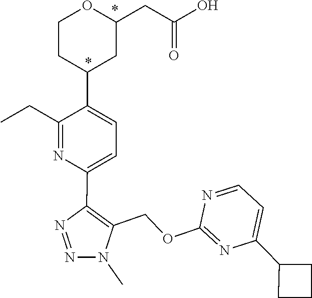 [SMILES: CCc1nc(-c2nnn(C)c2COc2nccc(C3CCC3)n2)ccc1C1CCOC(CC(=O)O)C1] | LC/MS (ESI) m/z: 493 (M + H)⁺. ¹H NMR (400 MHz, CD₃OD) δ 8.40 (d, J = 5.1 Hz, 1H), 7.88 (d, J = 8.1 Hz, 1H), 7.72 (d, J = 8.1 Hz, 1H), 6.97 (d, J = 5.1 Hz, 1H), 6.20 (s, 2H), 4.23 (s, 3H), 4.07 (dd, J = 11.3, 2.6 Hz, 1H), 3.97 − 3.89 (m, 1H), 3.70 − 3.62 (m, 1H), 3.54 − 3.45 (m, 1H), 3.20 − 3.11 (m, 1H), 2.87 (q, J = 7.5 Hz, 2H), 2.56 − 2.43 (m, 2H), 2.26 − 2.16 (m, 4H), 2.09 − 1.94 (m, 1H), 1.86 − 1.64 (m, 4H), 1.57 − 1.46 (m, 1H), 1.22 (t, J = 7.5 Hz, 3H). | Example 1 & Example 30 |
| | diastereomer-1 | | |
| | From Example 30 | | |
| | compound 7-1 | | |
| | 2-(4-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)tetrahydro-2H-223,423- | | |
| | pyran-2-yl)acetic acid | | |
| 35 | 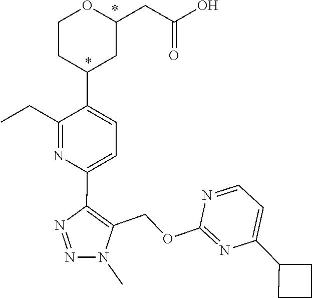 [SMILES: CCc1nc(-c2nnn(C)c2COc2nccc(C3CCC3)n2)ccc1C1CCOC(CC(=O)O)C1] | LC/MS (ESI) m/z: 494 (M + 1)⁺. ¹H NMR (400 MHz, CD₃OD) δ 8.41 (d, J = 5.1 Hz, 1H), 7.88 (d, J = 8.1 Hz, 1H), 7.74 (d, J = 8.2 Hz, 1H), 6.98 (d, J = 5.1 Hz, 1H), 6.21 (s, 2H), 4.23 (s, 3H), 4.10 − 4.05 (m, 1H), 3.96 − 3.89 (m, 1H), 3.70 − 3.62 (m, 1H), 3.50 (dd, J= 17.0, 8.5 Hz, 1H), 3.19 − 3.12 (m, 1H), 2.88 (q, J = 7.5 Hz, 2H), 2.56 − 2.45 (m, 2H), 2.25 − 2.21 (m, 2H), 2.21 − 2.18 (m, 2H), 2.06 − 1.96 (m, 1H), 1.87 − 1.74 (m, 3H), 1.73 − 1.66 (m, 1H), 1.52 (dd, J = 23.8, 12.3 Hz, 1H), 1.23 (t, J = 7.5 Hz, 3H). | Example 1 & Example 30 |
| | 1 diastereomer-2 | | |
| | From Example 30 | | |
| | compound 7-2 | | |
| | 2-(4-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)tetrahydro-2H-223,423- | | |
| | pyran-2-yl)acetic acid | | |
| 36 | 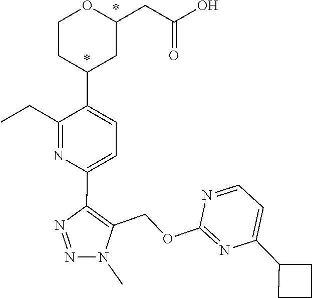 [SMILES: CCc1nc(-c2nnn(C)c2COc2nccc(C3CCC3)n2)ccc1C1CCOC(CC(=O)O)C1] | LC/MS (ESI) m/z: 493 (M + H)⁺. H NMR (400 MHz, CD₃OD) δ 8.40 (d, J = 5.1 Hz, 1H), 7.88 (d, J = 8.1 Hz, 1H), 7.73 (d, J = 8.2 Hz, 1H), 6.98 (d, J = 5.1 Hz, 1H), 6.24 − 6.17 (m, 2H), 4.23 (s, 3H), 4.08 (dd, J = 11.2, 3.5 Hz, 1H), 3.95 − 3.90 (m, 1H), 3.65 − 3.63 (m, 1H), 3.55 − 3.46 (m, 1H), 3.18 − 3.12 (m, 1H), 2.88 (q, J = 7.5 Hz, 2H), 2.56 − 2.42 (m, 2H), 2.24 − 2.18 (m, 4H), 2.06 − 1.95 (m, 1H), 1.84 − 1.66 (m, 4H), 1.56 − 1.47 (m, 1H), 1.23 (t, J = 7.5 Hz, 3H). | Example 1 & Example 30 |
| | diastereomer-3 | | |
| | From Example 30 | | |
| | compound 7-3 | | |
| | 2-(4-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)tetrahydro-2H-223,423- | | |
| | pyran-2-yl)acetic acid | | |
| 37 | 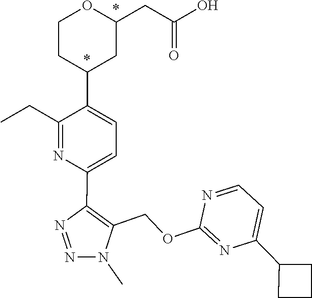 [SMILES: CCc1nc(-c2nnn(C)c2COc2nccc(C3CCC3)n2)ccc1C1CCOC(CC(=O)O)C1] | LC/MS (ESI) m/z: 493 (M + H)⁺. ¹H NMR (400 MHz, CD₃OD) δ 8.41 (d, J = 5.1 Hz, 1H), 7.88 (d, J = 8.1 Hz, 1H), 7.74 (d, J = 8.2 Hz, 1H), 6.98 (d, J = 5.1 Hz, 1H), 6.21 (s, 2H), 4.23 (s, 3H), 4.08 (dd, J = 11.5, 2.8 Hz, 1H), 3.93 (dd, J = 16.4, 5.7 Hz, 1H), 3.67 (dt, J = 11.8, 5.9 Hz, 1H), 3.52 (dd, J = 17.2, 8.7 Hz, 1H), 3.20 − 3.10 (m, 1H), 2.88 (q, J = 7.5 Hz, 2H), 2.56 − 2.44 (m, 2H), 2.26 − 2.17 (m, 4H), 2.02 (dt, J = 18.0, 8.5 Hz, 1H), 1.87 − 1.66 (m, 4H), 1.52 (dd, J = 23.9, 12.2 Hz, 1H), 1.23 (t, J = 7.5 Hz, 3H). | Example 1 & Example 30 |
| | diastereomer-4 | | |
| | From Example 30 | | |
| | compound 7-4 | | |
| | 2-(4-(6-(5-(((4- | | |
| | cyclobutylpyrimidin-2- | | |
| | yl)oxy)methyl)-1-methyl- | | |
| | 1H-1,2,3-triazol-4-yl)-2- | | |
| | ethylpyridin-3- | | |
| | yl)tetrahydro-2H-2λ³,4λ³- | | |
| | pyran-2-yl)acetic acid | | |

### Example 38: 2-[3-(2-methyl-6-{1-methyl-5-[(2-oxo-5-propyl-1,2-dihydropyridin-1-yl)methyl]-1H-1,2,3-triazol-4-yl}pyridin-3-yl)cyclohexyl]acetic acid

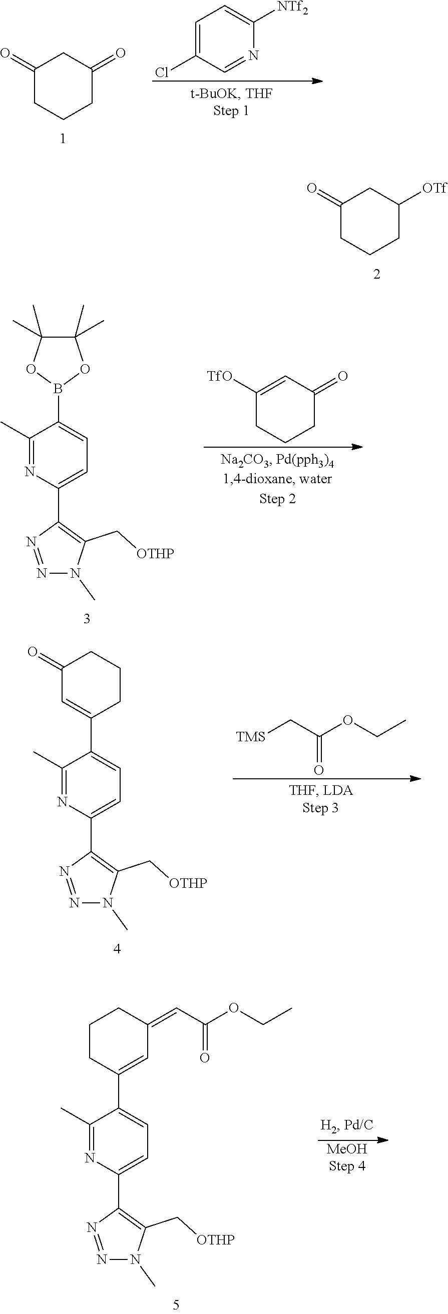

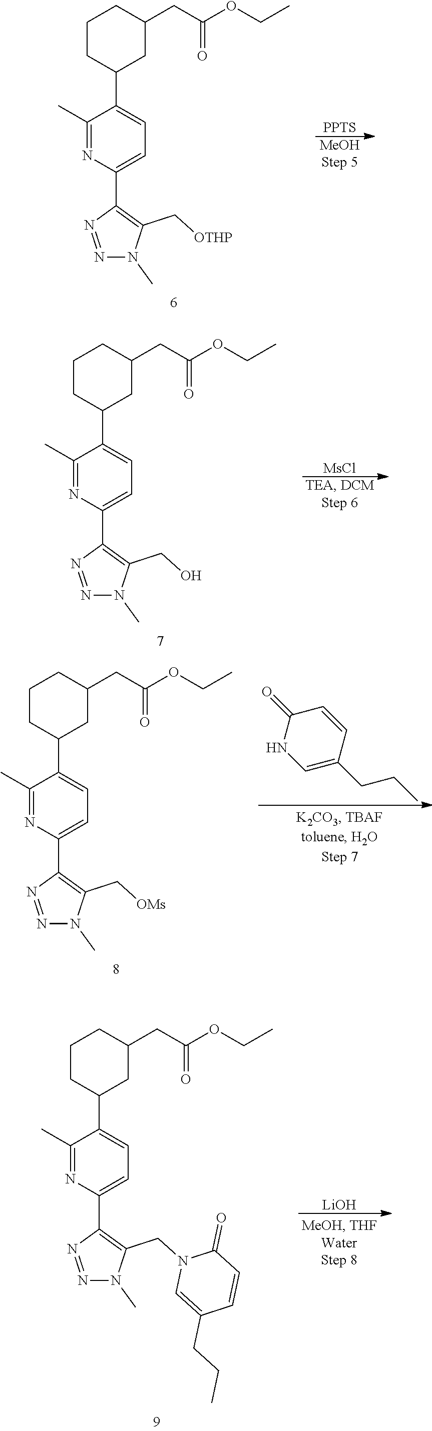

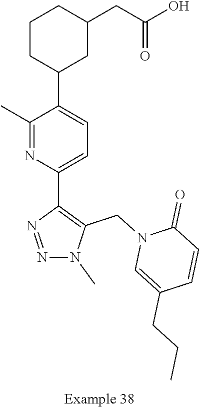 [SMILES: CCCc1ccc(=O)n(Cc2c(-c3ccc(C4CCCC(CC(=O)O)C4)c(C)n3)nnn2C)c1]

### Step 1: 3-oxocyclohex-1-en-1-yl trifluoromethanesulfonate

To a solution of cyclohexane-1,3-dione (2 g, 17.8 mmol) in THF (20 mL) was added t-BuOK (21.4 mL, 21.4 mmol) drop-wisely at 0° C. and the mixture was stirred at this temperature for 30 min followed by in portions addition of N-(5-chloropyridin-2-yl)-1,1,1-trifluoro-N-trifluoromethanesulfonylmethanesulfonamide (8.4 g, 21.4 mmol). The mixture was stirred at r.t. for 2 hrs. The mixture was diluted with EtOAc (100 mL), washed with saturated aq. NH₄Cl solution and brine, dried over anhydrous Na₂SO₄, filtered and concentrated to dryness. The residue was purified by flash chromatography (silica gel, 0˜10% of EtOAc in PE) to give the title compound (3.2 g, 73.5% yield) as yellow oil. ¹H NMR (400 MHz, CDCl₃) δ 6.07 (t, J=1.3 Hz, 1H), 2.69 (td, J=6.2, 1.3 Hz, 2H), 2.50-2.42 (m, 2H), 2.18-2.08 (m, 2H).

### Step 2: 5-(2-methyl-6-{1-methyl-5-[(oxan-2-yloxy)methyl]-1H-1,2,3-triazol-4-yl}pyridin-3-yl)-3,6-dihydro-2H-pyran-3-one

To a mixture of 5-oxo-5,6-dihydro-2H-pyran-3-yl trifluoromethanesulfonate (330 mg, 1.3 mmol) and 2-methyl-6-{1-methyl-5-[(oxan-2-yloxy)methyl]-1H-1,2,3-triazol-4-yl}-3-(tetramethyl-1,3,2-dioxaborolan-2-yl)pyridine (667 mg, 1.6 mmol) in 1,4-dioxane (8 mL) and water (2 mL) were added Na₂CO₃ (426 mg, 4.0 mmol), Pd(PPh₃)₄ (155 mg, 0.13 mmol) under N₂ atmosphere, after addition, the mixture was degassed under N₂ atmosphere for three times and stirred at 60° C. for 2 hrs. The mixture was diluted with water and extracted with EtOAc (2×10 mL). The combined organic layers were washed with brine, dried over anhydrous Na₂SO₄, filtered and concentrated to dryness. The residue was purified by flash chromatography (silica gel, 0˜40% of EtOAc in PE) to give the title compound (480 mg, 93.1% yield) as yellow oil. LC/MS (ESI) m/z: 385 (M+H)⁺.

### Step 3: 2-[(1Z)-3-(2-methyl-6-{1-methyl-5-[(oxan-2-yloxy)methyl]-1H-1,2,3-triazol-4-yl}pyridin-3-yl)cyclohex-2-en-1-ylidene]acetate

To a solution of ethyl 2-(trimethylsilyl)acetate (0.3 mL, 1.6 mmol) in THE (10 mL) was added LDA (2.4 mL, 4.7 mmol) drop-wisely at −70° C. After stirring at this temperature for 30 min, a solution of 3-(2-methyl-6-(1-methyl-5-(((tetrahydro-2H-pyran-2-yl)oxy)methyl)-1H-1,2,3-triazol-4-yl)pyridin-3-yl)cyclohex-2-en-1-one (600 mg, 1.6 mmol) in THE (5 mL) was added and the resulting mixture was stirred at −70° C. for 30 mins. The reaction mixture was quenched with ice-cooled saturated aq. NH₄Cl solution and brine, dried over anhydrous Na₂SO₄, filtered and concentrated to dryness. The residue was purified by flash chromatography (silica gel, 0˜40% of EtOAc in PE) to give the title compound (500 mg, 70.4% yield) as light yellow solid. LC/MS (ESI) m/z: 453 (M+H)⁺.

### Step 4: ethyl 2-[3-(2-methyl-6-{1-methyl-5-[(oxan-2-yloxy)methyl]-1H-1,2,3-triazol-4-yl}pyridin-3-yl)cyclohexyl]acetate

To a solution of ethyl 2-[(1Z)-3-(2-methyl-6-{1-methyl-5-[(oxan-2-yloxy)methyl]-1H-1,2,3-triazol-4-yl}pyridin-3-yl)cyclohex-2-en-1-ylidene]acetate (500 mg, 1.1 mmol) in MeOH (10 mL) was added Pd/C (30 mg, 0.2 mmol, 10% wt) at 0° C. and the mixture was degassed under N₂ atmosphere for three times and stirred under a H₂ balloon at room temperature for 16 hrs. The mixture was filtered and the filtrate was concentrated to dryness to give the title compound (460 mg, 91.2% yield) as white semi-solid. LC/MS (ESI) m/z: 457 (M+H)⁺.

### Step 5: ethyl 2-(3-{6-[5-(hydroxymethyl)-1-methyl-1H-1,2,3-triazol-4-yl]-2-methylpyridin-3-yl}cyclohexyl)acetate

To a solution of ethyl 2-[(1Z)-3-(2-methyl-6-{1-methyl-5-[(oxan-2-yloxy)methyl]-1H-1,2,3-triazol-4-yl}pyridin-3-yl)cyclohex-2-en-1-ylidene]acetate (460 mg, 1 mmol) in MeOH (10 mL) was added PPTS (1.2 g, 5.1 mmol) and the mixture was stirred at 50° C. for 16 hrs. The mixture was concentrated under reduced pressure to dryness and the residue was purified by chromatography on silica gel (eluted with PE:EtOAc=1:1) to give the title compound (270 mg, 72.3% yield) as light yellow solid. LC/MS (ESI) m/z: 373 (M+H)⁺. ¹H NMR (400 MHz, CDCl₃) δ 8.11 (dd, J=8.1, 1.3 Hz, 1H), 7.70-7.64 (m, 1H), 4.85 (s, 2H), 4.18-4.11 (m, 2H), 4.08 (d, J=7.4 Hz, 3H), 3.00-2.74 (m, 1H), 2.64-2.58 (m, 3H), 2.54 (d, J=6.7 Hz, 1H), 2.31-2.20 (m, 1H), 2.01-1.81 (m, 3H), 1.78-1.58 (m, 4H), 1.43-1.30 (m, 1H), 1.28-1.23 (m, 4H), 1.20-1.04 (m, 1H).

### Step 6: 2-(4-{5-[3-(2-ethoxy-2-oxoethyl)cyclohexyl]-6-methylpyridin-2-yl}-1-methyl-1H-1,2,3-triazol-5-yl)ethane-1-sulfonic acid

To a solution of ethyl 2-(3-{6-[5-(hydroxymethyl)-1-methyl-1H-1,2,3-triazol-4-yl]-2-methylpyridin-3-yl}cyclohexyl)acetate (30 mg, 0.08 mmol) in DCM (5 mL) was added TEA (0.1 mL, 0.24 mmol) followed by MsCl (0.1 mL, 0.1 mmol) at 0° C. and the reaction mixture was stirred at 0° C. for 30 min. TLC showed the reaction was completed, the mixture was diluted with ice water and extracted with DCM (2×5 mL). The combined organic layers were washed with brine, dried over anhydrous Na₂SO₄, filtered and concentrated to dryness give the title compound (33 mg, 90.4% yield) as yellow solid. LC/MS (ESI) m/z: 451 (M+H)⁺.

### Step 7: 2-(4-{5-[3-(2-ethoxy-2-oxoethyl)cyclohexyl]-6-methylpyridin-2-yl}-1-methyl-1H-1,2,3-triazol-5-yl)ethane-1-sulfonic acid

To a mixture of ethyl 2-[3-(6-{5-[(methanesulfonyloxy)methyl]-1-methyl-1H-1,2,3-triazol-4-yl}-2-methylpyridin-3-yl)cyclohexyl]acetate (35 mg, 0.07 mmol) and 5-propyl-1,2-dihydropyridin-2-one (13 mg, 0.1 mmol) in toluene (4 mL) and water (1 mL) was added K₂CO₃ (32 mg, 0.2 mmol) and TBAF (2.0 mg, 0.01 mmol) and the mixture was stirred at 100° C. for 16 hrs. The reaction mixture was diluted with EtOAc (10 mL) and washed with water and brine, dried over anhydrous Na₂SO₄, filtered and concentrated to dryness to give the title compound (30 mg, 78.5% yield) as yellow solid. LC/MS (ESI) m/z: 492 (M+H)⁺.

### Step 8: 2-[3-(2-methyl-6-{1-methyl-5-[(2-oxo-5-propyl-1,2-dihydropyridin-1-yl)methyl]-1H-1,2,3-triazol-4-yl}pyridin-3-yl)cyclohexyl]acetic acid

To a solution of ethyl 2-[3-(2-methyl-6-{1-methyl-5-[(2-oxo-5-propyl-1,2-dihydropyridin-1-yl)methyl]-1H-1,2,3-triazol-4-yl}pyridin-3-yl)cyclohexyl]acetate (35 mg, 0.07 mmol) in MeOH (1 mL), water (1 mL) and THF (2 mL) was added LiOH (30 mg, 0.7 mmol) and the mixture was stirred at 25° C. for 1 hr. The reaction mixture was acidified with 1M aq. HCl and extracted with DCM (2×5 mL). The combined organic layers were washed with brine, dried over Na₂SO₄, filtered and concentrated to dryness. The residue was purified by prep. HPLC (C18, 0˜90% acetonitrile in H₂O with 0.1% TFA) to give the title compound (20 mg, 60.6% yield) as white solid. LC/MS (ESI) m/z: 464 (M+H)⁺. ¹H NMR (400 MHz, DMSO-d₆) δ 7.84 (d, J=8.1 Hz, 1H), 7.82 (s, 1H), 7.75-7.68 (m, 1H), 7.29 (dd, J=9.3, 2.5 Hz, 1H), 6.35 (d, J=9.2 Hz, 1H), 5.62 (d, J=16.3 Hz, 2H), 4.19 (d, J=11.8 Hz, 3H), 2.88 (dd, J=48.2, 11.8 Hz, 1H), 2.61 (d, J=1.3 Hz, 3H), 2.48-2.30 (m, 1H), 2.17 (dd, J=9.3, 5.4 Hz, 3H), 1.90-1.70 (m, 3H), 1.60 (dd, J=26.6, 14.9 Hz, 3H), 1.45 (dd, J=26.5, 14.4 Hz, 1H), 1.38-1.13 (m, 3H), 1.13-0.82 (m, 1H), 0.71 (t, J=7.3 Hz, 3H).

### Example 39: 2-{3-[6-(5-{[(4-cyclobutylpyrimidin-2-yl)oxy]methyl}-1-methyl-1H-1,2,3-triazol-4-yl)-2-methylpyridin-3-yl]cyclohexyl}acetic acid

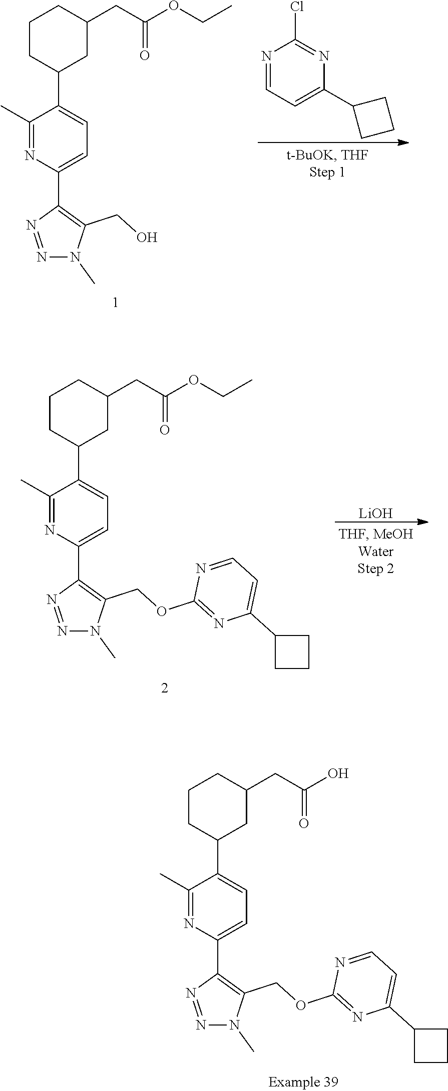 [SMILES: CCOC(=O)CC1CCCC(c2ccc(-c3nnn(C)c3CO)nc2C)C1 ; Clc1nccc(C2CCC2)n1 ; CCOC(=O)CC1CCCC(c2ccc(-c3nnn(C)c3COc3nccc(C4CCC4)n3)nc2C)C1 ; "LiOH" ; Cc1nc(-c2nnn(C)c2COc2nccc(C3CCC3)n2)ccc1C1CCCC(CC(=O)O)C1]

### Step 1: ethyl 2-{3-[6-(5-{[(4-cyclobutylpyrimidin-2-yl)oxy]methyl}-1-methyl-1H-1,2,3-triazol-4-yl)-2-methylpyridin-3-yl]cyclohexyl}acetate

To a mixture of ethyl 2-(3-{6-[5-(hydroxymethyl)-1-methyl-1H-1,2,3-triazol-4-yl]-2-methylpyridin-3-yl}cyclohexyl)acetate (40 mg, 0.11 mmol) and 2-chloro-4-cyclobutylpyrimidine (27 mg, 0.16 mmol) in THF (5 mL) was added t-BuOK (0.16 mL, 0.16 mmol, 1 M in THF) at 0° C. and the mixture was stirred at room temperature for 1 hr. The mixture was diluted with EtOAc and washed with saturated aq. NH₄Cl solution and brine, dried over anhydrous Na₂SO₄, filtered and concentrated to dryness to give the title compound (48 mg, 88.6% yield) as yellow solid. LC/MS (ESI) m/z: 505 (M+H)⁺.

### Step 2: 2-{3-[6-(5-{[(4-cyclobutylpyrimidin-2-yl)oxy]methyl}-1-methyl-1H-1,2,3-triazol-4-yl)-2-methylpyridin-3-yl]cyclohexyl}acetic acid

To a solution of ethyl 2-{3-[6-(5-{[(4-cyclobutylpyrimidin-2-yl)oxy]methyl}-1-methyl-1H-1,2,3-triazol-4-yl)-2-methylpyridin-3-yl]cyclohexyl}acetate (48 mg, 0.1 mmol) in MeOH (1 mL), water (1 mL) and THE (4 mL) was added LiOH (40 mg, 0.9 mmol) and the mixture was stirred at 25° C. for 1 hr. The reaction mixture was concentrated to dryness. The residue was diluted with water (10 mL) and washed with EtOAc (2×5 mL). The aqueous layer was acidified with 1M aq. HCl to pH-3 and extracted with DCM (2×5 mL). The combined organic layers were washed with brine, dried over Na₂SO₄, filtered and concentrated to dryness. The residue was purified by prep. HPLC to give the title compound (10 mg, 22.1% yield) as white solid. ¹H NMR (400 MHz, CD₃OD) δ 8.39 (d, J=5.1 Hz, 1H), 7.83 (d, J=8.1 Hz, 1H), 7.71 (t, J=9.3 Hz, 1H), 6.96 (d, J=5.1 Hz, 1H), 6.21-6.11 (m, 2H), 4.23 (s, 3H), 3.48 (q, J=8.6 Hz, 1H), 2.92 (dt, J=66.1, 11.7 Hz, 1H), 2.70-2.53 (m, 1H), 2.51 (s, 3H), 2.47 (d, J=13.8 Hz, 1H), 2.22 (ddd, J=17.3, 7.8, 3.1 Hz, 5H), 2.04-1.94 (m, 2H), 1.91-1.84 (m, 2H), 1.83-1.72 (m, 2H), 1.68 (s, 1H), 1.61-1.35 (m, 2H), 1.30-1.02 (m, 2H). LC/MS (ESI) m/z: 477 (M+H)⁺.

### Example 40 and Example 41: (R) or (S)-2-(3,3-difluoro-5-(2-methyl-6-(1-methyl-5-((2-oxo-5-propylpyridin-1(2H)-yl)methyl)-1H-1,2,3-triazol-4-yl)pyridin-3-yl)piperidin-1-yl)acetic acid & (S) or (R)-2-(3,3-difluoro-5-(2-methyl-6-(1-methyl-5-((2-oxo-5-propylpyridin-1(2H)-yl)methyl)-1H-1,2,3-triazol-4-yl)pyridin-3-yl)piperidin-1-yl)acetic acid

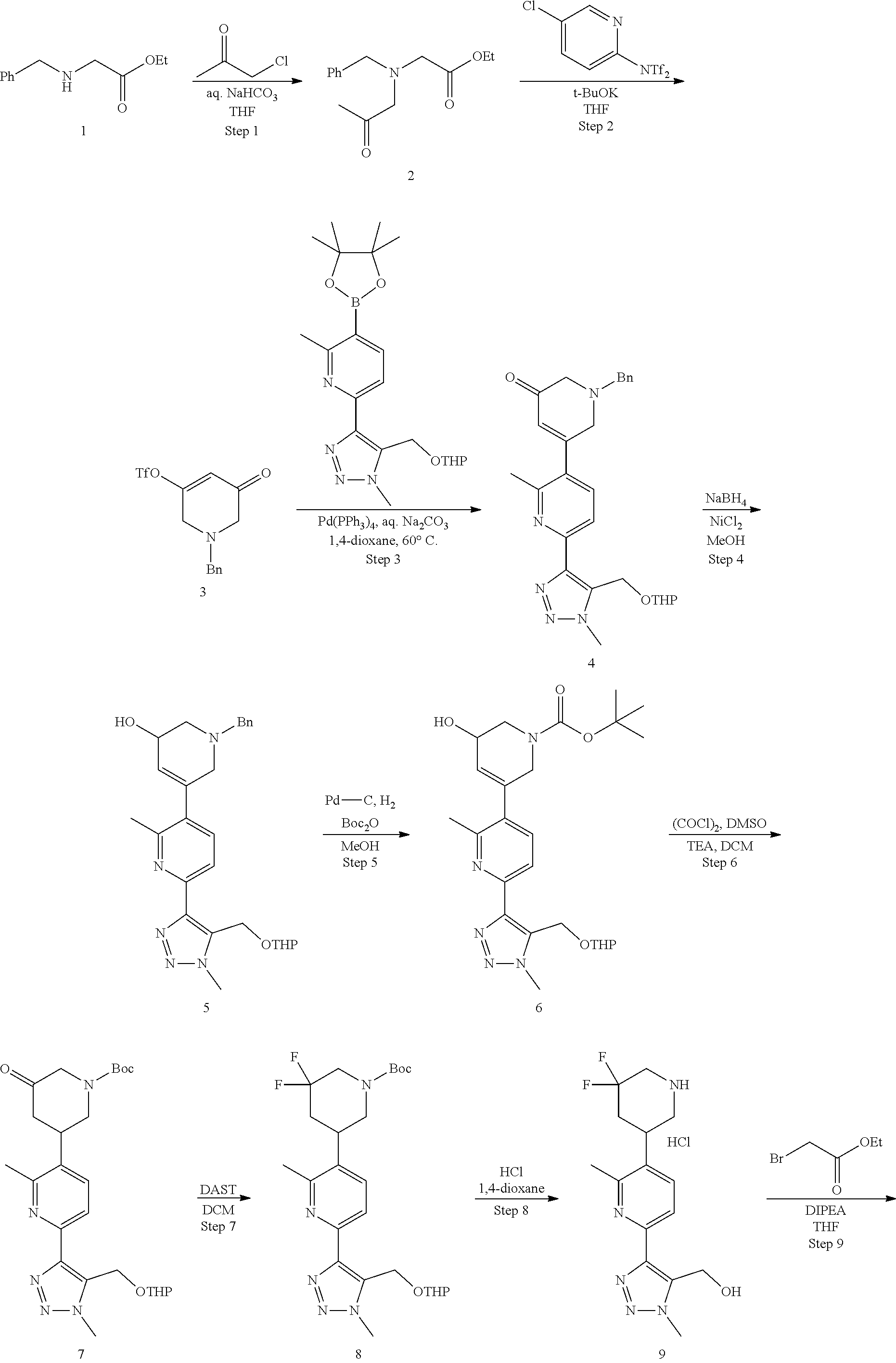

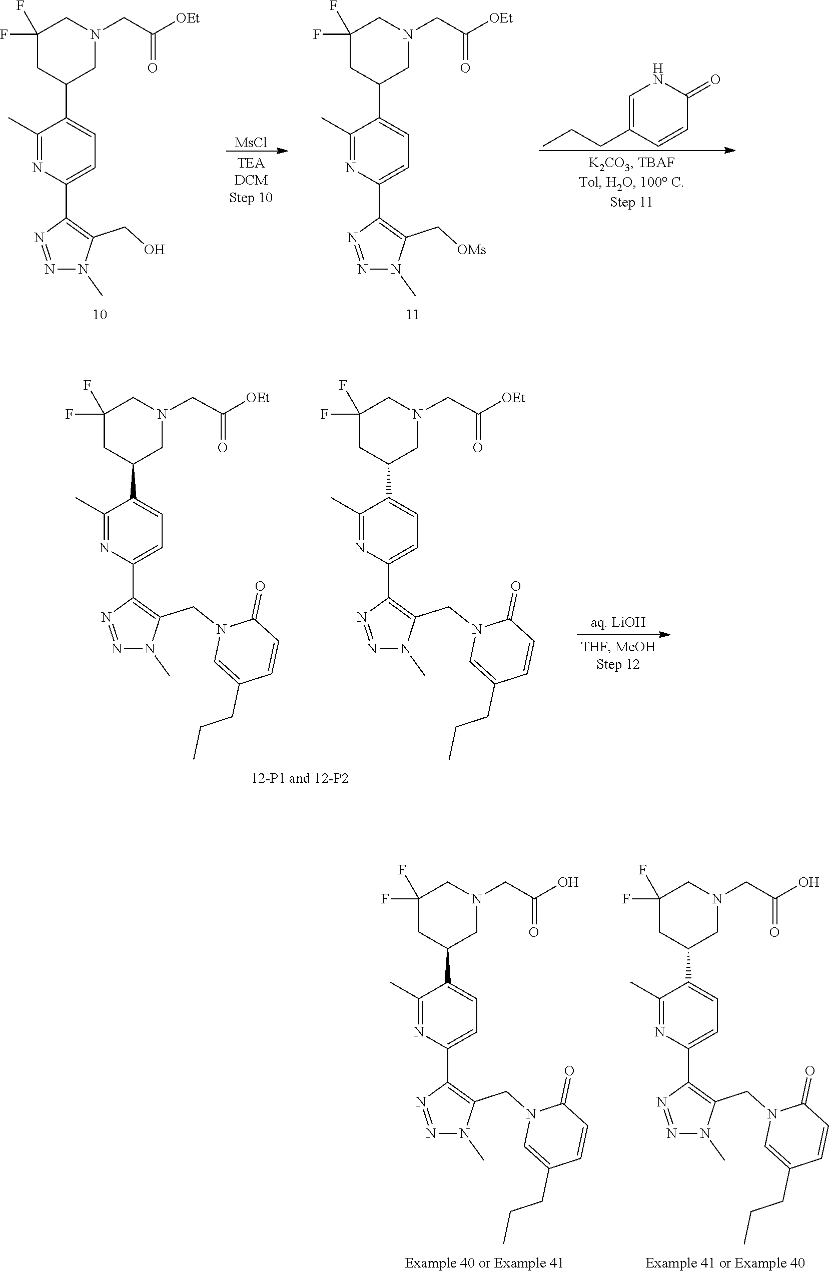

### Step 1: ethyl 2-[benzyl(2-oxopropyl)amino]acetate

To a mixture of ethyl 2-(benzylamino)acetate (9.7 mL, 51.7 mmol) and aq. NaHCO₃ solution (25.8 mL, 103.2 mmol, 4 M in water) in THF (100 mL) was added drop-wisely 1-chloropropan-2-one (4.9 mL, 62.1 mmol) at 0° C. for 15 mins. After the addition, the resulting solution was stirred at 80° C. overnight. After cooling to 0° C., the reaction mixture was treated with EtOAc (100 mL) and water (100 mL) and the organic layer was separated and the aqueous layer was extracted with EtOAc (150 mL). The combined organic was concentrated under vacuum to dryness and the residue was purified by flash chromatography (silica gel, 0˜50% of EtOAc in PE) to give the title compound (6.6 g, 51.2% yield) as colorless oil. LC/MS (ESI) m/z: 250 (M+H)⁺.

### Step 2: 1-benzyl-5-oxo-1,2,5,6-tetrahydropyridin-3-yl trifluoromethanesulfonate

To a solution of ethyl 2-[benzyl(2-oxopropyl)amino]acetate (5.6 g, 22.5 mmol) in THF (60 mL) was added t-BuOK (22.5 mL, 22.5 mmol, 1M in THF) drop-wisely at 0° C. for 15 mins. After the addition, the mixture was stirred at 0° C. for another 0.5 hr. A solution of N-(5-chloropyridin-2-yl)-1,1,1-trifluoro-Ntrifluoromethanesulfonylmethanesulfonamide (8.8 g, 22.5 mmol) in THF (20 mL) was added to the above mixture at 0° C. for 15 mins and the resulting solution was stirred at 0° C. for 1 hr. The reaction mixture was treated with EtOAc (50 mL) and water (50 mL), the organic layer was separated and the aqueous layer was extracted with EtOAc (50 mL). The combined organic layers were concentrated under reduced pressure to dryness and the residue was purified by flash chromatography (silica gel, 0˜50% of EtOAc in PE) to give the title compound (1.8 g, 23.9% yield) as colorless oil. LC/MS (ESI) m/z: 336 (M+H)⁺.

### Step 3: 1-benzyl-2′-methyl-6′-(1-methyl-5-(((tetrahydro-2H-pyran-2-yl)oxy)methyl)-1H-1,2,3-triazol-4-yl)-1,6-dihydro-[3,3′-bipyridin]-5(2H)-one

To a mixture of 1-benzyl-5-oxo-1,2,5,6-tetrahydropyridin-3-yl trifluoromethanesulfonate (990 mg, 2.9 mmol), 2-methyl-6-{1-methyl-5-[(oxan-2-yloxy)methyl]-1H-1,2,3-triazol-4-yl}-3-(tetramethyl-1,3,2-dioxaborolan-2-yl)pyridine (1.1 g, 2.7 mmol) and Na₂CO₃ (853 mg, 8.1 mmol) in 1,4-dioxane (20 mL) was added Pd(PPh₃)₄ (155 mg, 0.13 mmol) at 0° C. under N₂ atmosphere. After the addition, the resulting solution was degassed under N₂ for three times and stirred at 60° C. for 3 hrs. The mixture was diluted with EtOAc (50 mL), washed with water and brine, dried over Na₂SO₄, filtered and concentrated to dryness. The residue was purified by flash chromatography (silica gel, 0-100% of EtOAc in PE) to give the title compound (760 mg, 59.5% yield) as white solid. LC/MS (ESI) m/z: 474 (M+H)⁺. ¹H NMR (400 MHz, CDCl₃) δ 8.03 (d, J=8.0 Hz, 1H), 7.48 (d, J=8.1 Hz, 1H), 7.35 (d, J=4.4 Hz, 4H), 7.33-7.27 (m, 1H), 6.14 (t, J=1.4 Hz, 1H), 5.41-5.25 (m, 2H), 4.79-4.73 (m, 1H), 4.16 (s, 3H), 3.91-3.82 (m, 1H), 3.75 (s, 2H), 3.55-3.47 (m, 1H), 3.44 (s, 2H), 3.30 (s, 2H), 2.50 (s, 3H), 1.70-1.49 (m, 6H).

### Step 4: 1-benzyl-2′-methyl-6′-(1-methyl-5-(((tetrahydro-2H-pyran-2-yl)oxy)methyl)-1H-1,2,3-triazol-4-yl)-1,2,5,6-tetrahydro-[3,3′-bipyridin]-5-ol

To a solution of 1-benzyl-5-(2-methyl-6-{1-methyl-5-[(oxan-2-yloxy)methyl]-1H-1,2,3-triazol-4-yl}pyridin-3-yl)-1,2,3,6-tetrahydropyridin-3-one (630 mg, 1.3 mmol) in MeOH (10 mL) was added NiCl₂ (428 mg, 2.7 mmol) followed by in portions addition of NaBH₄ (113 mg, 2.7 mmol) at 0° C. After the addition, the mixture was stirred at r.t. for another 3 hrs. The reaction mixture was poured into ice-cooled saturated aq. NH₄Cl solution and extracted with EtOAc (2×10 mL). The combined organic layers were washed with water and brine, dried over Na₂SO₄, filtered and concentrated to dryness. The residue was purified by flash chromatography (silica gel, 0˜50% of EtOAc in PE) to give the title compound (430 mg, 68% yield) as colorless oil. LC/MS (ESI) m/z: 476 (M+H)⁺.

### Step 5: tert-butyl 3-hydroxy-5-(2-methyl-6-{1-methyl-5-[(oxan-2-yloxy)methyl]-1H-1,2,3-triazol-4-yl}pyridin-3-yl)piperidine-1-carboxylate

To a mixture of 1-benzyl-5-(2-methyl-6-{1-methyl-5-[(oxan-2-yloxy)methyl]-1H-1,2,3-triazol-4-yl}pyridin-3-yl)-1,2,3,6-tetrahydropyridin-3-ol (430 mg, 0.9 mmol) and Boc₂O (456 mg, 2.0 mmol) in MeOH (20 mL) was added Pd/C (50 mg, 10% wt), the mixture was degassed under N₂ atmosphere for three times and stirred under a H₂ balloon at r.t. for 16 hrs. The mixture was filtered and the filtrate was concentrated under reduced pressure to dryness. The residue was purified by flash chromatography (0˜40% of EtOAc in PE) to give the title compound (320 mg, 72.9% yield) as colorless oil. LC/MS (ESI) m/z: 488 (M+H)⁺.

### Step 6: (S)—N-(6-bromo-3-methylpyridin-2-yl)-2-(N-methyl-4-nitrophenylsulfonamido)hex-5-enamide

To a solution of DMSO (0.2 mL, 2.5 mmol) in DCM (5 mL) was added Oxalyl Chloride (0.1 mL, 1.3 mmol) drop-wisely at −70° C. After the addition, the mixture was stirred at −70° C. for another 10 mins. A solution of tert-butyl 3-hydroxy-5-(2-methyl-6-{1-methyl-5-[(oxan-2-yloxy)methyl]-1H-1,2,3-triazol-4-yl}pyridin-3-yl)piperidine-1-carboxylate (250 mg, 0.5 mmol) in DCM (2 mL) was added to the above mixture drop-wisely at −70° C. for 10 mins and the reaction mixture was stirred at this temperature for 1 hr. TEA (0.4 mL, 2.8 mmol) was added drop-wisely to the above mixture at −70° C. and the resulting mixture was stirred at −70° C. to r.t. for 1 hr. The mixture was diluted with DCM (10 mL), washed with water and brine, dried over Na₂SO₄, filtered and concentrated to dryness. The residue was purified by flash chromatography (silica gel, 0˜40% of EtOAc in PE) to give the title compound (210 mg, 80.3% yield) as colorless oil. LC/MS (ESI) m/z: 486 (M+H)⁺. ¹H NMR (400 MHz, CDCl₃) δ 7.94 (d, J=8.1 Hz, 1H), 7.44 (d, J=8.2 Hz, 1H), 5.34-5.24 (m, 2H), 4.73-4.67 (m, 1H), 4.22 (m, 1H), 4.08 (s, 3H), 3.92-3.77 (m, 2H), 3.49-3.40 (m, 2H), 3.27 (s, 1H), 2.97 (m, 1H), 2.77-2.61 (m, 2H), 2.57 (s, 3H), 1.73-1.60 (m, 2H), 1.54-1.45 (m, 4H), 1.40 (s, 9H).

### Step 7: tert-butyl 3,3-difluoro-5-(2-methyl-6-{1-methyl-5-[(oxan-2-yloxy)methyl]-1H-1,2,3-triazol-4-yl}pyridin-3-yl)piperidine-1-carboxylate

To a solution of tert-butyl 3-(2-methyl-6-{1-methyl-5-[(oxan-2-yloxy)methyl]-1H-1,2,3-triazol-4-yl}pyridin-3-yl)-5-oxopiperidine-1-carboxylate (180 mg, 0.37 mmol) in DCM (5 mL) was added DAST (187 mg, 0.8 mmol) drop-wisely at 0° C. After the addition, the reaction mixture was stirred at r.t. for 1 hr. The mixture was poured into ice-cooled saturated aq. NaHCO₃ solution and extracted with DCM (2×10 mL). The combined organic layers were washed with brine, dried over Na₂SO₄, filtered and concentrated to dryness. The residue was purified by flash chromatography (0˜50% of EtOAc in PE) to give the title compound (145 mg, 77.1% yield) as colorless oil. LC/MS (ESI) m/z: 508 (M+H)⁺. ¹H NMR (400 MHz, CDCl₃) δ 8.02 (dd, J=8.1, 4.0 Hz, 1H), 7.50 (t, J=6.7 Hz, 1H), 5.45-5.32 (m, 2H), 4.78-4.76 (m, 1H), 4.36 (dd, J=24.0, 8.2 Hz, 1H), 4.16 (s, 3H), 3.89 (t, J=9.3 Hz, 1H), 3.53 (dd, J=10.4, 4.5 Hz, 1H), 3.32-3.20 (m, 1H), 3.09-2.91 (m, 1H), 2.67 (s, 3H), 2.64 (s, 1H), 2.38 (s, 1H), 1.84-1.64 (m, 4H), 1.62-1.54 (m, 4H), 1.50 (s, 9H).

### Step 8: {4-[5-(5,5-difluoropiperidin-3-yl)-6-methylpyridin-2-yl]-1-methyl-1H-1,2,3-triazol-5-yl}methanol hydrochloride

To a solution of tert-butyl 3,3-difluoro-5-(2-methyl-6-{1-methyl-5-[(oxan-2-yloxy)methyl]-1H-1,2,3-triazol-4-yl}pyridin-3-yl)piperidine-1-carboxylate (150 mg, 0.3 mmol) in DCM (5 mL) was added HCl/1,4-dioxane (0.5 mL, 2 mmol, 4 M) at 0° C. After stirring at r.t. for 2 hrs, the mixture was concentrated under reduced pressure to dryness to give the title compound (110 mg, 100% yield) as yellow solid, which was used in next reaction directly. LC/MS (ESI) m/z: 304 (M+H)⁺.

### Step 9: ethyl 2-(3,3-difluoro-5-{6-[5-(hydroxymethyl)-1-methyl-1H-1,2,3-triazol-4-yl]-2-methylpyridin-3-yl}piperidin-1-yl)acetate

To a mixture of {4-[5-(5,5-difluoropiperidin-3-yl)-6-methylpyridin-2-yl]-1-methyl-1H-1,2,3-triazol-5-yl}methanol hydrochloride (94 mg, 0.26 mmol) and DIPEA (0.16 mL, 1 mmol) in THE (5 mL) was added ethyl 2-bromoacetate (48 mg, 0.3 mmol) at 0° C. and the mixture was stirred at r.t. for 3 hrs. The reaction was concentrated under reduced pressure to dryness and the residue was purified by flash chromatography (silica gel, 0˜60% of EtOAc in PE) to give the title compound (55 mg, 51.1% yield) as colorless oil. LC/MS (ESI) m/z: 410 (M+H)⁺. ¹H NMR (400 MHz, CDCl₃) δ 8.14 (d, J=8.2 Hz, 1H), 7.65 (d, J=8.2 Hz, 1H), 7.53 (d, J=10.5 Hz, 1H), 4.83 (s, 2H), 4.19 (m, 2H), 4.08 (s, 3H), 3.47 (s, 2H), 3.39 (m, 1H), 3.22 (m, 1H), 2.97 (m, 2H), 2.72 (m, 1H), 2.66 (s, 3H), 2.34 (m, 1H), 2.06-1.87 (m, 1H), 1.28 (t, J=7.1 Hz, 3H).

### Step 10: ethyl 2-[3,3-difluoro-5-(6-{5-[(methanesulfonyloxy)methyl]-1-methyl-1H-1,2,3-triazol-4-yl}-2-methylpyridin-3-yl)piperidin-1-yl]acetate

To a solution of ethyl 2-(3,3-difluoro-5-{6-[5-(hydroxymethyl)-1-methyl-1H-1,2,3-triazol-4-yl]-2-methylpyridin-3-yl}piperidin-1-yl)acetate (55 mg, 0.14 mmol) in DCM (5 mL) was added and TEA (0.1 mL, 0.7 mmol) and MsCl (24 mg, 0.2 mmol) at 0° C. and the mixture was stirred at r.t. for 2 hrs. The mixture was diluted with DCM (5 mL), washed with water and brine, dried over Na₂SO₄, filtered and concentrated to dryness to give the title compound (60 mg, 88.4% yield) as yellow solid. LC/MS (ESI) m/z: 488 (M+H)⁺.

### Step 11: ethyl (R) or (S)-2-(3,3-difluoro-5-(2-methyl-6-(1-methyl-5-((2-oxo-5-propylpyridin-1(2H)-yl)methyl)-1H-1,2,3-triazol-4-yl)pyridin-3-yl)piperidin-1-yl)acetate (12-P1) & ethyl (S) or (R)-2-(3,3-difluoro-5-(2-methyl-6-(1-methyl-5-((2-oxo-5-propylpyridin-1(2H)-yl)methyl)-1H-1,2,3-triazol-4-yl)pyridin-3-yl)piperidin-1-yl)acetate (12-P2)

To a mixture of ethyl 2-[3,3-difluoro-5-(6-{5-[(methanesulfonyloxy)methyl]-1-methyl-1H-1,2,3-triazol-4-yl}-2-methylpyridin-3-yl)piperidin-1-yl]acetate (60 mg, 0.12 mmol) and 5-propyl-1,2-dihydropyridin-2-one (25 mg, 0.18 mmol) in toluene (2 mL) and H₂O (1 mL) was added K₂CO₃ (55.2 mg, 0.4 mmol) and TBAF (5 mg, cat.) and the mixture was stirred at 110° C. for 3 hrs. The mixture was diluted with EtOAc (5 mL), washed with water and brine, dried over Na₂SO₄, filtered and concentrated to dryness to give the racemic product, which was purified by chiral SFC to give compound 12-P1 (Peak 1, retention time: 4.88 min) (16 mg, 25% yield) and compound 12-P2 (Peak 2, retention time: 5.08 min) (22 mg, 34.6% yield) as yellow solid. LC/MS (ESI) m/z: 529 (M+H)⁺. Absolute stereochemistry has not been assigned. SFC condition: Column: ChiralPak OJ, 250×21.2 mm I.D., 5 μm; Mobile phase: A for CO₂ and B for Methanol (0.1% NH₄OH); Gradient: B 30%; Flow rate: 50 mL/min; Column temperature: 35° C.

### Example 40: (R) or (S)-2-(3,3-difluoro-5-(2-methyl-6-(1-methyl-5-((2-oxo-5-propylpyridin-1(2H)-yl)methyl)-1H-1,2,3-triazol-4-yl)pyridin-3-yl)piperidin-1-yl)acetic acid

To a solution of compound 12-P1 (16 mg, 0.03 mmol) in THE (1 mL), MeOH (0.5 mL) and H₂O (0.5 mL) was added LiOH (5 mg, 0.1 mmol) and the mixture was stirred at r.t. for 3 hrs. The reaction was treated with 1N aq. HCl to pH˜4 and extracted with DCM (3×5 mL). The combined organic layers were washed with brine, dried over Na₂SO₄, filtered and concentrated to dryness. The residue was purified by prep. HPLC (C18, 0˜90% acetonitrile in H₂O with 0.1% HCOOH) to give the title compound (5.8 mg, 35.3% yield) as white solid. LC/MS (ESI) m/z: 501 (M+H)⁺. ¹H NMR (400 MHz, CD₃OD) 7.92 (d, J=8.3 Hz, 2H), 7.84 (d, J=8.2 Hz, 1H), 7.44-7.37 (m, 1H), 6.50 (d, J=9.1 Hz, 1H), 5.74 (s, 2H), 4.22 (s, 3H), 3.55 (s, 2H), 3.50 (s, 1H), 3.40 (d, J=10.0 Hz, 1H), 3.12 (s, 1H), 2.97 (m, 1H), 2.76-2.67 (m, 4H), 2.37-2.27 (m, 3H), 2.26-2.09 (m, 1H), 1.42 (m, 2H), 0.81 (t, J=7.3 Hz, 3H).

### Example 41: (S) or (R)-2-(3,3-difluoro-5-(2-methyl-6-(1-methyl-5-((2-oxo-5-propylpyridin-1(2H)-yl)methyl)-1H-1,2,3-triazol-4-yl)pyridin-3-yl)piperidin-1-yl)acetic acid

The title compound was prepared from compound 12-P2 of example 40 using the same method for the synthesis of example 40. LC/MS (ESI) m/z: 501 (M+H)⁺. ¹H NMR (400 MHz, CD₃OD) 7.92 (d, J=8.3 Hz, 2H), 7.84 (d, J=8.2 Hz, 1H), 7.44-7.37 (m, 1H), 6.50 (d, J=9.1 Hz, 1H), 5.74 (s, 2H), 4.22 (s, 3H), 3.55 (s, 2H), 3.50 (s, 1H), 3.40 (d, J=10.0 Hz, 1H), 3.12 (s, 1H), 2.97 (m, 1H), 2.76-2.67 (m, 4H), 2.37-2.27 (m, 3H), 2.26-2.09 (m, 1H), 1.42 (m, 2H), 0.81 (t, J=7.3 Hz, 3H).

### Example 42: (1S,3S)-3-((6-(5-((4-(cyclopropylmethyl)-3-methyl-2-oxoimidazolidin-1-yl)methyl)-1-methyl-1H-1,2,3-triazol-4-yl)-2-methylpyridin-3-yl)oxy)cyclohexane-1-carboxylic acid

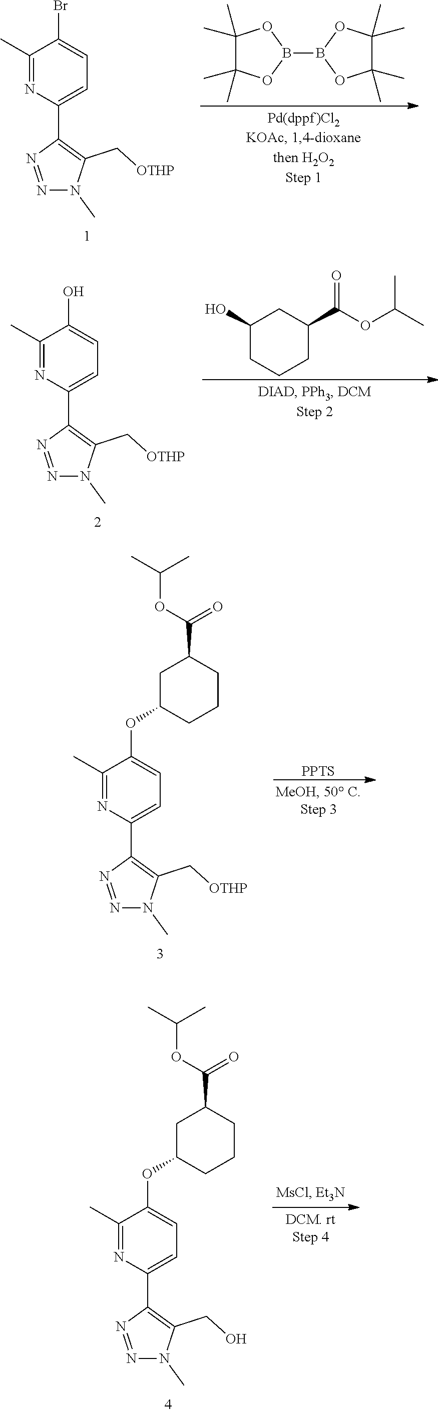

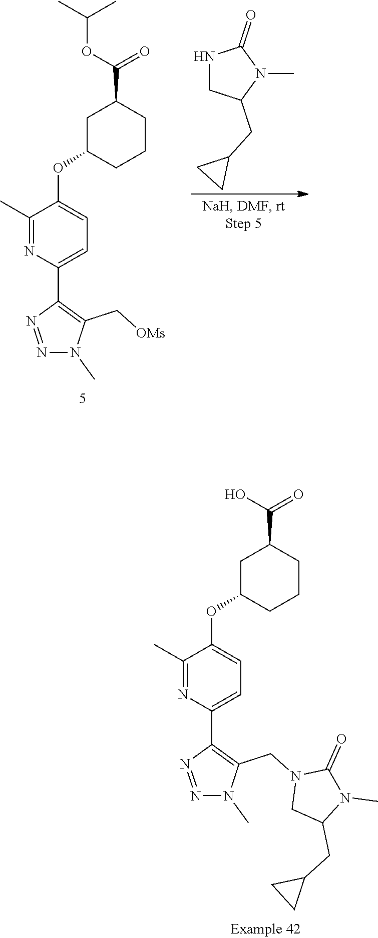

### Step 1: 2-methyl-6-(1-methyl-5-(((tetrahydro-2H-pyran-2-yl)oxy)methyl)-1H-1,2,3-triazol-4-yl)pyridin-3-ol

To a solution of 3-bromo-2-methyl-6-(1-methyl-5-(((tetrahydro-2H-pyran-2-yl)oxy)methyl)-1H-1,2,3-triazol-4-yl)pyridine (1 g, 2.72 mmol) and 4,4,4′,4′,5,5,5′,5′-octamethyl-2,2′-bi(1,3,2-dioxaborolane) (840 mg, 3.3 mmol) in 1,4-dioxane (20 mL) was added Pd(dppf)Cl₂ (99 mg, 0.136 mmol) and KOAc (400 mg, 4.08 mmol), the mixture was degassed under N₂ atmosphere for three times and stirred at 100° C. for 16 hrs. The mixture was cooled to 0° C. and aq. H₂O₂ (3 mL, 30% wt) was added drop-wisely and the resulting mixture was stirred 0° C. to r.t. for 2 hrs. The mixture was diluted with EtOAc (20 mL), washed with saturated aq. Na₂SO₃ solution and brine, dried over Na₂SO₄, filtered and concentrated to dryness. The residue was purified by flash chromatography (silica gel, 0-50% EtOAc in PE) to give the title compound (560 mg, 67.6% yield) as light yellow solid. LC/MS (ESI) m/z: 305 (M+H)⁺.

### Step 2: isopropyl (1S,3S)-3-((2-methyl-6-(1-methyl-5-(((tetrahydro-2H-pyran-2-yl)oxy)methyl)-11H-1,2,3-triazol-4-yl)pyridin-3-yl)oxy)cyclohexane-1-carboxylate

To a solution of 2-methyl-6-(1-methyl-5-(((tetrahydro-2H-pyran-2-yl)oxy)methyl)-1H-1,2,3-triazol-4-yl)pyridin-3-ol (500 mg, 1.64 mmol) in DCM (20 mL) were added propan-2-yl(1S,3R)-3-hydroxycyclohexane-1-carboxylate (612 mg, 3.29 mmol) and PPh₃ (1.29 g, 4.93 mmol) followed by drop-wise addition of DIAD (0.98 mL, 4.93 mmol) at 0° C. and the reaction mixture was stirred at room temperature for 16 hrs. The mixture was concentrated to dryness and the residue was purified by chromatography on silica gel (PE:EtOAc=3:1) to give the title compound (310 mg, 39.9% yield) as light yellow solid. LC/MS (ESI) m/z: 473 (M+H)⁺.

### Step 3: isopropyl (1S,3S)-3-((6-(5-(hydroxymethyl)-1-methyl-1H-1,2,3-triazol-4-yl)-2-methylpyridin-3-yl)oxy)cyclohexane-1-carboxylate

To a solution of isopropyl (1S,3S)-3-((2-methyl-6-(1-methyl-5-(((tetrahydro-2H-pyran-2-yl)oxy)methyl)-1H-1,2,3-triazol-4-yl)pyridin-3-yl)oxy)cyclohexane-1-carboxylate (310 mg, 0.66 mmol) in MeOH (10 mL) was added PPTS (330 mg, 1.3 mmol) and the mixture was stirred at 50° C. for 16 hrs. The reaction mixture was diluted with water (10 mL) and extracted with EtOAc (2×10 mL). The combined organic layers were washed with brine, dried over anhydrous Na₂SO₄, filtered and concentrated to dryness. The residue was purified by chromatography on silica gel (PE:EtOAc=1:1) to give the title compound (174 mg, 68.3% yield) as light yellow oil. LC/MS (ESI) m/z: 389 (M+H)⁺.

### Step 4: isopropyl (1S,3S)-3-((2-methyl-6-(1-methyl-5-(((methylsulfonyl)oxy)methyl)-1H-1,2,3-triazol-4-yl)pyridin-3-yl)oxy)cyclohexane-1-carboxylate

To a solution of isopropyl (1S,3S)-3-((6-(5-(hydroxymethyl)-1-methyl-1H-1,2,3-triazol-4-yl)-2-methylpyridin-3-yl)oxy)cyclohexane-1-carboxylate (170 mg, 0.438 mmol) in DCM (6 mL) were added TEA (0.18 mL, 1.3 mmol) and MsCl (0.068 mL, 0.88 mmol) at 0° C. and the mixture was stirred at room temperature for 2 hrs. The mixture was diluted with DCM (5 mL), washed with water and brine, dried over anhydrous Na₂SO₄, filtered and concentrated to dryness. The residue was purified by chromatography on silica gel (PE:EtOAc=2:1) to give the title compound (152 mg, 74.4% yield) as yellow solid. LC/MS (ESI) m/z: 467 (M+H)⁺.

### Step 5: (1S,3S)-3-((6-(5-((4-(cyclopropylmethyl)-3-methyl-2-oxoimidazolidin-1-yl)methyl)-1-methyl-1H-1,2,3-triazol-4-yl)-2-methylpyridin-3-yl)oxy)cyclohexane-1-carboxylic acid

To a solution of 5-(cyclopropylmethyl)-1-methylimidazolidin-2-one (44.6 mg, 0.29 mmol) in DMF (2 mL) was added portion-wisely NaH (19.3 mg, 0.48 mmol, 60% dispersion in mineral oil) at 0° C., and the mixture was stirred at 0° C. under N₂ atmosphere for 30 mins. A solution of isopropyl (1S,3S)-3-((2-methyl-6-(1-methyl-5-(((methylsulfonyl)oxy)methyl)-1H-1,2,3-triazol-4-yl)pyridin-3-yl)oxy)cyclohexane-1-carboxylate (150 mg, 0.32 mmol) was added at 0° C. and the mixture was stirred at room temperature under N₂ atmosphere for 2 hrs. The reaction mixture was quenched with ice-water and the mixture was stirred at r.t. for 2 hrs. The mixture was concentrated to dryness and the residue was dissolved in H₂O (10 mL). The mixture was washed with MTBE (2×5 mL), acidified with 1 N aq. HCl to pH-4 and extracted with DCM (3×5 mL). The combined organic layers were washed with brine, dried over anhydrous Na₂SO₄, filtered and concentrated to dryness. The residue was purified by prep-HPLC (C18, 10-80%, MeCN in H₂O with 0.1% HCOOH) to give the title compound (50 mg, 32.2% yield) as white solid. LC/MS (ESI) m/z: 483 (M+H)⁺. ¹H-NMR (400 MHz, CD₃OD) δ 7.81 (d, J=8.5 Hz, 1H), 7.45-7.42 (m, 1H), 5.01 (s, 2H), 4.11 (s, 3H), 3.50-3.41 (m, 2H), 3.09-3.04 (m, 1H), 2.80-2.74 (m, 4H), 2.56-2.47 (m, 3H), 2.14-2.08 (m, 1H), 1.95-1.90 (m, 3H), 1.79-1.55 (m, 3H), 1.51-1.43 (m, 2H), 1.40-1.29 (m, 1H), 0.49-0.43 (m, 1H), 0.38-0.30 (m, 2H), 0.03-0.01 (m, 2H).

### Example 43: (1S,3S)-3-((2-ethyl-6-(1-methyl-5-((2-oxo-5-propylpyridin-1(2H)-yl)methyl)-1H-1,2,3-triazol-4-yl)pyridin-3-yl)oxy)cyclohexanecarboxylic acid

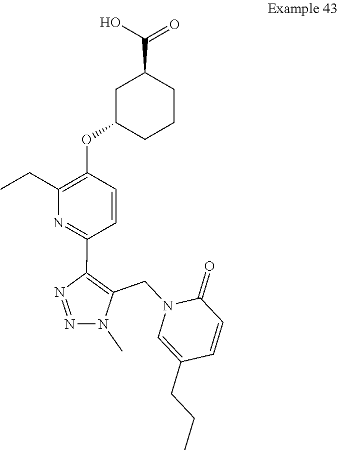 [SMILES: CCCc1ccc(=O)n(Cc2c(-c3ccc(O[C@H]4CCC[C@H](C(=O)O)C4)c(CC)n3)nnn2C)c1]

The title compound was synthesized using the same synthetic sequence that was used to synthesize Example 2 and Example 42. LC/MS (ESI) m/z: 480 (M+H)⁺. ¹H NMR (400 MHz, CD₃OD) δ 7.84-7.81 (d, J=8.6 Hz, 1H), 7.68-7.66 (d, J=2.0 Hz, 1H), 7.47-7.44 (d, J=8.7 Hz, 1H), 7.34-7.40 (m 1H), 6.51-6.49 (d, J=9.2 Hz, 1H), 5.82 (s, 2H), 4.82-4.78 (m, 1H), 4.18 (s, 3H), 2.97-2.93 (m, 2H), 2.80-2.75 (m, 1H), 2.23-2.19 (m, 2H), 2.13-2.09 (m, 1H), 1.98-1.92 (m, 3H), 1.79-1.64 (m, 4H), 1.41-1.33 (m, 2H), 1.31-1.28 (t, J=7.6 Hz, 3H), 0.77-0.73 (t, J=7.6 Hz, 3H).

### Example 44: (1S,3S)-3-((6-(5-((5-(cyclopropylmethyl)-2-oxopyridin-1(2H)-yl)methyl)-1-methyl-1H-1,2,3-triazol-4-yl)-2-ethylpyridin-3-yl)oxy)cyclohexanecarboxylic acid

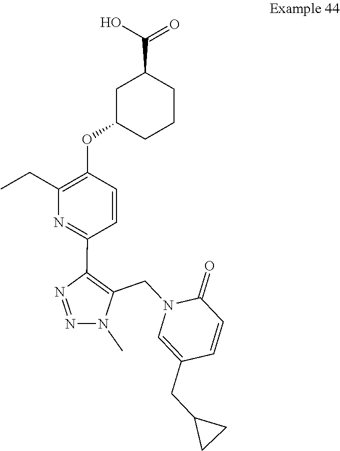 [SMILES: CCc1nc(-c2nnn(C)c2Cn2cc(CC3CC3)ccc2=O)ccc1O[C@H]1CCC[C@H](C(=O)O)C1]

The title compound was synthesized using the same synthetic sequence that was used to synthesize Example 2 and Example 42. LC/MS (ESI) m/z: 492 (M+H)⁺. ¹H NMR (400 MHz, CD₃OD) δ 7.88-7.85 (d, J=8.6 Hz, 1H), 7.71-7.69 (d, J=2.0 Hz, 1H), 7.48-7.44 (d, J=8.7 Hz, 1H), 7.43-7.40 (m, 1H), 6.53-6.50 (d, J=9.3 Hz, 1H), 5.90 (s, 2H), 4.79-4.82 (m, 1H), 4.16 (s, 3H), 2.95-2.91 (m, 2H), 2.81-2.72 (m, 1H), 2.16-2.14 (d, J=6.9 Hz, 2H), 2.14-2.07 (m, 1H), 2.00-1.89 (m, 3H), 1.79-1.60 (m, 4H), 1.26-1.32 (m, 3H), 0.70-0.56 (m, 1H), 0.31-0.23 (m, 2H), 0.10-0.07 (m, 2H).

### Biological Assays

### LPA1 Calcium Flux Assays

The effectiveness of compounds of the present invention as LPA1 inhibitors can be determined in an LPA1 functional antagonist assay as follows.

PathHunter® CHO-K1 EDG2 β-Arrestin Cell Line (Eurofins DiscoverX Corporation, Cat #93-0644C2) were plated overnight (11,500 cells/well) in poly-D-lysine coated 384-well microplates (Corning, Cat #356697) in AssayComplete™ Cell Plating 35 Reagent (Eurofins DiscoverX Corporation, Cat #93-0563R35). Following overnight culture, cells were loaded with calcium indicator dye from the HitHunter Calcium No WashPLUS Assay kit (Eurofins DiscoverX Corporation, Cat #93-0091) for 45 minutes at 37° C. Test compounds solubilized in DMSO (Sigma, Cat #276855) on 384-well polypropylene microplates (Greiner bio-one, Cat #781280) were then diluted to intermediate concentration with assay buffer [1×HBSS calcium/magnesium (Corning, Cat #21-023-CM) and 20 mM HEPES (Corning, Cat #25-060-Cl)] before addition to cells by Agilent Velocity 11 with a final concentration of 0.4% DMSO. After compound addition, assay plates were equilibrated to room temperature for 30 minutes. Cells were then stimulated by addition of EC80 concentration of LPA (Cat #, 10010093) in assay buffer containing [1×HBSS calcium/magnesium (Corning, Cat #21-023-CM), 20 mM HEPES (Corning, Cat #25-060-Cl), and 0.1% BSA (Sigma, Cat #A8806)] using Molecular Devices FLIPR Tetra PLUS®. IC50 values were determined by Chemical and Biological Information System from ChemInnovation Software, Inc.

The compounds of this invention were tested for their activity to inhibit LPA1 as determined in an LPA1 functional antagonist assay as described herein. Results of LPA1 assay are given in Table 4.

TABLE 4
Activity Data in LPA1 Assay*

| Example No. | LPA1 IC₅₀ |
|---|---|
| 1 | A |
| 2 | C |
| 3 | A |
| 4 | B |
| 5 | B |
| 6 | A |
| 7 | B |
| 8 | A |
| 9 | A |
| 10 | A |
| 11 | A |
| 12 | C |
| 13 | B |
| 14 | A |
| 15 | A |
| 16 | C |
| 17 | C |
| 18 | D |
| 19 | D |
| 20 | C |
| 21 | A |
| 22 | A |
| 23 | B |
| 24 | C |
| 25 | C |
| 26 | B |
| 27 | C |
| 28 | B |
| 29 | C |
| 30 | C |
| 31 | C |
| 32 | A |
| 33 | B |
| 34 | A |
| 35 | A |
| 36 | A |
| 37 | A |
| 38 | B |
| 39 | A |
| 40 | D |
| 41 | B |
| 42 | B |
| 43 | A |
| 44 | A |

*A: <100 nM; B: 100~500 nM; C: 500~5000 nM; D: 5000 nM~10 mM

The present technology may include, but is not limited to, the features and combinations of features recited in the following numbered paragraphs, it being understood that the following paragraphs should not be interpreted as limiting the scope of the claims as appended hereto or mandating that all such features must necessarily be included in such claims.

1) A compound of formula (I),

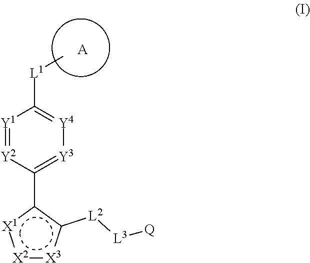

or a pharmaceutically acceptable salt, tautomer and/or stereoisomer thereof,
wherein:
A ring is a 5-membered heterocyclyl or 6-membered cyclohexyl or heterocyclyl selected from:

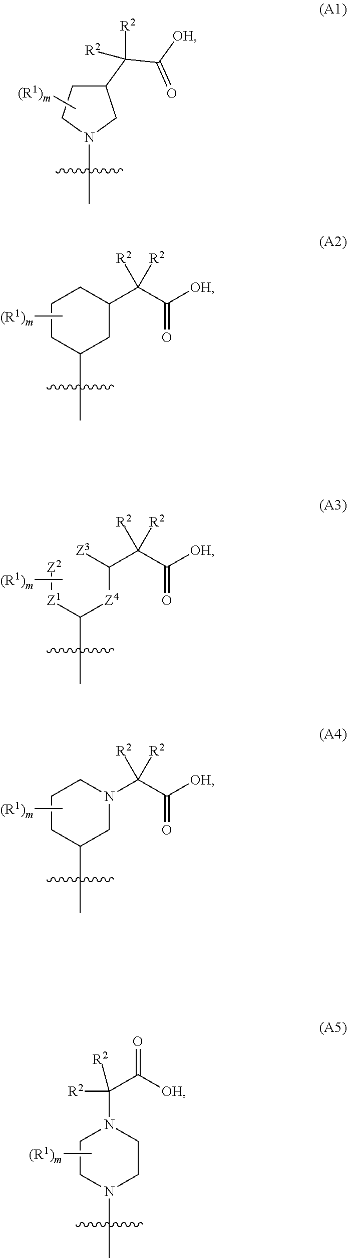

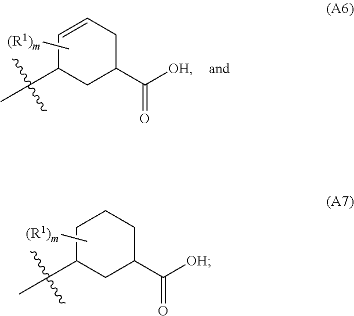

L¹ is a covalent bond, NH, O or S; provided that when L¹ is a covalent bond, A ring is selected from Formulae (A1), (A2), (A3), (A4) or (A5); further provided that when L¹ is NH or S, A ring is selected from Formulae (A6) or (A7); and further provided that when L¹ is O, A ring is selected from Formula (A6);
L² is a covalent bond or (CR⁷R⁷)ₚ;
L³ is a covalent bond, O or NR⁷, provided that at least one of L² and L³ is not a covalent bond;
Q is C(═O)NR⁹R¹⁰, C(═O)OR¹⁰, or a ring selected from a 5- or 6-membered heteroaryl group or a 5- or 6-membered heterocyclyl group, wherein the ring members comprises at least one carbon atom, at least one nitrogen atom, and optionally 1-4 additional heteroatoms selected from the group consisting of nitrogen, oxygen and sulfur, wherein oxygen may be a ring member and/or an oxo group attached to a ring member, and wherein the ring is substituted with (R³)ₙ and one R⁴;
X¹ is N, O or CR⁶a;
X² is N or NR⁶;
X³ is N, NR⁶ or CR⁶, wherein the dashed circle denotes bonds forming a five-membered aromatic ring;
Y¹, Y², Y³ and Y⁴ are each independently N or CR⁵, provided that at least one but no more than two of Y¹, Y², Y³ and Y⁴ are N;
Z¹, Z², Z³ and Z⁴ are each independently CH₂ or O, provided that only one of Z¹, Z², Z³ and Z⁴ is O;
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, (CH₂)_{q}-5-6-membered heteroaryl ring substituted with 1-4 R¹¹, or (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy; R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
each of R^{6a} and R⁶ is independently hydrogen, halogen, CN, C₁₋₄ alkyl, or cyclopropyl;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
each occurrence of R⁹ and R¹⁰ is independently hydrogen, C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, or O(CH₂)ₚ—C₃₋₇cycloalkyl, wherein each phenyl is independently optionally substituted with 1-3 halogen, C₁₋₆alkyl, or C₁₋₆alkoxy;
R¹² at each occurrence is independently hydrogen, C₁₋₄alkyl, or C₃₋₇cycloalkyl, or R¹² and R¹²,
together with the carbon atom to which they are attached, form a 3-6-membered cycloalkyl ring;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups, which may be the same or different, selected from the group consisting of C₁₋₄alkyl, phenyl and benzyl;
m is 0, 1, 2 or 3;
n is 0, 1, or 2;
p at each occurrence is independently 1, 2, 3 or 4;
q at each occurrence is independently 0, 1, 2, 3 or 4.

2) The compound of Paragraph 1, wherein A ring is selected from Formula (A1).

3) The compound of Paragraph 1, wherein A ring is selected from Formula (A2).

4) The compound of Paragraph 1, wherein A ring is selected from Formula (A3).

5) The compound of Paragraph 1, wherein A ring is selected from Formula (A4).

6) The compound of Paragraph 1, wherein A ring is selected from Formula (A5).

7) The compound of Paragraph 1, wherein A ring is selected from Formula (A6).

8) The compound of Paragraph 1, wherein A ring is selected from Formula (A7).

9) The compound of any one of Paragraphs 1-8, wherein L¹ is a covalent bond.

10) The compound of any one of Paragraphs 1-8, wherein L¹ is NH.

11) The compound of any one of Paragraphs 1-8, wherein L¹ is O.

12) The compound of any one of Paragraphs 1-8, wherein L¹ is S.

13) The compound of any one of Paragraphs 1-12, wherein R¹ at each occurrence is independently hydrogen.

14) The compound of any one of Paragraphs 1-12, wherein R¹ at each occurrence is independently halogen.

15) The compound of any one of Paragraphs 1-12, wherein m is 2, one R¹ is hydrogen, and the other R¹ is halogen.

16) The compound of any one of Paragraphs 1-12, wherein m is 2, one R¹ is hydrogen, and the other R¹ is F.

17) The compound of any one of Paragraphs 1-12, wherein m is 2, R¹ at each occurrence is independently F.

18) The compound of any one of Paragraphs 1-17, wherein R² at each occurrence is independently hydrogen.

19) The compound of any one of Paragraphs 1-17, wherein R² at each occurrence is independently C₁₋₄alkyl.

20) The compound of any one of Paragraphs 1-17, wherein R² at each occurrence is independently C₃₋₅cycloalkyl.

21) The compound of any one of Paragraphs 1-17, wherein R² and R², together with the carbon atom to which they are attached, form a 3-5-membered cycloalkyl ring.

22) The compound of any one of Paragraphs 1-17, wherein one R² is hydrogen, and the other R² is methyl.

23) The compound of any one of Paragraphs 1-17, wherein one R² is hydrogen, and the other R² is ethyl or propyl.

24) The compound of any one of Paragraphs 1-17, wherein one R² is hydrogen, and the other R² is cyclopropyl.

25) The compound of any one of Paragraphs 1-17, wherein R² and R², together with the carbon atom to which they are attached, form a cyclopropyl ring.

26) The compound of any one of Paragraphs 1-25, wherein Y² is N, and each of Y¹, Y³ and Y⁴ is independently CR⁵.

27) The compound of any one of Paragraphs 1-25, wherein Y¹ is CR⁵, Y² is N, and each of Y³ and Y⁴ is independently CH.

28) The compound of any one of Paragraphs 1-25, wherein Y¹ is CR⁵, Y² is N, Y³ is N, and Y⁴ CH.

29) The compound of any one of Paragraphs 1-28, wherein R⁵ is methyl or ethyl.

30) The compound of any one of Paragraphs 1-28, wherein R⁵ is CHF₂ or CF₃.

31) The compound of any one of Paragraphs 1-28, wherein R⁵ is hydrogen, or CN.

32) The compound of any one of Paragraphs 1-31, wherein X¹ is N, X² is N, and X³ is NR⁶.

33) The compound of any one of Paragraphs 1-31, wherein X¹ is CH, X² is N, and X³ is NR⁶.

34) The compound of any one of Paragraphs 1-31, wherein X¹ is O, X² is N, and X³ is CR⁶.

35) The compound of any one of Paragraphs 1-34, wherein R⁶ is methyl.

36) The compound of any one of Paragraphs 1-35, wherein L² is a covalent bond.

37) The compound of any one of Paragraphs 1-35, wherein L² is (CR⁷R⁷)ₚ.

38) The compound of any one of Paragraphs 1-35, wherein L² is CH₂.

39) The compound of any one of Paragraphs 1-38, wherein L³ is a covalent bond.

40) The compound of any one of Paragraphs 1-38, wherein L³ is O.

41) The compound of any one of Paragraphs 1-38, wherein L³ is NR₇.

42) The compound of any one of Paragraphs 1-41, wherein q is 0.

43) The compound of any one of Paragraphs 1-41, wherein q is 1.

44) The compound of any one of Paragraphs 1-41, wherein q is 2.

45) The compound of any one of Paragraphs 1-44, wherein R⁹ is C₁₋₄alkyl.

46) The compound of any one of Paragraphs 1-45, wherein R¹⁰ is C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹.

47) The compound of any one of Paragraphs 1-45, wherein R¹⁰ is C₁₋₆alkyl.

48) The compound of any one of Paragraphs 1-45, wherein R¹⁰ is (CH₂)ₚ—C₃₋₇cycloalkyl.

49) The compound of any one of Paragraphs 1-45, wherein R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur.

50) The compound of any one of Paragraphs 1-49, wherein Q is C(═O)NR⁹R¹⁰.

51) The compound of any one of Paragraphs 1-49, wherein Q is a ring selected from a 5- or 6-membered heteroaryl group or a 5- or 6-membered heterocyclyl group, wherein the ring members comprises at least one carbon atom, at least one nitrogen atom, and optionally 1-4 additional heteroatoms selected from nitrogen, oxygen and sulfur, wherein oxygen may be a ring member and/or an oxo group attached to a ring member, and wherein the ring is substituted with (R³)ₙ and one R⁴.

52) The compound of Paragraph 51, wherein the Q ring is:

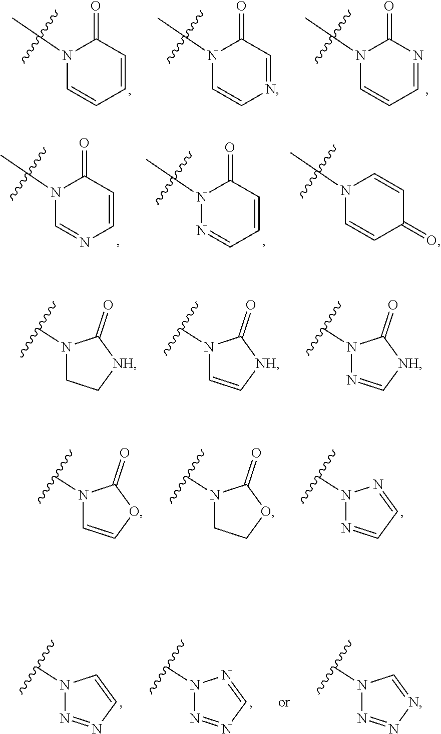

each of which is substituted with (R³)ₙ and one R⁴ at any available carbon or nitrogen position.

53) The compound of Paragraph 51, wherein the Q ring is:

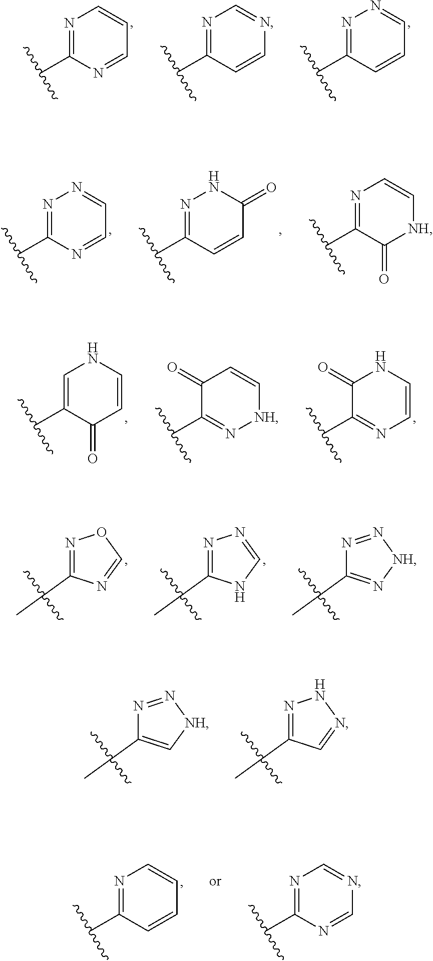

each of which is substituted with (R³)ₙ and one R⁴ at any available carbon or nitrogen position.

54) The compound of any one of Paragraphs 1-53, wherein R³ at each occurrence is independently hydrogen, halogen, or C₁₋₄alkyl.

55) The compound of any one of Paragraphs 1-53, wherein R³ at each occurrence is independently C₁₋₄alkyl.

56) The compound of any one of Paragraphs 1-53, wherein R³ at each occurrence is methyl.

57) The compound of any one of Paragraphs 1-56, wherein R⁴ is hydrogen, C₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy.

58) The compound of any one of Paragraphs 1-56, wherein R⁴ is (CH₂)_{q}-5-6-membered heteroaryl ring substituted with 1-4 R¹¹, or (CH₂)_{q}-5-7-membered heterocyclyl ring substituted with 1-4 R¹¹.

59) The compound of any one of Paragraphs 1-56, wherein R⁴ is C₁₋₆alkyl, C₂₋₆alkynyl, C₁₋₆alkoxy, (CH₂)ₚ—C₁₋₆alkoxy, C₃₋₇cycloalkyl, (CH₂)ₚ—C₃₋₇cycloalkyl, or C₂₋₆alkenyl-C₃₋₇cycloalkyl.

60) The compound of Paragraph 1, having structure of Formula (II),

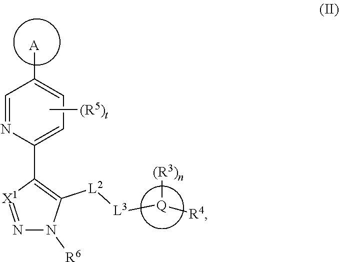

or a pharmaceutically acceptable salt, tautomer and/or stereoisomer thereof,
wherein:
A ring is a 5-membered heterocyclyl or 6-membered cyclohexyl or heterocyclyl selected from:

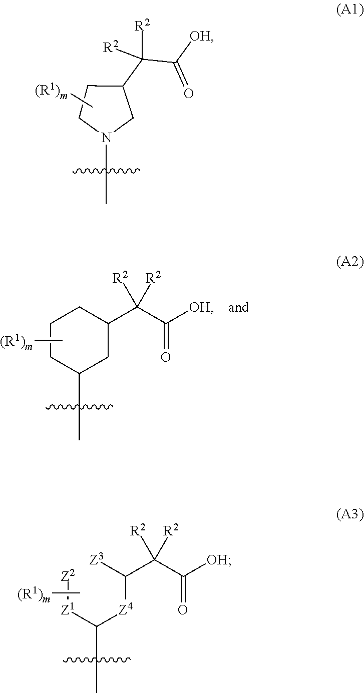

X¹ is N, or CR⁶a;
R^{6a} is hydrogen, or methyl;
R⁶ is hydrogen, halogen, CN, methyl, ethyl, propyl, or cyclopropyl;
the Q ring is selected from 5-membered heteroaryl or heterocyclyl and 6-membered heteroaryl or heterocyclyl, wherein the Q ring contains one nitrogen atom and optionally contains 1-4 additional heteroatoms selected from nitrogen, oxygen and sulfur, and wherein the Q ring is substituted with (R³)ₙ and one R⁴; and
t is 0, 1, 2 or 3.

61) The compound of Paragraph 60, having the structure of Formula (IIa)

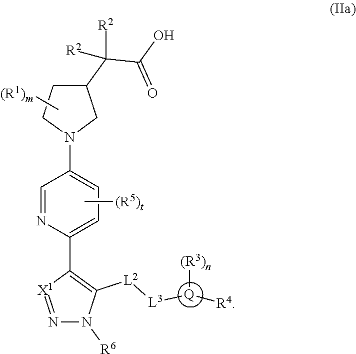

62) The compound of Paragraph 60, having the structure of Formula (IIb),

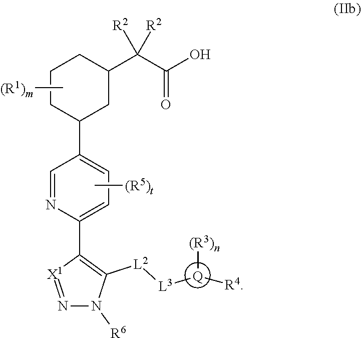

63) The compound of Paragraph 60, having the structure of Formula (IIc),

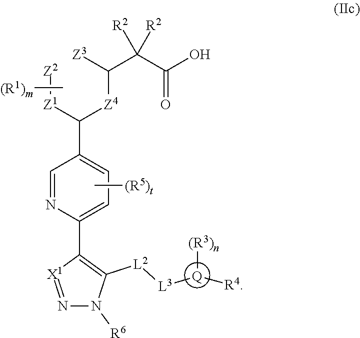

64) The compound of any one of Paragraphs 60-63, wherein R¹ at each occurrence is independently hydrogen.

65) The compound of any one of Paragraphs 60-63, wherein R¹ at each occurrence is independently halogen.

66) The compound of any one of Paragraphs 60-63, wherein m is 2, one R¹ is hydrogen, and the other R¹ is halogen.

67) The compound of any one of Paragraphs 60-63, wherein m is 2, one R¹ is hydrogen, and the other R¹ is F.

68) The compound of any one of Paragraphs 60-63, wherein m is 2, and R¹ at each occurrence is independently F.

69) The compound of any one of Paragraphs 60-68, wherein R² at each occurrence is independently hydrogen.

70) The compound of any one of Paragraphs 60-68, wherein R² at each occurrence is independently C₁₋₄alkyl.

71) The compound of any one of Paragraphs 60-68, wherein R² at each occurrence is independently C₃₋₅cycloalkyl.

72) The compound of any one of Paragraphs 60-68, wherein R² and R², together with the carbon atom to which they are attached, form a 3-5-membered cycloalkyl ring.

73) The compound of any one of Paragraphs 60-68, wherein one R² is hydrogen, and the other R² is methyl.

74) The compound of any one of Paragraphs 60-68, wherein one R² is hydrogen, and the other R² is ethyl or propyl.

75) The compound of any one of Paragraphs 60-68, wherein one R² is hydrogen, and the other R² is cyclopropyl.

76) The compound of any one of Paragraphs 60-68, wherein R² and R², together with the carbon atom to which they are attached, form a cyclopropyl ring.

77) The compound of any one of Paragraphs 60-76, wherein the moiety

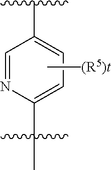

is

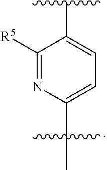

78) The compound of any one of Paragraphs 60-77, wherein R⁵ at each occurrence is independently hydrogen, halogen, C₁₋₆alkyl or haloC₁₋₆alkyl.

79) The compound of any one of Paragraphs 60-77, wherein R⁵ at each occurrence is independently C₁₋₆alkyl.

80) The compound of any one of Paragraphs 60-77, wherein R⁵ at each occurrence is independently methyl or ethyl.

81) The compound of any one of Paragraphs 60-77, wherein R⁵ at each occurrence is independently CHF₂ or CF₃.

82) The compound of any one of Paragraphs 60-80, wherein X¹ is N.

83) The compound of any one of Paragraphs 60-80, wherein X¹ is CH.

84) The compound of any one of Paragraphs 60-83, wherein R⁶ is methyl.

85) The compound of any one of Paragraphs 60-84, wherein L² is a (CR⁷R⁷)ₚ.

86) The compound of any one of Paragraphs 60-84, wherein L² is a CH₂.

87) The compound of any one of Paragraphs 60-86, wherein L³ is a covalent bond.

88) The compound of any one of Paragraphs 60-86, wherein L³ is O.

89) The compound of any one of Paragraphs 60-86, wherein L³ is NR⁷.

90) The compound of any one of Paragraphs 60-89, wherein the Q ring is:

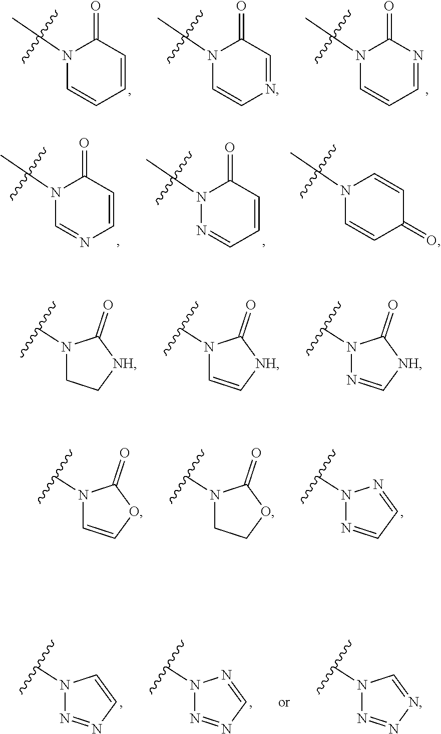

each of which is substituted with (R³)ₙ and one R⁴ at any available carbon or nitrogen position, and n is 0, 1 or 2.

91) The compound of Paragraph 90, wherein the Q ring is

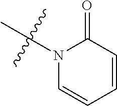 [SMILES: *n1ccccc1=O]

substituted with (R³)ₙ and one R⁴ at any available carbon position, and n is 0, 1 or 2.

92) The compound of any one of Paragraphs 60-89, wherein the Q ring is:

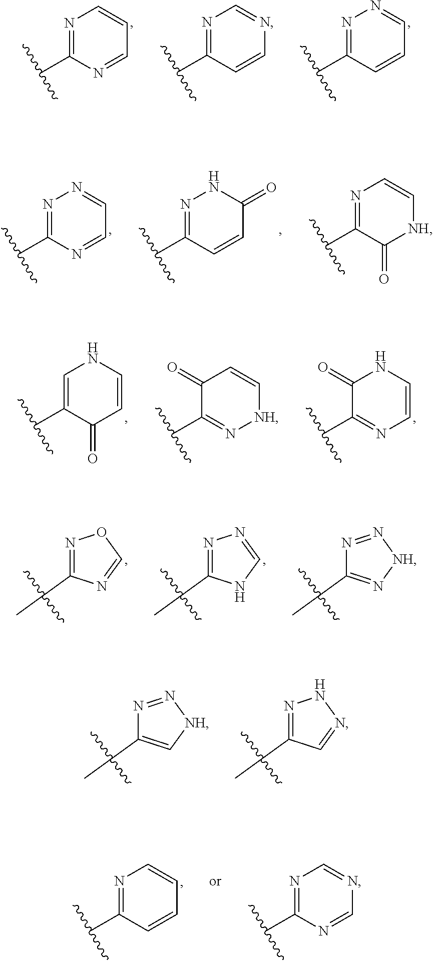

each of which is substituted with (R³)ₙ and one R⁴ at any available carbon or nitrogen position, and n is 0, 1 or 2.

93) The compound of any one of Paragraphs 60-92, wherein R³ at each occurrence is independently hydrogen, halogen, or C₁₋₄alkyl.

94) The compound of any one of Paragraphs 60-92, wherein R³ at each occurrence is independently C₁₋₄alkyl.

95) The compound of any one of Paragraphs 60-92, wherein R³ at each occurrence is independently methyl.

96) The compound of any one of Paragraphs 60-95, wherein R⁴ is hydrogen, C₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy.

97) The compound of any one of Paragraphs 60-95, wherein R⁴ is (CH₂)_{q}-5-6-membered heteroaryl ring substituted with 1-4 R¹¹, or (CH₂)_{q}-5-7-membered heterocyclyl ring substituted with 1-4 R¹¹.

98) The compound of any one of Paragraphs 60-95, wherein R⁴ is C₁₋₆alkyl, C₂₋₆alkynyl, C₁₋₆alkoxy, (CH₂)ₚ—C₁₋₆alkoxy, C₃₋₇cycloalkyl, (CH₂)ₚ—C₃₋₇cycloalkyl, or C₂₋₆ alkynyl-C₃₋₇cycloalkyl.

99) The compound of Paragraph 1, having the structure of Formula (III),

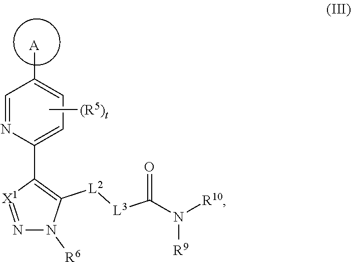

or a pharmaceutically acceptable salt, tautomer and/or stereoisomer thereof,
wherein:
A ring is a 5-membered heterocyclyl or 6-membered cyclohexyl or heterocyclyl selected from:

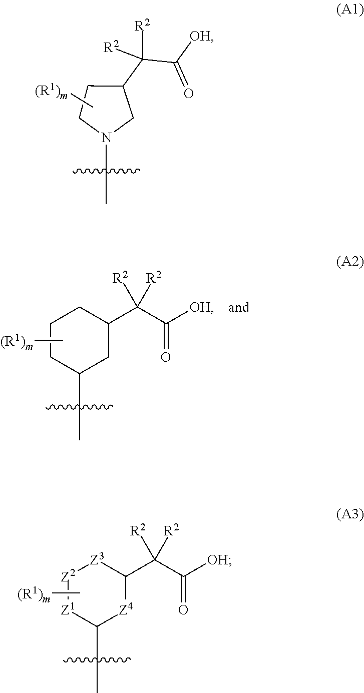

R¹ at each occurrence is independently hydrogen, halogen, keto (═O), C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl;
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
each one of Z¹, Z², Z³ and Z⁴ is independently CH₂ or O, provided that only one of Z¹, Z², Z³ and Z⁴ is O;
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR^{6a};
R^{6a} is hydrogen, or methyl;
R⁶ is hydrogen, halogen, CN, methyl, ethyl, propyl, or cyclopropyl;
L² is a covalent bond or (CR⁷R⁷)ₚ;
L³ is a covalent bond, O or NR⁷, provided that at least one of L² and L³ is not a covalent bond;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
each occurrence of R⁹ and R¹⁰ is independently hydrogen, C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, wherein each phenyl is independently optionally substituted with 1-3 halogen, C₁₋₆alkyl, or C₁₋₆alkoxy;
R¹² at each occurrence is independently hydrogen, C₁₋₄alkyl, C₃₋₇cycloalkyl, or R¹² and R¹², together with the carbon atom to which they are attached, form a 3-6-membered cycloalkyl ring;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
t is 0, 1, 2 or 3;
p at each occurrence is independently 1, 2, 3 or 4;
and q at each occurrence is independently 0, 1, 2, 3 or 4.

100) The compound of Paragraph 99, having the structure of Formula (IIIa),

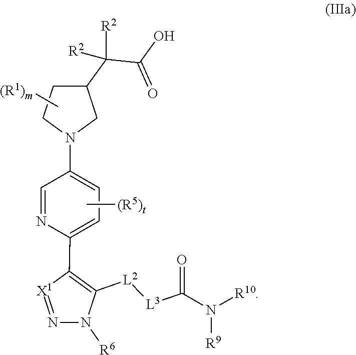

101) The compound of Paragraph 99, having the structure of Formula (IIIb).

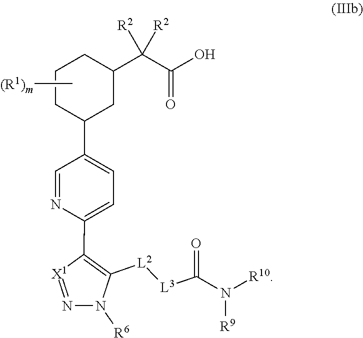

102) The compound of Paragraph 99, having the structure of Formula (IIIc),

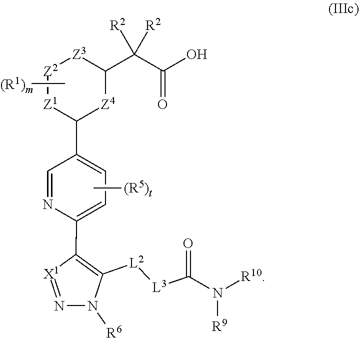

103) The compound of any one of Paragraphs 99-102, wherein R¹ at each occurrence is independently hydrogen.

104) The compound of any one of Paragraphs 99-102, wherein R¹ at each occurrence is independently halogen.

105) The compound of any one of Paragraphs 99-102, wherein one R¹ is hydrogen, and the other R¹ is halogen.

106) The compound of any one of Paragraphs 99-102, wherein one R¹ is hydrogen, and the other R¹ is F.

107) The compound of any one of Paragraphs 99-102, wherein R¹ at each occurrence is independently F.

108) The compound of any one of Paragraphs 99-107, wherein the moiety

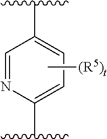

is

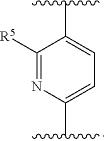

109) The compound of any one of Paragraphs 99-108, wherein R⁵ at each occurrence is independently hydrogen, halogen, or C₁₋₆alkyl.

110) The compound of any one of Paragraphs 99-108, wherein R⁵ at each occurrence is independently C₁₋₆alkyl.

111) The compound of any one of Paragraphs 99-108, wherein R⁵ at each occurrence is independently methyl or ethyl.

112) The compound of any one of Paragraphs 99-111, wherein X¹ is N.

113) The compound of any one of Paragraphs 99-111, wherein X¹ is CH.

114) The compound of any one of Paragraphs 99-113, wherein R⁶ is methyl.

115) The compound of any one of Paragraphs 99-114, wherein L² is a (CR⁷R⁷)ₚ.

116) The compound of any one of Paragraphs 99-114, wherein L² is a CH₂.

117) The compound of any one of Paragraphs 99-116, wherein L³ is a covalent bond.

118) The compound of any one of Paragraphs 99-116, wherein L³ is O.

119) The compound of any one of Paragraphs 99-116, wherein L³ is NR⁷.

120) The compound of any one of Paragraphs 99-119, wherein R⁹ is C₁₋₄alkyl.

121) The compound of any one of Paragraphs 99-120, wherein R¹⁰ is C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹.

122) The compound of any one of Paragraphs 99-120, wherein R¹⁰ is C₁₋₆alkyl.

123) The compound of any one of Paragraphs 99-120, wherein R¹⁰ is (CH₂)_{q}—C₃₋₇cycloalkyl.

124) The compound of any one of Paragraphs 99-120, wherein R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur.

125) The compound of Paragraph 1, selected from:

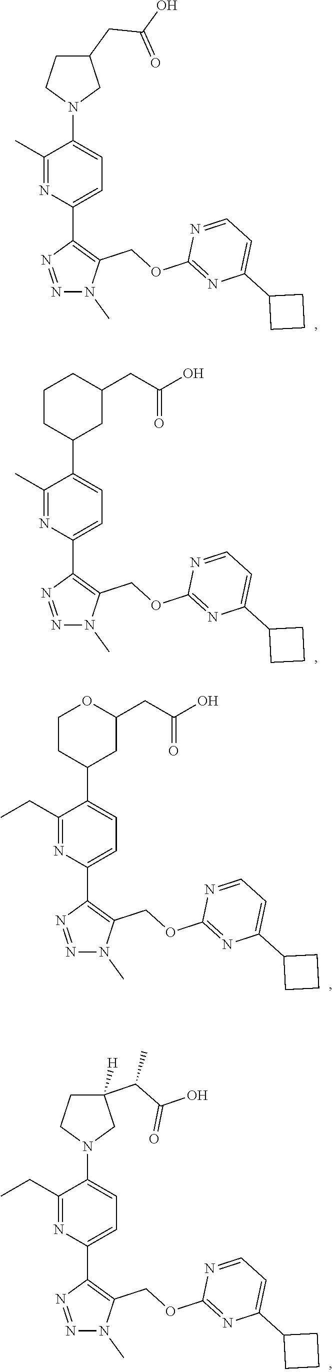 [SMILES: Cc1nc(-c2nnn(C)c2COc2nccc(C3CCC3)n2)ccc1N1CCC(CC(=O)O)C1 ; Cc1nc(-c2nnn(C)c2COc2nccc(C3CCC3)n2)ccc1C1CCCC(CC(=O)O)C1 ; CCc1nc(-c2nnn(C)c2COc2nccc(C3CCC3)n2)ccc1C1CCOC(CC(=O)O)C1 ; CCc1nc(-c2nnn(C)c2COc2nccc(C3CCC3)n2)ccc1N1CC[C@@H]([C@H](C)C(=O)O)C1]

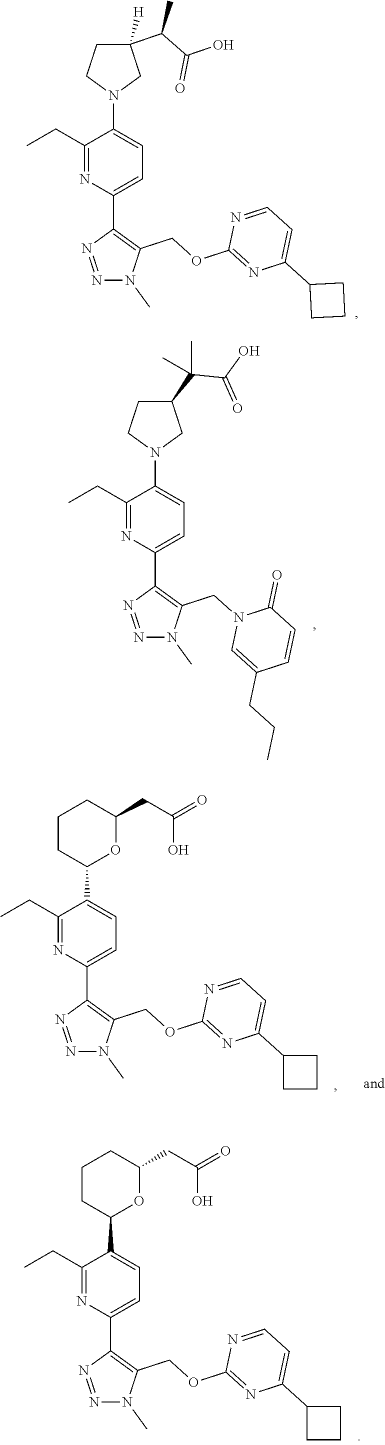 [SMILES: CCc1nc(-c2nnn(C)c2COc2nccc(C3CCC3)n2)ccc1N1CC[C@@H]([C@@H](C)C(=O)O)C1 ; CCCc1ccc(=O)n(Cc2c(-c3ccc(N4CC[C@@H](C(C)(C)C(=O)O)C4)c(CC)n3)nnn2C)c1 ; CCc1nc(-c2nnn(C)c2COc2nccc(C3CCC3)n2)ccc1[C@@H]1CCC[C@@H](CC(=O)O)O1 ; CCc1nc(-c2nnn(C)c2COc2nccc(C3CCC3)n2)ccc1[C@H]1CCC[C@H](CC(=O)O)O1]

126) A compound of Formula (IV),

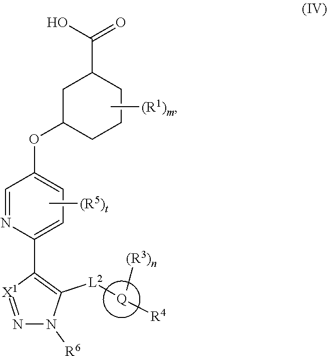

or a pharmaceutically acceptable salt, tautomer and/or stereoisomer thereof,
wherein:
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR⁶a;
R^{6a} is hydrogen, or methyl;
R⁶ is hydrogen, halogen, CN, methyl, ethyl, propyl, or cyclopropyl;
L² is (CR⁷R⁷)ₚ;
Q ring is

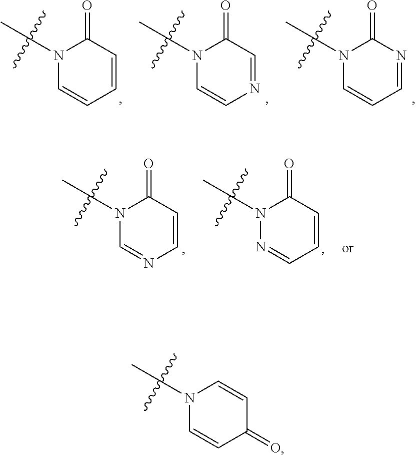 [SMILES: *n1ccccc1=O ; *n1ccncc1=O ; *n1cccnc1=O ; *n1cnccc1=O ; *n1ncccc1=O ; *n1ccc(=O)cc1]

each of which is substituted with (R³)ₙ and one R⁴ at any available carbon position;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, (CH₂)_{q}-5-6-membered heteroaryl ring substituted with 1-4 R¹¹, (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, wherein each phenyl is independently optionally substituted with 1-3 halogen, C₁₋₆alkyl, or C₁₋₆alkoxy;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
n is 0, 1 or 2;
t is 0, 1, 2 or 3;
p at each occurrence is independently 1, 2, 3 or 4;
and q at each occurrence is independently 0, 1, 2, 3 or 4.

127) The compound of Paragraph 126, having the structure of Formula (IVa),

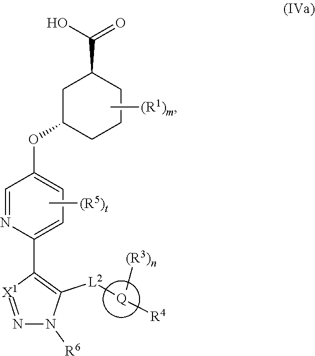

128) The compound of Paragraph 126 or 127, wherein m is 0.

129) The compound of any one of Paragraphs 126-128, wherein the moiety

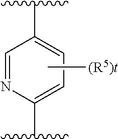

is

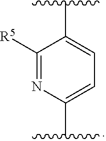

130) The compound of any one of Paragraphs 126-129, wherein R⁵ at each occurrence is independently hydrogen, halogen, C₁₋₆alkyl or haloC₁₋₆alkyl.

131) The compound of any one of Paragraphs 126-129, wherein R⁵ at each occurrence is independently C₁₋₆alkyl.

132) The compound of any one of Paragraphs 126-129, wherein R⁵ at each occurrence is independently methyl or ethyl.

133) The compound of any one of Paragraphs 126-129, wherein R⁵ at each occurrence is independently CHF₂ or CF₃.

134) The compound of any one of Paragraphs 126-133, wherein X¹ is N.

135) The compound of any one of Paragraphs 126-133, wherein X¹ is CH.

136) The compound of any one of Paragraphs 126-135, wherein R⁶ is methyl.

137) The compound of any one of Paragraphs 126-136, wherein L² is a CH₂.

138) The compound of any one of Paragraphs 126-137, wherein the Q ring is

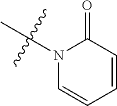 [SMILES: *n1ccccc1=O]

substituted with (R³)ₙ and one R⁴ at any available carbon position.

139) The compound of any one of Paragraphs 126-138, wherein R³ at each occurrence is independently hydrogen, halogen, or C₁₋₄alkyl.

140) The compound of any one of Paragraphs 126-138, wherein R³ at each occurrence is independently C₁₋₄alkyl.

141) The compound of any one of Paragraphs 126-138, wherein R³ at each occurrence is independently methyl.

142) The compound of any one of Paragraphs 126-141, wherein R⁴ is hydrogen, C₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy.

143) The compound of any one of Paragraphs 126-141, wherein R⁴ is (CH₂)_{q}-5-6-membered heteroaryl ring substituted with 1-4 R¹¹, or (CH₂)_{q}-5-7-membered heterocyclyl ring substituted with 1-4 R¹¹.

144) The compound of any one of Paragraphs 126-141, wherein R⁴ is C₁₋₆alkyl, C₂₋₆alkynyl, C₁₋₆alkoxy, (CH₂)ₚ—C₁₋₆alkoxy, C₃₋₇cycloalkyl, (CH₂)ₚ—C₃₋₇cycloalkyl, or C₂₋₆ alkynyl-C₃₋₇cycloalkyl.

145) The compound of Paragraph 126, selected from:

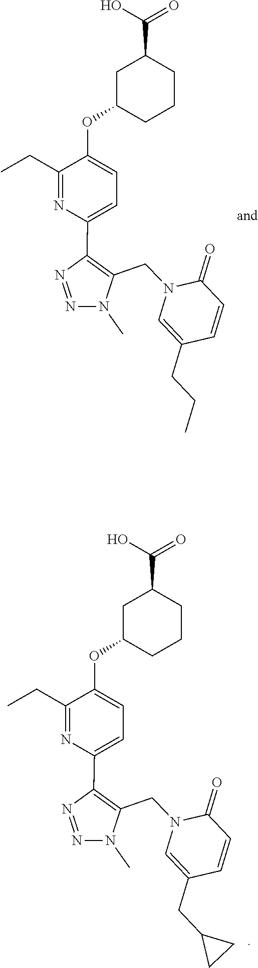 [SMILES: CCCc1ccc(=O)n(Cc2c(-c3ccc(O[C@H]4CCC[C@H](C(=O)O)C4)c(CC)n3)nnn2C)c1 ; CCc1nc(-c2nnn(C)c2Cn2cc(CC3CC3)ccc2=O)ccc1O[C@H]1CCC[C@H](C(=O)O)C1]

146) A pharmaceutical composition comprising the compound of any one of Paragraphs 1-145, and a pharmaceutically acceptable carrier.

147) A method for treating a disease associated with dysregulation of lysophosphatidic acid receptor 1 (LPAi) in a subject in need thereof, comprising administering an effective amount of a compound of any one of Paragraphs 1-145 to the subject.

148) The method of Paragraph 147, wherein the disease is pathological fibrosis (e.g., pulmonary, liver, renal, cardiac, dernal, ocular, or pancreatic fibrosis), idiopathic pulmonary fibrosis (IPF), non-alcoholic steatohepatitis (NASH), non-alcoholic fatty liver disease (NAFLD), chronic kidney disease, diabetic kidney disease, or systemic sclerosis.

1. A compound of formula (I), 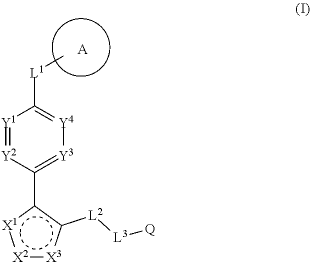
or a pharmaceutically acceptable salt, tautomer and/or stereoisomer thereof,
wherein:
A ring is a 5-membered heterocyclyl or 6-membered cyclohexyl or heterocyclyl selected from: 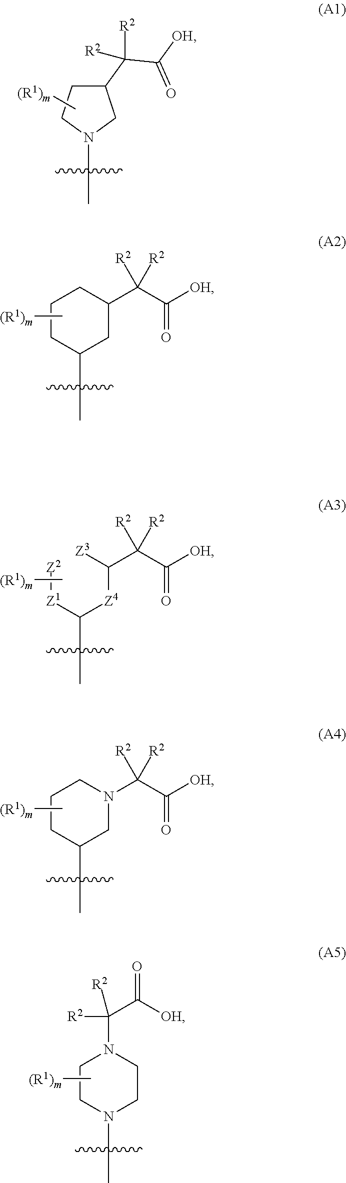 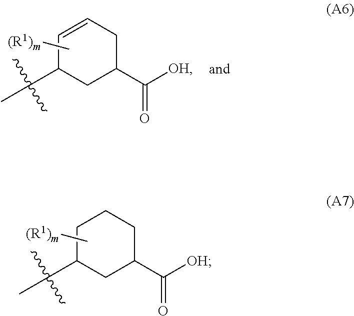
L¹ is a covalent bond, NH, O or S; provided that when L¹ is a covalent bond, A ring is selected from Formulae (A1), (A2), (A3), (A4) or (A5); further provided that when L¹ is NH or S, A ring is selected from Formulae (A6) or (A7); and further provided that when L¹ is O, A ring is selected from Formula (A6);
L² is a covalent bond or (CR⁷R⁷)ₚ;
L³ is a covalent bond, O or NR⁷, provided that at least one of L² and L³ is not a covalent bond;
Q is C(═O)NR⁹R¹⁰, C(═O)OR¹⁰, or a ring selected from a 5- or 6-membered heteroaryl group or a 5- or 6-membered heterocyclyl group, wherein the ring members comprises at least one carbon atom, at least one nitrogen atom, and optionally 1-4 additional heteroatoms selected from the group consisting of nitrogen, oxygen and sulfur, wherein oxygen may be a ring member and/or an oxo group attached to a ring member, and wherein the ring is substituted with (R³)ₙ and one R⁴;
X¹ is N, O or CR⁶a;
X² is N or NR⁶;
X³ is N, NR⁶ or CR⁶, wherein the dashed circle denotes bonds forming a five-membered aromatic ring;
Y¹, Y², Y³ and Y⁴ are each independently N or CR⁵, provided that at least one but no more than two of Y¹, Y², Y³ and Y⁴ are N;
Z¹, Z², Z³ and Z⁴ are each independently CH₂ or O, provided that only one of Z¹, Z², Z³ and Z⁴ is O;
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, (CH₂)_{q}-5-6-membered heteroaryl ring substituted with 1-4 R¹¹, or (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
each of R^{6a} and R⁶ is independently hydrogen, halogen, CN, C₁₋₄ alkyl, or cyclopropyl;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
each occurrence of R⁹ and R¹⁰ is independently hydrogen, C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, or O(CH₂)ₚ—C₃₋₇cycloalkyl, wherein each phenyl is independently optionally substituted with 1-3 halogen, C₁₋₆alkyl, or C₁₋₆alkoxy;
R¹² at each occurrence is independently hydrogen, C₁₋₄alkyl, or C₃₋₇cycloalkyl, or R¹² and R¹², together with the carbon atom to which they are attached, form a 3-6-membered cycloalkyl ring;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups, which may be the same or different, selected from the group consisting of C₁₋₄alkyl, phenyl and benzyl;
m is 0, 1, 2 or 3;
n is 0, 1, or 2;
p at each occurrence is independently 1, 2, 3 or 4;
q at each occurrence is independently 0, 1, 2, 3 or 4.

2. The compound of claim 1, wherein A ring is selected from one of Formula (A1).

3-8. (canceled)

9. The compound of claim 1, wherein L¹ is selected from a covalent bond, NH, O and S.

10.-12. (canceled)

13. The compound of claim 1, wherein R¹ at each occurrence is independently hydrogen or halogen.

14. (canceled)

15. The compound of claim 1, wherein m is 2, and (a) one R¹ is hydrogen, and the other R¹ is halogen; (b) one R¹ is hydrogen, and the other R¹ is F; or (c) R¹ at each occurrence is independently F.

16.-17. (canceled)

18. The compound of claim 1, wherein R² at each occurrence is independently hydrogen, C₁₋₄alkyl or C₃₋₅cycloalkyl.

19.-20. (canceled)

21. The compound of claim 1, wherein R² and R², together with the carbon atom to which they are attached, form a 3-5-membered cycloalkyl ring.

22. The compound of claim 1, wherein (a) one R² is hydrogen, and the other R² is methyl; (b) one R² is hydrogen, and the other R² is ethyl or propyl; or (c) one R² is hydrogen, and the other R² is cyclopropyl.

23.-24. (canceled)

25. The compound of claim 1, wherein R² and R², together with the carbon atom to which they are attached, form a cyclopropyl ring.

26. The compound of claim 1, wherein (a) Y² is N, and each of Y¹, Y³ and Y⁴ is independently CR⁵; or (b) Y¹ is CR⁵, Y² is N, and each of Y³ and Y⁴ is independently CH; Y¹ is CR⁵, Y² is N, Y³ is N, and Y⁴ CH.

27.-28. (canceled)

29. The compound of claim 1, wherein (a) R⁵ is methyl or ethyl; or (b) R⁵ is CHF₂ or CF₃; or (c) R⁵ is hydrogen, or CN.

30.-31. (canceled)

32. The compound of claim 1, wherein (a) X¹ is N, X² is N, and X³ is NR⁶; or (b) X¹ is CH, X² is N, and X³ is NR⁶; or (c) X¹ is O, X² is N, and X³ is CR⁶.

33.-34. (canceled)

35. The compound of claim 1, wherein R⁶ is methyl.

36. The compound of claim 1, wherein L² is a covalent bond or CH₂.

37.-38. (canceled)

39. The compound of claim 1, wherein L³ is a covalent bond.

40.-41. (canceled)

42. The compound of claim 1, wherein q is 0, 1, or 2.

43.-44. (canceled)

45. The compound of claim 1, wherein R⁹ is C₁₋₄alkyl.

46. The compound of claim 1, wherein (a) R¹⁰ is C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or (b) R¹⁰ is C₁₋₆alkyl; or (c) (CH₂)ₚ—C₃₋₇cycloalkyl.

47.-48. (canceled)

49. The compound of claim 1, wherein R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur.

50. The compound of claim 1, wherein Q is
(a) C(═O)NR⁹R¹⁰;
(b) a ring selected from a 5- or 6-membered heteroaryl group or a 5- or 6-membered heterocyclyl group, wherein the ring members comprises at least one carbon atom, at least one nitrogen atom, and optionally 1-4 additional heteroatoms selected from nitrogen, oxygen and sulfur, wherein oxygen may be a ring member and/or an oxo group attached to a ring member, and wherein the ring is substituted with (R³)ₘ and one R⁴; 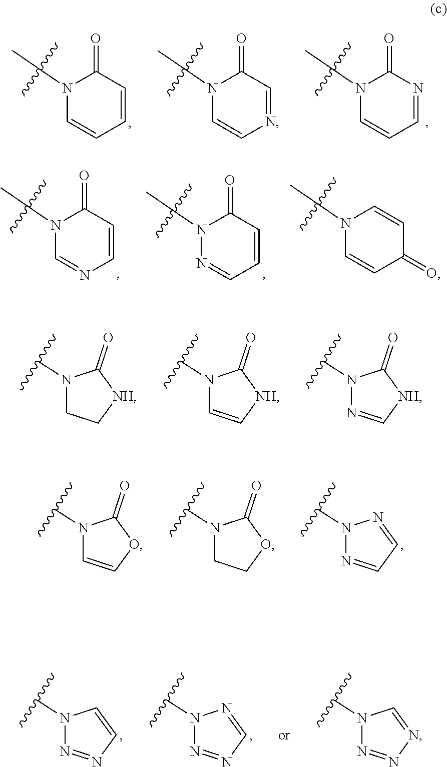
each of which is substituted with (R³)ₙ and one R⁴ at any available carbon or nitrogen position, 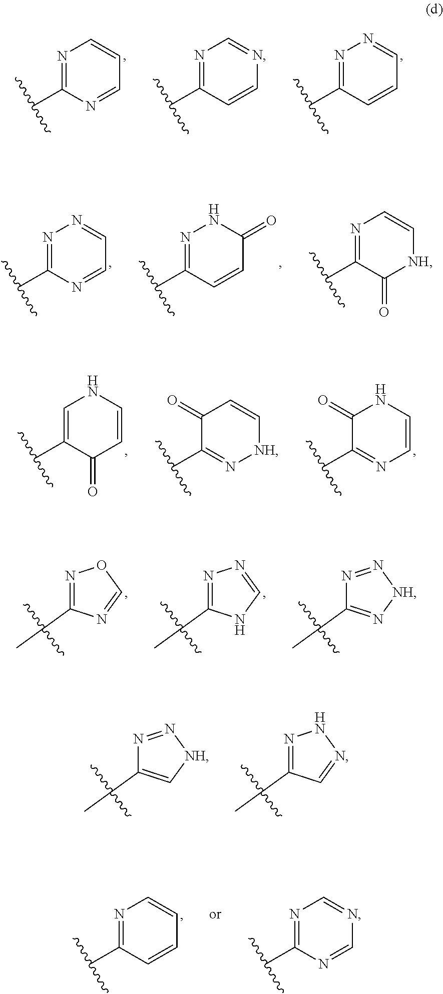
each of which is substituted with (R³)ₙ and one R⁴ at any available carbon or nitrogen position.

51.-53. (canceled)

54. The compound of claim 1, wherein R³ at each occurrence is independently hydrogen, halogen, or C₁₋₄alkyl.

55.-56. (canceled)

57. The compound of claim 1, wherein (a) R⁴ is hydrogen, C₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl-C₃₋₇cycloalkyl, O(CH₂)ₚ—C₃₋₇cycloalkyl, wherein each phenyl is independently optionally substituted with 1-3 halogen, C₁₋₆alkyl, or C₁₋₆alkoxy; or (b) R⁴ is (CH₂)_{q}-5-6-membered heteroaryl ring substituted with 1-4 R¹¹, or (CH₂)_{q}-5-7-membered heterocyclyl ring substituted with 1-4 R¹¹; or (c) R⁴ is C₁₋₆alkyl, C₂₋₆alkynyl, C₁₋₆alkoxy, (CH₂)ₚ—C₁₋₆alkoxy, C₃₋₇cycloalkyl, (CH₂)ₚ—C₃₋₇cycloalkyl, or C₂₋₆alkenyl-C₃₋₇cycloalkyl.

58.-59. (canceled)

60. The compound of claim 1, having structure of Formula (II), 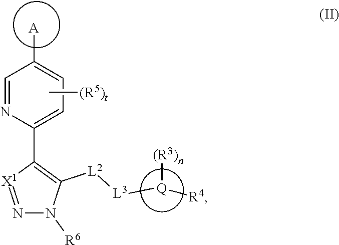
or a pharmaceutically acceptable salt, tautomer and/or stereoisomer thereof,
wherein:
A ring is a 5-membered heterocyclyl or 6-membered cyclohexyl or heterocyclyl selected from: 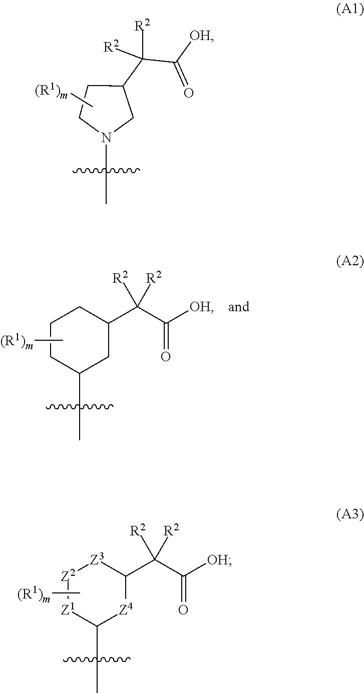
X¹ is N, or CR⁶a;
R^{6a} is hydrogen, or methyl;
R⁶ is hydrogen, halogen, CN, methyl, ethyl, propyl, or cyclopropyl;
the Q ring is selected from 5-membered heteroaryl or heterocyclyl and 6-membered heteroaryl or heterocyclyl, wherein the Q ring contains one nitrogen atom and optionally contains 1-4 additional heteroatoms selected from nitrogen, oxygen and sulfur, and wherein the Q ring is substituted with (R³)ₙ and one R⁴; and
t is 0, 1, 2 or 3.

61. The compound of claim 60, having the structure of Formula (IIa) 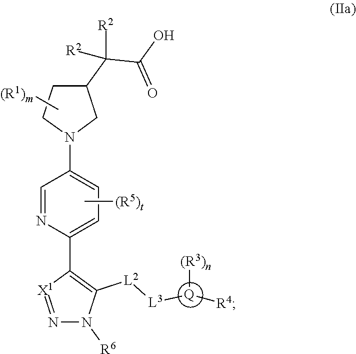
or having the structure of Formula (IIb), 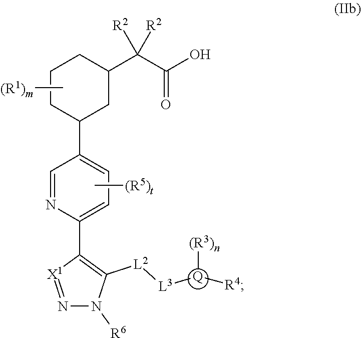
or having the structure of Formula (IIc),

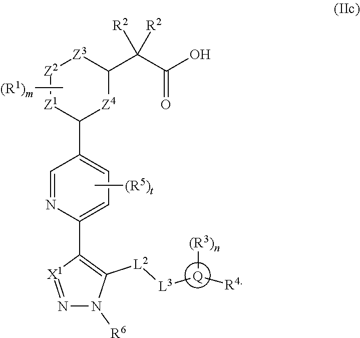

62.-63. (canceled)

64. The compound of claim 1, having the structure of Formula (III), 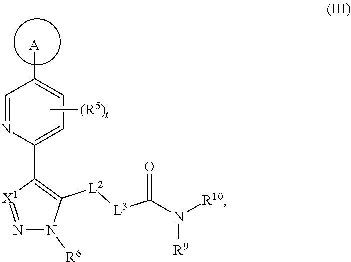
or a pharmaceutically acceptable salt, tautomer and/or stereoisomer thereof,
wherein:
A ring is a 5-membered heterocyclyl or 6-membered cyclohexyl or heterocyclyl selected from: 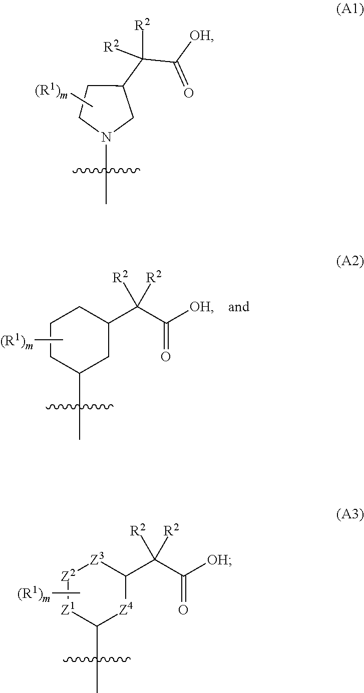
R¹ at each occurrence is independently hydrogen, halogen, keto (═O), C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl;
R² at each occurrence is independently hydrogen, deuterium, C₁₋₄alkyl, C₃₋₅cycloalkyl, or R² and R², together with the carbon atom to which they are attached, form a 3-5-membered cycloalkyl ring;
each one of Z¹, Z², Z³ and Z⁴ is independently CH₂ or O, provided that only one of Z¹, Z², Z³ and Z⁴ is O;
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR⁶a;
R^{6a} is hydrogen, or methyl;
R⁶ is hydrogen, halogen, CN, methyl, ethyl, propyl, or cyclopropyl;
L² is a covalent bond or (CR⁷R⁷)ₚ;
L³ is a covalent bond, O or NR⁷, provided that at least one of L² and L³ is not a covalent bond;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
each occurrence of R⁹ and R¹⁰ is independently hydrogen, C₁₋₆alkyl substituted with 1-4 R¹¹, (CR¹²R¹²)_{q}—C₂₋₆alkenyl substituted with 1-4 R¹¹, (CR¹²R¹²)_{q}—C₂₋₆alkynyl substituted with 1-4 R¹¹, (CR¹²R¹²)_{q}—C₃₋₇cycloalkyl substituted with 1-4 R¹¹, (CR¹²R¹²)_{q}-phenyl substituted with 1-4 R¹¹, (CR¹²R¹²)_{q}-5-6-membered heteroaryl ring substituted with 1-4 R¹¹, (CR¹²R¹²)_{q}-5-7-membered heterocyclyl ring substituted with 1-4 R¹¹; or R⁹ and R¹⁰, together with the nitrogen atom to which they are attached, form a saturated or unsaturated 3-7-membered heterocyclic ring substituted with 1-4 R¹¹, which ring may optionally contain one or two additional heteroatoms selected from the group consisting of nitrogen, oxygen and sulfur;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, wherein each phenyl is independently optionally substituted with 1-3 halogen, C₁₋₆alkyl, or C₁₋₆alkoxy;
R¹² at each occurrence is independently hydrogen, C₁₋₄alkyl, C₃₋₇cycloalkyl, or R¹² and R¹², together with the carbon atom to which they are attached, form a 3-6-membered cycloalkyl ring;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
t is 0, 1, 2 or 3;
p at each occurrence is independently 1, 2, 3 or 4;
and q at each occurrence is independently 0, 1, 2, 3 or 4.

65. The compound of claim 64, having the structure of Formula (IIIa), 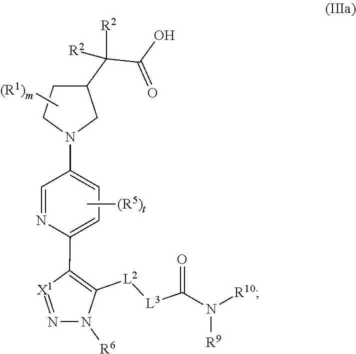
or having the structure of Formula (IIIb), 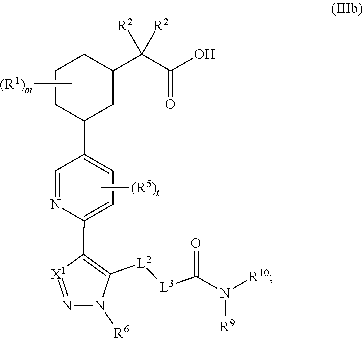
or having the structure of Formula (IIIc),

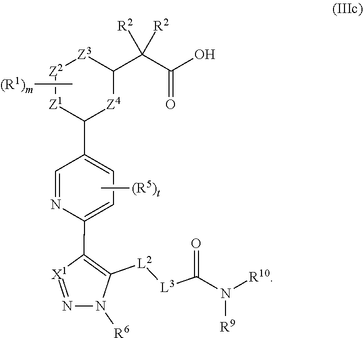

66.-67. (canceled)

68. The compound of claim 1, selected from:

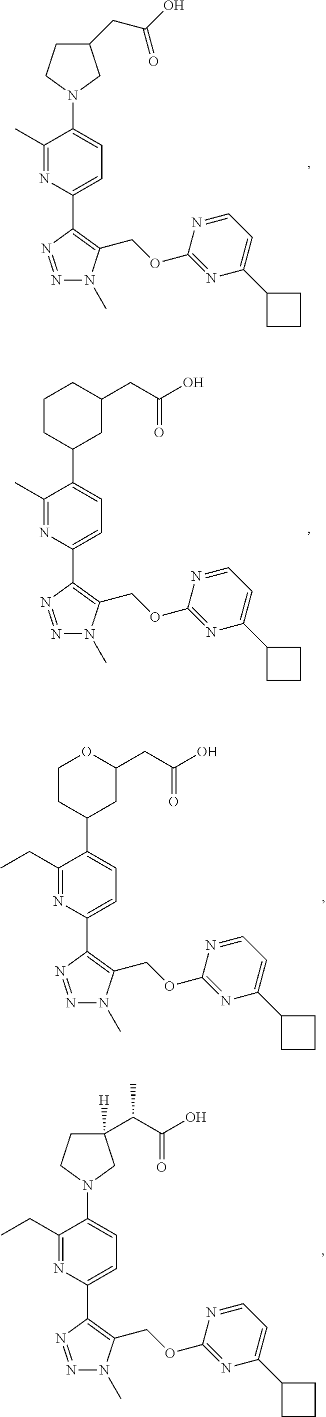 [SMILES: Cc1nc(-c2nnn(C)c2COc2nccc(C3CCC3)n2)ccc1N1CCC(CC(=O)O)C1 ; Cc1nc(-c2nnn(C)c2COc2nccc(C3CCC3)n2)ccc1C1CCCC(CC(=O)O)C1 ; CCc1nc(-c2nnn(C)c2COc2nccc(C3CCC3)n2)ccc1C1CCOC(CC(=O)O)C1 ; CCc1nc(-c2nnn(C)c2COc2nccc(C3CCC3)n2)ccc1N1CC[C@@H]([C@H](C)C(=O)O)C1]

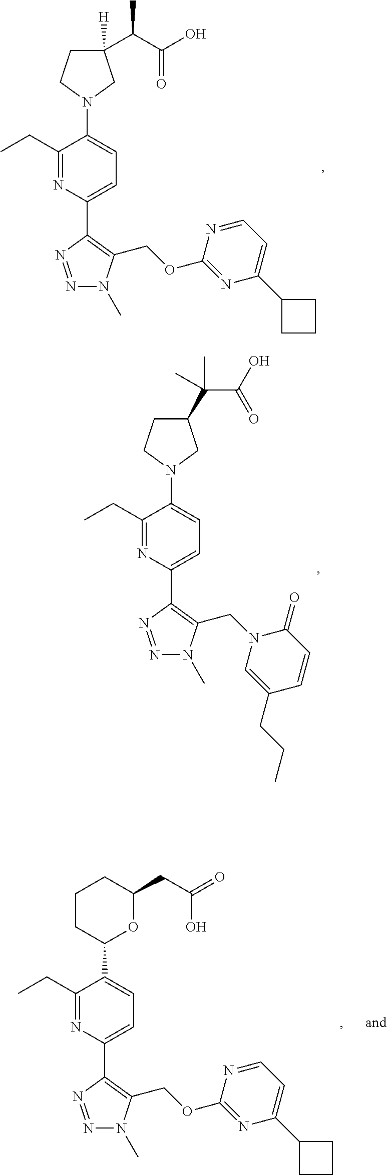

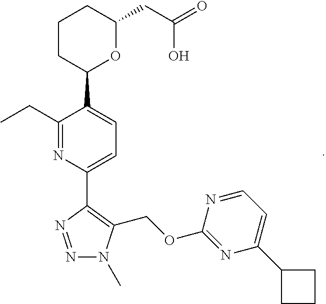 [SMILES: CCc1nc(-c2nnn(C)c2COc2nccc(C3CCC3)n2)ccc1[C@H]1CCC[C@H](CC(=O)O)O1]

69. A compound of Formula (IV), 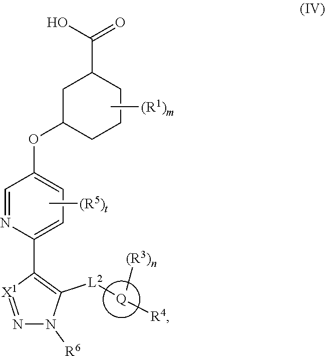
or a pharmaceutically acceptable salt, tautomer and/or stereoisomer thereof,
wherein:
R¹ at each occurrence is independently hydrogen, halogen, C₁₋₆alkyl, haloC₁₋₆alkyl, OH, C₁₋₆alkyl-OH, C₁₋₆alkoxy, haloC₁₋₆alkoxy, CN, C₃₋₇cycloalkyl, NRₐR_{b}, C₁₋₆alkyl-NRₐR_{b}, or 4-6-membered heterocylyl, or R¹ and R¹, together with the carbon atom to which they are attached, form a ketone (C═O);
R⁵ at each occurrence is independently hydrogen, halogen, C₁₋₆alkyl, haloC₁₋₆alkyl, OH, C₁₋₆alkyl-OH, C₁₋₆alkoxy, C₁₋₆alkyl-C₁₋₆alkoxy, haloC₁₋₆alkoxy, CN, C₃₋₇cycloalkyl, NRₐR_{b}, or C₁₋₆alkyl-NRₐR_{b};
X¹ is N, or CR⁶a;
R^{6a} is hydrogen, or methyl;
R⁶ is hydrogen, halogen, CN, methyl, ethyl, propyl, or cyclopropyl;
L² is (CR⁷R⁷)ₚ;
Q ring is 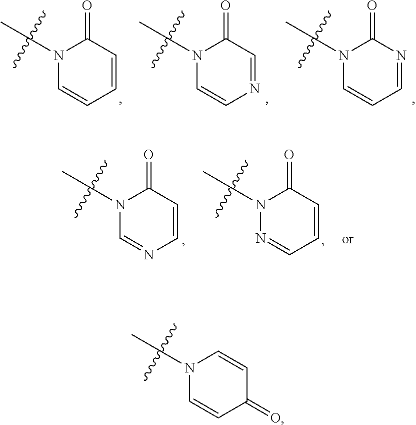 [SMILES: *n1ccccc1=O ; *n1ccncc1=O ; *n1cccnc1=O ; *n1cnccc1=O ; *n1ncccc1=O ; *n1ccc(=O)cc1]
each of which is substituted with (R³)ₙ and one R⁴ at any available carbon position;
R³ at each occurrence is independently hydrogen, halogen, CN, C₁₋₆alkyl, or C₃₋₇cycloalkyl;
R⁴ is hydrogen, halogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, (CH₂)_{q}-5-6-membered heteroaryl ring substituted with 1-4 R¹¹, (CH₂)_{q}-5-7-membered heterocyclyl ring substituted with 1-4 R¹¹, wherein each phenyl is independently optionally substituted with 1-3 halogen, C₁₋₆alkyl, or C₁₋₆alkoxy;
R⁷ at each occurrence is independently hydrogen, C₁₋₄alkyl, C₃₋₅cycloalkyl, or R⁷ and R⁷, together with the carbon atom to which they are attached, form a 3-5-membered cycloalkyl ring;
R¹¹ at each occurrence is independently hydrogen, C₁₋₆alkyl, haloC₁₋₆alkyl, C₂₋₆alkenyl, C₂₋₆alkynyl, C₁₋₆alkoxy, (CH₂)ₚ—C₁₋₆alkoxy, phenyl, (CH₂)ₚ-phenyl, O(CH₂)ₚ-phenyl, CN, C₃₋₇cycloalkyl, (CH₂)ₚ—C₃₋₇cycloalkyl, C₂₋₆alkenyl-C₃₋₇cycloalkyl, C₂₋₆alkynyl, O(CH₂)ₚ—C₃₋₇cycloalkyl, wherein each phenyl is independently optionally substituted with 1-3 halogen, C₁₋₆alkyl, or C₁₋₆alkoxy;
each occurrence of Rₐ and R_{b} is independently hydrogen or C₁₋₆alkyl, or Rₐ and R_{b}, together with the nitrogen atom to which they are attached, form a saturated or unsaturated heterocyclic ring containing from three to seven ring atoms, which ring may optionally contain one or two additional heteroatoms selected from the group consisting of nitrogen, oxygen and sulfur and may be optionally substituted by from one to three groups which may be the same or different selected from the group consisting of C₁₋₄alkyl, phenyl and benzyl;
m is 0, 1, 2 or 3;
n is 0, 1 or 2;
t is 0, 1, 2 or 3;
p at each occurrence is independently 1, 2, 3 or 4;
and q at each occurrence is independently 0, 1, 2, 3 or 4.

70. The compound of claim 69, having the structure of Formula (IVa),

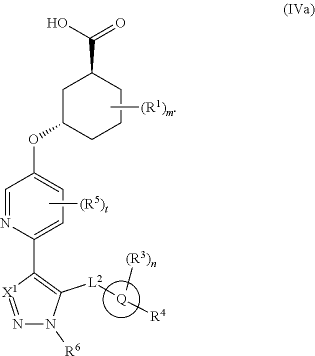

71. The compound of claim 70, selected from:

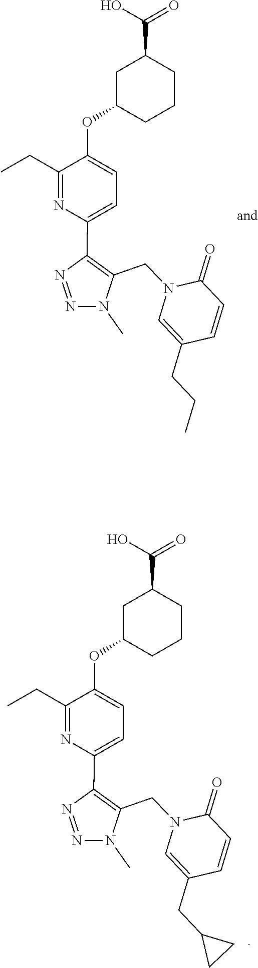 [SMILES: CCCc1ccc(=O)n(Cc2c(-c3ccc(O[C@H]4CCC[C@H](C(=O)O)C4)c(CC)n3)nnn2C)c1 ; CCc1nc(-c2nnn(C)c2Cn2cc(CC3CC3)ccc2=O)ccc1O[C@H]1CCC[C@H](C(=O)O)C1]

72. A pharmaceutical composition comprising the compound of claim 1, and a pharmaceutically acceptable carrier.

73. A method for treating a disease associated with dysregulation of lysophosphatidic acid receptor 1 (LPAi) in a subject in need thereof, comprising administering an effective amount of a compound of claim 1 to the subject.

74. The method of claim 73, wherein the disease is pathological fibrosis (e.g., pulmonary, liver, renal, cardiac, dernal, ocular, or pancreatic fibrosis), idiopathic pulmonary fibrosis (IPF), non-alcoholic steatohepatitis (NASH), non-alcoholic fatty liver disease (NAFLD), chronic kidney disease, diabetic kidney disease, or systemic sclerosis.
